(19) United States
(12) Patent Application Publication
(10) Pub. No.: US 2005/0107293 A1
(43) Pub. Date: May 19, 2005

(54) Protein modification and maintenance molecules

(76) Inventors: William W Sprague, Sacramento, CA (US); Narinder K. Chawla, Union City, CA (US); Bridget A. Warren, San Marcos, CA (US); Y. Tom Tang, San Jose, CA (US); Vicki S. Elliott, San Jose, CA (US); Joseph P. Marquis, San Jose, CA (US); Joana X. Li, Millbrae, CA (US); Jennifer A. Griffin, Fremont, CA (US); Kimberly J Gietzen, San Jose, CA (US); Junming Yang, San Jose, CA (US); Dyung Aina M Lu, San Jose, CA (US); Brooke m Emerling, Chicago, IL (US); Brendan M Duggan, Sunnyvale, CA (US); Thomas W Richardson, Redwood City, CA (US); Soo Yeun Lee, Mountain View, CA (US); Jayalaxmi Ramkumar, Fremont, CA (US); Shanya D Becha, San Francisco, CA (US); Patricia M Mason, Morgan Hill, CA (US); Anita Swarnakar, San Francisco, CA (US); Uyen K Tran, San Jose, CA (US); Amy E Kable, Silver Spring, MD (US); April J.A. Hafalia, Daly City, CA (US); Reena Khare, Saratoga, CA (US)

Correspondence Address: FOLEY AND LARDNER;SUITE 500, 3000 K STREET NW, WASHINGTON, DC, 20007, US

(21) Appl. No.: 10/489,695

(22) PCT Filed: Sep. 13, 2002
(86) PCT No.: PCT/US02/29221

Related U.S. Application Data
(60) Provisional application No. 60/322,196, filed on Sep. 14, 2001.
(60) Provisional application No. 60/324,134, filed on Sep. 21, 2001.
(60) Provisional application No. 60/327,233, filed on Oct. 5, 2001.
(60) Provisional application No. 60/346,198, filed on Oct. 26, 2001.
(60) Provisional application No. 60/343,980, filed on Nov. 2, 2001.
(60) Provisional application No. 60/348,887, filed on Nov. 9, 2001.
(60) Provisional application No. 60/332,423, filed on Nov. 16, 2001.
(60) Provisional application No. 60/334,145, filed on Nov. 28, 2001.
(60) Provisional application No. 60/334,229, filed on Nov. 28, 2001.
(60) Provisional application No. 60/337,451, filed on Dec. 6, 2001.
(60) Provisional application No. 60/351,928, filed on Jan. 25, 2002.
(60) Provisional application No. 60/366,837, filed on Mar. 21, 2002.

(51) Int. Cl. A61K 38/45; C07H 21/04; C12N 9/12

(52) U.S. Cl. 514/12; 435/194; 435/69.1; 435/320.1; 435/325; 536/23.2

(57) ABSTRACT

Various embodiments of the invention provide human protein modification and maintenance molecules (PMMM) and polynucleotides which identify and encode PMMM. Embodiments of the invention also provide expression vectors, host cells, antibodies, agonists, and antagonists. Other embodiments provide methods for diagnosing, treating, or preventing disorders associated with aberrant expression of PMMM.a.

### TECHNICAL FIELD

The invention relates to novel nucleic acids, protein modification and maintenance molecules encoded by these nucleic acids, and to the use of these nucleic acids and proteins in the diagnosis, treatment, and prevention of gastrointestinal, cardiovascular, autoimmune/inflammatory, cell proliferative, developmental, epithelial, neurological, reproductive, endocrine, metabolic, pancreatic disorders, disorders associated with the adrenals, disorders associated with gonadal steroid hormones, cancers, and infections. The invention also relates to the assessment of the effects of exogenous compounds on the expression of nucleic acids and protein modification and maintenance molecules.

### BACKGROUND OF THE INVENTION

The cellular processes regulating modification and maintenance of protein molecules coordinate their function, conformation, stabilization, and degradation. Each of these processes is mediated by key enzymes or proteins such as kinases, phosphatases, proteases, protease iihbitors, isomerases, transferases, and molecular chaperones.

Kinases

Kinases catalyze the transfer of high-energy phosphate groups from adenosine triphosphate (ATP) to target proteins on the hydroxyamino acid residues serine, threonine, or tyrosine. Addition of a phosphate group alters the local charge on the acceptor molecule, causing internal conformational changes and potentially influencing intermolecular contacts. Reversible protein phosphorylation is the ubiquitous strategy used to control many of the intracellular events in eukaryotic cells. It is estimated that more than ten percent of proteins active in a typical mammalian cell are phosphorylated. Extracellular signals including hormones, neurotransmitters, and growth and differentiation factors can activate kinases, which can occur as cell surface receptors or as the activators of the final effector protein, as well as elsewhere along the signal transduction pathway. Kinases are involved in all aspects of a cell's function, from basic metabolic processes, such as glycolysis, to cell-cycle regulation, differentiation, and communication with the extracellular environment through signal transduction cascades. Inappropriate phosphorylation of proteins in cells has been linked to changes in cell cycle progression and cell differentiation. Changes in the cell cycle have been lnked to induction of apoptosis or cancer. Changes in cell differentiation have been lined to diseases and disorders of the reproductive system, immune system, and skeletal muscle.

There are two classes of proteinkinases. One class, protein tyrosine kinases (PTKs), phosphorylates tyrosine residues, and the other class, protein serine/threonine kinases (STKs), phosphorylates serine and threonine residues. Some PTKs and STKs possess structural characteristics of both families and have dual specificity for both tyrosine and serine/threonne residues. Almost all kinases contain a conserved 250-300 amino acid catalytic domain containing specific residues and sequence motifs characteristic of the kinase family. (Reviewed in Hardie, G. and S. Hanks (1995) The Protein Kinase Facts Book, Vol I, Academic Press, San Diego, Calif., pp. 17-20).

Phosphatases

Phosphatases hydrolytically removephosphate groups from proteins. Phosphatases are essential in determining the extent of phosphorylation in the cell and, together with kinases, regulate key cellular processes such as metabolic enzyme activity, proliferation, cell growth and differentiation, cell adhesion, and cell cycle progression. Protein phosphatases are characterized as either serine/threonine- or tyrosine-specific based on their preferred phospho-amino acid substrate. Some phosphatases (DSPs, for dual specificity phosphatases) can act on phosphorylated tyrosine, serine, or threonine residues. The protein serine/threonine phosphatases PSPs) are important regulators of many cAMP-mediated hormone responses in cells. Protein tyrosine phosphatases (PTPs) play a significant role in cell cycle and cell signaling processes.

Proteases

Proteases cleave proteins and peptides at the peptide bond that forms the backbone of the protein or peptide chain. Proteolysis is one of the most important and frequent enzymatic reactions that occurs both within and outside of cells. Proteolysis is responsible for the activation and maturation of nascent polypeptides, the degradation of misfolded and damaged proteins, and the controlled turnover of peptides within the cell Proteases participate in digestion, endocrine function, tissue remodeling during embryonic development, wound healing, and normal growth. Proteases can play a role in regulatory processes by affecting the half life of regulatory proteins. Proteases are involved in the etiology or progression of disease states such as inflammation, angiogenesis, tumor dispersion and metastasis, cardiovascular disease, neurological disease, and bacterial, parasitic, and viral infections.

Proteases canbe categorized on the basis of where they cleave their substrates. Exopeptidases, which include aminopeptidases, dipeptidyl peptidases, tripeptidases, carboxypeptidases, peptidyl-di-peptidases, dipeptidases, and omega peptidases, cleave residues at the termini of their substrates. Endopeptidases, including serine proteases, cysteine proteases, and metalloproteases, cleave at residues within the peptide. Four principal categories of mammalian proteases have been identified based on active site structure, mechanism of action, and overall three-dimensional structure. (See Beynon, R. J. and J. S. Bond (1994) Proteolytic Enzymes: A Practical Approach, Oxford University Press, New York N.Y., pp. 1-5.)

Serine Proteases

The serine proteases (SPs) are a large, widespread family of proteolytic enzymes that include the digestive enzymes trypsin and chymotrypsin, components of the complement and blood-clotting cascades, and enzymes that control the degradation and turnover of macromolecules within the cell and in the extracellular matrix. Most of the more than 20 subfamilies canbe grouped into six clans, each with a common ancestor. These six clans are hypothesized to have descended from at least four evolutionaily distinct ancestors. SPs are named for the presence of a serine residue found in the active catalytic site of most families. The active site is defined by the catalytic triad, a set of conserved asparagine, histidine, and serine residues critical for catalysis. These residues form a charge relay network that facilitates substrate binding. Other residues outside the active site form an oxyanion hole that stabilizes the tetrahedral transition intermediate formed during catalysis. SPs have a wide range of substrates and canbe subdivided into subfamilies on the basis of their substrate specificity. The main subfamilies are named for the residue(s) after which they cleave: trypases (after arginine or lysine), aspases (after aspartate), chymases (after phenylalanine or leucine), metases (methionine), and serases (after serine) (Rawlings, N. D. and A. J. Barrett (1994) Methods Enzymol. 244:19-61).

Most mammalian serine proteases are synthesized as zymogens, inactive precursors that are activated by proteolysis. For example, trypsinogen is converted to its active form, trypsin by enteropeptidase. Enteropeptidase is an intestinal protease that removes an N-terminal fragment from trypsinogen. The remaining active fragment is trypsin which in turn activates the precursors of the other pancreatic enzymes. Likewise, proteolysis of protfrombin, the precursor of thrombin, generates three separate polypeptide fragments. The N-terminal fragment is released while the other two fragments, which comprise active thrombin, remain associated through disulfide bonds.

The two largest SP subfamilies are the chymotrypsin (S1) and subtilisin (S8) families. Some members of the chymotrypsin family contain two structural domains unique to this family. Kringle domains are triple-looped, disulfide cross-linked domains found in varying copy number. Kringle domains are thought to play a role in binding mediators such as membranes, other proteins or phospholipids, and in the regulation of proteolytic activity (PROSlTE PDOC00020). Apple domains are 90 amino-acid repeated domains, each containing six conserved cysteines. Three disulfide bonds link the first and sixth, second and fifth, and third and fourth cysteines (PROSITE PDOC00376). Apple domains are involved in protein-protein interactions. S1 family members include trypsin, chymotrypsin, coagulation factors IX-XII, complement factors B, C, and D, granzymes, kallikrein, and tissue- and urokinase-plasminogen activators. The subtilisin family has members found in the eubacteria, archaebacteria, eukaryotes, and viruses. Subtilisins include the proprotein-processing endopeptidases kexin and fuin and the pituitary prohormone convertases PC1, PC2, PC3, PC6, and PACE4 (Rawlings and Barrett, supra).

SPs have functions in many normal processes and some have been implicated in the etiology or treatment of disease. Enterokinase, the initiator of intestinal digestion, is found in the intestinal brush border, where it cleaves the acidic propeptide from trypsinogen to yield active trypsin (Kitamoto, Y. et al. (1994) Proc. Natl. Acad. Sci USA 91:7588-7592).

Prolylcarboxypeptidase, a lysosomal serine peptidase that cleaves peptides such as angiotensin II and m and [des-Arg9] bradykiin, shares sequence homology withmembers of both the serine carboxypeptidase and prolylendopeptidase families (Tan, F. et al. (1993) J. Biol. Chem. 268:16631-16638). The protease neuropsin may influence synapse formation and neuronal connectivity in the hippocampus in response to neural signing (Chen, Z.-L. et al. (1995) J. Neurosci. 15:5088-5097). Tissue plasminogen activator is useful for acute management of stroke (Zivin, J. A. (1999) Neurology 53:14-19) and myocardial infarction (Ross, A. M. (1999) Clin. Cardiol. 22:165-171). Some receptors (PAR, for proteinase-activated receptor), highly expressed throughout the digestive tract, are activated by proteolytic cleavage of an extracellular domain. The major agonists for PARs, thrombin, trypsin, and mast cell tryptase, are released in allergy and inflammatory conditions. Control of PAR activationbyproteases has been suggested as a promising therapeutic target (Vergnolle, N. (2000) Aliment. Pharmacol Ther. 14:257-266; Rice, K. D. et al. (1998) Curr. Pharm. Des. 4:381-396). Prostate-specific antigen (PSA) is a kallikrein-like serine protease synthesized and secreted exclusively by epithelal cells in the prostate gland. Serum PSA is elevated in prostate cancer and is the most sensitive physiological marker for monitoring cancer progression and response to therapy. PSA can also identify the prostate as the origin of a metastatic tumor (Brawer, M. K. and P. H. Lange (1989) Urology 33:11-16).

The signal peptidase is a specialized class of SP found in all prokaryotic and eukaryotic cell types that serves in the processing of signal peptides from certain proteins. Signal peptides are amiino-termial domains of a protein which direct the protein from its ribosomal assembly site to a particular cellular or extracelllar location. Once the protein has been exported, removal of the signal sequence by a signal peptidase and posttranslational processing, e.g., glycosylation or phosphorylation, activate the protein. Signal peptidases exist as multi-subunit complexes in both yeast and mammals. The canine signal peptidase complex is composed of five subunits, all associated with the microsomal membrane and containing hydrophobic regions that span the membrane one or more times (Shelness, G. S. and G. Blobel (1990) J. Biol. Chem 265:9512-9519). Some of these subunits serve to fix the complex in its proper position on the membrane while others contain the actual catalytic activity.

The mechanism for the translocation process into the ER involves the recognition of an N-terminal signal peptide on the elongating protein. The signal peptide directs the protein and attached ribosome to a receptor on the ER membrane. The polypeptide chain passes through a pore in the ER membrane into the lumen while the N-terminal signal peptide remains attached at the membrane surface. The process is completed when signal peptidase located inside the ER cleaves the signal peptide from the protein and releases the protein into the lumen.

Thrombin is a serine protease with an essential role in the process of blood coagulation. Prothrombin, synthesized in the liver, is converted to active thrombin by Factor Xa. Activated thrombin then cleaves soluble fibrinogen to polymer-forming fibrin, a primary component of blood clots. In addition, thrombin activates Factor XIIIa, which plays a role in cross-linking fibrin.

Thrombin also stimulates platelet aggregation through proteolytic processing of a 41-residue amino-terminal peptide from protease-activated receptor 1 (PAR-1), formerly known as the tbrombin receptor. The cleavage of the amino-terminal peptide exposes a new amino terminus and may also be associated with PAR-1 internalization (Stubbs, M. T. and W. Bode (1994) Curr. Opin. Struct. Biol. 4:823-832; and Ofoso, F. A. et al. (1998) Biochem J. 336:283-285). In addition to stimulating platelet activation through cleavage of the PAR-1 receptor, thrombin also induces platelet aggregation following cleavage of glycoprotein V, also on the surface of platelets. Glycoprotein V appears to be the major thrombin substrate on intact platelets. Platelets deficient for glycoprotein V are hypersensitive to thrombin, which is still required to cleave PAR-1. While platelet aggregation is required for normal hemostasis in mammals, excessive platelet aggregation can result in arterial thrombosis, atherosclerotic arteries, acute myocardial infarction, and stroke (Ramakrishnan, V. et al. (1999) Proc. Natl. Acad. Sci. U.S.A. 96:13336-13341 and references within).

Proteases in another family have a serine in their active site and are dependent on the hydrolysis of ATP for their activity. These proteases contain proteolytic core domains and regulatory ATPase domains which can be identified by the presence of the P-loop, an ATP/GTP-binding motif (PROSRM PDOC00803). Members of this family include the eukaryotic mitochondrial matrix proteases, Clp protease and the proteasome. Clp protease was originally found in plant chloroplasts but is believed to be widespread in both prokaryotic and eukaryotic cells. The gene for early-onset torsion dystonia encodes a protein related to Clp protease (Ozelius, L. J. et al. (1998) Adv. Neurol. 78:93-105).

The proteasome is an intracellular protease complex found in some bacteria and in all eukaryotic cells, and plays an important role in cellular physiology. The proteasome is a large (˜2000 kDa) multisubunit complex composed of a central catalytic core containing a variety of proteases arranged in four seven-membered rings with the active sites facing inwards into the central cavity, and terrinal ATPase subunits covering the outer port of the cavity and regulating substrate entry (for review, see Schmidt, M. et al. (1999) Curr. Opin. Chem. Biol. 3:584-591). Proteasomes are associated with the ubiquitin conjugation system (UCS), a major pathway for the degradation of cellular proteins of all types, including proteins that function to activate or repress cellar processes such as transcription and cell cycle progression (Ciechanover, A. (1994) Cell 79:13-21). In the UCS pathway, proteins targeted for degradation are conjugated to ubiquitin, a small heat stable protein. The ubiquitinated protein is then recognized and degraded by the proteasome. The resultant ubiquitin-peptide complex is hydrolyzed by a ubiquitin carboxyl terminal hydrolase, and free ubiquitin is released for reutilization by the UCS. Ubiquitin-proteasome systems are implicated in the degradation of mitotic cyclic kinases, oncoproteins, tumor suppressor genes (p53), cell surface receptors associated with signal transduction, transcriptional regulators, and mutated or damaged proteins (Ciechanover, supra). This pathway has been implicated in a number of diseases, including cystic fibrosis, Angelman's syndrome, and Liddle syndrome (reviewed in Schwartz, A. L. and A. Ciechanover (1999) Annul. Rev. Med. 50:57-74). A murine proto-oncogene, Unp, encodes a nuclear ubiquitin protease whose overexpression leads to oncogenic transformation of NIH3T3 cells. The humanhomolog of this gene is consistently elevated in small cell tumors and adenocarcinomas of the lung (Gray, D. A. (1995) Oncogene 10:2179-2183). Ubiquitin carboxyl terminal hydrolase is involved in the differentiation of a lymphoblastic leukemia cell line to a non-dividing mature state (Maki, A. et al. (1996) Differentiation 60:59-66). Ineurons, ubiquitin carboxyl terninal hydrolase (PGP 9.5) expression is strong in the abnormal structures that occur in human neurodegenerative diseases (Lowe, J. et al. (1990) J. Pathol. 161:153-160). The proteasome is a large (˜2000 kDa) multisubunit complex composed of a central catalytic core containing a variety of proteases arranged in four seven-membered rings with the active sites facing inwards into the central cavity, and terminal ATPase subunits covering the outer port of the cavity and regulating substrate entry (for review, see Scbmidt, M. et al. (1999) Curr. Op. Chem. Biol. 3:584-591).

Cysteine Proteases

Cysteine proteases (CPs) are involved in diverse cellular processes ranging from the processing of precursor proteins to intracelluar degradation. Nearly half of the CPs known are present only in viruses. CPs have a cysteine as the major catalytic residue at the active site where catalysis proceeds via a thioester intermediate and is facilitated by nearby histidine and asparagine residues. A glutamine residue is also important, as it helps to form an oxyanion hole. Two important CP families include the papain-like enzymes (C1) and the calpains (C2). Papain-like family members are generally lysosomal or secreted and therefore are synthesized with signal peptides as well as propeptides. Most members bear a conserved motif in the propeptide that may have structural significance (Karrer, K. M. et al. (1993) Proc. Natl. Acad. Sci. USA 90:3063-3067). Three-dimensional structures of papain family members show a bilobed molecule with the catalytic site located between the two lobes. Papains include cathepsins B, C, H, L, and S, certain plant allergens and dipeptidyl peptidase (for a review, see Rawlings, N. D. and A. J. Barrett (1994) Methods Enzymol. 244:461-486).

Some CPs are expressed ubiquitously, while others are produced only by cells of the immune system. Of particular note, CPs are produced by monocytes, macrophages and other cells which migrate to sites of inflammation and secrete molecules involved in tissue repair. Overabundance of these repair molecules plays a role in certain disorders. In autoimmune diseases such as rheumatoid arthritis, secretion of the cysteine peptidase cathepsin C degrades collagen, laminin, elastin and other structural proteins found in the extracellular matrix of bones. Bone weakened by such degradation is also more susceptible to tumor invasion and metastasis. Cathepsin L expression may also contribute to the influx of mononuclear cells which exacerbates the destruction of the rheumatoid synovium (Keyszer, G. M. (1995) Arthritis Rheum. 38:976-984).

Calpains are calcium-dependent cytosolic endopeptidases which contain both an N-terminal catalytic domain and a C-terminal calcium-binding domain. Calpain is expressed as a proenzyme heterodimer consisting of a catalytic subunit unique to each isoform and a regulatory subunit common to different isoforms. Each subunit bears a calcium-binding EF-hand domain The regulatory subunit also contains a hydrophobic glycine-rich domain that allows the enzyme to associate with cell membranes. Calpains are activated by increased intracellular calcium concentration, which induces a change in conformation and limited autolysis. The resultant active molecule requires a lower calcium concentration for its activity (Chan, S. L. and M. P. Mattson (1999) J. Neurosci. Res. 58:167-190). Calpain expression is predominitly neuronal, although it is present in other tissues. Several chronic neurodegenerative disorders, including ALS, Parkinson's disease and Alzheimer's disease are associated with increased calpain expression (Chan and Mattson, supra). Calpain-mediated breakdown of the cytoskeleton has been proposed to contribute to brain damage resulting from head injury (McCracken, E. et al (1999) J. Neurotrauma 16:749-761). Calpain-3 is predominantly expressed in skeletal muscle, and is responsible for limb-girdle muscular dystrophy type 2A (Minami, N. et al. (1999) J. Neurol. Sci. 171:31-37).

Another family of thiol proteases is the caspases, which are involved in the initiation and execution phases of apoptosis. A pro-apoptotic signal can activate initiator caspases that trigger a proteolytic caspase cascade, leading to the hydrolysis of target proteins and the classic apoptotic death of the cell. Two active site residues, a cysteine and a histidine, have been implicated in the catalytic mechanism. Caspases are among the most specific endopeptidases, cleaving after aspartate residues. Caspases are synthesized as inactive zymogens consisting of one large (p20) and one small (p10) subunit separated by a small spacer region, and a variable N-terminal prodomain. This prodomain interacts with cofactors that can positively or negatively affect apoptosis. An activating signal causes autoproteolytic cleavage of a specific aspartate residue (D297 in the caspase-1 numbering convention) and removal of the spacer and prodomain, leaving a p10/p20 heterodimer. Two of these heterodimers interact via their small subunits to form the catalytically active tetramer. The long prodomains of some caspase family members have been shown to promote dimerization and auto-processing of procaspases. Some caspases contain a “death effector domain” in their prodomain by which they can be recruited into self-activating complexes with other caspases and FADD protein associated death receptors or the TNF receptor complex. In addition, two dimers from different caspase family members can associate, changing the substrate specificity of the resultant tetramer. Endogenous caspase innbitors (inhibitor of apoptosis proteins, or IAPs) also exist. All these interactions have clear effects onthe control of apoptosis (reviewed in Chan and Mattson, supra; Salveson, G. S. and V. M. Dixit (1999) Proc. Natl. Acad. Sci. USA 96:10964-10967).

Caspases have been implicated in a number of diseases. Mice lacking some caspases have severe nervous system defects due to failed apoptosis in the neuroepithelium and suffer early lethality. Others show severe defects in the inflammatory response, as caspases are responsible for processing IL-1b and possibly other inflammatory cytoldnes (Chan and Mattson, supra). Cowpox virus and baculoviruses target caspases to avoid the death of their host cell and promote successful infection. In addition, increases in inappropriate apoptosis have been reported in AIDS, neurodegenerative diseases and ischemic injury, while a decrease in cell death is associated with cancer (Salveson and Dixit, supra; Thompson, C. B. (1995) Science 267:1456-1462).

Aspartyl Proteases

Aspartyl proteases (APs) include the lysosomal proteases cathepsins D and E, as well as chymosin, reni, and the gastric pepsins. Most retroviruses encode an AP, usually as part of the pol polyprotein. APs, also called acid proteases, are monomeric enzymes consisting of two domains, each domain containing one half of the active site with its own catalytic aspartic acid residue. APs are most active in the range of pH 2-3, at which one of the aspartate residues is ionized and the other neutral. The pepsin famnily of APs contains many secreted enzymes, and all are likely to be synthesized with signal peptides and propeptides. Most family members have three disulfide loops, the first ˜5 residue loop following the first aspartate, the second 5-6 residue loop preceding the second aspartate, and the third and largest loop occurring toward the C terminus. Retropepsins, on the other hand, are analogous to a single domain of pepsin, and become active as homodimers with each retropepsin monomer contributing one half of the active site. Retropepsins are required for processing the viral polyproteins.

APs have roles in various tissues, and some have been associated with disease. Renin mediates the first step in processing the hormone angiotensin, which is responsible for regulating electrolyte balance and blood pressure (reviewed in Crews, D. E. and S. R. Williams (1999) Hum. Biol. 71:475-503). Abnormal regulation and expression of cathepsins are evident in various inflammatory disease states. Expression of cathepsin D is elevated in synovial tissues from patients with rheumatoid arthritis and osteoarthritis. The increased expression and differential regulation of the cathepsins are linked to the metastatic potential of a variety of cancers (Chambers, A. F. et al. (1993) Crit. Rev. Oncol. 4:95-114).

Metalloproteases

Metalloproteases require a metal ion for activity, usually manganese or zinc. Examples of manganese metalloenzymes include aminopeptidase P and human proline dipeptidase (PEPD). Aminopeptidase P can degrade bradykinin, a nonapeptide activated in a variety of inflammatory responses. Aminopeptidase P has been implicated in coronary ischemia/reperfusion injury. Administration of aminopeptidase P imiubitors has been shown to have a cardioprotective effect in rats (Ersalm, C. et al (1999) J. Cardiovasc. Pharmacol. 34:604-611).

Most zinc-dependent metalloproteases share a common sequence in the zinc-binding domain. The active site is made up of two histidines which act as zinc ligands and a catalytic glutamic acid C-terminal to the first histidine. Proteins containing this signature sequence are known as the metzincins and include aminopeptidase N, angiotensin-converting enzyme, neurolysin, the matrix metalloproteases and the adamalysins (ADAMS). An alternate sequence is found in the zinc carboxypeptidases, in which all three conserved residues—two histidines and a glutamic acid—are involved in zinc binding.

A number of the neutral metalloendopeptidases, including angiotensin converting enzyme and the aminopeptidases, are involved in the metabolism of peptide hormones. High aminopeptidase B activity, for examaple, is found in the adrenal glands and neurohypophyses of hypertensive rats (Prieto, I. et al. (1998) Horm. Metab. Res. 30:246-248). Oligopeptidase M/neurolysin can hydrolyze bradykinin as well as neurotensin (Serizawa, A. et al. (1995) J. Biol. Chem 270:2092-2098). Neurotensin is a vasoactive peptide that can act as a neurotransmitter in the brain, where it has been implicated in limiting food intake (Tritos, N. A. et al. (1999) Neuropeptides 33:339-349).

The matrix metalloproteases (MMPs) are a family of at least 23 enzymes that can degrade components of the extracellular matrix (ECM). They are Zn²⁺endopeptidases with an N-terminal catalytic domain. Nearly all members of the family have a hinge peptide and a C-terminal domain which can bind to substrate molecules in the ECM or to iibibitors produced by the tissue (TIMPs, for tissue inhibitor of metalloprotease; Campbell, I. L. and A. Pagenstecher (1999) Trends Neurosci. 22:285-287). The presence of fibronectin-like repeats, transmembrane domains, or C-terminal hemopexinase-like domains can be used to separate MMPs into collagenase, gelatinase, stromelysin and membrane-type MMP subfamilies. In the inactive form, the Zn²⁺ ion in the active site interacts with a cysteine in the pro-sequence. Activating factors disrupt the Zn²⁺-cysteine interaction, or “cysteine switch,” exposing the active site. This partially activates the enzyme, which then cleaves off its propeptide and becomes filly active. MMPs are often activated by the serine proteases plasmin and furin. MMPs are often regulated by stoichiometric, noncovalent interactions with inihibitors; the balance of protease to inhibitor, then, is very important in tissue homeostasis (reviewed in Yong, V. W. et al. (1998) Trends Neurosci. 21:75-80).

MMPs are implicated in a number of diseases including osteoarthritis (Mitchell, P. et al. (1996) J. Clin. Invest. 97:761-768), atherosclerotic plaque rupture (Sukhova, G. K. et al. (1999) Circulation 99:2503-2509), aortic aneurysm (Schneiderman, J. et al. (1998) Am. J. Path. 152:703-710), non-healing wounds (Saarialho-Kere, U.K. et al. (1994) J. Clin. Invest. 94:79-88), bone resorption (Blavier, L. and J. M. Delaisse (1995) J. Cell Sci. 108:3649-3659), age-related macular degeneration (Steen, B. et al. (1998) Invest. Ophthalmol. Vis. Sci. 39:2194-2200), emphysema (Finlay, G. A. et al. (1997) Thorax 52:502-506), myocardial infarction (Rohde, L. E. et al. (1999) Circulation 99:3063-3070) and dilated cardiomyopathy (Thomas, C. V. et al. (1998) Circulation 97:1708-1715). MMP inhibitors prevent metastasis of mammary carcinoma and experimental tumors in rat, and Lewis lung carcinoma, hemangioma, and human ovarian carcinoma xenografts in mice (Eccles, S. A. et al. (1996) Cancer Res. 56:2815-2822; Anderson et al. (1996) Cancer Res. 56:715-718; Volpert, O. V. et al. (1996) J. Clin. Invest. 98:671-679; Taraboletti, G. et al. (1995) J. Natl. Cancer Inst. 87:293-298; Davies, B. et al. (1993) Cancer Res. 53:2087-2091). MMPs may be active in Alzheimer's disease. A number of MMPs are implicated in multiple sclerosis, and administration of MMP inhibitors can relieve some of its symptoms (reviewed in Yong et al., supra).

The astacin family of metalloendopeptidases have been detected in species ranging from hydra to humans, in mature and in developmental systems, performing functions involved in activation of growth factors, degradation of polypeptides, and processing of extracellular proteins. Astacin family proteases are synthesized with NH2-terminal signal and proenzyme sequences, and many (such as meprins, BMP-1, tolloid) contain multiple domains COOH-terminal to the protease domain. They may be secreted from cells or are plasma membrane-associated enzymes. They have a signature sequence in the protease domain and a unique type of zinc binding, with pentacoordination, as well as a protease domain tertiary structure that contains common attributes with serralysins, matrix metalloendopeptidases, and snake venom proteases. Astacins cleave peptide bonds in polypeptides such as insulin B chain and bradykinin and in proteins such as casein and gelatin; and they have arylamidase activity. Meprins are unique proteases in the astacin family, due to their oligomeric structure; they are dimers of disulfide-linked dimers and are highly glycosylated, type I integral membrane proteins that have many attributes of receptors or integrins with adhesion, epidermal growth factor-like, and transmembrane domains. The alpha and beta subunits are differentially expressed and processed to yield latent and active proteases as well as membrane-associated and secreted forms. Meprins are regulated at the transcriptional and posttranslational levels (Bond, J. S. and Beynon, R. J. (1995) Protein Sci. 4:1247-1261).

Another family of metalloproteases is the ADAMs, for A Disintegrin and Metalloprotease Domain, which they share with their close relatives the adamalysins, snake venom metailoproteases (SVMPs). ADAMs combine features of both cell surface adhesion molecules and proteases, containing a prodomain, a protease domain, a disintegrin domain, a cysteine rich domain, an epidermal growth factor repeat, a transmembrane domain, and a cytoplasmic tail. The first three domains listed above are also found in the SVMPs. The ADAMs possess four potential functions: proteolysis, adhesion, signaling and fusion. The ADAMs share the metzincin zinc binding sequence and are inhibited by some MMP antagonists such as TIMP-1.

ADAMs are implicated in such processes as sperm-egg binding and fusion, myoblast fusion, and protein-ectodomain processing or shedding of cytokines, cytokine receptors, adhesion proteins and other extracellular protein domains (Schlöndorff, J. and C. P. Blobel (1999) J. Cell. Sci. 112:3603-3617). The Kuzbanian protein cleaves a substrate in the NOTCH pathway (possibly NOTCH itself), activating the program for lateral inhibition in Drosophila neural development. Two ADAMs, TACE (ADAM 17) and ADAM 10, are proposed to have analogous roles in the processing of amyloid precursor protein in the brain (Schlöndorff and Blobel, supra). TACE has also been identified as the TNF activating enzyme (Black, R. A. et al. (1997) Nature 385:729-733). TNF is a pleiotropic cytokine that is important inmobilizing host defenses in response to infection or trauma, but can cause severe damage in excess and is often overproduced in autoimmune disease. TACE cleaves membrane-bound pro-TNF to release a soluble form. Other ADAMs maybe involved in a sinilar type of processing of other membrane-bound molecules.

Proteins of the ADAMTS sub-family have all of the features of ADAM family metalloproteases and contain an additional thrombospondin domain (TS). The prototypic ADAMTS was identified in mouse, and found to be expressed in heart and kidney and upregulated by proinflammatory stimuli (Kuno, K. et al. (1997) J. Biol. Chem. 272:556-562). To date eleven members are recognized by the Human Genome Organization (HUGO; http://www.gene.ucl.ac.uk/users/hester/adamts.html#Approved). Members of this family have the ability to degrade aggrecan, a high molecular weight proteoglycan which provides cartilage with important mechanical properties including compressibility, and which is lost during the development of arthritis. Enzymes which degrade aggrecan are thus considered attractive targets to prevent and slow the degradation of articular cartilage (See, e.g., Tortorella, M. D. (1999) Science 284:1664-1666; Abbaszade, I. (1999) J. Biol. Chem. 274:23443-23450). Other members are reported to have antiangiogenic potential (Kuno et al., supra) and/or procollagen processing (Colige, A. et al. (1997) Proc. Natl. Acad. Sci. USA 94:2374-2379).

Protease inhibitors

Protease inhibitors and other regulators of protease activity control the activity and effects of proteases. Protease inhibitors have been shown to control pathogenesis in animal models of proteolytic disorders (Murphy, G. (1991) Agents Actions Suppl. 35:69-76). In patients with HIV disease protease inhibitors have been shown to be effective in preventing disease progression and reducing mortality (Barry, M. et al. (1997) Clin. Pharmacokinet. 32:194-209). Low levels of the cystatins, low molecular weight inhibitors of the cysteine proteases, correlate with malignant progression of tumors (Calkins, C. et al. (1995) Biol. Biochem. Hoppe Seyler 376:71-80). The cystatin superfamily of protease inhibitors is characterized by a particular pattern of linearly arranged and tandemly repeated disulfide loops (Kellermann, J. et al. (1989) J. Biol. Chem. 264:14121-14128). An example of a representative of a structural prototype of a novel family among the cystatin superfamily is human alpha 2-HS glycoprotein (AHSG), a plasma protein synthesized in liver and selectively concentrated in bone matrix, dentine, and other mineralized tissues (Triffitt, J. T. (1976) Calcif. Tissue Res. 22:27-33), which is also classified as belonging to the fetuin family. Fetuins are characterized by the presence of 2 N-terniy located cystatin-like repeats and a unique C-terminal domain which is not present in other proteins of the cystatin superfamily (PROSITE PDOC00966). AHSG has been reported to be involved in bone formation and resorption as well as immune responses (Yang, F. et al. (1992) 1130:149-156; Lee, C. C. et al. (1987) PNAS USA 84:4403-4407; Nakamura, O. et al. (1999) Biosci. Biotechnol. Biochem. 63:1383-1391). Additionally, AHSG has been implicated in infertility associated with endometriosis (Mathur, S. P. (2000) Am. J. Reprod. Immunol. 44:89-95; Mathur, S. P. et al. (1999) Autoimmunity 29:121-127) and inhibition of osteogenesis (Binkert, C. et al, (1999) J. Biol Chem. 274:28514-28520). Decreased serum levels of AHSG have been detected inpatients with acute leukemias, chronic granulocyte and myelomonocyte leukemias, lymphomas, myelofibrosis, multiple myeloma, metastatizing solid tumors, systemic lupus erythematosus, rheumatoid arthritis, acute alcoholic hepatitis, fatty liver, chronic active hepatitis, liver cirrhosis, acute and chronic pancreatitis, and Crohn's disease (Kalabay, L. et al. (1992) Orv. Hetil. 133:1553-1554; 1559-1560).

Serpins are inhibitors of mammalian plasma serine proteases. Many serpins serve to regulate thle blood clotting cascade and/or the complement cascade in mammals. Sp32 is a positive regulator of the mammalian acrosomal protease, acrosin, that binds the proenzyme, proacrosin, and thereby aides in packaging the enzyme into the acrosomal matrix (Baba, T. et al. (1994) J. Biol. Chem. 269:10133-10140). The Kunitz family of serine protease iniiitors are characterized by one or more “Kunitz domains” containing a series of cysteine residues that are regularly spaced over approximately 50 amino acid residues and form three intrachain disulfide bonds. Members of this family include aprotinin, tissue factor pathway inhibitor (TFPI-1 and TFPI-2), inter-a-trypsin inhibitor, and bikunin (Marlor, C. W. et al. (1997) J. Biol. Chem. 272:12202-12208). Members of this family are potent inhibitors (in the nanomolar range) against serine proteases such as kalikrein and plasmin. Aprotinin has clinical utility in reduction of perioperative blood loss. ITI has been found to inactivate human trypsin, chymotrypsin, neutrophil elastase and cathepsin G (Morii, M. et al. (1985) Biol. Chem. Hoppe Seyler 366:19-21); and is suspected of playing a key role in the biology of the extracellular matrix and in the pathophysiology of chronic bronchopulmonary diseases or lung cancer progression (Cuvelier, A. et al. (2000) Rev. Mal. Respir. 17:437-446).

A major portion of all proteins synthesized in eukaryotic cells are synthesized on the cytosolic surface of the endoplasmic reticulum (ER). Before these immature proteins are distributed to other organelles in the cell or are secreted, they must be transported into the interior lumen of the ER where post-translational modifications are performed. These modifications include protein folding and the formation of disulfide bonds, and N-hinked glycosylations.

Protein Isomerases

Protein folding in the ER is aided by two principal types of protein isomerases, protein disulfide isomerase (PDI), and peptidyl-prolyl isomerase (PPI). PDI catalyzes the oxidation of free sulfhydryl groups in cysteine residues to form intramolecular disulfide bonds in proteins. PPI, an enzyme that catalyzes the isomerization of certain proline imidic bonds in oligopeptides and proteins, is considered to govern one of the rate limiting steps in the folding of many proteins to their final functional conformation. The cyclophilins represent a major class of PPI that was originally identified as the major receptor for the immunosuppressive drug cyclosporin A (Handschumacher, R. E. et al. (1984) Science 226: 544-547).

Protein Glycosylation

The glycosylation of most soluble secreted and membrane-bound proteins by oligosaccharides lied to asparagine residues in proteins is also performed in the ER. This reaction is catalyzed by a membrane-bound enzyme, oligosaccharyl transferase. Although the exact purpose of this “N-linked” glycosylation is unknown, the presence of oligosaccharides tends to make a glycoprotein resistant to protease digestion. In addition, oligosaccharides attached to cell-surface proteins called selectins are known to fumction in cell-cell adhesion processes (Alberts, B. et al. (1994) Molecular Biology of the Cell Garland Publishing Co., New York, N.Y., p. 608). “O-linked” glycosylation of proteins also occurs in the ER by the addition of N-acetylgalactosamine to the hydroxyl group of a serine or threonine residue followed by the sequential addition of other sugar residues to the first. This process is catalyzed by a series of glycosyltransferases, each specific for a particular donor sugar nucleotide and acceptor molecule (Lodish, H. et al. (1995) Molecular Cell Biology, W. H. Freeman and Co., New York, N.Y., pp. 700-708). For example, one of the glycosyltransferases in the dolichol pathway, dolichol phosphate mannose synthase, is required in N:-glycosylation, O-mannosylation, and glycosylphosphatidylinositol membrane anchoring of protein (Tomita, S. et al. (1998) J. Biol. Chem. 9249-9254). Thus, in many cases, both N— and O-linked oligosaccharides appear to be required for the secretion of proteins or the movement of plasma membrane glycoproteins to the cell surface.

An additional glycosylation mechanism operates in the ER specifically to target lysosomal enzymes to lysosomes and prevent their secretion. Lysosomal enzymes in the ER receive an N-linked oligosaccharide, like plasma membrane and secreted proteins, but are then phosphorylated on one or two mannose residues. The phosphorylation of mannose residues occurs in two steps, the first step being the addition of an N-acetylglucosamine phosphate residue by N-acetylglucosamine phosphotransferase, and the second the removal of the N-acetylglucosamine group by phosphodiesterase. The phosphorylated mannose residue then targets the lysosomal enzyme to a mannose 6-phosphate receptor which transports it to a lysosome vesicle (Lodish et al. supra, pp. 708-711).

Chaperones

Molecular chaperones are proteins that aid in the proper folding of immature proteins and refolding of improperly folded ones, the assembly of protein subunits, and in the transport of unfolded proteins across membranes. Chaperones are also called heat-shock proteins (hsp) because of their tendency to be expressed in dramatically increased amounts following brief exposure of cells to elevated temperatures. This latter property most likely reflects their need in the refolding of proteins that have become denatured by the high temperatures. Chaperones may be divided into several classes according to their location, function, and molecular weight, and include hsp60, TCP1, hsp70, hsp40 (also called DnaJ), and hsp90. For example, hsp90 binds to steroid hormone receptors, represses transcription in the absence of the ligand, and provides proper folding of the ligand-binding domain of the receptor in the presence of the hormone (Burston, S. G. and A. R. Clarke (1995) Essays Biochem. 29:125-136). Hsp60 and hsp70 chaperones aid in the transport and folding of newly synthesized proteins. Hsp70 acts early in protein folding, binding a newly synthesized protein before it leaves the ribosome and transporting the protein to the mitochondria or ER before releasing the folded protein. Hsp60, along with hsp10, binds misfolded proteins and gives them the opportunity to refold correctly. All chaperones share an affinity for hydrophobic patches on incompletely folded proteins and the ability to hydrolyze ATP. The energy of ATP hydrolysis is used to release the hsp-bound protein in its properly folded state (Alberts et al., supra, pp. 214, 571-572). Hsp40/DnaJ homologs include mDj3, mDj4, mDj5, mDj6, mDj7, mDj8, mDj9, mDj10, and mDj11 (Ohtsuka, K. and Hata, M. (2000) Cell Stress Chaperones 5:98-112).

Lysyl Hydroxylases

Lysyl hydroxylase is an enzyme involved in collagen biosynthesis. Collagens are a family of fibrous structural proteins that are found in essentially all tissues. Collagens are the most abundant proteins in mammals, and are essential for the formation of connective tissue such as skin, bone, tendon, cartilage, blood vessels and teeth. Members of the collagen family can be distinguished from one another by the degree of cross-linking between collagen fibers and by the number of carbohydrate units (e.g., galactose or glucosylgalactose) attached to the collagen fibers. Hydroxylated lysine residues (hydroxylysine) are essential for stability of cross-linking and as attachment points for carbohydrate units.

The enzyme lysyl hydroxylase catalyzes the hydroxylation of lysine residues to form hydroxylysine. Lysyl hydroxylase targets the lysine residue of the sequence, X-lys-gly (lys=lysine, gly=glycine, and X=any amino acid residue). Three isoforms of lysyl hydroxylase have been characterized, termed LH1 (or PLOD; procollagen-lysine, 2-oxoglutarate 5-dioxygenase), LH2 (or PLOD2), and LH3. The three enzymes share 60% sequence identity overall, with even higher similarity in the C-terminal region. In addition, there are regions in the middle of the molecule that have an identity of more than 80% (Valtavaara, M. et al. (1998) J. Biol. Chem. 273:12881-12886).

Diminished lysyl hydroxylase activity is involved in certain connective tissue disorders. In particular mutations, including a truncation and duplications within the coding region of the gene for PLOD, have been described in patients with type VI Ehlers-Danos syndrome (Hyland, J. et al. (1992) Nature Genet. 2:228-31; Hautala, T. et al. (1993) Genomics 15:399-404).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular

Alzheimer's Disease

The potential application of gene expression profiling is also relevant to improving diagnosis, prognosis, and treatment of diseases such as Alzheimer's disease. For exanple, both the levels and sequences expressed in tissues from subjects with Alzheimer's disease may be compared with the levels and sequences expressed in normal brain tissue. Alzheimer's disease is a progressive neurodegenerative disorder that is characterized by the formation of senile plaques and neurofibrillary tangles containing amyloid beta peptide. These plaques are found in limbic and association cortices of the brain. The hippocampus is part of the limbic system and plays an important role in learning and memory. In subjects with Alzheimer's disease, accumulating plaques damage the neuronal architecture in limbic areas and eventually cripple the memory process.

Steroid Hormones

The potential application of gene expression profiling is relevant to measuring the toxic response to potential therapeutic compounds and of the metabolic response to therapeutic agents. For instance, diseases treated with steroids and disorders caused by the metabolic response to treatment with steroids include adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease. It is desirable to measure the toxic response to potential therapeutic compounds and of the metabolic response to therapeutic agents.

Steroids are a class of lipid-soluble molecules, including cholesterol, bile acids, vitamin D, and hormones, that share a common four-ring structure based on cyclopentanoperhydrophenanthrene and that carrry out a wide variety of functions. Steroid hormones, produced by the adrenal cortex, ovaries, and testes, include glucocorticoids, mineralocorticoids, androgens, and estrogens. Steroid hormones are widely used for fertility control and in anti-inflammatory treatments for physical injuries and diseases such as arthritis, asthma, and auto-immune disorders. Progesterone, a naturally occurring progestin, is primarily used to treat amenorrhea, abnormal uterine bleeding, or as a contraceptive. Medroxyprogesterone (MAH), also known as 6α-methyl-17-hydroxyprogesterone, is a synthetic progestin with a pharmacological activity about 15 times greater than progesterone. MAH is used for the treatment of renal and endometrial carcinomas, amenorrhea, abnormal uterine bleeding, and endometriosis associated with hormonal imbalance. MAH has a stimulatory effect on respiratory centers and has been used in cases of low blood oxygenation caused by sleep apnea, chronic obstructive pulmonary disease, or hypercapnia. Beclomethasone is a synthetic glucocorticoid that is used to treat steroid-dependent asthma, to relieve symptoms associated with allergic or nonallergic (vasomotor) rhinitis, or to prevent recurrent nasal polyps following surgical removal. Budesonide is a corticosteroid used to control symptoms associated with allergic rhinitis or asthma. Dexamethasone is a synthetic glucocorticoid used in anti-inflammatory or immunosuppressive compositions. Prednisone is metabolized in the liver to its active form, prednisolone, a glucocorticoid with anti-inflammatory properties. Betamethasone is a synthetic glucocorticoid with anti-inflammatory and immunosuppressive activity and is used to treat psoriasis and fungal infections, such as athlete's foot and ringworm. By comparing both the levels and sequences expressed in tissues from subjects exposed to or treated with steroid compounds with the levels and sequences expressed in normal untreated tissue it is possible to determine tissue responses to steroids.

Breast Cancer

Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

The potential application of gene expression profiling is particularly relevant to improving diagnosis, prognosis, and treatment of cancers, such as breast cancer, colon cancer, lung cancer, ovarian cancer and prostate cancer. Breast cancer is the most frequently diagnosed type of cancer in American women and the second most frequent cause of cancer death. The lifetime risk of an American woman developing breast cancer is 1 in 8, and one-third of women diagnosed with breast cancer die of the disease. A number of risk factors have been identified, including hormonal and genetic factors. One genetic defect associated with breast cancer results in a loss of heterozygosity (LOH) at multiple loci such as p53, Rb, BRCA1, and BRCA2. Another genetic defect is gene amplification involving genes such as c-myc and c-erbB2 (Her2-neu gene). Steroid and growth factor pathways are also altered in breast cancer, notably the estrogen, progesterone, and epidermal growth factor (EGF) pathways. Breast cancer evolves through a multi-step process whereby premalignant mammary epithelial cells undergo a relatively defined sequence of events leading to tumor formation. An early event in tumor development is ductal hyperplasia. Cells undergoing rapid neoplastic growth gradually progress to invasive carcinoma and become metastatic to the lung, bone, and potentially other organs. Variables that may influence the process of tumor progression and malignant transformation include genetic factors, environmental factors, growth factors, and hormones.

Colon Cancer

Colon cancer evolves through a multi-step process whereby pre-malignant colonocytes undergo a relatively defined sequence of events leading to tumor formation. While soft tissue sarcomas are relatively rare, more than 50% of new patients diagnosed with the disease will die from it. The molecular pathways leading to the development of sarcomas are relatively unknown, due to the rarity of the disease and variation in pathology. Several factors participate in the process of tumor progression and malignant transformation including genetic factors, mutations, and selection.

To understand the nature of gene alterations in colorectal cancer, a number of studies have focused on the inherited syndromes. Familial adenomatous polyposis (FAP), is caused by mutations in the adenomatous polyposis coli gene (APC), resulting in truncated or inactive forms of the protein. This tumor suppressor gene has been mapped to chromosome 5q. Hereditary nonpolyposis colorectal cancer (HNPCC) is caused by mutations in mis-match repair genes. Although hereditary colon cancer syndromes occur in a small percentage of the population and most colorectal cancers are considered sporadic, knowledge from studies of the hereditary syndromes can be generally applied. For instance, somatic mutations in APC occur in at least 80% of sporadic colon tumors. APC mutations are thought to be the initiating event in the disease. Other nutations occur subsequently. Approximately 50% of colorectal cancers contain activating mutations in ras, while 85% contain inactivating mutations in p53. Changes in all of these genes lead to gene expression changes in colon cancer.

Lung Cancer

Lung cancer is the leading cause of cancer death in the United States, affecting more than 100,000 men and 50,000 women each year. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of all lung cancer cases. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also commnon. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda et al. (1999; Genomics 51:5-14) identified five genes differentially expressed in lung cancer cell lines compared to normal bronchial epithelial cells. Among the known genes, pulmonary surfactant apoprotein A and alpha 2 macroglobulin were down regulated whereas nm23H1 was upregulated. Petersen et al. (2000; Int J. Cancer, 86:512-517) used suppression subtractive hybridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang et al. (2000; Oncogene 19:1519-1528) used a combination of microarray analysis and subtractive hybridization to identify 17 genes differentially overexpresssed in squamous cell carcinoma compared with normal lung epithelium. Among the known genes they identified were keratin isoform 6, KOC, SPRC, IGFb2, connexin 26, plakofillin 1 and cytokeratin 13.

Ovarian Cancer

Ovarian cancer is the leading cause of death from a gynecologic cancer. The majority of ovarian cancers are derived from epithelial cells, and 70% of patients with epithelial ovarian cancers present with late-stage disease. As a result, the long-term survival rates for this disease is very low. Identification of early-stage markers for ovarian cancer would significantly increase the survival rate. Genetic variations involved in ovarian cancer development include mutation of p53 and microsatellite instability. Gene expression patterns likely vary when normal ovary is compared to ovarian tumors.

Prostate Cancer

Prostate cancer is a common malignancy in men over the age of 50, and the incidence increases with age. In the US, there are approximately 132,000 newly diagnosed cases of prostate cancer and more than 33,000 deaths from the disorder each year. Once cancer cells arise in the prostate, they are stimulated by testosterone to a more rapid growth. Thus, removal of the testes can indirectly reduce both rapid growth and metastasis of the cancer. Over 95 percent of prostatic cancers are adenocarcinomas which originate in the prostatic acini. The remaining 5 percent are divided between squamous cell and transitional cell carcinomas, both of which arise in the prostatic ducts or other parts of the prostate gland.

As with most tumors, prostate cancer develops through a multistage progression ultimately resulting in an aggressive tumor phenotype. The initial step in tumor progression involves the hyperproliferation of normal luminal and/or basal epithelial cells. Androgen responsive cells become hyperplastic and evolve into early-stage tumors. Although early-stage tumors are often androgen sensitive and respond to androgen ablation, a population of androgen independent cells evolve from the hyperplastic population. These cells represent a more advanced form of prostate tumor that may become invasive and potentially become metastatic to the bone, brain, or lung. A variety of genes may be differentially expressed during tumor progression. For example, loss of heterozygosity (LOH) is frequently observed on chromosome 8p in prostate cancer. Fluorescence in situ hybridization (FISH) revealed a deletion for at least 1 locus on 8p in 29 (69%) tumors, with a significantly higher frequency of the deletion on 8p21.2-p21.1 in advanced prostate cancer than inlocalized prostate cancer, implying that deletions on 8p22-p21.3 play an important role in tumor differentiation, while 8p21.2-p21.1 deletion plays a role in progression of prostate cancer (Oba, K. et al. (2001) Cancer Genet. Cytogenet. 124:20-26).

A primary diagnostic marker for prostate cancer is prostate specific antigen (PSA). PSA is a tissue-specific serine protease almost exclusively produced by prostatic epithelial cells. The quantity of PSA correlates with the number and volume of the prostatic epithelial cells, and consequently, the levels of PSA are an excellent indicator of abnormal prostate growth. Men with prostate cancer exhibit an early linear increase in PSA levels followed by an exponential increase prior to diagnosis. However, since PSA levels are also influenced by factors such as inflammation, androgen and other growth factors, some scientists maintain that changes in PSA levels are not useful in detecting individual cases of prostate cancer.

Leukocytes

Leukocytes comprise lymphocytes, granulocytes, and monocytes. Lymphocytes include T- and B-cells, which specifically recognize and respond to foreign pathogens. T-cells fight viral infections and activate other leukocytes, while B-cells secrete antibodies that neutralize bacteria and other microbes. Granulocytes and monocytes are primarily migratory, phagocytic cells that exit the bloodstream to fight infection in tissues. Monocytes, which are derived from imnature promonocytes, further differentiate into macrophages that engulf and digest microorganisms and damaged or dead cells. Monocytes and macrophages modulate the immune response by secreting signaling molecules such as growth factors and cytokines. Tumor necrosis factor-α (TNF-α), for example, is a macrophage-secreted protein with anti-tumor and anti-viral activity. In addition, monocytes and macrophages are recruited to sites of infection and inflammation by signaling proteins secreted by other leukocytes. The differentiation of the monocyte blood cell lineage can be studied in vitro using cultured cell lines. For example, THP-1 is a human promonocyte cell line that can be activated by treatment with both phorbol ester such as phorbol nyristate acetate (PMA), and lipopolysaccharide (LPS). PMA is a broad activator of the protein kinase C-dependent pathways.

Monocytes are involved in the initiation and maintenance of inflammatory immune responses. The outer membrane of gram-negative bacteria expresses lipopolysaccharide (LPS) complexes called endotoxins. Toxicity is associated with the lipid component (Lipid A) of LPS, and immunogenicity is associated with the polysaccharide components of LPS. LPS elicits a variety of inflammatory responses, and because it activates complement by the alternative (properdin) pathway, it is often part of the pathology of gram-negative bacterial infections. For the most part, endotoxins remain associated with the cell wall until the bacteria disintegrate. LPS released into the bloodstream by lysing gram-negative bacteria is first bound by certain plasma proteins identified as LPS-binding proteins. The LPS-binding protein complex interacts with CD14 receptors on monocytes, macrophages, B cells, and other types of receptors on endothelial cells. Activation of human B cells with LPS results in mitogenesis as well as immunoglobulin synthesis. In monocytes and macrophages three types of events are triggered during their interaction with LPS: 1) Production of cytokines, including IL-1, IL-6, IL-8, TNF-α, and platelet-activating factor, which stimlate production of prostaglandins and leukotrienes that mediate inflammation and septic shock; 2) Activation of the complement cascade; and 3) Activation of the coagulation cascade.

There is a need in the art for new compositions, including nucleic acids and proteins, for the diagnosis, prevention, and treatment of gastrointestinal, cardiovascular, autoimmune/inflammatory, cell proliferative, developmental, epithelial, neurological, reproductive, endocrine, metabolic, pancreatic disorders, disorders associated with the adrenals, disorders associated with gonadal steroid hormones, cancers, and infections.

### SUMMARY OF THE INVENTION

Various enmbodiments of the invention provide purified polypeptides, protein modification and maintenance molecules, referred to collectively as ‘PMMM’ and individually as ‘PMMM-1,’ ‘PMMM-2,’ ‘PMMM-3,’ ‘PMMM-4,’ ‘PMMM-5,’ ‘PMMM-6,’ ‘PMMM-7,’ ‘PMMM-8,’ ‘PMMM-9,’ ‘PMMM-10,’ ‘PMMM-11,’ ‘PMMM-12,’ ‘PMMM-13,’ ‘PMMM-14,’ ‘PMMM-15,’ ‘PMMM-16,’ ‘PMMM-17,’ ‘PMMM-18,’ ‘PMMM-19,’ ‘PMMM-20,’ ‘PMMM-21,’ ‘PMMM-22,’ ‘PMMM-23,’ ‘PMMM-24,’ ‘PMMM-25,’ ‘PMMM-26,’ ‘PMMM-27,’ ‘PMMM-28,’ ‘PMMM-29,’ ‘PMMM-30,’ and ‘PMMM-31’ and methods for using these proteins and their encoding polynucleotides for the detection, diagnosis, and treatment of diseases and medical conditions. Embodiments also provide methods for utilizing the purified protein modification and maintenance molecules and/or their encoding polynucleotides for facilitating the drug discovery process, including determination of efficacy, dosage, toxicity, and pharmacology. Related embodiments provide methods for utilizing the purified protein modification and maintenance molecules and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-31, b) a polypeptide comprising a naturally occurring amino acid sequence at least 90% identical or at least about 90% identical to an amino acid sequence selected from the group consisting of SEQ ID NO: 1-31, c) a biologically active fragment of a polypeptide having an amino acid sequence selected from the group consisting of SEQ ID NO:1-31, and d) an immunogenic fragment of a polypeptide having an amino acid sequence selected from the group consisting of SEQ ID NO:1-31. Another embodiment provides an isolated polypeptide comprising an amino acid sequence of SEQ ID NO:1-31.

Still another embodiment provides an isolated polynucleotide encoding a polypeptide selected from the group consisting of a) a polypeptide comprising an amino acid sequence selected from the group consisting of SEQ ID NO:1-31, b) a polypeptide comprising a naturally occurring amino acid sequence at least 90% identical or at least about 90% identical to an amino acid sequence selected from the group consisting of SEQ ID NO:1-31, c) a biologically active fragment of a polypeptide having an amino acid sequence selected from the group consisting of SEQ ID NO:1-31, and d) an immuonogenic fragment of a polypeptide having an amino acid sequence selected from the group consisting of SEQ ID NO:1-31. In another embodiment, the polynucleotide encodes a polypeptide selected fromthe group consisting of SEQ ID NO:1-31. In an alternative embodiment, the polynucleotide is selected from the group consisting of SEQ ID NO:32-62.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-31, b) a polypeptide comprising a naturally occurring amino acid sequence at least 90% identical or at least about 90% identical to an amino acid sequence selected from the group consisting of SEQ ID NO:1-31, c) a biologically active fragment of a polypeptide having an amino acid sequence selected from the group consisting of SEQ ID NO:1-31, and d) an immunogenic fragment of a polypeptide having an amino acid sequence selected from the group consisting of SEQ ID NO:1-31. Another embodiment provides a cell transformed with the recombinant polynucleotide. Yet another enbodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-31, b) a polypeptide comprising a naturally occurring amino acid sequence at least 90% identical or at least about 90% identical to an amino acid sequence selected from the group consisting of SEQ ID NO:1-31, c) a biologically active fragment of a polypeptide having an amino acid sequence selected from the group consisting of SEQ ID NO:1-31, and d) an immunogenic fragment of a polypeptide having an amino acid sequence selected from the group consisting of SEQ ID NO:1-31. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-31, b) a polypeptide comprising a naturally occurring amino acid sequence at least 90% identical or at least about 90% identical to an amino acid sequence selected from the group consisting of SEQ ID NO:1-31, c) a biologically active fragment of a polypeptide having an amino acid sequence selected from the group consisting of SEQ ID NO:1-31, and d) an immunogenic fragment of a polypeptide having an amino acid sequence selected from the group consisting of SEQ ID NO:1-31.

Still yet another enmbodiment provides an isolated polynucleotide selected from the group consisting of a) a polynucleotide comprising a polynucleotide sequence selected from the group consisting of SEQ ID NO:32-62, b) a polynucleotide comprising a naturally occurring polynucleotide sequence at least 90% identical or at least about 90% identical to a polynucleotide sequence selected from the group consisting of SEQ ID NO:32-62, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:32-62, b) a polynucleotide comprising a naturally occurring polynucleotide sequence at least 90% identical or at least about 90% identical to a polynucleotide sequence selected from the group consisting of SEQ ID NO:32-62, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other enbodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:32-62, b) a polynucleotide comprising a naturally occurring polynucleotide sequence at least 90% identical or at least about 90% identical to a polynucleotide sequence selected from the group consisting of SEQ ID NO:32-62, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-31, b) a polypeptide comprising a naturally occurring amino acid sequence at least 90% identical or at least about 90% identical to an amino acid sequence selected from the group consisting of SEQ ID NO: 1-31, c) a biologically active fragment of a polypeptide having an amino acid sequence selected from the group consisting of SEQ ID NO:1-31, and d) an irmunogenic fragment of a polypeptide having an amino acid sequence selected fromthe group consisting of SEQ ID NO:1-31, and a pharmaceutically acceptable excipient In one embodiment, the composition can comprise an amino acid sequence selected from the group consisting of SEQ ID NO:1-31. Other embodiments provide a method of treating a disease or condition associated with decreased or abnormal expression of functional PMMM, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-31, b) a polypeptide comprising a naturally occurring amino acid sequence at least 90% identical or at least about 90% identical to an amino acid sequence selected fromthe group consisting of SEQ ID NO:1-31, c) a biologically active fragment of a polypeptide having an amino acid sequence selected from the group consisting of SEQ ID NO:1-31, and d) an immunogenic fragment of a polypeptide having an amino acid sequence selected from the group consisting of SEQ ID NO:1-31. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional PMMM, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-31, b) a polypeptide comprising a naturally occurring amino acid sequence at least 90% identical or at least about 90% identical to an amino acid sequence selected from the group consisting of SEQ ID NO:1-31, c) a biologically active fragment of a polypeptide having an amino acid sequence selected from the group consisting of SEQ ID NO:1-31, and d) an immunogenic fragment of a polypeptide having an amino acid sequence selected from the group consisting of SEQ ID NO:1-31. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional PMMM, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-31, b) a polypeptide comprising a naturally occurring amino acid sequence at least 90% identical or at least about 90% identical to an amino acid sequence selected from the group consisting of SEQ ID NO:1-31, c) a biologically active fragment of a polypeptide having an amino acid sequence selected from the group consisting of SEQ ID NO:1-31, and d) an immunogenic fragment of a polypeptide having an amino acid sequence selected from the group consisting of SEQ ID NO:1-31. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-31, b) a polypeptide comprising a naturally occurring amino acid sequence at least 90% identical or at least about 90% identical to an amino acid sequence selected from the group consisting of SEQ ID NO:1-31, c) a biologically active fragment of a polypeptide having an amino acid sequence selected from the group consisting of SEQ ID NO:1-31, and d) an immunogenic fragment of a polypeptide having an amino acid sequence selected from the group consisting of SEQ ID NO:1-31. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:32-62, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:32-62, ii) a polynucleotide comprising a naturally occurring polynucleotide sequence at least 90% identical or at least about 90% identical to a polynucleotide sequence selected from the group consisting of SEQ ID NO:32-62, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:32-62, ii) a polynucleotide comprising a naturally occurring polynucleotide sequence at least 90% identical or at least about 90% identical to a polynucleotide sequence selected from the group consisting of SEQ ID NO:32-62, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“PMMM” refers to the amino acid sequences of substantially purified PMMM obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of PMMM. Agonists may include proteins, nucleic acids, carbohydrates, small molecules, or any other compound or composition which modulates the activity of PMMM either by directly interacting with PMMM or by acting on components of the biological pathway in which PMMM participates.

An “allelic variant” is an alternative form of the gene encoding PMMM. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding PMMM include those sequences with deletions, insertions, or substitutions of different nucleotides, resulting in a polypeptide the same as PMMM or a polypeptide with at least one functional characteristic of PMMM. Included within this definition are polymorphisms which may or may not be readily detectable using a particular oligonucleotide probe of the polynucleotide encoding PMMM, and improper or unexpected hybridization to allelic variants, with a locus other than the normal chromosomal locus for the polynucleotide encoding PMMM. The encoded protein may also be “altered,” and may contain deletions, insertions, or substitutions of amino acid residues which produce a silent change and result in a functionally equivalent PMMM. Deliberate amino acid substitutions may be made on the basis of one or more similarities in polarity, charge, solubility, hydrophobicity, hydrophilicity, and/or the amphipathic nature of the residues, as long as the biological or immunological activity of PMMM is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Anplification may be carried out using polymerase chain reaction (PCR) technologies or other nucleic acid amplification technologies well known in the art.

The term “antagonist” refers to a molecule which inhibits or attenuates the biological activity of PMMM. Antagonists may include proteins such as antibodies, anticalins, nucleic acids, carbohydrates, small molecules, or any other compound or composition which modulates the activity of PMMM either by directly interacting with PMMM or by acting on components of the biological pathway in which PMMM participates.

The term “antibody” refers to intact immunoglobulin molecules as well as to fragments thereof, such as Fab, F(ab′)₂, and Fv fragments, which are capable of binding an epitopic determinant. Antibodies that bind PMMM polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression systemhas been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic PMMM, or of any oligopeptide thereof, to induce a specific imne response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formlation or an aqueous solution. Compositions comprising polynucleotides encoding PMMM or fragments of PMMM may be employed as hybridization probes. The probes may be stored in freeze-dried form and may be associated with a stabilizing agent such as a carbohydrate. In hybridizations, the probe may be deployed in an aqueous solution containing salts (e.g., NaCI), detergents (e.g., sodium dodecyl sulfate; SDS), and other components (e.g., Denhardt's solution, dry milk, samn sperm DNA, etc.).

“Consensus sequence” refers to a nucleic acid sequence which has been subjected to repeated DNA sequence analysis to resolve uncalled bases, extended using the XL-PCR kit (Applied Biosystems, Poster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (Accelrys, Burlington Mass.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of PMMM or a polynucleotide encoding PMMM which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:32-62 can comprise a region of unique polynucleotide sequence that specifically identifies SEQ ID NO:32-62, for example, as distinct from any other sequence in the genome from which the fragment was obtained. A fragment of SEQ ID NO:32-62 can be employed in one or more embodiments of methods of the invention, for example, in hybridization and amplification technologies and in analogous methods that distinguish SEQ ID NO:32-62 from related polynucleotides. The precise length of a fragment of SEQ ID NO:32-62 and the region of SEQ ID NO:32-62 to which the fragment corresponds are routinely determinable by one of ordinary skill in the art based on the intended purpose for the fragment.

A fragment of SEQ ID NO:1-31 is encoded by a fragment of SEQ ID NO:32-62. A fragment of SEQ ID NO:1-31 can comprise a region of unique amino acid sequence that specifically identifies SEQ ID NO:1-31. For example, a fragment of SEQ ID NO:1-31 canbe used as an immunogenic peptide for the development of antibodies that specifically recognize SEQ ID NO:1-31. The precise length of a fragment of SEQ ID NO:1-31 and the region of SEQ ID NO:1-31 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise conparison of two nucleotide sequences. “BLAST 2 Sequences” can be accessed and used interactively at http://www.ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr.-21-2000) set at default parameters. Such default parameters may be, for example:
Matrix: BLOSUM62
Reward for match: 1
Penalty for mismatch: −2
Open Gap: 5 and Extension Gap: 2 penalties
Gap x drop-off: 50
Expect: 10
Word Size: 11
Filter: on

Percent identity may be measured over the length of an entire defined sequence, for example, as defined by a particular SEQ ID nunmber, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode simar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” tool Version 2.0.12 (Apr.-21-2000) with blastp set at default parameters. Such default parameters may be, for example:
Matrix: BLOSUM62
Open Gap: 11 and Extension Gap: 1 penalties
Gap x drop-off: 50
Expect: 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skll in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions win be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“lmmune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of PMMM which is capable of eliciting an immune response when introduced into a living organism, for example, a mamnmal. The term “immunogenic fragment” also includes any polypeptide or oligopeptide fragment of PMMM which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of PMMM. For example, modulation may cause an increase or a decrease in protein activity, binding characteristics, or any other biological, functional, or immunological properties of PMMM.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an PMMM may involve lipidation, glycosylation, phosphorylation, acetylation, racemization, proteolytic cleavage, and other modifications known in the art. These processes may occur synthetically or biochemically. Biochemical modifications will vary by cell type depending on the enzymtic milieu of PMMM.

“Probe” refers to nucleic acids encoding PMMM, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4^{th} ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; iniubitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing PMMM, nucleic acids encoding PMMM, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or aniino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate′ refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91 %, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May-07-1999) set at default parameters. Such a pair of polypeptides may show, for example, at least 50%, at least 60%, at least 70%, at least 80%, at least 85 %, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

The Invention

Various embodiments of the invention include new human protein modification and maintenance molecules (PMMM), the polynucleotides encoding PMMM, and the use of these compositions for the diagnosis, treatment, or prevention of gastrointestinal, cardiovascular, automimnefiflammatory, cell proliferative, developmental, epithelial, neurological, reproductive, endocrine, metabolic, pancreatic disorders, disorders associated with the adrenals, disorders associated with gonadal steroid hormones, cancers, and infections.

Table 1 summrizes the nomenclature for the fiul length polynucleotide and polypeptide enibodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to the polypeptides of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (hicyte Polypeptide ID) for polypeptides of the invention Column 3 shows the GenBak identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column S shows potential glycosylation sites, as deterined by the MOTOIFS program of the GCG sequence analysis software package (Accelrys, Burlington Mass.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are protein modification and maintenance molecules. For example, SEQ ID NO:2 is 86% identical, from residue M1 to residue E738 and 96% identical, from residue K607 to residue L900, to human inter-alpha-trypsin inhibitor family heavy chain-related protein (GenBank ID g4096840) as determined by the Basic Local Alignment Search Tool (BLAST). (See Table 2.) The BLAST probability scores are 0.0 and 7.3e-152, which indicate the probability of obtaining the observed polypeptide sequence alignment by chance. SEQ ID NO:2 also contains a von Wilebrand factor type A domain as determined by searching for statistically significant matches in the hidden Markov model (HMM)-based PFAM database of conserved protein family domains. (See Table 3.) Data from BLIMPS, MOTIFS, and additional BLAST analyses provide further corroborative evidence that SEQ ID NO:2 is a protease inhibitor.

In another example, SEQ ID NO:9 is 50% identical, from residue Ml to residue G378, to Mus musculus mDj10 (GenBank ID g6567172) as determined by BLAST. The BLAST probability score is 9.7e-102. SEQ ID NO:9 also contains a DnaJ domain as determined by searching for statistically significant matches in the hidden Markov model (1{M)-based PFAM database. Data from BLIMPS, MOTIPS, and PROFILESCAN analyses provide further corroborative evidence that SEQ ID NO:9 is a molecular chaperone.

In another example, SEQ ID NO:12 is 100% identical, from residue Ml to residue N344, to human phosphatidyl inositol glycan class T (GenBank ID g14456615) as determined by BLAST. The BLAST probability score is 5.4e280. Data from BLAST-PRODOM analysis provides further corroborative evidence that SEQ ID NO:12 is a phosphatidyl inositol glycail In an alternative example, SEQ ID NO:13 is 100% identical, from residue D63 to residue L476, to human phosphatidyl inositol glycan class T (GenBank ID g14456615) as determined by BLAST. The BLAST probability score is 4.7e-261. Data from BLAST-PRODOM analysis provides further corroborative evidence that SEQ ID NO:13 is a phosphatidyl inositol glycan.

In yet another example, SEQ ID NO:15 is 97% identical, from residue D50 to residue D121, to human ubiquitin-conjugating enzyme HR6B (GenBank ID g11037550) as determined by BLAST. The BLAST probability score is 2.1e-58. SEQ ID NO:15 is localized to the subcellular region, has ubiquitination function, and is a protein conjugation factor as determined by BLAST analysis using the PROTEOME database. SEQ ID NO:15 also contains an ubiquitin-conjugating enzyme domain as determined by searching for statistically significant matches in the hidden Markov model (HMM-based PFAM database. Data from BLAST-PRODOM, BLAST-DOMO, and PROFILESCAN analyses provide further corroborative evidence that SEQ ID NO:15 is a ubiquitin-conjugating enzyme.

In a further example, SEQ ID NO:19 is 100% identical, from residue Ml to residue G82, and 100% identical, from residue G82 to residue A652, to the large subunit of human CANP (GenBank ID g29664, residues M1-G82 and G144-A714 respectively) as determined by BLAST. The BLAST probability score is 0.0. SEQ ID NO:19 is homologous to other proteins, such as calpain, the large subunit of a cysteine protease, having cysteine protease activity and localized to the plasma membrane, as determined by BLAST analysis using the PROTEOME database. SEQ ID NO:19 also contains calpain and EF hand domains as determined by searching for statistically significant matches in the bidden Markov model (HMM)-based PFAM database of conserved protein family domins. Data from BLIMPS, MOTIFS, and BLAST analyses provide further corroborative evidence that SEQ ID NO:19 is a calpain cysteine protease. SEQ ID NO:1, SEQ ID NO:3-8, SEQ ID NO:10-11, SEQ ID NO:14, SEQ ID NO:16-18, and SEQ ID NO:20-31 were analyzed and annotated in a similar manner. The algorithms and parameters for the analysis of SEQ ID NO:1-31 are described in Table 7.

As shown in Table 4, the full length polynucleotide enbodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the fill length polynucleotide embodiments, and of fragments of the polynucleotides which are useful, for example, in hybridization or amplification technologies that identify SEQ ID NO:32-62 or that distinguish between SEQ ID NO:32-62 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (ie., those sequences including the designation “NP”). Alternatively, the polynucleotide fragments described in column 2 may refer to assemblages of both cDNA and Genscan-predicted exons brought together by an “exon stitching” algoritm For example, a polynucleotide sequence identified as FL_XXXXXX_N₁_{^{—}}N₂_{^{—}}YYYYY_N₃_{^{—}}N₄ represents a “stitched” sequence in which XXXXXX is the identification number of the cluster of sequences to which the algorithm was applied, and YYYYY is the nuniber of the prediction generated by the algorithm and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genornic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences using, for |
| ENST | example, GENSCAN (Stanford University, CA, USA) |
| | or FGENES (Computer Genomics Group, The Sanger |
| | Centre, Cambridge, UK). |
| GBI | Hand-edited analysis of genomic sequences. |
| FL | Stitched or stretched genomic sequences (see Example V). |
| INCY | Full length transcript and exon prediction from mapping |
| | of EST sequences to the genome. Genomic location |
| | and EST composition data are combined to predict the |
| | exons and resulting transcript. |

In some cases, Incyte cDNA coverage redundant with the sequence coverage shown in Table 4 was obtained to confirm the final consensus polynucleotide sequence, but the relevant Incyte cDNA identification nunibers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification nunber for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses PMMM variants. Various embodiments of PMMM variants can have at least about 80%, at least about 90%, or at least about 95% amino acid sequence identity to the PMMM amino acid sequence, and can contain at least one functional or structural characteristic of PMMM.

Various embodiments also encompass polynucleotides which encode PMMM. In a particular embodiment, the invention encompasses a polynucleotide sequence comprising a sequence selected from the group consisting of SEQ ID NO:32-62, which encodes PMMM. The polynucleotide sequences of SEQ ID NO:32-62, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding PMMM. In particular, such a variant polynucleotide will have at least about 70%, or alternatively at least about 85%, or even at least about 95% polynucleotide sequence identity to a polynucleotide encoding PMMM. A particular aspect of the invention encompasses a variant of a polynucleotide comprising a sequence selected from the group consisting of SEQ ID NO:32-62 which has at least about 70%, or alternatively at least about 85%, or even at least about 95% polynucleotide sequence identity to a nucleic acid sequence selected from the group consisting of SEQ ID NO:32-62. Any one of the polynucleotide variants described above can encode a polypeptide which contains at least one functional or structural characteristic of PMMM.

In addition, or in the alternative, a polynucleotide variant of the invention is a splice variant of a polynucleotide encoding PMMM. A splice variant may have portions which have significant sequence identity to a polynucleotide encoding PMMM, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding PMMM over its entire length; however, portions of the splice variant will have at least about 70%, or alternatively at least about 85%, or alternatively at least about 95%, or alternatively 100% polynucleotide sequence identity to portions of the polynucleotide encoding PMMM. For example, a polynucleotide comprising a sequence of SEQ ID NO:43 and a polynucleotide comprising a sequence of SEQ ID NO:44 are splice variants of each other. Any one of the splice variants described above can encode a polypeptide which contains at least one functional or structural characteristic of PMMM.

It will be appreciated by those skilled in the art that as a result of the degeneracy of the genetic code, a multitude of polynucleotide sequences encoding PMMM, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring PMMM, and all such variations are to be considered as being specifically disclosed.

Although polynucleotides which encode PMMM and its variants are generally capable of hybridizing to polynucleotides encoding naturally occurring PMMM under appropriately selected conditions of stringency, it may be advantageous to produce polynucleotides encoding PMMM or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding PMMM and its derivatives without altering the encoded amino acid sequences include the production of RNA transcripts having more desirable properties, such as a greater half-life, than transcripts produced from the naturally occurring sequence.

The invention also encompasses production of polynucleotides which encode PMMM and PMMM derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding PMMM or any fragment thereof.

Embodiments of the invention can also include polynucleotides that are capable of hybridizing to the claimed polynucleotides, and, in particular, to those having the sequences shown in SEQ ID NO:32-62 and fragments thereof, under various conditions of stringency (Wabl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the enmbodimnents of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding PMMM may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify uninown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences inhuman and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of iuknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Outputlight intensity may be converted to electrical signal using appropriate software (e.g., GENOTYPER and SEQUENCE NAVIGATOR, Applied Biosystems), and the entire process from loading of samples to computer analysis and electronic data display maybe computer controlled. Capillary electrophoresis is especially preferable for sequencing small DNA fragments which may be present in limited amounts in a particular sample.

In another embodiment of the invention, polynucleotides or fragments thereof which encode PMMM may be cloned in recombinant DNA molecules that direct expression of PMMM, or fragments or functional equivalents thereof, in appropriate host cells. Due to the inherent degeneracy of the genetic code, other polynucleotides which encode substantially the same or a functionally equivalent polypeptides may be produced and used to express PMMM.

The polynucleotides of the invention can be engineered using methods generally known in the art in order to alter PMMM-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuflling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, P. C. et al. (1999) Nat. Biotechnol. 17:259-264; and Crameri, A. et al. (1996) Nat. Biotechnol. 14:315-319) to alter or improve the biological properties of PMMM, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and furher subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding PMMM may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, PMMM itself or a fragment thereof may be synthesized using chemical methods known in the art. For example, peptide synthesis can be performed using various solution-phase or solid-phase techniques (Creighton, T. (1984) Proteins, Structures and Molecular Properties, WH Freeman, New York N.Y., pp. 55-60; Roberge, J. Y. et al. (1995) Science 269:202-204). Automated synthesis maybe achieved using the ABI 431A peptide synthesizer (Applied Biosystems). Additionally, the amino acid sequence of PMMM, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active PMMM, the polynucleotides encoding PMMM or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding PMMM. Such elements may vary in their strength and specificity. Specific initiation signals may also be used to achieve more efficient translation of polynucleotides encoding PMMM. Such signals include the ATG initiation codon and adjacent sequences, e.g. the Kozak sequence. In cases where a polynucleotide sequence encoding PMMM and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding PMMM and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding PMMM. These include, but are not limited to, microorganisms such as bacteria transformed with recombinant bacteriophage, plasrnid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, orherpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a nunmber of cloning and expression vectors maybe selected depending upon the use intended for polynucleotides encoding PMMM. For example, routine cloning, subdloning, and propagation of polynucleotides encoding PMMM can be achieved using a multifunctional E. coli vector such as PBLUESCRIPT (Stratagene, La Jolla Calif.) or PSPORT1 plasmid (Invitrogen). Ligation of polynucleotides encoding PMMM into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of PMMM are needed, e.g. for the production of antibodies, vectors which direct high level expression of PMMM may be used. For example, vectors containing the strong, inducible SP6 or T7 bacteriophage promoter may be used.

Yeast expression systems may be used for production of PMMM. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A et al. (1987) Methods Enzymol. 153:516-544; Scorer, C. A. et al. (1994) Bio/Technology 12:181-184).

Plant systems may also be used for expression of PMMM. Transcription of polynucleotides encoding PMMM may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heatshock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding PMMM may be ligated into an adenovirus transcription/translation complex consisting of the late promoter and tripartite leader sequence. Insertion in a non-essential E1 or E3 region of the viral genome may be used to obtain infective virus which expresses PMMM in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of PMMM in cell lines is preferred. For example, polynucleotides encoding PMMM can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any numaber of selection systems may be used to recover transformed cell lines. These include, but are not limited to, the herpes simplex virus thymidine kinase and adenine phosphoribosyltransferase genes, for use in tk⁻ and apr⁻ cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For exarnple, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., tipB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be coneirmed. For example, if the sequence encoding PMMM is inserted within a marker gene sequence, transformed cells containing polynucleotides encoding PMMM can be identified by the absence of marker gene function. Alternatively, a marker gene can be placed in tandem with a sequence encoding PMMM under the control of a single promoter. Expression of the marker gene in response to induction or selection usually indicates expression of the tandem gene as well.

In general, host cells that contain the polynucleotide encoding PMMM and that express PMMM may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of PMMM using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on PMMM is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amaino acid assays. Means for producing labeled hybridization or PCR probes for detecting sequences related to polynucleotides encoding PMMM include oligolabeling, nick translation, end-labeling, or PCR amplification using a labeled nucleotide. Alternatively, polynucleotides encoding PMMM, or any fragments thereof, may be cloned into a vector for the production of an mRNA probe. Such vectors are known in the art, are commercially available, and may be used to synthesize RNA probes in vitro by addition of an appropriate RNA polyrerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding PMMM may be cultured under conditions suitable for the expression and recovery of the protein from cell culture. The protein produced by a transformed cell may be secreted or retained intracellularly depending on the sequence and/or the vector used. As win be understood by those of skill in the art, expression vectors containing polynucleotides which encode PMMM may be designed to contain signal sequences which direct secretion of PMMM through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding PMMM may be ligated to a heterologous sequence resulting in translation of a fusion protein in any of the aforementioned host systems. For example, a chimeric PMMM protein containing a heterologous moiety that can be recognized by a commercially available antibody may facilitate the screening of peptide libraries for inhibitors of PMMM activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the PMMM encoding sequence and the heterologous protein sequence, so that PMMM may be cleaved away from the heterologous moiety following purification Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled PMMM may be achieved inz vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-Methionie.

PMMM, fragments of PMMM, or variants of PMMM may be used to screen for compounds that specifically bind to PMMM. One or more test compounds may be screened for specific binding to PMMM. In various embodiments, 1, 2, 3, 4, 5, 10, 20, 50, 100, or 200 test compounds can be screened for specific binding to PMMM. Examples of test compounds can include antibodies, anticalins, oligonucleotides, proteins (e.g., ligands or receptors), or small molecules.

In related embodiments, variants of PMMM can be used to screen for binding of test compounds, such as antibodies, to PMMM, a variant of PMMM, or a combination of PMMM and/or one or more variants PMMM. In an embodiment, a variant of PMMM can be used to screen for compounds that bind to a variant of PMMM, but not to PMMM having the exact sequence of a sequence of SEQ ID NO:1 -31. PMMM variants used to perform such screening can have a range of about 50% to about 99% sequence identity to PMMM, with various embodiments having 60%, 70%, 75%, 80%, 85%, 90%, and 95% sequence identity.

In an embodiment, a compound identified in a screen for specific binding to PMMM canbe closely related to the natural ligand of PMMM, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor PMMM (Howard, A. D. et al. (2001) Trends Pharmacol. Sci.22:132-140; Wise, A. et al. (2002) Drug Discovery Today 7:235-246).

In other embodiments, a compound identified in a screen for specific binding to PMMM can be closely related to the natural receptor to which PMMM binds, at least a fragment of the receptor, or a fragment of the receptor including all or a portion of the ligand binding site or binding pocket. For example, the compound may be a receptor for PMMM which is capable of propagating a signal, or a decoy receptor for PMMM which is not capable of propagating a signal (Ashkenazi, A and V. M. Divit (1999) Curr. Opin. Cell Biol. 11:255-260; Mantovani, A. et al. (2001) Trends Immunol. 22:328-336). The compound canbe rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (OF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to PMMM, fragments of PMMM, or variants of PMMM. The binding specificity of the antibodies thus screened can thereby be selected to identify particular fragments or variants of PMMM. In one embodiment, an antibody can be selected such that its binding specificity allows for preferential identification of specific fragments or variants of PMMM. In another embodiment, an antibody can be selected such that its binding specificity allows for preferential diagnosis of a specific disease or condition having increased, decreased, or otherwise abnormal production of PMMM.

In an embodiment, anticalins can be screened for specific binding to PMMM, fragments of PMMM, or variants of PMMM. Anticalins are ligand-binding proteins that have been constructed based on a lipocalin scaffold (Weiss, G. A. and H. B. Lowman (2000) Chem. Biol. 7:R177-R184; Skefra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit PMMM involves producing appropriate cells which express PMMM, either as a secreted protein or on the cell membrane. Preferred cells can include cells from mammals, yeast, Drosophila, or E. coli. Cells expressing PMMM or cell membrane fractions which contain PMMM are then contacted with a test compound and binding, stimulation, or inhibition of activity of either PMMM or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with PMMM, either in solution or affixed to a solid support, and detecting the binding of PMMM to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H.B. et al. (1991) J. Biol. Chem. 266:10982-10988).

PMMM, fragments of PMMM, or variants of PMMM may be used to screen for compounds that modulate the activity of PMMM. Such compounds may include agonists, antagonists, or partial or inverse agonists. In one embodiment, an assay is performed under conditions permissive for PMMM activity, wherein PMMM is combined with at least one test compound, and the activity of PMMM in the presence of a test compound is compared with the activity of PMMM in the absence of the test compound. A change in the activity of PMMM in the presence of the test compound is indicative of a compound that modulates the activity of PMMM. Alternatively, a test compound is combined with an int vitro or cell-free system comprising PMMM under conditions suitable for PMMM activity, and the assay is performed. In either of these assays, a test compound which modulates the activity of PMMM may do so indirectly and need not come in direct contact with the test compound. At least one and up to a plurality of test compounds may be screened.

In another embodiment, polynucleotides encoding PMMM or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (reo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding PMMM may also be manipulated int vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding PMMM can also be used to create “knockin” humanized animals (pigs) or transgenic animals (mice or rats) to model human disease. With knockin technology, a region of a polynucleotide encoding PMMM is injected into animal ES cells, and the injected sequence integrates into the anial cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress PMMM, e.g., by secreting PMMM in its mill, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of PMMM and protein modification and maintenance molecules. In addition, examples of tissues expressing PMMM can be found in Table 6 and can also be found in Example XI. Therefore, PMMM appears to play a role in gastrointestinal, cardiovascular, autoimmune/inflammatory, cell proliferative, developmental, epithelial, neurological, reproductive, endocrine, metabolic, pancreatic disorders, disorders associated with the adrenals, disorders associated with gonadal steroid hormones, cancers, and infections. In the treatment of disorders associated with increased PMMM expression or activity, it is desirable to decrease the expression or activity of PMMM. In the treatment of disorders associated with decreased PMMM expression or activity, it is desirable to increase the expression or activity of PMMM.

Therefore, in one embodiment, PMMM or a fragment or derivative thereof may be administered to a subject to treat or prevent a disorder associated with decreased expression or activity of PMMM. Examples of such disorders include, but are not limited to, a gastrointestinal disorder, such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a cardiovascular disorder, such as arteriovenous fistula, atherosclerosis, hypertension, vasculitis, Raynaud's disease, aneurysms, arterial dissections, varicose veins, thrombopblebitis and pblebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation; an autoimmune/inflammatory disease, such as acquired immunodeficiency syndrome (AIDS), Addison's disease, adult respiratory distress syndrome, allergies, ankylosing spondylitis, amyloidosis, anemia, asthma, atherosclerosis, atherosclerotic plaque rupture, autoimrnne hemolytic anemia, autoimnne thyroiditis, autoimmnne polyendocrinopathy-candidiasis-ectodermal dystrophy (APECED), bronchitis, cholecystitis, contact dermatitis, Crohn's disease, atopic dermatitis, denmatomyositis, diabetes mellitus, emphysema, episodic lynphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, degradation of articular cartilage, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia; a developmental disorder, such as renal tubular acidosis, anemia, Cushing's syndrome, achondroplastic dwarfism, Duchenne and Becker muscular dystrophy, bone resorption, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, age-related macular degeneration, and sensorineural hearing loss; an epithelial disorder, such as dyshidrotic eczema, allergic contact dermatitis, keratosis pilaris, melasma, vitiligo, actinic keratosis, basal cell carcinoma, squamous cell carcinoma, seborrheic keratosis, folliculitis, herpes simplex, herpes zoster, varicella, candidiasis, dermatophytosis, scabies, insect bites, cherry angioma, keloid, dermatofibroma, acrochordons, urticaria, transient acantholytic dermatosis, xerosis, eczema, atopic dermatitis, contact dermatitis, hand eczema, nummular eczema, lichen simplex chronicus, asteatotic eczema, stasis dermatitis and stasis ulceration, seborrheic dermatitis, psoriasis, lichen planus, pityriasis rosea, impetigo, ecthyma, dermatophytosis, tinea versicolor, warts, acne vulgaris, acne rosacea, pemphigus vulgaris, pemphigus foliaceus, paraneoplastic pemphigus, bullous pemphigoid, herpes gestationis, dermatitis herpetiformis, linear IgA disease, epidermolysis bullosa acquisita, dermatonyositis, lupus erythematosus, scleroderma and morphea, erytiroderma, alopecia, figurate skin lesions, telangiectasias, hypopigmentation, hyperpigmentation, vesicles/bullae, exanthems, cutaneous drug reactions, papulonodular skin lesions, chronic non-healing wounds, photosensitivity diseases, epidermolysis bullosa simplex, epidermolytic hyperkeratosis, epidermolytic and nonepidermolytic palmoplantar keratoderma, ichthyosis bullosa of Siemens, ichthyosis exfoliativa, keratosis paimaris et plantaris, keratosis palmoplantaris, palmoplantar keratoderma, keratosis punctata, Meesmann's corneal dystrophy, pachyonychia congenita, white sponge nevus, steatocystoma multiplex, epidermal nevi/epidermolytic hyperkeratosis type, monilethrix, trichothiodystrophy, chronic hepatitis/cryptogenic cirrhosis, and colorectal hyperplasia; a neurological disorder, such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningtis, brain abscess, subdural empyema, epidural abscess, suppurative intracranial thrombopblebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a reproductive disorder, such as infertility, including tubal disease, ovulatory defects, and endometriosis, a disorder of prolactin production, a disruption of the estrous cycle, a disruption of the menstrual cycle, polycystic ovary syndrome, ovarian hyperstimulation syndrome, an endometrial or ovarian tumor, a uterine fibroid, autoimmune disorders, an ectopic pregnancy, and teratogenesis; cancer of the breast, fibrocystic breast disease, and galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, and gynecomastia; an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma; a disorder associated with hypopituitarism including hypogonadism, Sheehan syndrome, diabetes insipidus, Kallman's disease, Hand-Schuller-Christian disease, Letterer-Siwe disease, sarcoidosis, empty sella syndrome, and dwarfism; a disorder associated with hyperpituitarism including acromegaly, giantism, and syndrome of inappropriate antidiuretic hormone (ADH) secretion (SIADH) often caused by benign adenoma; a disorder associated with hypothyroidism including goiter, myxedema, acute thyroiditis associated with bacterial infection, subacute thyroiditis associated with viral infection, autoimmune thyroiditis (Hashimoto's disease), and cretinism; a disorder associated with hyperthyroidism including thyrotoxicosis and its various forms, Grave's disease, pretibial myxedema, toxic multinodular goiter, thyroid carcinoma, and Plummer's disease; a disorder associated with hyperparathyroidism including Conn disease (chronic hypercalemia); a metabolic disorder such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, pentosuria phenylketonuria, pseudovitamin D-deficiency rickets; a disorder of carbohydrate metabolism such as congenital type II dyserythropoietic anemia, diabetes, insulin-dependent diabetes mellitus, non-insulin-dependent diabetes mellitus, fructose-1,6-diphosphatase deficiency, galactosemia, glucagonoma, hereditary fructose intolerance, hypoglycemia, mannosidosis, neuraminidase deficiency, obesity, galactose epimerase deficiency, glycogen storage diseases, lysosomal storage diseases, fructosuria, pentosuria, and inherited abnormalities of pyruvate metabolism; a disorder of lipid metabolism such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, lipid myopathies, and obesity; and a disorder of copper metabolism such as Menke's disease, Wilson's disease, and Ehlers-Danlos syndrome type IX; a pancreatic disorder such as Type I or Type II diabetes mellitus and associated complications; a disorder associated with the adrenals such as hyperplasia, carcinoma, or adenoma of the adrenal cortex, hypertension associated with alkalosis, amyloidosis, hypokalemia, Cushing's disease, Liddle's syndrome, and Arnold-Healy-Gordon syndrome, pheochromocytoma tumors, and Addison's disease; a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis; and, in men, Leydig cell deficiency, male climacteric phase, and germinal cell aplasia, a hypergonadal disorder associated with Leydig cell tumors, androgen resistance associated with absence of androgen receptors, syndrome of 5 α-reductase, and gynecomastia; a cancer such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; and an infection caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picomavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fungal agent classified as aspergillus, blastomyces, dermatophytes, cryptococcus, coccidioides, malasezzia, histoplasma, or other mycosis-causing fungal agent; and an infection caused by a parasite classified as plasmodium or malaria-causing, parasitic entamoeba, leishmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestrode such as tapeworm.

In another embodiment, a vector capable of expressing PMMM or a fragment or derivative thereof may be administered to a subject to treat or prevent a disorder associated with decreased expression or activity of PMMM including, but not limited to, those described above.

In a further embodiment, a composition comprising a substantially purified PMMM in conjunction with a suitable pharmaceutical carrier may be administered to a subject to treat or prevent a disorder associated with decreased expression or activity of PMMM including, but not limited to, those provided above.

In still another embodiment, an agonist which modulates the activity of PMMM may be administered to a subject to treat or prevent a disorder associated with decreased expression or activity of PMMM including, but not limited to, those listed above.

In a further embodiment, an antagonist of PMMM may be administered to a subject to treat or prevent a disorder associated with increased expression or activity of PMMM. Examples of such disorders include, but are not limited to, those gastrointestinal, cardiovascular, autoimmune/inflammatory, cell proliferative, developmental, epithelial, neurological, reproductive, endocrine, metabolic, pancreatic disorders, disorders associated with the adrenals, disorders associated with gonadal steroid hormones, cancers, and infections described above. In one aspect, an antibody which specifically binds PMMM may be used directly as an antagonist or indirectly as a targeting or delivery mechanism for bringing a pharmaceutical agent to cells or tissues which express PMMM.

In an additional embodiment, a vector expressing the complement of the polynucleotide encoding PMMM may be administered to a subject to treat or prevent a disorder associated with increased expression or activity of PMMM including, but not limited to, those described above.

In other embodiments, any protein, agonist, antagonist, antibody, complementary sequence, or vector embodiments may be administered in combination with other appropriate therapeutic agents. Selection of the appropriate agents for use in conmbination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of PMMM may be produced using methods which are generally known in the art. In particular, purified PMMM may be used to produce antibodies or to screen libraries of pharmaceutical agents to identify those which specifically bind PMMM. Antibodies to PMMM may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enyyme inhibitors and may have application in the design of peptide mimetics, and in the development of immino-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with PMMM or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to PMMM have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein. Short stretches of PMMM amino acids may be fused with those of another protein, such as KLH, and antibodies to the chimeric molecule may be produced.

Monoclonal antibodies to PMMM may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce PMMM-specific single chain antibodies. Antibodies with related specificity, but of distinct idiotypic composition, maybe generated by chain shuffing from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening imunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for PMMM may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between PMMM and its specific antibody. A two-site, monoclonal-based immunoassay utilizing monoclonal antibodies reactive to two non-interfering PMMM epitopes is generally used, but a competitive binding assay may also be employed (Pound, supra).

Various methods such as Scatchard analysis in conjunction with radioimmunoassay techniques may be used to assess the affinity of antibodies for PMMM. Affinity is expressed as an association constant, Kₐ, which is defined as the molar concentration of PMMM-antibody complex divided by the molar concentrations of free antigen and free antibody under equilibrium conditions. The Kₐ determined for a preparation of polyclonal antibodies, which are heterogeneous in their affinities for multiple PMMM epitopes, represents the average affinity, or avidity, of the antibodies for PMMM. The Kₐ determined for a preparation of monoclonal antibodies, which are monospecific for a particular PMMM epitope, represents a true measure of affinity. High-affinity antibody preparations with Kₐ ranging from about 10⁹ to 10¹² L/mole are preferred for use in immunoassays in which the PMMM-antibody complex must withstand rigorous manipulations. Low-affinity antibody preparations with Kₐ ranging from about 10⁶ to 10⁷ L/mole are preferred for use in immunopurification and similar procedures which ultimately require dissociation of PMMM, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to deternine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of PMMM-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding PMMM, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding PMMM. Such technology is well known in the art, and antisense oligonucleotides or larger fragments can be designed from various locations along the coding or control regions of sequences encoding PMMM (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding PMMM may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in PMMM expression or regulation causes disease, the expression of PMMM from an appropriate population of transduced cells may alleviate the clinical manifestations caused by the genetic deficiency.

In a further embodiment of the invention, diseases or disorders caused by deficiencies in PMMM are treated by constructing mammalian expression vectors encoding PMMM and introducing these vectors by mechanical means into PMMM-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Récipon (1998) Curr. Opin. Biotechnol. 9:445-450).

Expression vectors that may be effective for the expression of PMMM include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). PMMM may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, P. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F.M.V. and H.M. Blau, supra)), or (iii) a tissue-specific promoter or the native promoter of the endogenous gene encoding PMMM from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to PMMM expression are treated by constructing a retrovirus vector consisting of (i) the polynucleotide encoding PMMM under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding PMMM to cells which have one or more genetic abnormalities with respect to the expression of PMMM. The construction and packaging of adenovirus-based vectors are well known to those with ordinary skill in the art. Replication defective adenovirus vectors have proven to be versatile for importing genes encoding innunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding PMMM to target cells which have one or more genetic abnormalities with respect to the expression of PMMM. The use of herpes simplex virus (HSV)-based vectors may be especially valuable for introducing PMMM to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding PMMM to target cells. The biology of the prototypic alphavirus, Seniln Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for PMMM into the alphavirus genome in place of the capsid-coding region results in the production of a large number of PMMM-coding RNAs and the synthesis of high levels of PMMM in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:7483). The wide host range of alphaviruses will allow the introduction of PMMM into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to iihibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding PMMM.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding PMMM. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flaning sequences at the 5′ andlor 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

In other embodiments of the invention, the expression of one or more selected polynucleotides of the present invention can be altered, inhibited, decreased, or silenced using RNA interference (RNAi) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PTGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in mammalian cells by the use of small interfering RNA also known as siRNA. SiRNA are shorter segments of dsRNA (typically about 21 to 23 nucleotides in length) that result in vivo from cleavage of introduced dsRNA by the action of an endogenous ribonuclease. SiRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

SiRNA can either be generated indirectly by introduction of dsRNA into the targeted cell, or directly by mammalian transfection methods and agents described herein or known in the art (such as liposome-mediated transfection, viral vector methods, or other polynucleotide delivery/introductory methods). Suitable SiRNAs can be selected by exam iing a transcript of the target polynucleotide (e.g., mRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (UTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected SiRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brummelkamp, T. R. et al. (2002) Science 296:550-553; and Paddison, P. J. et al. (2002) Genes Dev. 16:948-958). In these and related enbodiments, shRNAs can be delivered to target cells using expression vectors known in the art. An example of a suitable expression vector for delivery of siRNA is the PSILENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene, can be determined by northern analysis methods using, for example, the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined by Western analysis using standard techniques known in the art.

An additional embodiment of the invention encompasses a method for screening for a compound which is effective in altering expression of a polynucleotide encoding PMMM. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased PMMM expression or activity, a compound which specifically inhibits expression of the polynucleotide encoding PMMM may be therapeutically useful, and in the treatment of disorders associated with decreased PMMM expression or activity, a compound which specifically promotes expression of the polynucleotide encoding PMMM may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding PMMM is exposed to at least one test compound thus obtained. The sample may comprise, for example, an intact or permeabilized cell, or an in vitro cell-free or reconstituted biochemical system. Alterations in the expression of a polynucleotide encoding PMMM are assayed by any method commonly known in the art. Typically, the expression of a specific nucleotide is detected by hybridization with a probe having a nucleotide sequence complementary to the sequence of the polynucleotide encoding PMMM. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commnonly known and are thoroughly discussed in the latest edition of Remington's Pharmaceutical Sciences (Maack Publishing, Easton Pa.). Such compositions may consist of PMMM, antibodies to PMMM, and mimetics, agonists, antagonists, or inhibitors of PMMM.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form These compositions are generally aerosolized immediately prior to inhalation by the patient. In the case of small molecules (e.g. traditional low molecular weight organic drugs), aerosol delivery of fast-acting formulations is well-known in the art In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising PMMM or fragments thereof. For example, liposome preparations containing a cell-impermeable macromolecule may promote cell fusion and intracellular delivery of the macromolecule. Alternatively, PMMM or a fragment thereof may be joined to a short cationic N-terminal portion fromthe HIV Tat-1 protein. Fusion proteins thus generated havebeen found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example PMMM or fragments thereof, antibodies of PMMM, and agonists, antagonists or inhibitors of PMMM, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED50 ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formelations for nucleotides than for proteins or their inhibitors. Similarly, delivery of polynucleotides or polypeptides will be specific to particular cells, conditions, locations, etc.

Diagnostics

In another emnbodiment, antibodies which specifically bind PMMM may be used for the diagnosis of disorders characterized by expression of PMMM, or in assays to monitor patients being treated with PMMM or agonists, antagonists, or inhibitors of PMMM. Antibodies useful for diagnostic purposes may be prepared in the same manner as described above for therapeutics. Diagnostic assays for PMMM include methods which utilize the antibody and a label to detect PMMM in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring PMMM, including ELISAs, RIAs, and FACS, are known in the art and provide a basis for diagnosing altered or abnormal levels of PMMM expression. Normal or standard values for PMMM expression are established by combining body fluids or cell extracts taken from normal lanzlian subjects, for example, human subjects, with antibodies to PMMM under conditions suitable for complex formation. The amount of standard complex formation may be quantitated by various methods, such as photometric means. Quantities of PMMM expressed in subject, control, and disease samples from biopsied tissues are compared with the standard values. Deviation between standard and subject values establishes the parameters for diagnosing disease.

In another enibodiment of the invention, polynucleotides encoding PMMM may be used for diagnostic purposes. The polynucleotides which may be used include oligonucleotides, complementary RNA and DNA molecules, and PNAs. The polynucleotides may be used to detect and quantify gene expression in biopsied tissues in which expression of PMMM may be correlated with disease. The diagnostic assay may be used to determine absence, presence, and excess expression of PMMM, and to monitor regulation of PMMM levels during therapeutic intervention.

In one aspect, hybridization with PCR probes which are capable of detecting polynucleotides, including genomic sequences, encoding PMMM or closely related molecules may be used to identify nucleic acid sequences which encode PMMM. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding PMMM, allelic variants, or related sequences.

Probes may also be used for the detection of related sequences, and may have at least 50% sequence identity to any of the PMMM encoding sequences. The hybridization probes of the subject invention may be DNA or RNA and may be derived from the sequence of SEQ ID NO:32-62 or from genomic sequences including promoters, enhancers, and introns of the PMMM gene.

Means for producing specific hybridization probes for polynucleotides encoding PMMM include the cloning of polynucleotides encoding PMMM or PMMM derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding PMMM may be used for the diagnosis of disorders associated with expression of PMMM. Examples of such disorders include, but are not limited to, a gastrointestinal disorder, such as dysphagia, peptic esophagitis, esophageal spasm, esophageal stricture, esophageal carcinoma, dyspepsia, indigestion, gastritis, gastric carcinoma, anorexia, nausea, enesis, gastroparesis, antral or pyloric edema, abdoninal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired imnmunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a cardiovascular disorder, such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation; an autoimmune/inflammatory disease, such as acquired immunodeficiency syndrome (AIDS), Addison's disease, adult respiratory distress syndrome, allergies, ankylosing spondylitis, amyloidosis, anemia, asthma, atherosclerosis, atherosclerotic plaque rupture, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflarrmation, osteoarthritis, degradation of articular cartilage, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia; a developmnental disorder, such as renal tubular acidosis, anemia, Cushing's syndrome, achondroplastic dwarfism, Duchenne and Becker muscular dystrophy, bone resorption, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, age-related macular degeneration, and sensorineural hearing loss; an epithelial disorder, such as dyshidrotic eczema, allergic contact dermatitis, keratosis pilaris, melasma, vitiligo, actinic keratosis, basal cell carcinoma, squamous cell carcinoma, seborrheic keratosis, folliculitis, herpes simplex, herpes zoster, varicella, candidiasis, dermatophytosis, scabies, insect bites, cherry angioma, keloid, dermatofibroma, acrochordons, urticaria, transient acantholytic dermatosis, xerosis, eczema, atopic dermatitis, contact dermatitis, hand eczema, nummular eczema, lichen simplex chronicus, asteatotic eczema, stasis dermatitis and stasis ulceration, seborrheic dermatitis, psoriasis, lichen planus, pityriasis rosea, impetigo, ecthyma, dermatophytosis, tinea versicolor, warts, acne vulgaris, acne rosacea, pemphigus vulgaris, pemphigus foliaceus, paraneoplastic pemphigus, bullous pemphigoid, herpes gestationis, dermatitis herpetiformis, linear IgA disease, epidermolysis bullosa acquisita, dermatonyositis, lupus erythematosus, scleroderma and morphea, erythroderma, alopecia, figurate skin lesions, telangiectasias, hypopigmentation, hyperpigientation, vesicles/bullae, exanthems, cutaneous drug reactions, papulonodular skin lesions, chronic non-healing wounds, photosensitivity diseases, epidermolysis bullosa simplex, epidermolytic hyperkeratosis, epidermolytic and nonepidermolytic palmoplantar keratoderma, ichthyosis bullosa of Siemens, ichthyosis exfoliativa, keratosis palmaris et plantaris, keratosis palnoplantaris, palmoplantar keratoderma, keratosis punctata, Meesmann's corneal dystrophy, pachyonychia congenita, white sponge nevus, steatocystoma multiplex, epidermal nevi/epidermolytic hyperkeratosis type, monilethrix, trichothiodystrophy, chronic hepatitis/cryptogenic cirrhosis, and colorectal hyperplasia; a neurological disorder, such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrore, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polynyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a reproductive disorder, such as infertility, including tubal disease, ovulatory defects, and endometriosis, a disorder of prolactin production, a disruption of the estrous cycle, a disruption of the menstrual cycle, polycystic ovary syndrome, ovarian hyperstimulation syndrome, an endometrial or ovarian tumor, a uterine fibroid, autoimmune disorders, an ectopic pregnancy, and teratogenesis; cancer of the breast, fibrocystic breast disease, and galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, and gynecomastia; an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma; a disorder associated with hypopituitarism including hypogonadism, Sheehan syndrome, diabetes insipidus, Kallman's disease, Hand-Schuller-Christian disease, Letterer-Siwe disease, sarcoidosis, empty sella syndrome, and dwarfism; a disorder associated with hyperpituitarism including acromegaly, giantism, and syndrome of inappropriate antidiuretic hormone (ADH) secretion (SIADM) often caused by benign adenoma; a disorder associated with hypothyroidism including goiter, myxedema, acute thyroiditis associated with bacterial infection, subacute thyroiditis associated with viral infection, autoimmune thyroiditis (Hashimoto's disease), and cretinism; a disorder associated with hyperthyroidism including thyrotoxicosis and its various forms, Grave's disease, pretibial myxedema, toxic multinodular goiter, thyroid carcinoma, and Plummer's disease; a disorder associated with hyperparathyroidism including Conn disease (chronic hypercalemia); a metabolic disorder such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, pentosuria phenylketonuria, pseudovitamin D-deficiency rickets; a disorder of carbohydrate metabolism such as congenital type UI dyserythropoietic anemia, diabetes, insulin-dependent diabetes mellitus, non-insulin-dependent diabetes mellitus, fructose-1,6-diphosphatase deficiency, galactosemia, glucagonoma, hereditary fructose intolerance, hypoglycemia, mannosidosis, neuraminidase deficiency, obesity, galactose epimerase deficiency, glycogen storage diseases, lysosomal storage diseases, fructosuria, pentosuria, and inherited abnormalities of pyruvate metabolism; a disorder of lipid metabolism such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolernia, hypercholesterolemia with hypertriglyceridemia, primary hypoalphalipoproteinemia, hypothyroidism, renal disease, liver disease, lecitiin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, lipid myopathies, and obesity; and a disorder of copper metabolism such as Menke's disease, Wilson's disease, and Ehlers-Danlos syndrome type IX; a pancreatic disorder such as Type I or Type II diabetes mellitus and associated complications; a disorder associated with the adrenals such as hyperplasia, carcinoma, or adenoma of the adrenal cortex, hypertension associated with alkalosis, amyloidosis, hypokalemia, Cushing's disease, Liddle's syndrome, and Arnold-Healy-Gordon syndrome, pheochromocytoma tumors, and Addison's disease; a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis; and, in men, Leydig cell deficiency, male climacteric phase, and germinal cell aplasia, a hypergonadal disorder associated with Leydig cell tumors, androgen resistance associated with absence of androgen receptors, syndrome of 5 α-reductase, and gynecomastia; a cancer such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; and an infection caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fungal agent classified as aspergillus, blastomyces, dermatophytes, cryptococcus, coccidioides, malasezzia, histoplasma, or other mycosis-causing fungal agent; and an infection caused by a parasite classified as plasmodium or malaria-causing, parasitic entamoeba, leishmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestrode such as tapeworm. Polynucleotides encoding PMMM may be used in Southern or northern analysis, dot blot, or other menbrane-based technologies; in PCR technologies; in dipstick, pin, and multiformat ELISA-like assays; and in microarrays utilizing fluids or tissues from patients to detect altered PMMM expression. Such qualitative or quantitative methods are well known in the art.

In a particular emnbodiment, polynucleotides encoding PMMM may be used in assays that detect the presence of associated disorders, particularly those mentioned above. Polynucleotides complementary to sequences encoding PMMM may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding PMMM in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of PMMM, a normal or standard profile for expression is established. This may be accomplished by combining body fluids or cell extracts taken from normal subjects, either animal or human, with a sequence, or a fragment thereof, encoding PMMM, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determnine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding PMMM may involve the use of PCR. These oligomers may be chemically synthesized, generated enzymatically, or produced in vitro. Oligomers will preferably contain a fragment of a polynucleotide encoding PMMM, or a fragment of a polynucleotide complementary to the polynucleotide encoding PMMM, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding PMMM may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding PMMM are used to amplify DNA using thepolymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These coniputer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For examuple, at least 16 common SNPs have been associated with non-insulin-dependent diabetes mellitus. SNPs are also useful for exaning differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenonics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in dimished clinical response to treatment with an anti-asthma drug that targets the 5-lipoxygenase pathway. Analysis of the distribution of SNPs in different populations is useful for investigating genetic drift, mutation, recomibination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of PMMM include radiolabeliug or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further enmbodiments, oligonucleotides or longer fragments derived from any of the polynucleotides descnbed herein may be used as elements on a microarray. The microarray can be used in transcript imaging techniques which monitor the relative expression levels of large numbers of genes simultaneously as described below. The microarray may also be used to identify genetic variants, nutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another enbodiment, PMMM, fragments of PMMM, or antibodies specific for PMMM may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizg the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genomewide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifing the number of expressed proteins and their relative abundance under given conditions and at a given time. A profile of a cell's proteome may thus be generated by separating and analyzing the polypeptides of a particular tissue or cell type. In one emnbodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for PMMM to quantify the levels of PMMM expression. In one emnbodiment, the antibodies are used as elements on a microarray, and protein expression levels are quantified by exposing the microarray to the sample and detecting the levels of protein bound to each array element (Lueking, A et al. (1999) Aaal. Biochem. 270:103-111; Mendoze, L. G. et al. (1999) Biotechniques 27:778-788). Detection maybe performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins fromthebiological sample are incubated with antibodies specific to the polypeptides of the present invention. The amount of protein recognized by the antibodies is quantified. The amount of protein in the treated biological sarmple is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding PMMM may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding PMMM on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, PMMM, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between PMMM and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with PMMM, or fragments thereof, and washed. Bound PMMM is then detected by methods well known in the art. Purified PMMM can also be coated directly onto plates for use in the aforementioned drug screening techniques. Alternatively, non-neutralizing antibodies can be used to capture the peptide and immobilize it on a solid support.

In another embodirent, one may use competitive drug screening assays in which neutralizing antibodies capable of binding PMMM specifically compete with a test compound for binding PMMM. In this manner, antibodies can be used to detect the presence of any peptide which shares one or more antigenic determinants with PMMM.

In additional embodiments, the nucleotide sequences which encode PMMM may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications, and publications mentioned above and below, including U.S. Ser. No. 60/322,196, U.S. Ser. No. 60/324,134, U.S. Ser. No. 60/327,233, U.S. Ser. No. 60/332,423, U.S. Ser. No. 60/334,145, U.S. Ser. No. 60/334,229, U.S. Ser. No. 60/337,451, U.S. Ser. No. 60/343,980, U.S. Ser. No. 60/346,198, U.S. Ser. No. 60/348,887, U.S. Ser. No. 60/351,928, and U.S. Ser. No. 60/366,837, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic. solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Anibion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (nvitrogen), PCMV-ICIS plasmid (Stratagene), pIGEN (Incyte Genomics, Palo Alto Calif.), pRARE (hncyte Genomics), or pINCY (hncyte Genomnics), or derivatives thereof. Recombinant plasmids were transformed into competent E. coli cells including XL1-Blue, XL1-BlueMRP, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasnids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsysterns Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer systemi cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosacchayomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (EMM)-based protein family databases such as PFAM, INCY, and TIGRFAM; and HMM-based protein domain databases such as SMART. Pull length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN mutisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithls used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:32-62. Fragments from about 20 to about 4000 nucleotides which are useful in hybridization and amplification technologies are described in Table 4, column 2.

IV. Identification and Editing of Coding Sequences from Genomic DNA

Putative protein modification and maintenance molecules were initially identified by running the Genscan gene identification program against public genoric sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a PASTA database of polynucleotide and polypeptide sequences. The maxinmm range of sequence for Genscan to analyze at once was set to 30 kb. To determine which of these Genscan predicted cDNA sequences encode protein modification and maintenance molecules, the encoded polypeptides were analyzed by querying against PFAM models for protein modification and maintenance molecules. Potential protein modification and maintenance molecules were also identified by homology to Incyte cDNA sequences that had been annotated as protein modification and maintenance molecules. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genoinic Sequence Data with cDNA Sequence Data

“Stitched” Sequences

Partial cDNA sequences were extended with exons predicted by the Genscan gene identification program described in Example IV. Partial cDNAs assenmbled as described in Example III were mapped to genornic DNA and parsed into clusters containing related cDNAs and Genscan exon predictions from one or more genomic sequences. Each cluster was analyzed using an algorithm based on graph theory and dynamic programnring to integrate cDNA and genomic information, generating possible splice variants that were subsequently confined, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genonic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eulraryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of PMMM Encoding Polynucleotides

The sequences which were used to assemble SEQ ID NO:32-62 were compared with sequences from the Incyte LIFESEQ database and public domain databases using BLAST and other implementations of the Smith-Waterman algorithm. Sequences from these databases that matched SEQ ID NO:32-62 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sanbrook and Russell, supra, ch. 7; Ausubel et al., supra, ch. 4).

Analogous computer techniques applying BLAST were used to search for identical or related molecules in databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding PMMM are analyzed with respect to the tissue sources from which they were derived. For exaipple, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; henic and immune system; liver; musculoskeletal system; nervous system; pancreas; respiratory system; sense organs; skin; stomatognathic system; unclassified/mixed; or urinary tract. The nuniber of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total nuniber of libraries across all categories. The resulting percentages reflect the tissue- and disease-specific expression of cDNA encoding PMMM. cDNA sequences and cDNA library/tissue information are found in the LIFESEQ GOLD database (Incyte Genonmics, Palo Alto Calif.).

VIII. Extension of PMMM Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or mnore, and to anneal to the target sequence at temperatures of about 68° C. to about 72° C. Any stretch of nucleotides which would result in hairpin structures and primer-primer dimerizations was avoided.

Selected human cDNA hbraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (hivitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1× TE and 0.5 μl of undiluted PCR product into each well of an opaque fluorimeter plate (Corming Costar, Acton Mass.), allowing the DNA to bind to the reagent. The plate was scanmed in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terninator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in PMMM Encoding Polynucleotides

Common DNA sequence variants known as single nucleotide polymorphisms (SNPs) were identified in SEQ ID NO:32-62 using the LIFESEQ database (Incyte Genomics). Sequences from the same gene were clustered together and assembled as described in Example III, allowing the identification of all sequence variants in the gene. Am algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contaminationby non-human sequences. A final set of filters removed duplicates and SNPs found in immunoglobulins or T-cell receptors.

Certain SNPs were selected for flrther characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not furter tested in the other three populations.

X. Labeling and Use of Individual Hybridization Probes

Hybridization probes derived from SEQ ID NO:32-62 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical mnenabrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for exarple, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or ohgomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray maybe assessed. In one embodiment, microarray preparation and usage is described in detail below.

Tissue or Cell Sample Preparation

Total RNA is isolated from tissue samples using the guanidinium thiocyanate method and poly(A)⁺RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM DATP, 500 AM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHI kits (Incyte Genomics). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thitty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleanedby ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Micro arrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first washbuffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X—Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit Rn-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte Genomics). Array elements that exhibit at least about a two-fold change in expression, a signal-to-background ratio of at least about 2.5, and an element spot size of at least about 40%, are considered to be differentially expressed.

Expression

For example, SEQ ID NO:40 showed decreased expression in peripheral blood mononuclear cells (PBMCs) treated with PMA and ionomycin versus untreated PBMCs as determined by microarray analysis. Peripheral blood mononuclear cells (PBMCs) are isolated from freshly obtained peripheral blood. PBMCs are stimulated in vitro with soluble PMA and ionomycin for 1, 2, 4, 8, and 20 hours. These treated cells are compared to untreated PBMCs kept in culture. Therefore, in various embodiments, SEQ ID NO:40 can be used for one or more of the following: i) monitoring treatment of immune disorders and related diseases and conditions, ii) diagnostic assays for immune disorders and related diseases and conditions, and iii) developing therapeutics and/or other treatments for immune disorders and related diseases and conditions.

In another example, SEQ ID NO:43 was differentially expressed in human breast tumor cells lines as compared to a nonmalignant breast epithelial cell line, MCF-10A. Histological and molecular evaluation of breast tumors reveals that the development of breast cancer evolves through a multi-step process whereby pre-malignant mammary epithelial cells undergo a relatively defined sequence of events leading to tumor formation. An early event in tumor development is ductal hyperplasia. Cells undergoing rapid neoplastic growth gradually progress to invasive carcinoma and become metastatic to the lung, bone, and potentially other organs. Several variables that may influence the process of tumor progression and malignant transformation include genetic factors, environmental factors, growth factors, and hormones. Based on the complexity of this process, it is critical to study a population of human mammary epithelial cells undergoing the process of malignant transformation, and to associate specific stages of progression with phenotypic and molecular characteristics. In a cross-comparison study, two cell lines out of nine tested exhibited differential expression as compared to controls. BT-20 is a breast carcinoma cell line derived in vitro from cells emigrating out of thin slices of the tumor mass isolated from a 74-year old female. MDA-mb-435S is a spindle shaped strain derived from the pleural effusion of a 31-year old female with metastatic, ductal adenocarcinoma of the breast. In this experiment, the expression of SEQ ID NO:43 was increased by at least two-fold in these breast tumor cell lines. Therefore, in various embodiments, SEQ ID NO:43 can be used for one or more of the following: i) monitoring treatment of breast cancer, ii) diagnostic assays for breast cancer, and iii) developing therapeutics and/or other treatments for breast cancer.

In another example, SEQ ID NO:43-44 were differentially expressed in three separate experiments in which human lung tumor cells were tested in a pair comparison with normal lung from the same donor. Lung cancers are divided into four histopathologically distinct groups. Three groups (squamous cell carcinoma, adenocarcinoma, and large cell carcinoma) are classified as non-small cell lung cancers (NSCLCs). The fourth group of cancers is referred to as small cell lung cancer (SCLC). Collectively, NSCLCs account for approximately 70% of cases while SCLCs account for approximately 18% of cases. The molecular and cellular biology underlying the development and progression of lung cancer are incompletely understood. Deletions on chromosome 3 are connmon in this disease and are thought to indicate the presence of a tumor suppressor gene in this region. Activating mutations in K-ras are commonly found in lung cancer and are the basis of one of the mouse models for the disease. Analysis of gene expression patterns associated with the development and progression of the disease will yield tremendous insight into the biology underlying this disease, and will lead to the development of improved diagnostics and therapeutics. In these experiments, the expression of SEQ ID NO:43-44 were increased by at least two-fold in the lung tumor cells as compared to the normal lung tissue cells from the same donor.

These experiments indicate that SEQ ID NO:43 and SEQ ID NO:44 exhibited significant differential expression patterns using microarray techniques. Therefore, in various embodiments, SEQ ID NO:43-44 canbe used for one or more of the following: i) monitoring treatment of lung cancer, ii) diagnostic assays for lung cancer, and iii) developing therapeutics and/or other treatments for lung cancer.

In another example, SEQ ID NO:45 was differentially expressed in human breast tumor cell lines compared to nonmalignant breast epithelial cell lines. Histological and molecular evaluation of breast tumors reveals that the development of breast cancer evolves through a multi-step process whereby pre-malignant mammary epithelial cells undergo a relatively defined sequence of events leading to tumor formation. An early event in tumor development is ductal hyperplasia. Cells undergoing rapid neoplastic growth gradually progress to invasive carcinoma and become metastatic to the lung, bone, and potentially other organs. Several variables that may influence the process of tumor progression and malignant transformation include genetic factors, environmental factors, growth factors, and hormones. Based on the complexity of this process, it is critical to study a population of human mammary epithelial cells undergoing the process of malignant transformation.

In one set of experiments, human primary epithelial breast cells (HMECs) isolated from a normal donor were compared to various types of breast cancer cell lines. Of six breast cancer cell lines tested, two of these cell lines, MCF-7 (breast adenocarcinoma) and SK-BR-3 (human breast adenocarcinoma, which is also tumorigenic in nude mice) were underexpressed in SEQ ID NO:45 by at least two-fold as compared to HMEC cells.

SEQ ID NO:45 was also underexpressed by at least two-fold in MCF-7 breast adeonocarcinoma cells as compared to nonmalignant MCF10A cells isolated from normal breast epithelial tissue.

These experiments indicate that SEQ ID NO:45 exhibits significant differential expression patterns using microarray techniques. Therefore, in various embodiments, SEQ ID NO:45 can be used for one or more of the following: i) monitoring treatment of breast cancer, ii) diagnostic assays for breast cancer, and iii) developing therapeutics and/or other treatments for breast cancer.

In another example, SEQ ID NO:49 showed differential expression in breast cancer tissue, as determined by microarray analysis. In order to better determine the molecular and phenotypic characteristics associated with different stages of breast cancer, breast carcinoma cell lines at various stages of tumor progression were compared to primary human breast epithelial cells. The breast carcinoma cell lines include MCF7, a breast adenocarcinoma cell line derived from the pleural effusion of a 69-year-old female; Sk-BR-3, a breast adenocarcinoma cell line isolated from a malignant pleural effusion of a 43-year-old female; and BT-20, a breast adenocarcinoma isolated in vitro from cells emigrating out of thin slices of a tumor mass isolated from a 74-year-old female. The primary mammary epithelial cell line HMEC was derived from normal human mammary tissue (Clonetics, San Diego, Calif.). All cell cultures were propagated in a chemically-defined medium, according to the supplier's recommendations and grown to 70-80% confluence prior to RNA isolation. The microarray experiments showed that expression of SEQ ID NO:49 was decreased by at least two fold in all three breast carcinoma lines (MCF7, Sk-BR-3, and BT20) relative to primary mammary epithelial cells. Therefore, in various enibodiments, SEQ ID NO:49 can be used for one or more of the following: i) monitoring treatment of breast cancer, ii) diagnostic assays for breast cancer, and iii) developing therapeutics and/or other treatments for breast cancer.

SEQ ID NO:49 also showed differential expression, as determined by microarray analysis, in liver C3A cells treated with one of the following steroids: beclomethasone, dexamethasone, progesterone, budesonide. The human C3A cell line is a clonal derivative of HepG2/C3 and has been established as an in vitro model of the mature human liver (Mickelson et al. (1995) Hepatology 22:866-875; Nagendra et al. (1997) Am J Physiol 272:G408-G416). SEQ ID NO:5 showed at least a two-fold decrease in expression at a minmum of two out of the three time points in early confluent C3A cells treated with beclomethasone, budesonide, dexamethasone, or betamethasone, for 1, 3, or 6 hours. These experiments indicate that SEQ ID NO:49 is useful in diagnostic assays for liver diseases and as a potential biological marker and therapeutic agent in the treatment of liver diseases and disorders. Therefore, in various embodiments, SEQ ID NO:49 can be used for one or more of the following: i) monitoring treatment of liver diseases and disorders, ii) diagnostic assays for liver diseases and disorders, and iii) developing therapeutics and/or other treatments for liver diseases and disorders.

In another example, SEQ ID NO:51 showed differential expression, as determined by microarray analysis, in Alzheimer's Disease (AD). In a comparison of cerebellum tissue from a 76-year-old male with severe AD to cerebellum tissue from a normal 67-year-old male, the expression of SEQ ID NO:51 was decreased at least two-fold. Therefore, in various embodiments, SEQ ID NO:51 can be used for one or more of the following: i) monitoring treatment of Alzheimer's Disease, ii) diagnostic assays for Alzheimer's Disease, and iii) developing therapeutics and/or other treatments for Alzheimer's Disease.

SEQ ID NO:51 also showed differential expression associated with colon cancer, as determined by microarray analysis. Normal colon tissue was compared to colon tumor tissue from a 67-year-old donor with moderately differentiated adenocarcinoma. The expression of SEQ ID NO:51 was decreased at least two-fold in the tumor tissue as compared to the normal tissue. Therefore, in various embodiments, SEQ ID NO:51 can be used for one or more of the following: i) monitoring treatment of colon cancer, ii) diagnostic assays for colon cancer, and iii) developing therapeutics and/or other treatments for colon cancer.

In another example, the expression of SEQ ID NO:56 in a primary prostate epithelial cell line isolated from a normal donor, PrEC, was compared to that in three prostate carcinoma cell lines. DU 145 is a prostate carcinoma cell line isolated from a metastatic site in the brain of a 69 year old male with widespread metastatic prostate carcinoma. DU 145 has no detectable sensitivity to hormones; forms colonies in semi-solid medium, is only weakly positive for acid phosphatase, and is negative for prostate specific antigen. LNCaP is a prostate carcinoma cell line isolated from a lymph node biopsy of a 50 year old male with metastatic prostate carcinoma. LNCaP cells express prostate specific antigens, produce prostatic acid phosphatase, and express androgen receptors. PC-3 is a prostate adenocarcinoma cell line isolated from a metastatic site in the bone of a 62 year old male with grade IV prostate adenocarcinoma. The expression of SEQ ID NO:56 was increased by at least two-fold in DU 145 cells grown under restrictive conditions as compared to PrEC cells grown under restrictive conditions. Therefore, in various embodiments, SEQ ID NO:56 can be used for one or more of the following: i) monitoring treatment of prostate cancer, ii) diagnostic assays for prostate cancer, and iii) developing therapeutics and/or other treatments for prostate cancer.

In another example, SEQ ID NO:58, SEQ ID NO:59 and SEQ ID NO:60 showed differential expression associated with breast cancer, as determined by nicroarray analysis. The gene expression profile of a nonmalignant mammary epithelial cell line was compared to the gene expression profiles of breast carcinoma lines at different stages of tumor progression. Cell lines compared included: a) BT-20, a breast carcinoma cell line derived in vitro from the cells emigrating out of thin slices of tumor mass isolated from a 74-year-old female, b) BT-474, a breast ductal carcinoma cell line that was isolated from a solid, invasive ductal carcinoma of the breast obtained from a 60-year-old woman, c) BT-483, a breast ductal carcinoma cell line that was isolated from a papillary invasive ductal tumor obtained from a 23-year-old normal, menstruating, parous female with a family history of breast cancer, d) Hs578T, a breast ductal carcinoma cell line isolated from a 74-year-old female with breast carcinoma, e) MCF7, a nonmalignant breast adenocarcinoma cell line isolated from the pleural effusion of a 69-year-old female, f) MCF-10A, a breast mammary gland (luminal ductal characteristics) cell line isolated. from a 36-year-old woman with fibrocystic breast disease, g) MDA-mb-435S, a spindle-shaped strain that evolved from the parent line (435) isolated by R. Cailleau from pleural effusion of a 31-year-old female with metastatic, ductal adenocarcinoma of the breast, h) Sk-BR-3, a breast adenocarcinoma cell line isolated from a malignant pleural effusion of a 43-year-old female, i) T-47D, a breast carcinoma cell line isolated from a pleural effusion obtained from a 54-year-old female with an infiltrating ductal carcinoma of the breast and j) HMEC, a primary breast epithelial cell line isolated from a normal donor. SEQ ID NO:58 expression was reduced by at least two-fold in BT20 and MCF7 cells as compared to HMEC cells. The expression of SEQ ID NO:59 was decreased by at least two-fold in carcinoma cell lines BT20, Sk-BR-3, T-47D, MDA-mb-435S and MCF7 as compared to HMEC cells. SEQ ID NO:60 expression was upregulated by at least two-fold in the carcinoma cell line Hs578T as compared to the HMEC cell line. Therefore, in various embodiments, SEQ ID NO:58, SEQ ID NO:59 and SEQ ID NO:60 can be used for one or more of the following: i) monitoring treatment of breast cancer, ii) diagnostic assays for breast cancer, and iii) developing therapeutics and/or other treatments for breast cancer.

In another example, SEQ ID NO:60 showed differential expression associated with lung cancer, as determined by microarray analysis. Expression was compared in matched samples of normal and lung tumor tissue from individual donors. Tissue samples were provided by the Roy Castle International Centre for Lung Cancer Research. SEQ ID NO:60 expression was upregulated by at least two-fold in lung squamous cell carcinoma tissue derived from a 68-year-old female donor as compared to normal lung tissue from the same donor. Therefore, in various enibodiments, SEQ ID NO:60 can be used for one or more of the following: i) monitoring treatment of lung cancer, ii) diagnostic assays for lung cancer, and iii) developing therapeutics and/or other treatments for lung cancer.

In another example, SEQ ID NO:58 and SEQ ID NO:59 showed differential expression associated with ovarian cancer, as determined by microarray analysis. A normal ovary from a 79 year-old female donor was compared to an ovarian tumor from the same donor (Huntsman Cancer Institute, Salt Lake City, Utah). The expression of SEQ ID NO:58 and SEQ ID NO:59 was decreased by at least two-fold in the tumor tissue as compared to the normal tissue. Therefore, SEQ ID NO:58 and SEQ ID NO:59 are useful in monitoring treatment of, and diagnostic assays for ovarian cancer. Therefore, in various embodiments, SEQ ID NO:58-59 can be used for one or more of the following: i) monitoring treatment of ovarian cancer, ii) diagnostic assays for ovarian cancer, and iii) developing therapeutics and/or other treatments for ovarian cancer.

In another example, SEQ ID NO:59 showed differential expression associated with steroid hormone responses, as determined by microarray analysis. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with α-fetoprotein iii) conversion of ammonia to urea and glutamine; iv) metabolism of aromatic amino acids; and v) proliferation in glucose-free and insulin-free medium. The C3A cell line is now well established as an in vitro model of the mature human liver (Mickelson et al. (1995) Hepatology 22:866-875; Nagendra et al. (1997) Am J Physiol 272:G408-G416). Early Confluent C3A cells were treated with progesterone or budenoside at 1, 10, and 100 μM for 1, 3, and 6 hours. The treated cells were compared to untreated early confluent C3A cells. At each of the time points, the expression of SEQ ID NO:59 was decreased by at least two-fold in C3A cells treated with 10 or 100 μM budenoside, and in C3A cells treated wth 10 μM progesterone. Therefore, SEQ ID NO:59 may be useful in monitoring of, and diagnostic assays for steroid hormone-induced responses. Therefore, in various embodiments, SEQ ID NO:59 can be used for one or more of the following: i) monitoring treatment of steroid hormone-induced responses, ii) diagnostic assays for steroid hormone-induced responses, and iii) developing therapeutics and/or other treatments for steroid hormone-induced responses.

In another example, SEQ ID NO:61 showed differential expression associated with lung cancer, as determined by microarray analysis. Pair comparisons of lung tumor tissue and microscopically-normal tissue from the same donor were made. The expression of SEQ ID NO:61 was increased by at least two-fold in lung squamous cell carcinoma tissue from a 68 year-old female as compared to normal lung tissue from the same donor (Roy Castle International Centre for Lung Cancer Research, Liverpool, UK). Therefore, in various emnbodiments, SEQ ID NO:61 can be used for one or more of the following: i) monitoring treatment of lung cancer, ii) diagnostic assays for lung cancer, and iii) developing therapeutics and/or other treatments for lung cancer.

XII. Complementary Polynucleotides

Sequences complementary to the PMMM-encoding sequences, or any parts thereof, are used to detect, decrease, or inhibit expression of naturally occurring PMMM. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of PMMM. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the PMMM-encoding transcript.

XIII. Expression of PMMM

Expression and purification of PMMM is achieved using bacterial or virus-based expression systems. For expression of PMMM in bacteria, cDNA is subcloned into an appropriate vector containing an antibiotic resistance gene and an inducible promoter that directs high levels of cDNA transcription Examples of such promoters include, but are not limited to, the trp-lac (tac) hybrid promoter and the T5 or T7 bacteriophage promoter in conjunction with the lac operator regulatory element. Reconmbinant vectors are transformed into suitable bacterial hosts, e.g., BL21(DE3). Antibiotic resistant bacteria express PMMM upon induction with isopropyl beta-D-thiogalactopyranoside (IPTG). Expression of PMMM in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding PMMM by either homologous reconmbination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, PMMM is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from PMMM at specifically engineered sites. FLAG, an 8-amino acid peptide, enables immunoaffnity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified PMMM obtained by these methods can be used directly in the assays shown in Examples XVII, XVIII, XIX, and XX, where applicable.

XIV. Functional Assays

PMMM function is assessed by expressing the sequences encoding PMMM at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma menibrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of PMMM on gene expression can be assessed using highly purified populations of cells transfected with sequences encoding PMMM and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding PMMM and other genes of interest can be analyzed by northern analysis or microarray techniques.

XV. Production of PMMM Specific Antibodies

PMMM substantially purified using polyacrylarnide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the PMMM aniino acid sequence is analyzed using LASERGENE software (DNASTAR) to determine regions of high inununogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., sutpra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are imnmunized with the oligopeptide-KLH cornplex in complete Freund's adjuvant. Resulting antisera are tested for antipeptide and anti-PMMM activity by, for example, binding the peptide or PMMM to a substrate, blocking with 1% BSA, reacting with rabbit antisera, washing, and reacting with radio-iodinated goat anti-rabbit IgG.

XVI. Purification of Naturally Occurring PMMM Using Specific Antibodies

Naturally occurring or recombinant PMMM is substantially purified by imnunoaffinity chromatography using antibodies specific for PMMM. An imnmnoaffinity column is constructed by covalently coupling anti-PMMM antibody to an activated chromatographic resin, such as CNBr-activated SEPHAROSE (Amersham Biosciences). After the coupling, the resin is blocked and washed according to the manufacturer's instructions.

Media containing PMMM are passed over the imnunoaffinity column, and the column is washed under conditions that allow the preferential absorb ance of PMMM (e.g., high ionic strength buffers in the presence of detergent). The column is eluted under conditions that disrupt antibody/PMMM binding (e.g., a buffer of pH 2 to pH 3, or a high concentration of a chaotrope, such as urea or thiocyanate ion), and PMMM is collected.

XVII. Identification of Molecules Which Interact with PMMM

PMMM, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled PMMM, washed, and any wells with labeled PMMM complex are assayed. Data obtained using different concentrations of PMMM are used to calculate values for the number, affinity, and association of PMMM with the candidate molecules.

Alternatively, molecules interacting with PMMM are analyzed using the yeast two-hybrid system as described in Fields, S. and O. Song (1989; Nature 340:245-246), or using commercially available kits based on the two-hybrid system, such as the MATCHMAKER system (Clontech).

PMMM may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of PMMM Activity

PMMM activity can be demonstrated using a generic imamnoblotting strategy or through a variety of specific activity assays, some of which are outlined below. As a general approach, cell lines or tissues transformed with a vector containing PMMM coding sequences can be assayed for PMMM activity by immunoblotting. Transformed cells are denatured in SDS in the presence of b-mercaptoethanol, nucleic acids are removed by ethanol precipitation, and proteins are purified by acetone precipitation. Pellets are resuspended in 20 nM Tris buffer at pH 7.5 and incubated with Protein G-Sepharose pre-coated with an antibody specific for PMMM. After washing, the Sepharose beads are boiled in electrophoresis sample buffer, and the eluted proteins subjected to SDS-PAGE. The SDS-PAGE is transferred to a membrane for immunoblotting, and the PMMM activity is assessed by visualizing and quantifying bands on the blot using the antibody specific for PMMM as the primary antibody and ¹²⁵I-labeled IgG specific for the primary antibody as the secondary antibody.

PMMM kinase activity is measured by quantifying the phosphorylation of a protein substrate by PMMM in the presence of gamma-labeled ³²P-ATP. PMMM is incubated with the protein substrate, ³²P-ATP, and an appropriate kinase buffer. The ³²P incorporated into the substrate is separated from free ³²P-ATP by electrophoresis and the incorporated ³²P is counted using a radioisotope counter. The amount of incorporated ³²P is proportional to the activity of PMMM. A determination of the specific amino acid residue phosphorylated is made by phosphoamino acid analysis of the hydrolyzed protein.

In one alternative, PMMM activity is demonstrated by a test for galactosyltransferase activity. This can be determined by measuring the transfer of radiolabeled galactose from UDP-galactose to a GlcNAc-terminated oligosaccharide chain (Kolbinger, F. et al. (1998) J. Biol. Chem. 273:58-65). The sample is incubated with 14 μl of assay stock solution (180 mM sodium cacodylate, pH 6.5, 1 mg/ml bovine serum albumin, 0.26 mM UDP-galactose, 2 μl of UDP-[³H]galactose), 1 μl of MnCl₂ (500 mM), and 2.5 μl of GlcNAcβO-(CH₂)₈—CO₂Me (37 mg/ml in dimethyl sulfoxide) for 60 minutes at 37° C. The reaction is quenched by the addition of 1 ml of water and loaded on a C18 Sep-Pak cartridge (Waters), and the column is washed twice with 5 ml of water to remove unreacted UDP-[³H]galactose. The [³H]galactosylated GlcNAcβO-(CH₂)₈—CO₂Me remains bound to the column during the water washes and is eluted with 5 ml of methanol. Radioactivity in the eluted material is measured by liquid scintillation counting and is proportional to galactosyltransferase activity in the starting sample.

PMMM phosphatase activity is measured by the hydrolysis of p-nitrophenyl phosphate (PNPP). PMMM is incubated together with PNPP in HEPES buffer, pH 7.5, in the presence of 0.1% β-mercaptoethanol at 37° C. for 60 min. The reaction is stopped by the addition of 6 ml of 10 N NaOH and the increase inlight absorbance at 410 nm resulting fromthe hydrolysis of PNPP is measured using a spectrophotometer. The increase in light absorbance is proportional to the activity of PMMM in the assay (Diamond, R. H. et al. (1994) Mol. Cell. Biol. 14:3752-3762).

In the alternative, PMMM phosphatase activity is determined by measuring the amount of phosphate removed from a phosphorylated protein substrate. Reactions are performed with 2 or 4 nM enzye in a final volume of 30 μl containing 60 mM Tris, pH 7.6, 1 mM EDTA, 1 mM EGTA, 0.1% 2-mercaptoethanol and 10 μM substrate, ³²P-labeled on serine/threonine or tyrosine, as appropriate. Reactions are initiated with substrate and incubated at 30° C. for 10-15 min. Reactions are quenched with 450 μl of 4% (wv) activated charcoal in 0.6 M HCl, 90 mM Na₄P₂O₇, and 2 nM NaH₂PO₄, then centrifuged at 12,000×g for 5 min. Acid-soluble ³²Pi is quantified by liquid scintillation counting (Sinclair, C. et al. (1999) J. Biol. Chem. 274:23666-23672).

PMMM protease activity is measured by the hydrolysis of appropriate synthetic peptide substrates conjugated with various chromogenic molecules in which the degree of hydrolysis is quantified by spectrophotometric (or fluorometric) absorption of the released chromophore (Beynon, R. J. and J. S. Bond (1994) Proteolytic Enzymes: A Practical Approach, Oxford University Press, New York, N.Y., pp. 25-55). Peptide substrates are designed according to the category of protease activity as endopeptidase (serine, cysteine, aspartic proteases, or metalloproteases), aminopeptidase (leucine aminopeptidase), or carboxypeptidase (carboxypeptidases A and B, procollagen C-proteinase). Commonly used chromogens are 2-naphthylamine, 4-nitroaniline, and furylacrylic acid. Assays are performed at ambient temperature and contain an aliquot of the enzyme and the appropriate substrate in a suitable buffer. Reactions are carried out in an optical cuvette, and the increase/decrease in absorbance of the chromogen released during hydrolysis of the peptide substrate is measured. The change in absorbance is proportional to the enzyme activity in the assay.

In the alternative, an assay for PMMM protease activity takes advantage of fluorescence resonance energy transfer (FRET) that occurs when one donor and one acceptor fluorophore with an appropriate spectral overlap are in close proximity. A flexible peptide linker containing a cleavage site specific for PMMM is fused between a red-shifted variant (RSGFP4) and a blue variant (BFP5) of Green Fluorescent Protein. This fusion protein has spectral properties that suggest energy transfer is occurring from BFP5 to RSGFP4. When the fusion protein is incubated with PMMM, the substrate is cleaved, and the two fluorescent proteins dissociate. This is accompanied by a marked decrease in energy transfer which is quantified by comparing the emission spectra before and after the addition of PMMM (Mitra, R. D. et al (1996) Gene 173:13-17). This assay can also be performed in living cells. In this case the fluorescent substrate protein is expressed constitutively in cells and PMMM is introduced on an inducible vector so that FRET can be monitored in the presence and absence of PMMM (Sagot, I. et al (1999) FEBS Letters 447:53-57).

An assay for ubiquitin hydrolase activity measures the hydrolysis of a ubiquitin precursor. The assay is performed at ambient temperature and contains an aliquot of PMMM and the appropriate substrate in a suitable buffer. Chemically synthesized human ubiquitin-valine may be used as substrate. Cleavage of the C-terminal valine residue from the substrate is monitored by capillary electrophoresis (Franlin, K. et al. (1997) Anal. Biochem. 247:305-309).

PMMM protease inhibitor activity for alpha 2-HS-glycoprotein (AHSG) can be measured as a decrease in osteogenic activity in dexamethasone-treated rat bone marrow cell cultures (dex-RBMC). Assays are carried out in 96-well culture plates containing minimal essential medium supplemented with 15% fetal bovine serum, ascorbic acid (50 mg/ml), antibiotics (100 mg/ml penicillin G, 50 mg/ml gentamicin, 0.3 mg/ml fungizone), 10 mM B-glycerophosphate, dexamethasone (10⁻⁸ M) and various concentrations of PMMM for 12-14 days. Mineralized tissue formation in the cultures is quantified by measuring the absorbance at 525 nm using a 96-well plate reader (Binkert, C. et al. (1999) J. Biol. Chem 274:28514-28520).

PMMM protease inhibitor activity for inter-alpha-trypsin inhibitor (ITI) can be measured by a continuous spectrophotometric rate determination of trypsin activity. The assay is performed at ambient temperature in a quartz cuvette in pH 7.6 assay buffer containing 63 mM sodium phosphate, 0.23 mM N a-benzoyle-L-arginine ethyl ester, 0.06 mM hydrochloric acid, 100 units trypsin, and various concentrations of PMMM. Immediately after mixing by inversion, the increase in A_{253 nm} is recorded for approximately 5 minutes and the enzyme activity is calculated (Bergmeyer, H. U. et al. (1974) Meth. Enzym. Anal. 1:515-516).

PMMM isomerase activity such as peptidyl prolyl cis/trans isomerase activity can be assayed by an enzyme assay described by Rahfeld, J. U., et al. (1994; FEBS Lett. 352:180-184). The assay is performed at 10° C. in 35 mM HEPES buffer, pH 7.8, containing chymotrypsin (0.5 mg/ml) and PMMM at a variety of concentrations. Under these assay conditions, the substrate, Suc-Ala-Xaa-Pro-Phe-4-NA, is in equilibrium with respect to the prolyl bond, with 80-95% in trans and 5-20% in cis conformation. An aliquot (2 ml) of the substrate dissolved in dimethyl sulfoxide (10 mg/ml) is added to the reaction mixture described above. Only the cis isomer of the substrate is a substrate for cleavage by chymotrypsin. Thus, as the substrate is isomerized by PMMM, the product is cleaved by chymotrypsin to produce 4-nitroanilide, which is detected by it's absorbance at 390 nm. 4-nitroanilide appears in a time-dependent and a PMMM concentration-dependent manner.

PMMM galactosyltransferase activity can be determined by measuring the transfer of radiolabeled galactose from UDP-galactose to a GlcNAc-terminated oligosaccharide chain (Kolbinger, F. et al. (1998) J. Biol. Chemn 273:58-65). The sample is incubated with 14 μl of assay stock solution (180 mM sodium cacodylate, pH 6.5, 1 mg/ml bovine serum albumin, 0.26 mM UDP-galactose, 2 μl of UDP-[³H]galactose), 1 μl of MnCl₂ (500 mM), and 2.5 μl of GlcNAcβO-(CH₂)₈—CO₂Me (37 mg/ml in dimethyl sulfoxide) for 60 minutes at 37° C. The reaction is quenched by the addition of 1 ml of water and loaded on a C18 Sep-Pak cartridge (Waters), and the column is washed twice with 5 ml of water to remove unreacted UDP-[³H]galactose. The [³H]galactosylated GlcNAcβO-(CH₂)₈—CO₂Me remains bound to the column during the water washes and is eluted with 5 ml of methanol. Radioactivity in the eluted material is measured by liquid scintillation counting and is proportional to galactosyltransferase activity in the starting sample.

PMMM induction by heat or toxins may be demonstrated using primary cultures of human fibroblasts or human cell lines such as CCL-13, HEK293, or HEP G2 (ATCC). To heat induce PMMM expression, aliquots of cells are incubated at 42° C. for 15, 30, or 60 minutes. Control aliquots are incubated at 37° C. for the same time periods. To induce PMMM expression by toxins, aliquots of cells are treated with 100 μM arsenite or 20 mM azetidine-2-carboxylic acid for 0, 3, 6, or 12 hours. After exposure to heat, arsenite, or the amino acid analogue, samples of the treated cells are harvested and cell lysates prepared for analysis by western blot Cells are lysed in lysis buffer containing 1% Nonidet P-40, 0.15 M NaCl, 50 mM Tris-HCl, 5 mM EDTA, 2 mM N-ethylmaleimide, 2 mM phenylmethylsulfonyl fluoride, 1 mg/ml leupeptin, and 1 mg/ml pepstatin. Twenty micrograms of the cell lysate is separated on an 8% SDS-PAGE gel and transferred to a membrane. After blocking with 5% nonfat dry milk/phosphate-buffered saline for 1 h, the membrane is incubated overnight at 4° C. or at room temperature for 2-4 hours with an appropriate dilution of anti-PMMM serum in 2% nonfat dry milk/phosphate-buffered saline. The membrane is then washed and incubated with a 1:1000 dilution of horseradish peroxidase-conjugated goat anti-rabbit IgG in 2% dry milk/phosphate-buffered saline. After washing with 0.1% Tween 20 in phosphate-buffered saline, the PMMM protein is detected and compared to controls using chemiluminescence.

PMMM lysyl hydroxylase activity is determined by measuring the production of hydroxy[¹⁴C]lysine from [¹⁴C]lysine. Radiolabeled protocollagen is incubated with PMMM in buffer containing ascorbic acid, iron sulfate, dithiothreitol, bovine serum albumin, and catalase. Following a 30 minute incubation, the reaction is stopped by the addition of acetone, and centrifuged. The sedimented material is dried, and the hydroxy[¹⁴C]lysine is converted to [¹⁴C]formaldehyde by oxidation with periodate, and then extracted into toluene. The amount of ¹⁴C extracted into toluene is quantified by scintillation counting, and is proportional to the activity of PMMM in the sample (Kivirikko, K., and R. Myllyla (1982) Methods Enzymol. 82:245-304).

XIX. Identification of PMMM Substrates

Phage display libraries can be used to identify optimal substrate sequences for PMMM. A random hexamer followed by a linker and a known antibody epitope is cloned as an N-terminal extension of gene III in a filamentous phage library. Gene III codes for a coat protein, and the epitope will be displayed on the surface of each phage particle. The library is incubated with PMMM under proteolytic conditions so that the epitope will be removed if the hexamer codes for a PMMM cleavage site. An antibody that recognizes the epitope is added along with immobilized protein A. Uncleaved phage, which still bear the epitope, are removed by centrifugation. Phage in the supernatant are then amplified and undergo several more rounds of screening. Individual phage clones are then isolated and sequenced. Reaction kinetics for these peptide substrates can be studied using an assay in Example XVIII, and an optimal cleavage sequence can be derived (Ke, S. H. et al. (1997) J. Biol. Chem. 272:16603-16609).

To screen for in vivo PMMM substrates, this method can be expanded to screen a cDNA expression library displayed on the surface of phage particles (T7SELECT10-3 Phage display vector, Novagen, Madison, Wis.) or yeast cells (pYD1 yeast display vector kit, Invitrogen, Carlsbad, Calif.). In this case, entire cDNAs are fused between Gene III and the appropriate epitope.

XX. Identification of PMMM Inhibitors

Compounds to be tested are arrayed in the wells of a multi-well plate in varying concentrations along with an appropriate buffer and substrate, as described in the assays in Example XVIII. PMMM activity is measured for each well and the ability of each compound to inhibit PMMM activity can be determined, as well as the dose-response kinetics. This assay could also be used to identify molecules which enhance PMMM activity.

In the alternative, phage display libraries can be used to screen for peptide PMMM inhibitors. Candidates are found among peptides which bind tightly to a protease. In this case, multi-well plate wells are coated with PMMM and incubated with a random peptide phage display library or a cyclic peptide library (Koivunen, E. et al. (1999) Nature Biotech 17:768-774). Unbound phage are washed away and selected phage amplified and rescreened for several more rounds. Candidates are tested for PMMM inhibitory activity using an assay described in Example XVIII.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elertznts from one or more other embodiments. Such combinations can forn a number of embodiments within the scope of the invention. It is intended that the scope of the invention be defined by the following claims and their equivalents.

TABLE 1

| | | | | Incyte | |
|---|---|---|---|---|---|
| | Polypeptide | Incyte | Polynucleotide | Polynucleotide | Incyte Full Length |
| Incyte Project ID | SEQ ID NO: | Polypeptide ID | SEQ ID NO: | ID | Clones |
| 8268274 | 1 | 8268274CD1 | 32 | 8268274CB1 | |
| 7500515 | 2 | 7500515CD1 | 33 | 7500515CB1 | 90020436CA2 |
| 2256826 | 3 | 2256826CD1 | 34 | 2256826CB1 | |
| 7686186 | 4 | 7686186CD1 | 35 | 7686186CB1 | |
| 72617436 | 5 | 72617436CD1 | 36 | 72617436CB1 | |
| 7501945 | 6 | 7501945CD1 | 37 | 7501945CB1 | |
| 7500264 | 7 | 7500264CD1 | 38 | 7500264CB1 | |
| 7499935 | 8 | 7499935CD1 | 39 | 7499935CB1 | |
| 7982285 | 9 | 7982285CD1 | 40 | 7982285CB1 | 4872051CA2 |
| 7758505 | 10 | 7758505CD1 | 41 | 7758505CB1 | |
| 6885756 | 11 | 6885756CD1 | 42 | 6885756CB1 | |
| 7500748 | 12 | 7500748CD1 | 43 | 7500748CB1 | |
| 7500749 | 13 | 7500749CD1 | 44 | 7500749CB1 | |
| 7503401 | 14 | 7503401CD1 | 45 | 7503401CB1 | 2774614CA2 |
| 7503485 | 15 | 7503485CD1 | 46 | 7503485CB1 | 5500371CA2 |
| 7504076 | 16 | 7504076CD1 | 47 | 7504076CB1 | 90173111CA2, |
| | | | | | 90173203CA2, |
| | | | | | 90173227CA2 |
| 7500926 | 17 | 7500926CD1 | 48 | 7500926CB1 | 90205586CA2 |
| 7503216 | 18 | 7503216CD1 | 49 | 7503216CB1 | 6440464CA2 |
| 7503233 | 19 | 7503233CD1 | 50 | 7503233CB1 | |
| 7726576 | 20 | 7726576CD1 | 51 | 7726576CB1 | |
| 7503507 | 21 | 7503507CD1 | 52 | 7503507CB1 | |
| 7503506 | 22 | 7503506CD1 | 53 | 7503506CB1 | 90069502CA2 |
| 7503509 | 23 | 7503509CD1 | 54 | 7503509CB1 | 90208262CA2 |
| 7505800 | 24 | 7505800CD1 | 55 | 7505800CB1 | 3475431CA2 |
| 7503141 | 25 | 7503141CD1 | 56 | 7503141CB1 | |
| 7500362 | 26 | 7500362CD1 | 57 | 7500362CB1 | |
| 7503328 | 27 | 7503328CD1 | 58 | 7503328CB1 | |
| 7510464 | 28 | 7510464CD1 | 59 | 7510464CB1 | |
| 7510394 | 29 | 7510394CD1 | 60 | 7510394CB1 | |
| 7500745 | 30 | 7500745CD1 | 61 | 7500745CB1 | |
| 7500929 | 31 | 7500929CD1 | 62 | 7500929CB1 | |

TABLE 2

| | | GenBank | | |
|---|---|---|---|---|
| | Incyte | ID NO: or | | |
| Polypeptide | Polypeptide | PROTEOME | Probability | |
| SEQ ID NO: | ID | ID NO: | Score | Annotation |
| 1 | 8268274CD1 | g10441427 | 4.10E−126 | [Drosophila melanogaster] Partner of Paired |
| | | | | Raj, L. et al. (2000) Targeted localized degradation of Paired protein in |
| | | | | Drosophila development. Curr. Biol. 10: 1265-1272 |
| 2 | 7500515CD1 | g4096840 | 0.0 | [Homo sapiens] inter-alpha-trypsin inhibitor family heavy chain-related protein |
| | | | | Saguchi K. et al. (1996) Isolation and characterization of the human inter-alpha- |
| | | | | trypsin inhibitor family heavy chain-related protein (IHRP) gene (ITIHL1). J. |
| | | | | Biochem. 119: 898-905 |
| 3 | 2256826CD1 | g5919219 | 1.20E−188 | [Homo sapiens] leucine-rich repeats containing F-box protein FBL3 |
| | | | | Ilyin, G. P. et al. (2000) cDNA cloning and expression analysis of new members |
| | | | | of the mammalian F-box protein family. Genomics 67: 40-47 |
| 4 | 7686186CD1 | g11994498 | 5.70E−72 | [Arabidopsis thaliana] DegP protease precursor |
| | | | | Kaneko, T. et al. (2000) Structural analysis of Arabidopsis thaliana chromosome |
| | | | | 3. II. Sequence features of the 4,251,695 bp regions covered by 90 P1, TAC and |
| | | | | BAC clones. DNA Res. 7: 217-221 |
| 5 | 72617436CD1 | g2190297 | 1.10E−45 | [Oryzias latipes] choriolysin H |
| | | | | Yasumasu, S. et al. (1996) Eur. J. Biochem. 237: 752-758 |
| | | | | Different exon-intron organizations of the genes for two astacin-like proteases, |
| | | | | high choriolytic enzyme (choriolysin H) and low choriolytic enzyme (choriolysin |
| | | | | L), the constituents of the fish hatching enzyme. |
| 6 | 7501945CD1 | g213504 | 5.40E−37 | [Oryzias latipes] protease |
| | | | | Yasumasu, S. et al. (1992) Dev. Biol. 153: 250-258 |
| | | | | Isolation of cDNAs for LCE and HCE, two constituent proteases of the hatching |
| | | | | enzyme of Oryzias Latipes, and concurrent expression of their mRNAs during |
| | | | | development |
| 7 | 7500264CD1 | g2924601 | 7.60E−12 | [Homo sapiens] hepatocyte growth factor activator inhibitor |
| | | | | Shimomura, T. et al. (1997) J. Biol. Chem. 272: 6370-6376 |
| | | | | Hepatocyte growth factor activator inhibitor, a novel Kunitz-type serine protease |
| | | | | inhibitor. |
| 8 | 7499935CD1 | g189842 | 7.00E−247 | [Homo sapiens] prolidase |
| | | | | Endo, F. et al. (1989) J. Biol. Chem. 264: 4476-4481 |
| | | | | Primary structure and gene localization of human prolidase. |
| 9 | 7982285CD1 | g6567172 | 9.70E−102 | [Mus musculus] mDj10 |
| | | | | Ohtsuka K, and Hata M. (2000) Cell Stress Chaperones 5: 98-112 |
| | | | | Mammalian HSP40/DNAJ homologs: cloning of novel cDNAs and a proposal for |
| | | | | their classification and nomenclature. |
| 10 | 7758505CD1 | g1167860 | 1.10E−51 | [Spodoptera frugiperda] Endoprotease FURIN |
| 11 | 6885756CD1 | g14994718 | 3.20E−103 | [Mus musculus] (AF393638) deubiquitinating enzyme 2A |
| | | | | Baek, K.-H. et al. (2001) Blood 98: 636-42 |
| 12 | 7500748CD1 | g14456615 | 5.40E−280 | [Homo sapiens] phosphatidyl inositol glycan class T |
| | | | | Ohishi, K. et al. (2001) PIG-S and PIG-T, essential for GPI-anchor attachment to |
| | | | | proteins, form a complex with GAA1 and GPI8. EMBO J. 20: 4088-4098 |
| 13 | 7500749CD1 | g14456615 | 4.70E−261 | [Homo sapiens] phosphatidyl inositol glycan class T |
| | | | | Ohishi, K. et al. (supra) |
| 14 | 7503401CD1 | g292031 | 1.30E−117 | [Homo sapiens] farnesyl-protein transferase alpha-subunit |
| | | | | Omer, C. A., et al. (1993) Biochemistry 32: 5167-76 |
| | | | | Characterization of recombinant human farnesyl-protein transferase: Cloning, |
| | | | | expression, farnesyl diphosphate binding and functional homology with yeast |
| | | | | prenyl-protein transferases. |
| | 7503401CD1 | 335360\|FNTA | 1.10E−118 | [Homo sapiens][Transferase][Cytoplasmic] Alpha subunit of CAAX |
| | | | | farnesyltransferase (FPTase) and geranylgeranyltransferase type-I (GGTase-I), |
| | | | | transfers farnesyl and geranylgeranyl groups to proteins |
| 15 | 7503485CD1 | g11036950 | 3.30E−60 | [Homo sapiens] ubiquitin-conjugating enzyme HRGB |
| | | 338766\|UBE2A | 2.90E−61 | [Homo sapiens][Ligase; Protein conjugation factor] Human homolog of S. cerevisiae |
| | | | | Rad6p, a member of the ubiquitin-conjugating enzyme family that |
| | | | | catalyzes the ubiquitination of cellular proteins and marks them for degradation, |
| | | | | also plays a role in DNA repair |
| 16 | 7504076CD1 | g7677403 | 6.60E−105 | [Homo sapiens] F-box protein FBG2 |
| | | | | Ilyin, G. P., et al. (2000) Genomics 67: 40-47 |
| | | | | cDNA cloning and expression analysis of new members of the mammalian F-box |
| | | | | protein family |
| | | 598228\|FBXO6 | 5.80E−106 | [Homo sapiens][Ligase; Protein conjugation factor] Member of a family of F-box |
| | | | | containing proteins, a putative subunit of the SCF ubiquitin ligase involved in |
| | | | | protein degradation |
| 17 | 7500926CD1 | g3868871 | 2.60E−16 | [Clostridium histolyticum] Orf2u |
| | | | | Matsushita, O., et al. (1999) Gene duplication and multiplicity of collagenases in |
| | | | | Clostridium histolyticum. J. Bacteriol. 181: 923-933 |
| | | 377422\|pi053 | 6.90E−16 | [Schizosaccharomyces pombe] Conserved protein containing a DUF28 domain |
| 18 | 7503216CD1 | g8489879 | 0.0 | [Homo sapiens] (AF272981) cytosolic aminopeptidase P |
| | | | | Cottrell, G. S., et al. (2000) Cloning, expression, and characterization of human |
| | | | | cytosolic aminopeptidase P: a single manganese(II)-dependent enzyme. |
| | | | | Biochemistry 39: 15121-15128 |
| | | 739810\|XPNPEP1 | 0.0 | [Homo sapiens] X-prolyl aminopeptidase (aminopeptidase P)1, soluble |
| | | 567960\|XPNPEPL | 0.0 | [Homo sapiens][Hydrolase; Protease (other than proteasomal)] X-prolyl |
| | | | | aminopeptidase-like (aminopeptidase P-like), a putative X-prolyl aminopeptidase, |
| | | | | ubiquitously expressed |
| 18 | | 344930\|XPNPEP2 | 9.4E−123 | [Homo sapiens][Hydrolase; Protease (other than proteasomal)] X-prolyl |
| | | | | aminopeptidase (aminopeptidase P) 2 (membrane-bound), metallopeptidase which |
| | | | | catalyzes removal of N-terminal amino acids from peptides with N-terminal Xaa- |
| | | | | Pro sequences; inhibited by apstatin; may be associated with premature ovarian |
| | | | | failure |
| 19 | 7503233CD1 | g29664 | 0.0 | [Homo sapiens] CANP, large subunit (aa 1-714) |
| | | | | Aoki, K., et al. (1986) Complete amino acid sequence of the large subunit of the |
| | | | | low-Ca2+-requiring form of human Ca2+-activated neutral protease (muCANP) |
| | | | | deduced from its cDNA sequence. FEBS Lett. 205: 313-317 |
| | | 661158\|CAPN1 | 0.0 | [Homo sapiens][Hydrolase; Protease (other than proteasomal)][Plasma |
| | | | | membrane] Calpain I, catalytic subunit of mu-calpain, a calcium-dependent |
| | | | | cysteine (thiol) protease that requires micromolar concentrations of calcium in |
| | | | | vitro |
| | | 334452\|CAPN2 | 1.6E−227 | [Homo sapiens][Hydrolase; Protease (other than proteasomal)] Calpain 2, large |
| | | | | subunit of the cysteine-type protease m-calpain which may regulate the cell cycle, |
| | | | | apoptosis, and cellular differentiation, upregulated in muscle from progressive |
| | | | | muscular dystrophy and amyotrophic lateral sclerosis patients |
| 20 | 7726576CD1 | g4079809 | 2.8E−55 | [Homo sapiens] HERC2 |
| | | | | Ji, Y., et al. (1999) The ancestral gene for transcribed, low-copy repeats in the |
| | | | | Prader-Willi/Angelman region encodes a large protein implicated in protein |
| | | | | trafficking, which is deficient in mice with neuromuscular and spermiogenic |
| | | | | abnormalities. Hum. Mol. Genet. 8: 533-542 |
| | | 691012\| | 3.4E−110 | [Homo sapiens] has moderate similarity to a region of human HERC1, which is a |
| | | FLJ21156 | | guanine-nucleotide exchange factor that interacts with ARF1 and binds to Hsp70 |
| | | | | and clathrin heavy chain (CLTC) |
| | | 345082\|HERC2 | 2.4E−56 | [Homo sapiens][Guanine-nucleotide exchange factor] Homolog of murine |
| | | | | Mm.20929, which is a guanine-nucleotide exchange factor involved in |
| | | | | intracellular protein transport; duplicated and truncated copies of the |
| | | | | corresponding gene are associated with deletion breakpoints in Prader-Willi and |
| | | | | Angelman syndromes |
| | 7726576CD1 | 341506\|HERC1 | 4.4E−44 | [Homo sapiens][Guanine-nucleotide exchange factor][Golgi; Cytoplasmic] HECT |
| | | | | (homologous to E6-AP (UBE3A) carboxy terminus) domain and RCC1 (CHC1)- |
| | | | | like domain (RLD) 1, functions as a guanine-nucleotide exchange factor for Rab |
| | | | | related proteins and ARF1, may be involved in membrane transport processes |
| 21 | 7503507CD1 | g2924601 | 3.40E−30 | [Homo sapiens] hepatocyte growth factor activator inhibitor. Shimomura, T. et al. |
| | | | | (1997) Hepatocyte growth factor activator inhibitor, a novel Kunitz-type serine |
| | | | | protease inhibitor. J. Biol. Chem. 272 (10), 6370-6376 |
| 22 | 7503506CD1 | 341496\|SPINT1 | 5.9E−31 | [Homo sapiens][Inhibitor or repressor][Extracellular (excluding cell wall); |
| | | | | Unspecified membrane; Plasma membrane] Serine protease inhibitor (Kunitz type |
| | | | | 1), a Kunitz type serine protease inhibitor that inhibits hepatocyte growth factor |
| | | | | activator and is found in both membrane-associated and secreted forms |
| | | | | Shimomura, T. et al. J. Biol. Chem. 272: 6370-6 (1997) |
| | | | | Kataoka, H. et al. Cancer Res. 60: 6148-59 (2000) |
| | | 588049\|Spint1 | 7.7E−27 | [Mus musculus][Inhibitor or repressor; Small molecule-binding |
| | | | | protein][Unspecified membrane; Extracellular (excluding cell wall)] Serine |
| | | | | protease inhibitor (Kunitz type 1), a Kunitz type serine protease inhibitor that |
| | | | | inhibits hepatocyte growth factor activator; contains a transmembrane domain |
| 23 | 7503509CD1 | g13278723 | 2.40E−14 | [Homo sapiens] serine protease inhibitor, Kunitz type 1 |
| | | 341496\|SPINT1 | 2.10E−15 | [Homo sapiens][Inhibitor or repressor][Extracellular (excluding cell wall); Plasma |
| | | | | membrane] Serine protease inhibitor (Kunitz type 1), a Kunitz type serine protease |
| | | | | inhibitor that inhibits hepatocyte growth factor activator and is found in both |
| | | | | membrane-associated and secreted forms |
| | | 588049\|Spint1 | 4.60E−13 | [Mus musculus][Inhibitor or repressor; Small molecule-binding protein] |
| | | | | [Extracellular (excluding cell wall)] Serine protease inhibitor (Kunitz type 1), a |
| | | | | Kunitz type serine protease inhibitor that inhibits hepatocyte growth factor |
| | | | | activator; contains a transmembrane domain |
| 24 | 7505800CD1 | g292031 | 2.30E−134 | [Homo sapiens] farnesyl-protein transferase alpha-subunit |
| | | 335360\|FNTA | 2.00E−35 | [Homo sapiens][Transferase][Cytoplasmic] Farnesyl transferase alpha subunit, |
| | | | | transfers farnesyl and geranyl-geranyl groups to proteins, implicated to be |
| | | | | involved in TGF-beta and activin signaling |
| | | | | Zhang, F. L. et al. (1994) cDNA cloning and expression of rat and human protein |
| | | | | geranylgeranyltransferase type-I. J. Biol. Chem. 269: 3175-3180 |
| | | 720247\|1qbq_A | 2.60E−128 | [Protein Data Bank] Fpt Alpha-Subunit |
| | | 328782\|Fnta | 1.10E−127 | [Rattus norvegicus][Transferase] Farnesyl transferase alpha subunit, transfers |
| | | | | farnesyl groups to proteins |
| | | | | Chen, W. J. et al. (1991) Cloning and expression of a cDNA encoding the alpha |
| | | | | subunit of rat p21ras protein farnesyltransferase. Proc. Natl. Acad. Sci. USA |
| | | | | 88: 11368-11372 |
| | | 725889\|1d8d_A | 1.10E−127 | [Protein Data Bank] Farnesyltransferase (Alpha Subunit) |
| | | 582901\|Fnta | 3.80E−127 | [Mus musculus][Transferase] Protein with strong similarity to human FNTA, |
| | | | | which is the alpha subunit of CAAX farnesyl transferase (FPTase) and geranyl- |
| | | | | geranyl transferase type-I (GGTase-I), and that transfers farnesyl and geranyl- |
| | | | | geranyl groups to proteins |
| 25 | 7503141CD1 | g15929143 | 3.9E−248 | [Homo sapiens] Peptidase D |
| | | 339574\|PEPD | 1.1E−241 | [Homo sapiens][Hydrolase; Protease (other than proteasomal)] Peptidase D |
| | | | | (prolidase), catalyzes hydrolysis of dipeptides having a C-terminal proline, |
| | | | | functions in proline recycling during collagen synthesis, deficiency causes |
| | | | | iminodipeptiduria, mental retardation, collagenous tissue defects, and skin lesions |
| | | 429336\|Pep4 | 2.9E−221 | [Mus musculus][Hydrolase; Protease (other than proteasomal)] Peptidase D |
| | | | | (prolidase), putative dipeptidase that catalyzes hydrolysis of substrates having a C- |
| | | | | terminal proline, may function in collagen metabolism; deficiency of human |
| | | | | PEPD causes mental retardation, collagenous tissue defects, and skin lesions |
| | | 687787\|K12C11.1 | 3.3E−118 | [Caenorhabditis elegans][Hydrolase; Protease (other than proteasomal)] Small |
| | | | | protein with a region of strong similarity to peptidases |
| | | 9721\|YFR006W | 2.6E−54 | [Saccharomyces cerevisiae][Unknown] Protein with weak similarity to human X- |
| | | | | pro dipeptidase |
| | | 646084\|orf6.8163 | 2.9E−53 | [Candida albicans][Unknown] Member of the metallopeptidase family M24, has |
| | | | | high similarity to S. cerevisiae Yfr006p, which is a protein with weak similarity to |
| | | | | human X-prodipeptidase |
| 26 | 7500362CD1 | g20271451 | 0.0 | [Homo sapiens] peptidase D |
| | | 339574\|PEPD | 5.0E−250 | [Homo sapiens] [Hydrolase; Protease (other than proteasomal] Peptidase D |
| | | | | (prolidase), catalyzes hydrolysis of dipeptides having a C-terminal proline, |
| | | | | functions in proline recycling during collagen synthesis, deficiency causes |
| | | | | iminodipeptiduria, mental retardation, collagenous tissue defects, and skin lesions |
| | | | | Endo, F. et al. J Biol Chem 264, 4476-81 (1989). |
| | | | | Tanoue, A. et al. J Biol Chem 265, 11306-11 (1990). |
| | | 429336\|Pep4 | 8.9E−231 | [Mus musculus] [Hydrolase; Protease (other than proteasomal)] Peptidase D |
| | | | | (prolidase), putative dipeptidase that catalyzes hydrolysis of substrates having a C- |
| | | | | terminal proline, may function in collagen metabolism; deficiency of human |
| | | | | PEPD causes mental retardation, collagenous tissue defects, and skin lesions |
| | | | | Ishii, T. et al. Biochim Biophys Acta 1308, 15-6 (1996). |
| 27 | 7503328CD1 | g8489879 | 7.2E−210 | [Homo sapiens] cytosolic aminopeptidase P |
| | | | | Cottrell, G. S. et al. |
| | | | | Biochemistry 39, 15121-15128 (2000) |
| | | 567960\|XPNPEPL | 3.2E−210 | [Homo sapiens] [Hydrolase; Protease (other than proteasomal)] X-prolyl |
| | | | | aminopeptidase-like (aminopeptidase P-like), a putative X-prolyl aminopeptidase, |
| | | | | ubiquitously expressed |
| | | | | Vanhoof, G. et al. |
| | | | | Isolation and sequence analysis of a human cDNA clone (XPNPEPL) homologous |
| | | | | to X-prolyl aminopeptidase (aminopeptidase P). |
| | | | | Cytogenet Cell Genet 78, 275-80 (1997). |
| | | 332548\|Rn.25763 | 7.5E−202 | [Rattus norvegicus] [Hydrolase; Protease (other than proteasomal)] [Cytoplasmic] |
| | | | | X-prolyl aminopeptidase (aminopeptidase P)-1 (soluble), catalyzes removal of the |
| | | | | N-terminal amino acid from peptides with N terminal Xaa-Pro sequences, has |
| | | | | activity against bradykinin, substance P and other bioactive peptides |
| | | | | Czirjak, G. et al. |
| | | | | Cloning and functional expression of the cytoplasmic form of rat aminopeptidase |
| | | | | P. |
| | | | | Biochim Biophys Acta 1444, 326-36 (1999). |
| 28 | 7510464CD1 | g13477305 | 0.0 | [Homo sapiens] X-prolyl aminopeptidase (aminopeptidase P) 1, soluble |
| 29 | 7510394CD1 | g14456615 | 3.60E−64 | [Homo sapiens] phosphatidyl inositol glycan class T |
| | | | | Ohishi, K. et al. (supra) |
| 30 | 7500745CD1 | g14456615 | 1.30E−55 | [Homo sapiens] phosphatidyl inositol glycan class T |
| | | | | Ohishi, K. et al. (supra) |

TABLE 3

| SEQ | Incyte | Amino | Potential | Potential | | Analytical |
|---|---|---|---|---|---|---|
| ID | Polypeptide | Acid | Phosphorylation | Glycosylation | | Methods and |
| NO: | ID | Residues | Sites | Sites | Signature Sequences, Domains and Motifs | Databases |
| 1 | 8268274CD1 | 404 | S77 S118 S178 | N96 N102 N123 | F-box domain: T3-A50 | HMMER_{—} |
| | | | S196 S219 S254 | N155 N234 N265 | | PFAM |
| | | | S278 S282 S287 | | | |
| | | | S401 S402 T208 | | | |
| | | | T344 T359 T370 | | | |
| | | | T395 | | | |
| | | | | | PROTEIN GRR1 REPEAT SIMILAR C02F5.7 | BLAST_{—} |
| | | | | | CAENORHA GLUCOSE METABOLISM LEUCINE- | PRODOM |
| | | | | | REPEAT T13E15.9 PD003743: K128-I266, N102-C238 | |
| 2 | 7500515CD1 | 900 | S45 S185 S286 | N81 N207 N517 | signal_cleavage: M1-A28 | SPSCAN |
| | | | S305 S366 S407 | N577 | | |
| | | | S510 S525 S535 | | | |
| | | | S562 S564 S636 | | | |
| | | | S672 S731 S751 | | | |
| | | | S780 S808 T26 T54 | | | |
| | | | T63 T121 T231 | | | |
| | | | T384 T389 T594 | | | |
| | | | T756 T834 T866 | | | |
| | | | Y146 | | | |
| | | | | | Signal Peptides: M1-H23, M1-Q24, M1-A28, M1-T27 | HMMER |
| | | | | | von Willebrand factor type A domain: N274-V457 | HMMER_{—} |
| | | | | | | PFAM |
| | | | | | Inosine-uridine preferring nucleoside hydrolase family | BLIMPS_{—} |
| | | | | | signature BL01247: N353-S397, A531-N542, I298-L312 | BLOCKS |
| | | | | | INHIBITOR HEAVY CHAIN PD01101: Q65-K98, | BLIMPS_{—} |
| | | | | | N256-D308, G348-N367, R439-V493, W548-L557 | PRODOM |
| | | | | | HEAVY CHAIN H4 PRECURSOR INTER-ALPHA- | BLAST_{—} |
| | | | | | TRYPSIN INHIBITOR ITI FAMILY CHAIN | PRODOM |
| | | | | | RELATED PROTEIN PD017446: P669-L900 | |
| | | | | | HEAVY CHAIN PRECURSOR INTER-ALPHA- | BLAST_{—} |
| | | | | | TRYPSIN INHIBITOR ITI SERINE PROTEASE | PRODOM |
| | | | | | REPEAT SIGNAL PD004379: Q24-K273 | |
| | | | | | PD004369: A430-S620 | |
| | | | | | INTER-ALPHA-TRYPSIN INHIBITOR HEAVY | BLAST_{—} |
| | | | | | CHAIN H4 PRECURSOR ITI FAMILY CHAIN- | PRODOM |
| | | | | | RELATED PLASMA KALLIKREIN SENSITIVE | |
| | | | | | GLYCOPROTEIN 120 SERINE PROTEASE | |
| | | | | | PD120343: A621-P668 | |
| | | | | | INTER-ALPHA-TRYPSIN INHIBITOR COMPLEX | BLAST_{—} |
| | | | | | COMPONENT II DM03009 | DOMO |
| | | | | | \|JX0368\|372-855: L372-E738, K607-G826 | |
| | | | | | \|S30350\|378-841: L372-E610, P702-Q850 | |
| | | | | | \|P19823\|408-896: L372-S613, P645-E851 | |
| | | | | | HUMAN INTER-ALPHA-TRYPSIN INHIBITOR | BLAST_{—} |
| | | | | | HEAVY CHAIN-RELATED PROTEIN | DOMO |
| | | | | | PRECURSOR DM03690\|JX0368\|96-278: K96-I279 | |
| | | | | | ATP/GTP-binding site motif A (P-loop): A107-S114 | MOTIFS |
| 3 | 2256826CD1 | 436 | S11 S17 S38 S86 | N84 N158 N175 | signal_cleavage: M1-A54 | SPSCAN |
| | | | S97 S160 S177 | N259 | | |
| | | | S333 T206 | | | |
| | | | | | F-box domain: V23-L70, R99-R146 | HMMER_{—} |
| | | | | | | PFAM |
| | | | | | PROTEIN GRR1 REPEAT SIMILAR CAENORHA | BLAST_{—} |
| | | | | | GLUCOSE METABOLISM LEUCINE REPEAT | PRODOM |
| | | | | | PD003743: S155-M304, L208-E336, V104-L252, | |
| | | | | | N259-L375, L286-E385 | |
| | | | | | HYPOTHETICAL 76.5 KD PROTEIN EEED8.10 IN | BLAST_{—} |
| | | | | | CHROMOSOME II PD135828: S17-R344 | PRODOM |
| | | | | | DNA REPAIR PROTEIN PUTATIVE EXCISION | BLAST_{—} |
| | | | | | RAD7 PD135808: V104-L386 | PRODOM |
| | | | | | P45; CYCLIN; CDK2 DM08625\|P34284\|62-155: | BLAST_{—} |
| | | | | | K30-L124 | DOMO |
| | | | | | Cytochrome c family heme-binding site signature: | MOTIFS |
| | | | | | C243-K248 | |
| 4 | 7686186CD1 | 356 | S48 S201 T6 T58 | | signal_cleavage: M1-A45 | SPSCAN |
| | | | T69 T131 T172 | | | |
| | | | T278 Y140 | | | |
| | | | | | Trypsin: H116-V272 | HMMER_{—} |
| | | | | | | PFAM |
| | | | | | Cytosolic domain: M1-R18 Transmembrane domain: | TMHMMER |
| | | | | | F19-P41 Non-cytosolic domain: A42-C356 | |
| | | | | | HtrA/DegQ protease family signature PR00834: | BLIMPS_{—} |
| | | | | | S238-A255, G332-G344, G115-A127, D136-L156, | PRINTS |
| | | | | | V178-A202, I216-G233 | |
| | | | | | V8 serine protease family signature PR00839: V178-L191, | BLIMPS_{—} |
| | | | | | I220-L236, D237-I249 | PRINTS |
| | | | | | Alpha-lytic endopeptidase serine protease (S2A) | BLIMPS_{—} |
| | | | | | signature PR00861: H116-A130, L195-S212, I216-A239 | PRINTS |
| | | | | | PROTEASE SERINE PROTEIN PERIPLASMIC | BLAST_{—} |
| | | | | | SIGNAL PRECURSOR HTRA HYDROLASE | PRODOM |
| | | | | | PD001397: S173-T302 | |
| | | | | | PROTEASE DEGS CHAIN DM01722\|P45129\|3-373: | BLAST_{—} |
| | | | | | R103-A330 DM01722\|P09376\|1-383: G106-V328 | DOMO |
| | | | | | DM01722\|P54925\|11-395: P102-A346 | |
| | | | | | DM01722\|P39099\|1-361: G104-V334 | |
| 5 | 72617436CD1 | 432 | S5 S10 S38 S92 | N267 | signal_cleavage: M1-G33 | SPSCAN |
| | | | S110 S111 S245 | | | |
| | | | S269 S356 S360 | | | |
| | | | S370 S393 T138 | | | |
| | | | T196 T229 T299 | | | |
| | | | | | Signal Peptide: M1-A34, M1-G24, M1-S30 | HMMER |
| | | | | | CUB domain proteins profile BL01180: G190-D201 | BLIMPS_{—} |
| | | | | | | BLOCKS |
| | | | | | Astacin (Peptidase family M12A): N93-P284 | HMMER_{—} |
| | | | | | | PFAM |
| | | | | | Astacin family signature PR00480: P230-S245, L268-G281 | BLIMPS_{—} |
| | | | | | I121-Y139, Q175-H193, E194-I211 | PRINTS |
| | | | | | PROTEIN GLYCOPROTEIN EGFLIKE DOMAIN | BLAST_{—} |
| | | | | | HYDROLASE METALLOPROTEASE ZINC | PRODOM |
| | | | | | PRECURSOR SIGNAL ZYMOGEN PD000834: | |
| | | | | | S92-C282 | |
| | | | | | ASTACIN DM00570 | BLAST_{—} |
| | | | | | P31580\|5-269: E46-C282 | DOMO |
| | | | | | P31579\|6-271: L74-C282 | |
| | | | | | P42662\|1-183: G101-Y280 | |
| | | | | | P55112\|37-308: L87-C282 | |
| | | | | | Neutral zinc metallopeptidases, zinc-binding region | MOTIFS |
| | | | | | signature: I180-L189 | |
| 6 | 7501945CD1 | 248 | S5 S10 S38 S92 | | signal_cleavage: M1-G33 | SPSCAN |
| | | | S110 S111 T138 | | | |
| | | | T196 T229 | | | |
| | | | | | Signal Peptide: M1-A34, M1-G24, M1-S30 | HMMER |
| | | | | | CUB domain proteins profile BL01180: G190-D201 | BLIMPS_{—} |
| | | | | | | BLOCKS |
| | | | | | Astacin (Peptidase family M12A): N93-Q246 | HMMER_{—} |
| | | | | | | PFAM |
| | | | | | Neutral zinc metallopeptidases, zinc-binding region | PROFILE- |
| | | | | | signature: S161-N207 | SCAN |
| | | | | | Astacin family signature PR00480: E194-I211, P230-P245, | BLIMPS_{—} |
| | | | | | I121-Y139, Q175-H193 | PRINTS |
| | | | | | PROTEIN GLYCOPROTEIN EGFLIKE DOMAIN | BLAST_{—} |
| | | | | | HYDROLASE METALLOPROTEASE ZINC | PRODOM |
| | | | | | PRECURSOR SIGNAL ZYMOGEN PD000834: | |
| | | | | | S92-R241 | |
| | | | | | ASTACIN DM00570 | BLAST_{—} |
| | | | | | P31580\|5-269: E46-R241 | DOMO |
| | | | | | P31579\|6-271: L74-R241 | |
| | | | | | P42662\|1-183: G101-G240 | |
| | | | | | P55112\|37-308: L87-G240 | |
| | | | | | Neutral zinc metallopeptidases, zinc-binding region | MOTIFS |
| | | | | | signature: I180-L189 | |
| 7 | 7500264CD1 | 388 | S11 S34 S53 S201 | N164 N289 | signal_cleavage: M1-A37 | SPSCAN |
| | | | S234 S261 S265 | | | |
| | | | S270 S283 S310 | | | |
| | | | S323 T166 T197 | | | |
| | | | T285 T375 Y319 | | | |
| | | | | | Signal Peptide: M1-A37 | HMMER |
| | | | | | Cytosolic domain: R362-L388 | TMHMMER |
| | | | | | Transmembrane domain: A339-L361 | |
| | | | | | Non-cytosolic domain: M1-G338 | |
| | | | | | HEPATOCYTE GROWTH FACTOR ACTIVATOR | BLAST_{—} |
| | | | | | INHIBITOR GLYCOPROTEIN | PRODOM |
| | | | | | PD120361: G84-V254, S11-P44 | |
| | | | | | Leucine zipper pattern: L45-L66, L347-L368 | MOTIFS |
| 8 | 7499935CD1 | 467 | S138 S167 S224 | N13 N172 | Metallopeptidase family M24: V185-E447 | HMMER_{—} |
| | | | S312 S434 T15 T54 | | | PFAM |
| | | | T90 T146 T188 | | | |
| | | | T198 T445 Y128 | | | |
| | | | | | Aminopeptidase P and proline dipeptidase proteins | BLIMPS_{—} |
| | | | | | BL00491: A319-H330, H366-D378, G422-G435 | BLOCKS |
| | | | | | PROLIDASE HYDROLASE XAAPRO | BLAST_{—} |
| | | | | | DIPEPTIDASE XPRO PROLINE | PRODOM |
| | | | | | IMIDODIPEPTIDASE ACETYLATION | |
| | | | | | MANGANESE PEPTIDASE | |
| | | | | | PD013444: A2-V185 | |
| | | | | | AMINOPEPTIDASE HYDROLASE METHIONINE | BLAST_{—} |
| | | | | | PEPTIDASE PROTEIN COBALT M DIPEPTIDASE | PRODOM |
| | | | | | XPRO MAP | |
| | | | | | PD000555: V185-T440 | |
| | | | | | AMINOPEPTIDASE P AND PROLINE | BLAST_{—} |
| | | | | | DIPEPTIDASE DM00816 | DOMO |
| | | | | | P12955\|179-467: I180-I389, I389-P443 | |
| | | | | | P43590\|225-509: E182-P383, Y390-T440 | |
| | | | | | P15034\|169-423: E182-T440 | |
| | | | | | P44881\|163-417: E182-P443 | |
| | | | | | Aminopeptidase P and proline dipeptidase signature: | MOTIFS |
| | | | | | H366-D378 | |
| 9 | 7982285CD1 | 379 | S42 S67 S160 S234 | N53 N76 | DnaJ domain: N108-G172 | HMMER_{—} |
| | | | S319 T171 T197 | | | PFAM |
| | | | T286 Y324 | | | |
| | | | | | Nt-dnaJ domain proteins BL00636: D123-K139, | BLIMPS_{—} |
| | | | | | F149-D169 | BLOCKS |
| | | | | | DnaJ domains signatures and profile: R129-N187 | PROFILE- |
| | | | | | | SCAN |
| | | | | | DnaJ protein family signature PR00625: A119-D138, | BLIMPS_{—} |
| | | | | | F149-D169, S55-K74 | PRINTS |
| | | | | | PROTEIN HLJ1 B0035.14 CHROMOSOME IV | BLAST_{—} |
| | | | | | CHAPERONE PD036881: P198-L372 | PRODOM |
| | | | | | PROTEIN CHAPERONE DNAJ HEAT SHOCK | BLAST_{—} |
| | | | | | DNA REPLICATION REPEAT ANTIGENT | PRODOM |
| | | | | | PD000231: N108-D169 | |
| | | | | | NT-DNAJ DOMAIN DM00098 | BLAST_{—} |
| | | | | | P35515\|1-101: K107-G208 | DOMO |
| | | | | | P30725\|1-102: K107-G20 | |
| | | | | | P25685\|1-107: K107-G208 | |
| | | | | | S34632\|1-99: Y109-G209 | |
| | | | | | Cell attachment sequence: R242-D244 | MOTIFS |
| | | | | | Nt-dnaJ domain signature: F149-Y168 | MOTIFS |
| | | | | | N-6 Adenine-specific DNA methylases signature: | MOTIFS |
| | | | | | M269-Y275 | |
| 10 | 7758505CD1 | 737 | S48 S217 S301 | N361 | signal_cleavage: M1-G26 | SPSCAN |
| | | | S477 S559 S572 | | | |
| | | | S733 T390 T498 | | | |
| | | | T524 T577 T579 | | | |
| | | | T602 Y646 | | | |
| | | | | | Signal Peptide: M1-A15; M1-H23; M1-G26; M1-P21 | HMMER |
| | | | | | von Willebrand factor type C domain: C159-C216, | HMMER_{—} |
| | | | | | C285-C342, C28-C87, K366-C416, C95-C152, | PFAM |
| | | | | | C221-C278 | |
| | | | | | PRECURSOR SIGNAL RECEPTOR | BLAST_{—} |
| | | | | | GLYCOPROTEIN TRANSMEMBRANE KINASE | PRODOM |
| | | | | | TRANSFERASE TYROSINE PROTEIN ATP- | |
| | | | | | BINDING PHOSPHORYLATION PD000495: S395-C662 | |
| | | | | | PROTEIN PRECURSOR REPEAT | BLAST_{—} |
| | | | | | GLYCOPROTEIN SIGNAL NEL EGF-LIKE | PRODOM |
| | | | | | DOMAIN CHORDIN B0024.14 PD015143: C84-C152 | |
| | | | | | VON WILLEBRAND FACTOR TYPE C REPEAT | BLAST_{—} |
| | | | | | DM00551\|A38963\|649-756: W41-V153 | DOMO |
| | | | | | Cytochrome c family heme-binding site signature: | MOTIFS |
| | | | | | C510-E515, C605-N610, C652-S657 | |
| | | | | | VWFC domain signature: C46-C87, C113-C152, | MOTIFS |
| | | | | | C177-C216, C239-C278, C303-C342, C379-C416 | |
| 11 | 6885756CD1 | 530 | S22 S23 S36 S38 | N92 N444 N488 | Ubiquitin carboxyl-terminal hydrolases family: A80-R111 | HMMER_{—} |
| | | | S71 S72 S110 S232 | | | PFAM |
| | | | S272 S284 S376 | | | |
| | | | S432 S491 S511 | | | |
| | | | S522 T47 T399 | | | |
| | | | T404 T446 T495 | | | |
| | | | T512 Y306 Y347 | | | |
| | | | | | Ubiquitin carboxyl-terminal hydrolase family: G313-Q374 | HMMER_{—} |
| | | | | | | PFAM |
| | | | | | Ubiquitin carboxyl-terminal hydrolases family 2 | BLIMPS_{—} |
| | | | | | proteins BL00972: G81-L98, G156-L165, I193-Y207, | BLOCKS |
| | | | | | Y317-A341, E343-S364 | |
| | | | | | PROTEASE UBIQUITIN HYDROLASE | BLAST_{—} |
| | | | | | UBIQUITIN SPECIFIC ENZYME | PRODOM |
| | | | | | DEUBIQUITINATING CARBOXYL TERMINAL | |
| | | | | | THIOLESTERASE PROCESSING CONJUGATION | |
| | | | | | PD017412: F217-Q309 | |
| | | | | | UBIQUITIN CARBOXYL-TERMINAL | BLAST_{—} |
| | | | | | HYDROLASES FAMILY 2 DM00659 | DOMO |
| | | | | | \|P40818\|782-1103: H180-L370, I85-Y107 | |
| | | | | | \|P50102\|141-420: Q158-G327 | |
| | | | | | Ubiquitin carboxyl-terminal hydrolases family 2 | MOTIFS |
| | | | | | signature 2: Y317-Y335 | |
| 12 | 7500748CD1 | 511 | S29 S81 S108 S126 | N164 N291 N327 | signal_cleavage: M1-A23 | SPSCAN |
| | | | S156 S196 T52 | | | |
| | | | T119 T121 T267 | | | |
| | | | T303 T311 Y287 | | | |
| | | | Y346 | | | |
| | | | | | Signal Peptide: M5-C21; M5-A23; M1-A23; M1-P25; | HMMER |
| | | | | | Cytosolic domain: N478-L511 Transmembrane | TMHMMER |
| | | | | | domain: F455-Y477 Non-cytosolic domain: M1-D454 | |
| | | | | | PROTEIN F17C11.7 IKI1ERG9 INTERGENIC | BLAST_{—} |
| | | | | | REGION TRANSMEMBRANE PD043343: E32-V245 | PRODOM |
| | | | | | PROTEIN F17C11.7 IKI1ERG9 INTERGENIC | BLAST_{—} |
| | | | | | REGION TRANSMEMBRANE PD043344: H348-V399, | PRODOM |
| | | | | | L441-G474 | |
| 13 | 7500749CD1 | 476 | S29 S94 T52 T165 | N189 N225 | signal_cleavage: M1-A23 | SPSCAN |
| | | | T201 T209 T301 | | | |
| | | | Y67 Y185 | | | |
| | | | | | Signal Peptide: M5-C21; M5-A23; M1-C21; M1-A23; | HMMER |
| | | | | | M1-P25 | |
| | | | | | Cytosolic domain: N443-L476 Transmembrane | TMHMMER |
| | | | | | domain: F420-Y442 Non-cytosolic domain: M1-D419 | |
| | | | | | PROTEIN F17C11.7 IKI1ERG9 INTERGENIC | BLAST_{—} |
| | | | | | REGION TRANSMEMBRANE PD043344: H313-V364 | PRODOM |
| 14 | 7503401CD1 | 344 | S49 S93 S113 T315 | N130 N198 N211 | Protein prenyltransferase alpha subunit repe: Q149-K179, | HMMER_{—} |
| | | | T334 Y102 | N238 | R115-R145, R223-G253, N183-T213 | PFAM |
| | | | | | Protein prenyltransferases alpha subunit repeat | BLIMPS_{—} |
| | | | | | proteins proteins | BLOCKS |
| | | | | | BL00904: R115-S148, Q149-D189, | |
| | | | | | R223-G247, I248-E285, Q291-T315, | |
| | | | | | G14-Y63 | |
| | | | | | TRANSFERASE SUBUNIT ALPHA PROTEIN | BLAST_{—} |
| | | | | | PRENYLTRANSFERASE REPEAT | PRODOM |
| | | | | | FARNESYLTRANSFERASE PROTEINS CAAX | |
| | | | | | RAS PD005875: V99-Q343 | |
| | | | | | TRANSFERASE SUBUNIT | BLAST_{—} |
| | | | | | FARNESYLTRANSFERASE PROTEIN ALPHA | PRODOM |
| | | | | | CAAX RAS PROTEINS PRENYLTRANSFERASE | |
| | | | | | FTASE ALPHA PD011572: V64-V106 | |
| | | | | | FARNESYLTRANSFERASE; ALPHA; | BLAST_{—} |
| | | | | | DM07118\|P49354\|257-378: E222-Q344 | DOMO |
| | | | | | PROTEIN PRENYLTRANSFERASES ALPHA | BLAST_{—} |
| | | | | | SUBUNIT REPEAT DM01356\|P49354\|174-212: | DOMO |
| | | | | | V139-F178 | |
| | | | | | Protein prenyltransferases alpha subunit repeat | MOTIFS |
| | | | | | signature: A128-R137, A162-R171, V196-R205, | |
| | | | | | P236-L245 | |
| 15 | 7503485CD1 | 122 | S81 S112 S118 T46 | | Ubiquitin-conjugating enzyme: M1-Q117 | HMMER_{—} |
| | | | | | | PFAM |
| | | | | | signal_cleavage: M34-D84 | SPSCAN |
| | | | | | Ubiquitin-conjugating enzymes active site: P21-E85 | PROFILE- |
| | | | | | | SCAN |
| | | | | | UBIQUITIN LIGASE ENZYME PROTEIN | BLAST_{—} |
| | | | | | UBIQUITIN-CONJUGATING CONJUGATION | PRODOM |
| | | | | | CARRIER UBIQUITIN PROTEIN MULTIGENE | |
| | | | | | FAMILY PD000461: A53-R110, M1-E49 | |
| | | | | | UBIQUITIN-CONJUGATING ENZYMES | BLAST_{—} |
| | | | | | DM00225 | DOMO |
| | | | | | \|A41222\|2-149: D50-R120, S20-D50 | |
| | | | | | \|P52478\|2-149: D50-W119, S20-D50 | |
| | | | | | \|P25865\|2-149; D50-W119, S2-E99 | |
| | | | | | \|P23566\|2-149: D50-W119, S20-D50 | |
| 16 | 7504076CD1 | 255 | T153 T154 | N245 | F-box domain: G4-L52 | HMMER_{—} |
| | | | | | | PFAM |
| 17 | 7500926CD1 | 166 | S35 S120 S157 | N155 | signal_cleavage: M1-A19 | SPSCAN |
| | | | | | Signal Peptide: M1-A26 | HMMER |
| | | | | | PROTEIN INTERGENIC REGION CONSERVED | BLAST_{—} |
| | | | | | OF SECTION COAT MG332 VMA7RPS31A | PRODOM |
| | | | | | VMA7RPS25A PD004323: K62-K127 | |
| 18 | 7503216CD1 | 591 | S54 S182 S214 | N89 N371 | metallopeptidase family M24: A288-N520 | HMMER_{—} |
| | | | S251 S259 S329 | | | PFAM |
| | | | S375 S447 S473 | | | |
| | | | S544 T102 T156 | | | |
| | | | T370 T405 T476 | | | |
| | | | T546 T559 | | | |
| | | | | | Aminopeptidase P and proline dipeptidase proteins | BLIMPS_{—} |
| | | | | | BL00491: I352-H363, H453-V465, L482-E496, | BLOCKS |
| | | | | | G501-K514 | |
| | | | | | Proteasome A-type and B-type PF00227: I31-Y42 | BLIMPS_{—} |
| | | | | | | PFAM |
| | | | | | AMINOPEPTIDASE AMINOACYL PROLINE | BLAST_{—} |
| | | | | | HYDROLASE XAA PRO XPRO PROLINE P | PRODOM |
| | | | | | PROTEIN PRECURSOR P-LIKE PD009635: K514-S588 | |
| | | | | | AMINOPEPTIDASE HYDROLASE AMINOACYL | BLAST_{—} |
| | | | | | PROLINE XAA PRO P XPRO PROTEIN | PRODOM |
| | | | | | PUTATIVE PROLINE APP PD004954: L10-D120 | |
| | | | | | AMINOPEPTIDASE HYDROLASE METHIONINE | BLAST_{—} |
| | | | | | PEPTIDASE PROTEIN COBALT M DIPEPTIDASE | PRODOM |
| | | | | | XPRO MAP PD000555: E369-V510, V289-G493 | |
| | | | | | AMINOPEPTIDASE AMINOACYL PROLINE | BLAST_{—} |
| | | | | | HYDROLASE P XAA PRO XPRO PROLINE | PRODOM |
| | | | | | PRECURSOR PROTEIN P-LIKE PD008419: E148-A288 | |
| | | | | | AMINOPEPTIDASE P AND PROLINE | BLAST_{—} |
| | | | | | DIPEPTIDASE DM00816 | DOMO |
| | | | | | S64780\|449-697: I283-P534 | |
| | | | | | P54518\|121-347: K322-V510 | |
| | | | | | P46545\|131-358: E319-N506 | |
| | | | | | Q10698\|140-366: K287-V510 | |
| 19 | 7503233CD1 | 652 | S77 S147 S194 | N76 N137 N305 | Calpain large subunit, domain III: K303-V460 | HMMER_{—} |
| | | | S248 S275 S317 | N406 | | PFAM |
| | | | S422 S437 S453 | | | |
| | | | S477 S500 S527 | | | |
| | | | S574 S602 T202 | | | |
| | | | T284 T300 T307 | | | |
| | | | T312 T632 | | | |
| | | | | | Calpain family cysteine protease: L55-T292 | HMMER_{—} |
| | | | | | | PFAM |
| | | | | | EF hand: N557-A585, S527-I555, T622-T649, N484-I511 | HMMER_{—} |
| | | | | | | PFAM |
| | | | | | EF-hand calcium-binding domain proteins | BLIMPS_{—} |
| | | | | | BL00018: D536-F548 | BLOCKS |
| | | | | | Calpain cysteine protease (C2) family signature | BLIMPS_{—} |
| | | | | | PR00704: Y83-I108, L113-V136, G138-L165, | PRINTS |
| | | | | | E268-C289, T318-F335, R426-E454 | |
| | | | | | PROTEASE CALPAIN HYDROLASE SUBUNIT | BLAST_{—} |
| | | | | | NEUTRAL THIOL LARGE CALCIUM- | PRODOM |
| | | | | | ACTIVATED PROTEINASE CANP PD001545: | |
| | | | | | Y83-T292, L55-G82 | |
| | | | | | PROTEASE CALPAIN HYDROLASE SUBUNIT | BLAST_{—} |
| | | | | | LARGE NEUTRAL THIOL CALCIUM- | PRODOM |
| | | | | | ACTIVATED PROTEINASE CANP PD001874: | |
| | | | | | K303-T459 | |
| | | | | | CALPAIN SUBUNIT CALCIUM-BINDING | BLAST_{—} |
| | | | | | NEUTRAL PROTEASE CALCIUM-ACTIVATED | PRODOM |
| | | | | | PROTEINASE CANP HYDROLASE LARGE | |
| | | | | | PD003609: E480-F548 | |
| | | | | | CALPAIN SUBUNIT PROTEASE NEUTRAL | BLAST_{—} |
| | | | | | CALCIUM-BINDING CALCIUM-ACTIVATED | PRODOM |
| | | | | | PROTEINASE CANP HYDROLASE LARGE | |
| | | | | | PD002827: N549-V614 | |
| | | | | | CALPAIN CATALYTIC DOMAIN DM01305 | BLAST_{—} |
| | | | | | P07384\|11-517: G82-S456, C11-G82 | DOMO |
| | | | | | P00789\|3-507: Y83-K455, G13-G82 | |
| | | | | | P17655\|1-505: N76-K455, G13-G82, V434-Q491 | |
| | | | | | A48764\|1-507: Q66-K455, V14-G82, Y432-Q491, | |
| | | | | | K521-R554 | |
| | | | | | EF-hand calcium-binding domain: D536-F548, D566-M578 | MOTIFS |
| 20 | 7726576CD1 | 861 | S12 S29 S187 S239 | N257 N577 N779 | HECT-domain (ubiquitin-transferase): | HMMER_{—} |
| | | | S251 S264 S324 | | P565-S857 | PFAM |
| | | | S327 S372 S465 | | | |
| | | | S509 S547 S558 | | | |
| | | | S591 S628 S834 | | | |
| | | | S857 T106 T107 | | | |
| | | | T268 T284 T289 | | | |
| | | | T300 T399 T435 | | | |
| | | | T735 T758 T781 | | | |
| | | | T793 Y844 | | | |
| | | | | | Pyrokinins proteins BL00539: F470-L474 | BLIMPS_{—} |
| | | | | | | BLOCKS |
| | | | | | HECT-domain (ubiquitin-transferase) PF00632: | BLIMPS_{—} |
| | | | | | V680-G686, Y772-P799, L817-N848 | PFAM |
| | | | | | PROTEIN LIGASE UBIQUITIN CONJUGATION | BLAST_{—} |
| | | | | | REPEAT UBIQUITIN PROTEIN DNA-BINDING | PRODOM |
| | | | | | PROBABLE ONCOGENIC PD002225: V587-A845 | |
| | | | | | HERC2 RELEASING UBIQUITIN FACTOR | BLAST_{—} |
| | | | | | REPEAT CONJUGATION GUANINE- | PRODOM |
| | | | | | NUCLEOTIDE KIAA0076 HA0936 PD155960: | |
| | | | | | E245-I370 | |
| | | | | | HECT DOMAIN DM01690 | BLAST_{—} |
| | | | | | P51593\|9-306: R583-C849 | DOMO |
| | | | | | P40985\|578-891: Y598-A845 | |
| | | | | | P39940\|513-808: N589-A845 | |
| | | | | | A38919\|785-1082: N574-A851 | |
| | | | | | Leucine zipper pattern: L404-L425 | MOTIFS |
| 21 | 7503507CD1 | 447 | S11 S34 S53 S201 | N164 N291 | signal_cleavage: M1-A37 | SPSCAN |
| | | | S234 S339 S377 | | | |
| | | | S382 S395 S406 | | | |
| | | | T166 T197 T263 | | | |
| | | | T279 T397 | | | |
| | | | | | Signal Peptide: M1-A37 | HMMER |
| | | | | | HEPATOCYTE GROWTH FACTOR ACTIVATOR | BLAST_{—} |
| | | | | | INHIBITOR GLYCOPROTEIN PD120361: G84-L297 | PRODOM |
| 22 | 7503506CD1 | 468 | S11 S34 S53 S201 | N164 N291 N401 | signal_cleavage: M1-A37 | SPSCAN |
| | | | S234 S339 S377 | | | |
| | | | S382 S395 T166 | | | |
| | | | T197 T263 T279 | | | |
| | | | T397 T455 | | | |
| | | | | | Signal Peptide: M1-A37 | HMMER |
| | | | | | Low-density lipoprotein receptor domain: | HMMER_{—} |
| | | | | | H308-N346 | PFAM |
| | | | | | Cytosolic domain: A438-L468 | TMHMMER |
| | | | | | Transmembrane domain: V415-V437 | |
| | | | | | Non-cytosolic domain: M1-P414 | |
| | | | | | LDL-receptor class A (LDLRA) domain proteins | BLIMPS_{—} |
| | | | | | BL01209: C329-E341 | BLOCKS |
| | | | | | HEPATOCYTE GROWTH FACTOR ACTIVATOR | BLAST_{—} |
| | | | | | INHIBITOR GLYCOPROTEIN | PRODOM |
| | | | | | PD120361: G84-L297, S11-P44 | |
| | | | | | LDL-receptor class A (LDLRA) domain signature: | MOTIFS |
| | | | | | C322-C344 | |
| | | | | | Leucine zipper pattern: L45-L66, L427-L448 | MOTIFS |
| 23 | 7503509CD1 | 236 | S11 S34 S53 S201 | N164 | signal_cleavage: M1-A37 | SPSCAN |
| | | | S207 T166 T197 | | | |
| | | | | | Signal Peptide: M1-A37 | HMMER |
| | | | | | HEPATOCYTE GROWTH FACTOR ACTIVATOR | BLAST_{—} |
| | | | | | INHIBITOR GLYCOPROTEIN PD120361: G84-R188, | PRODOM |
| | | | | | S11-P44 | |
| | | | | | Leucine zipper pattern: L45-L66 | MOTIFS |
| 24 | 7505800CD1 | 312 | S49 T283 T302 | N166 N179 N206 | Protein prenyltransferase alpha subunit repe: Q117-K147, | HMMER_{—} |
| | | | Y87 | | E83-R113, R191-G221, N151-T181 | PFAM |
| | | | | | Protein prenyltransferases alpha subunit repeat | BLIMPS_{—} |
| | | | | | proteins proteins BL00904: E83-S116, Q117-D157, | BLOCKS |
| | | | | | R191-G215, I216-E253, Q259-T283 | |
| | | | | | TRANSFERASE SUBUNIT ALPHA PROTEIN | BLAST_{—} |
| | | | | | PRENYL TRANSFERASE REPEAT FARNESYL | PRODOM |
| | | | | | TRANSFERASE PROTEINS CAAX RAS | |
| | | | | | PD005875: A40-Q236, D59-Q311 | |
| | | | | | FARNESYL TRANSFERASE; ALPHA; | BLAST_{—} |
| | | | | | DM07118\|P49354\|257-378: E190-Q312 | DOMO |
| | | | | | PROTEIN PRENYL TRANSFERASES ALPHA | BLAST_{—} |
| | | | | | SUBUNIT REPEAT DM01356\|P49354\|174-212: | DOMO |
| | | | | | V107-F146 | |
| | | | | | Protein prenyltransferases alpha subunit repeat | MOTIFS |
| | | | | | signature: P96-R105, A130-R139, V164-R173, P204-L213 | |
| 25 | 7503141CD1 | 452 | S138 S167 S271 | N13 N172 | metallopeptidase family M24: V143-E432 | HMMER_{—} |
| | | | S355 S419 T15 T54 | | | PFAM |
| | | | T90 T146 T358 | | | |
| | | | T430 Y128 | | | |
| | | | | | Aminopeptidase P and proline dipeptidase proteins | BLIMPS_{—} |
| | | | | | BL00491: A278-H289, H325-D337, L362-F376, | BLOCKS |
| | | | | | G407-G420 | |
| | | | | | PROLIDASE HYDROLASE XAAPRO | BLAST_{—} |
| | | | | | DIPEPTIDASE XPRO PROLINE | PRODOM |
| | | | | | IMIDODIPEPTIDASE ACETYLATION | |
| | | | | | MANGANESE PEPTIDASE PD013444: A2-C183 | |
| | | | | | AMINOPEPTIDASE HYDROLASE METHIONINE | BLAST_{—} |
| | | | | | PEPTIDASE PROTEIN COBALT M DIPEPTIDASE | PRODOM |
| | | | | | XPRO MAP PD000555: E160-Y375; G404-T425 | |
| | | | | | AMINOPEPTIDASE P AND PROLINE | BLAST_{—} |
| | | | | | DIPEPTIDASE DM00816 | DOMO |
| | | | | | \|P12955\|179-467: V181-P428 | |
| | | | | | \|P43590\|225-509: V181-T425 | |
| | | | | | \|P15034\|169-423: H187-Y375; E397-T425 | |
| | | | | | \|P44881\|163-417: K168-Y375; E397-P428 | |
| | | | | | Aminopeptidase P and proline dipeptidase signature: | MOTIFS |
| | | | | | H325-D337 | |
| 26 | 7500362CD1 | 471 | S116 S145 S202 | N13 N150 | signal_cleavage: M1-T56 | SPSCAN |
| | | | S290 S374 S438 | | | |
| | | | T15 T68 T124 | | | |
| | | | T166 T176 T377 | | | |
| | | | T449 Y106 | | | |
| | | | | | metallopeptidase family M24: V163-E451 | HMMER_{—} |
| | | | | | | PFAM |
| | | | | | Aminopeptidase P and proline dipeptidase proteins | BLIMPS_{—} |
| | | | | | BL00491: A297-H308, H344-D356, L381-F395, | BLOCKS |
| | | | | | G426-G439 | |
| | | | | | Pyrokinins proteins BL00539: F74-L78 | BLIMPS_{—} |
| | | | | | | BLOCKS |
| | | | | | PROLIDASE HYDROLASE XAAPRO | BLAST_{—} |
| | | | | | DIPEPTIDASE XPRO PROLINE IMIDO- | PRODOM |
| | | | | | DIPEPTIDASE ACETYLATION MANGANESE | |
| | | | | | PEPTIDASE | |
| | | | | | PD013444: A2-F48, F48-V163 | |
| | | | | | AMINOPEPTIDASE HYDROLASE METHIONINE | BLAST_{—} |
| | | | | | PEPTIDASE PROTEIN COBALT M DIPEPTIDASE | PRODOM |
| | | | | | XPRO MAP | |
| | | | | | PD000555: V163-Y394, G423-T444 | |
| | | | | | AMINOPEPTIDASE P AND PROLINE | BLAST_{—} |
| | | | | | DIPEPTIDASE DM00816 | DOMO |
| | | | | | \|P12955\|179-467: I158-P447 | |
| | | | | | \|P43590\|225-509: E160-T444 | |
| | | | | | \|P15034\|169-423: E160-Y394, E416-T444 | |
| | | | | | \|P44881\|163-417: E160-Y394, E416-P447 | |
| | | | | | Aminopeptidase P and proline dipeptidase signature: | MOTIFS |
| | | | | | H344-D356 | |
| 27 | 7503328CD1 | 458 | S4 S97 S220 S257 | N132 | AMINOPEPTIDASE AMINOACYL-PROLINE | BLAST_{—} |
| | | | S289 S326 S334 | | HYDROLASE P XAA PRO XPRO PROLINE | PRODOM |
| | | | S404 S431 T36 | | PRECURSOR PROTEIN P-LIKE | |
| | | | T145 T199 T243 | | PD008419: G183-A363 | |
| | | | | | AMINOPEPTIDASE HYDROLASE | BLAST_{—} |
| | | | | | AMINOACYLPROLINE XAAPRO P XPRO | PRODOM |
| | | | | | PROTEIN PUTATIVE PROLINE APP PD004954: | |
| | | | | | L53-D163 | |
| 28 | 7510464CD1 | 695 | S4 S97 S220 S257 | N132 N446 | metallopeptidase family M24: A363-N595 | HMMER_{—} |
| | | | S289 S326 S334 | | | PFAM |
| | | | S404 S450 S522 | | | |
| | | | S548 S619 S670 | | | |
| | | | T36 T145 T199 | | | |
| | | | T243 T445 T480 | | | |
| | | | T551 T621 T643 | | | |
| | | | | | Aminopeptidase P and proline dipeptidase proteins | BLIMPS_{—} |
| | | | | | BL00491: I427-H438, H528-V540, L557-E571, | BLOCKS |
| | | | | | G576-K589 | |
| | | | | | Proteasome A-type and B-type PF00227: I74-Y85 | BLIMPS_{—} |
| | | | | | | PFAM |
| | | | | | AMINOPEPTIDASE AMINOACYL PROLINE | BLAST_{—} |
| | | | | | HYDROLASE P XAA PRO XPRO PROLINE | PRODOM |
| | | | | | PRECURSOR PROTEIN P-LIKE PD008419: G183-A363 | |
| | | | | | AMINOPEPTIDASE HYDROLASE AMINOACYL | BLAST_{—} |
| | | | | | PROLINE XAA PRO P XPRO PROTEIN | PRODOM |
| | | | | | PUTATIVE PROLINE APP PD004954: L53-D163 | |
| | | | | | AMINOPEPTIDASE HYDROLASE METHIONINE | BLAST_{—} |
| | | | | | PEPTIDASE PROTEIN COBALT M DIPEPTIDASE | PRODOM |
| | | | | | XPRO MAP PD000555: V364-G568, E444-V585 | |
| | | | | | AMINOPEPTIDASE P AND PROLINE | BLAST_{—} |
| | | | | | DIPEPTIDASE | DOMO |
| | | | | | DM00816\|S64780\|449-697: I358-P609 | |
| | | | | | DM00816\|P54518\|121-347: K397-V585 | |
| | | | | | DM00816\|P46545\|131-358: E394-N581 | |
| | | | | | DM00816\|Q10698\|140-366: K362-V585 | |
| 29 | 7510394CD1 | 140 | S29 S81 S108 T52 | | signal_cleavage: M1-A23 | SPSCAN |
| | | | T119 T129 | | | |
| | | | | | Signal Peptide: M5-C21, M5-A23, M1-C21, M1-A23, | HMMER |
| | | | | | M1-P25 | |
| | | | | | DEVELOPMENT-ASSOCIATED NEURONAL | BLAST_{—} |
| | | | | | T1B9.20 DJ453C12.7 CGI-06 | PRODOM |
| | | | | | PD043343: E32-A134 | |
| 30 | 7500745CD1 | 191 | S29 S81 T52 | | signal_cleavage: M1-A23 | SPSCAN |
| | | | | | Signal Peptide: M1-C21, M1-A23, M1-P25, M5-C21, | HMMER |
| | | | | | M5-A23 | |
| 31 | 7500929CD1 | 145 | S35 | | signal_cleavage: M1-A19 | SPSCAN |
| | | | | | Signal Peptide: M1-A26 | HMMER |

TABLE 4

| Polynucleotide | |
|---|---|
| SEQ ID NO:/ | |
| Incyte ID/Sequence | |
| Length | Sequence Fragments |
| 32/8268274CB1/ | 1-530, 53-231, 53-626, 81-845, 127-387, 174-746, 337-654, 369-766, 377-1014, 420-716, 443-887, 478-761, 505-745, |
| 2129 | 505-921, 505-1101, 523-1000, 545-838, 605-913, 645-864, 665-721, 671-962, 671-967, 714-922, 758-1019, |
| | 778-1249, 796-1046, 846-1358, 868-1104, 1146-1296, 1159-1613, 1236-1748, 1284-1319, 1316-1860, 1317-1714, |
| | 1361-1926, 1362-1597, 1421-1794, 1450-1747, 1471-1748, 1616-1913, 1616-2097, 1616-2119, 1618-1887, 1666-2129, |
| | 1699-1952, 1715-1960 |
| 33/7500515CB1/ | 1-799, 1-845, 1-851, 1-870, 1-880, 1-894, 1-907, 1-942, 1-2859, 8-171, 8-193, 11-229, 14-736, 108-845, 199-790, |
| 3489 | 217-577, 233-886, 288-898, 299-804, 302-942, 339-790, 350-859, 360-629, 391-890, 396-988, 461-739, 566-1183, |
| | 587-1301, 589-1360, 595-738, 621-1216, 632-981, 736-1009, 736-1288, 741-1363, 790-1019, 814-1581, 853-1339, |
| | 854-1114, 863-1536, 864-1206, 868-1340, 872-1479, 874-1195, 897-1596, 898-1165, 904-1173, 912-1626, 948-1626, |
| | 991-1220, 1001-1631, 1037-1250, 1047-1803, 1093-1751, 1101-1809, 1103-1738, 1127-1664, 1160-1409, |
| | 1189-1873, 1190-1874, 1235-1576, 1241-1506, 1297-1463, 1302-1880, 1370-1491, 1370-1624, 1390-1779, 1401-1574, |
| | 1404-1868, 1419-1651, 1437-1875, 1442-1854, 1456-1756, 1474-1880, 1495-1759, 1530-1810, 1542-1880, |
| | 1544-1797, 1609-2049, 1660-1933, 1676-1926, 1704-1866, 1710-1880, 1751-2098, 1835-2412, 1852-2366, 1866-2472, |
| | 1916-2859, |
| | 1940-2861, 1963-2861, 1985-2858, 1991-2859, 1995-2608, 2007-2861, 2008-2859, 2014-2375, 2032-2557, 2083-2690, |
| | 2104-2667, 2104-2838, 2106-2617, 2107-2389, 2111-2606, 2138-2687, 2141-2775, 2148-2744, 2158-2692, |
| | 2160-2467, 2176-2822, 2188-2857, 2199-2383, 2221-2798, 2260-2483, 2265-2827, 2267-2789, 2276-2536, 2281-2603, |
| | 2282-2526, 2294-2876, 2307-2845, 2310-2429, 2325-2848, 2340-2591, 2342-2845, 2349-2582, 2365-2788, |
| | 2367-2702, 2374-2632, 2386-2518, 2388-2654, 2388-2847, 2388-2873, 2397-2826, 2401-2849, 2428-2646, 2435-2862, |
| | 2435-2876, 2435-2877, 2454-2863, 2456-2673, 2481-2865, 2493-2858, 2497-2753, 2498-2751, 2525-3489, |
| | 2533-2698, 2536-2839, 2542-2792, 2543-2785, 2550-2790, 2550-2846, 2550-2857, 2550-2871, 2576-2824, 2608-2858, |
| | 2662-2856, 2669-2857, 2735-2855, 2737-2898 |
| 34/2256826CB1/ | 1-686, 424-2842, 443-1082, 452-978, 461-1074, 511-800, 511-867, 511-924, 530-597, 530-881, 567-987, 688-1062, |
| 2996 | 721-837, 721-956, 721-970, 721-1144, 721-1155, 721-1188, 721-1222, 721-1232, 721-1236, 721-1270, 721-1273, |
| | 721-1279, 721-1281, 721-1294, 721-1312, 721-1322, 722-1351, 725-1342, 785-1330, 851-1473, 919-1600, 971-1410, |
| | 982-1572, 985-1574, 998-1581, 1001-1478, 1055-1705, 1072-1724, 1105-1645, 1123-1745, 1142-1774, 1187-1828, |
| | 1217-1619, 1274-1827, 1280-1914, 1296-1936, 1341-1907, 1345-1836, 1363-1735, 1371-1814, 1379-1950, |
| | 1392-1852, 1440-1996, 1456-1935, 1458-1944, 1464-2017, 1469-1984, 1481-1987, 1488-1997, 1491-1967, 1491-1988, |
| | 1494-1988, 1504-1980, 1504-2035, |
| | 1517-2051, 1538-2209, 1547-2109, 1555-2183, 1580-2238, 1591-2236, 1610-2261, 1619-2099, 1657-2220, 1666-2180, |
| | 1671-2114, 1672-2315, 1675-2370, 1677-2222, 1686-2149, 1691-2250, 1697-2189, 1736-2292, 1755-2235, |
| | 1775-2375, 1789-2266, 1794-2363, 1828-2395, 1852-2368, 1859-2317, 1890-2529, 1911-2493, 1946-2352, 1953-2498, |
| | 1962-2435, 1977-2617, 2072-2610, 2093-2602, 2102-2498, 2113-2565, 2137-2498, 2141-2546, 2158-2706, |
| | 2173-2663, 2177-2803, 2191-2842, 2207-2719, 2208-2813, 2316-2799, 2330-2996, 2356-2817, 2375-2805, 2398-2822, |
| | 2452-2828 |
| 35/7686186CB1/ | 1-936, 4-820, 32-1002, 118-955, 135-957, 141-970, 142-951, 150-1094, 178-800, 208-1045, 223-1245, 235-1130, |
| 1860 | 244-1046, 254-1067, 271-1007, 314-1137, 318-946, 340-1181, 377-1000, 377-1005, 399-1218, 420-1190, 444-1017, |
| | 473-1086, 473-1321, 476-1219, 502-1002, 508-924, 542-1375, 580-1054, 594-1461, 620-1526, 625-1457, 649-1078, |
| | 732-1432, 735-1244, 744-1312, 769-945, 777-1648, 781-1314, 817-1629, 818-1618, 818-1626, 825-1721, 830-1667, |
| | 832-1814, 837-1381, 839-1216, 839-1389, 881-1668, 924-1756, 930-1696, 939-1844, 958-1844, 965-1830, 970-1814, |
| | 973-1457, 980-1814, 980-1826, 985-1814, 988-1737, 991-1814, 995-1834, 998-1814, 999-1770, 999-1846, |
| | 1003-1836, 1018-1400, 1022-1830, 1022-1860, |
| | 1024-1814, 1029-1842, 1035-1837, 1036-1830, 1037-1814, 1038-1845, 1039-1814, 1040-1833, 1042-1830, 1049-1828, |
| | 1055-1811, 1056-1814, 1060-1814, 1061-1814, 1064-1557, 1066-1814, 1069-1700, 1074-1844, 1082-1818, |
| | 1103-1841, 1111-1814, 1122-1814, 1129-1814, 1132-1840, 1141-1814, 1147-1814, 1153-1812, 1154-1814, 1160-1814, |
| | 1168-1814, 1176-1814, 1176-1823, 1186-1814, 1193-1842, 1196-1814, 1205-1814, 1222-1814, 1226-1814, |
| | 1257-1834, 1263-1814, 1266-1814, 1270-1814, 1271-1814, 1276-1814, 1277-1816, 1280-1814, 1288-1830, 1296-1814, |
| | 1310-1814, 1311-1600, 1311-1814, 1328-1842, 1336-1814, 1359-1814, 1446-1835, 1461-1844, 1462-1815, |
| | 1469-1814, 1536-1738 |
| 36/72617436CB1/ | 1-281, 1-1334, 77-432, 77-477, 78-375, 78-407, 78-456, 78-459, 78-485, 85-375, 94-317, 94-379, 94-380, 94-1334, |
| 1334 | 114-576, 159-622, 282-600, 282-917, 282-966, 282-1035, 376-758, 581-1334, 859-1334 |
| 37/7501945CB1/ | 1-655, 502-984, 792-2070, 892-1172, 969-1266, 969-1298, 985-1207, 985-1208, 985-1270, 1173-1832, 1173-1841, |
| 2070 | 1173-1867, 1173-1890, 1173-1918, 1173-1931, 1173-1966, 1173-1983, 1173-1995, 1173-1996, 1173-2044, 1173-2054, |
| | 1177-1979, 1233-2070, 1234-2069, 1236-1708, 1242-2070, 1277-2070, 1336-2070, 1417-1800, 1514-1801, |
| | 1524-2070, 1579-1899 |
| 38/7500264CB1/ | 1-471, 1-2265, 46-537, 113-743, 113-800, 113-850, 113-871, 114-589, 114-856, 117-857, 124-619, 649-1118, 649-1129, |
| 2265 | 653-785, 653-1234, 657-931, 657-1159, 657-1162, 660-1185, 665-1449, 667-1000, 672-908, 677-1137, 691-826, |
| | 691-880, 691-901, 693-891, 759-906, 915-1449, 937-1449, 948-1449, 1012-1290, 1025-1699, 1033-1457, 1034-1290, |
| | 1066-1363, 1144-1419, 1164-1396, 1238-1542, 1242-1471, 1242-1491, 1246-1496, 1251-1856, 1282-1539, |
| | 1282-1540, 1282-1807, 1282-1874, 1322-1813, 1328-1797, 1334-1847, 1334-1882, 1347-1808, 1381-1640, 1416-1707, |
| | 1417-1704, 1420-1641, 1420-2200, 1475-1950, 1486-1963, 1529-1832, 1551-1814, 1551-2113, 1589-1896, |
| | 1597-1876, 1603-1829, 1618-1858, 1638-1918, 1666-1914, 1666-1961, 1667-1931, 1673-1955, 1702-1968, 1716-2006, |
| | 1718-1905, 1747-2170, 1808-1952, 1808-2126, 1819-2035, 1819-2149, 1853-2064, 1853-2244, 1859-2149, |
| | 1859-2265, 1885-2167, 1892-2117, 1909-2176, 1941-2156, 1941-2161, 1954-2192, 1956-2222, 1956-2223, 2004-2251, |
| | 2012-2253, 2031-2197, 2031-2265, 2039-2265, 2075-2211 |
| 39/7499935CB1/ | 1-369, 1-654, 1-715, 1-1174, 2-285, 2-300, 2-429, 2-458, 2-514, 2-524, 2-583, 2-594, 2-608, 2-609, 2-637, 2-642, 2-646, |
| 1834 | 2-663, 2-691, 2-735, 2-772, 2-838, 3-290, 3-308, 3-362, 3-583, 5-146, 5-308, 5-343, 5-484, 5-536, 5-541, 6-609, |
| | 7-267, 7-285, 7-297, 8-199, 8-206, 8-236, 8-273, 8-547, 8-655, 9-633, 11-253, 11-585, 12-227, 12-243, 12-254, 12-283, |
| | 12-319, 14-524, 14-1834, 15-270, 15-272, 15-287, 16-247, 16-252, 16-277, 16-296, 16-297, 16-529, 16-652, 16-775, |
| | 17-282, 17-290, 17-294, 18-133, 18-260, 18-290, 18-311, 18-623, 25-260, 25-313, 25-320, 26-133, 26-147, 26-180, |
| | 26-259, 26-295, 26-296, 26-304, 26-305, 26-450, 26-532, 26-578, 26-593, 26-648, 28-497, 28-600, 31-373, 33-295, |
| | 34-646, 34-657, 37-452, 37-637, 37-676, 43-344, 51-305, 53-850, 89-583, 103-644, 105-576, 124-394, 143-305, |
| | 160-653, 164-470, 233-462, 234-848, 249-1031, 255-476, 255-541, 258-638, 278-490, 278-495, 289-925, 300-592, |
| | 306-700, |
| | 306-719, 306-816, 306-879, 306-887, 306-888, 306-900, 306-945, 306-947, 306-961, 306-986, 307-789, 307-824, |
| | 307-831, 307-840, 307-841, 307-855, 307-858, 307-865, 307-891, 307-899, 307-932, 307-944, 307-945, 307-952, |
| | 307-955, 307-978, 307-981, 307-1005, 308-884, 309-479, 309-977, 318-568, 322-563, 325-548, 328-641, 329-546, |
| | 346-933, 347-1045, 350-1032, 353-830, 354-1015, 364-607, 364-639, 376-1135, 413-673, 413-1012, 415-703, 415-980, |
| | 416-895, 422-1121, 423-833, 427-824, 427-933, 452-722, 465-1170, 507-757, 521-1242, 542-803, 547-1187, |
| | 563-891, 580-1181, 591-881, 592-889, 627-1000, 630-844, 653-874, 655-935, 663-886, 673-914, 683-953, 690-949, |
| | 693-925, 693-927, 693-1319, 693-1433, 693-1461, 856-1576, 884-1164, 884-1169, 896-1150, 939-1167, 947-1229, |
| | 947-1414, 947-1415, 947-1438, 947-1456, 947-1457, 947-1523, 947-1589, 947-1591, 947-1611, 947-1635, 947-1645, |
| | 952-1179, 953-1444, 1130-1667, 1170-1526, 1275-1830, 1352-1419, 1377-1830, 1450-1830, 1515-1831, 1534-1788 |
| 40/7982285CB1/ | 1-292, 2-421, 44-805, 57-109, 59-624, 61-272, 61-656, 61-697, 61-698, 67-216, 67-247, 68-749, 74-437, 74-544, 74-573, |
| 1524 | 74-579, 74-676, 74-679, 77-756, 81-388, 82-340, 83-388, 84-658, 89-352, 90-309, 90-530, 91-306, 91-313, 91-674, |
| | 93-216, 95-356, 95-432, 95-435, 95-500, 95-501, 95-534, 95-545, 95-548, 96-767, 97-919, 98-505, 98-754, 100-346, |
| | 101-635, 102-960, 118-368, 123-544, 123-617, 151-787, 154-409, 162-780, 203-880, 224-388, 239-815, 305-388, |
| | 369-1003, 392-853, 463-1070, 500-1100, 501-761, 519-1155, 536-565, 539-672, 608-1261, 628-1186, 678-1175, |
| | 697-1241, 804-1446, 839-1111, 845-1478, 916-1524, 929-1088, 938-1524, 957-1226, 957-1473, 972-1418, |
| | 1036-1085 |
| 41/7758505CB1/ | 1-635, 119-364, 119-742, 119-752, 244-735, 244-753, 244-755, 244-869, 666-755, 750-1300, 754-2973, 961-1142, |
| 2973 | 1033-1562, 1033-1938, 1034-1689, 1185-2973, 1327-1934, 1335-1847, 1353-1947, 1796-2305, 1937-1966 |
| 42/6885756CB1/ | 1-1789, 401-1789, 402-1789, 1674-2126 |
| 2126 | |
| 43/7500748CB1/ | 1-482, 1-657, 1-671, 1-686, 2-597, 2-657, 8-256, 9-240, 11-361, 14-257, 14-266, 14-278, 15-276, 15-321, 15-529, 15-579, |
| 1973 | 19-271, 19-281, 19-284, 19-286, 20-273, 20-282, 20-285, 20-287, 20-288, 20-315, 22-242, 22-267, 22-277, 23-255, |
| | 23-294, 23-304, 23-321, 34-284, 36-642, 37-649, 42-273, 60-299, 93-228, 240-392, 255-489, 283-828, 313-1049, |
| | 361-923, 367-524, 367-540, 367-559, 405-557, 502-842, 520-766, 701-934, 726-953, 732-974, 755-1182, 758-920, |
| | 758-1015, 777-1038, 779-1029, 781-1035, 805-1049, 838-977, 863-1049, 897-1080, 934-1239, 940-1208, 1070-1360, |
| | 1073-1872, 1077-1708, 1080-1629, 1086-1472, 1090-1305, 1102-1966, 1113-1395, 1115-1314, 1119-1351, |
| | 1119-1784, 1126-1765, 1133-1525, 1136-1539, 1138-1426, 1138-1912, 1146-1724, 1166-1438, 1166-1598, 1186-1444, |
| | 1191-1437, 1205-1541, 1210-1930, 1210-1944, 1220-1552, 1221-1973, 1226-1479, 1235-1488, 1235-1840, |
| | 1236-1822, |
| | 1241-1502, 1242-1536, 1242-1861, 1256-1494, 1256-1523, 1256-1813, 1259-1460, 1260-1854, 1262-1460, 1265-1722, |
| | 1279-1589, 1282-1909, 1287-1929, 1296-1834, 1299-1876, 1300-1927, 1303-1605, 1305-1787, 1307-1565, |
| | 1309-1555, 1316-1788, 1316-1953, 1317-1805, 1318-1718, 1319-1929, 1327-1594, 1333-1583, 1333-1737, 1334-1554, |
| | 1335-1590, 1336-1576, 1336-1588, 1339-1562, 1342-1604, 1345-1966, 1346-1723, 1349-1444, 1353-1808, |
| | 1356-1851, 1357-1958, 1357-1968, 1359-1631, 1361-1939, 1362-1956, 1363-1643, 1366-1973, 1367-1638, 1375-1637, |
| | 1387-1967, 1394-1679, 1394-1695, 1395-1631, 1397-1665, 1404-1621, 1404-1641, 1404-1801, 1406-1550, |
| | 1406-1661, 1406-1682, 1406-1967, 1411-1617, 1418-1669, 1418-1852, 1422-1953, 1431-1968, 1435-1681, 1441-1845, |
| | 1454-1968, 1465-1949, 1467-1952, 1467-1973, 1471-1775, 1475-1726, 1481-1938, 1493-1763, 1499-1781, |
| | 1504-1769, 1529-1624, 1529-1933, 1531-1794, 1539-1934, 1540-1930, 1555-1814, 1561-1898, 1628-1829, 1664-1901 |
| 44/7500749CB1/ | 1-190, 1-1846, 22-670, 158-358, 190-408, 190-416, 190-456, 190-507, 190-523, 190-661, 190-662, 190-697, 190-724, |
| 1884 | 190-775, 190-794, 194-626, 195-801, 199-772, 201-447, 201-598, 208-729, 218-472, 221-813, 223-780, 224-818, |
| | 229-767, 235-834, 236-764, 239-830, 242-511, 246-866, 253-944, 259-915, 261-492, 264-506, 266-411, 266-526, |
| | 267-566, 271-542, 271-883, 278-873, 281-773, 284-651, 285-916, 297-704, 309-579, 316-859, 335-581, 339-702, |
| | 339-733, 340-835, 342-611, 342-889, 347-702, 353-952, 359-932, |
| | 365-629, 365-640, 368-616, 370-859, 372-955, 374-1118, 376-668, 381-793, 382-615, 382-640, 382-652, 385-670, |
| | 385-784, 386-1210, 387-589, 387-883, 390-975, 392-513, 395-651, 398-944, 407-634, 407-702, 413-655, 413-1003, |
| | 433-1091, 436-730, 439-601, 439-696, 441-1065, 452-913, 452-961, 452-985, 452-1193, 452-1197, 452-1218, 452-1274, |
| | 452-1303, 455-1162, 458-719, 459-639, 460-710, 462-716, 469-937, 474-1042, 477-1074, 486-734, 489-777, |
| | 491-1090, 493-1023, 493-1066, 496-1158, 497-959, 509-1018, 519-658, 522-1167, 523-1090, 532-1061, 542-1079, |
| | 544-772, 549-811, 551-838, 561-794, 563-986, 564-1169, 565-814, 565-1227, 571-875, 573-1271, 574-1256, 578-727, |
| | 583-1204, 588-824, 588-831, |
| | 592-1213, 593-887, 603-1111, 606-887, 611-950, 611-1245, 612-895, 614-859, 620-1228, 625-870, 629-755, 629-817, |
| | 639-1345, 642-922, 644-884, 649-1310, 650-1225, 652-920, 653-1191, 654-899, 656-995, 661-933, 665-934, |
| | 665-942, 670-902, 671-1164, 673-925, 675-948, 675-952, 675-963, 675-1208, 678-908, 680-1319, 683-935, 683-1262, |
| | 683-1279, 693-988, 697-858, 697-943, 700-908, 708-1363, 710-978, 710-1142, 713-1331, 715-1291, 719-978, |
| | 720-972, 720-974, 720-1018, 723-1035, 726-926, 726-941, 726-988, 728-887, 734-1365, 736-983, 742-973, 744-978, |
| | 744-1316, 745-1332, 746-923, 746-1032, 770-1381, 773-1076, 783-1090, 785-1051, 788-977, 790-1013, 793-1122, |
| | 793-1329, 798-1051, 798-1080, 798-1093, 800-1072, 800-1421, 802-1451, 803-893, 803-1116, 814-1344, 819-1405, |
| | 822-1523, 823-1095, 831-1088, 831-1328, 831-1345, 833-1264, 837-1341, 837-1346, 840-965, 842-1126, 842-1480, |
| | 843-1430, 848-1415, 848-1492, |
| | 852-1388, 861-1095, 862-1115, 876-1054, 886-1130, 895-1387, 901-1158, 906-1155, 908-1191, 911-1614, 912-1114, |
| | 913-1604, 915-1053, 922-1572, 925-1189, 925-1387, 926-1618, 932-1045, 932-1178, 939-1207, 942-1458, |
| | 943-1512, 952-1242, 953-1195, 953-1638, 955-1754, 959-1590, 962-1511, 965-1177, 966-1213, 968-1354, 969-1254, |
| | 971-1258, 972-1187, 984-1848, 993-1798, 995-1277, 997-1196, 1001-1233, 1001-1666, 1003-1235, 1003-1402, |
| | 1007-1601, 1008-1647, 1015-1407, 1018-1421, 1020-1281, 1020-1308, 1020-1515, 1020-1794, 1021-1254, |
| | 1028-1606, 1040-1316, 1042-1288, 1043-1349, 1045-1574, 1045-1669, 1048-1320, 1048-1480, 1055-1316, 1055-1318, |
| | 1060-1282, 1068-1326, 1073-1319, 1080-1629, 1084-1340, 1087-1423, 1087-1801, 1088-1145, 1092-1812, |
| | 1092-1826, 1097-1338, 1102-1330, 1102-1434, 1103-1855, 1105-1322, 1105-1436, 1108-1361, 1110-1663, 1112-1369, |
| | 1117-1370, 1117-1413, 1117-1722, 1118-1704, 1122-1403, 1122-1416, 1122-1785, 1123-1384, 1124-1418, |
| | 1124-1743, |
| | 1137-1376, 1138-1371, 1138-1376, 1138-1405, 1138-1695, 1141-1342, 1142-1736, 1144-1342, 1146-1820, 1147-1604, |
| | 1153-1721, 1159-1803, 1161-1471, 1163-1794, 1164-1791, 1169-1811, 1172-1806, 1178-1716, 1180-1837, |
| | 1181-1758, 1182-1777, 1182-1809, 1185-1487, 1187-1669, 1189-1447, 1191-1437, 1195-1761, 1198-1670, 1198-1835, |
| | 1199-1687, 1200-1600, 1200-1881, 1201-1811, 1201-1879, 1208-1446, 1209-1476, 1215-1465, 1215-1503, |
| | 1215-1619, 1216-1436, 1217-1472, 1218-1458, 1218-1470, 1220-1806, 1221-1444, 1222-1849, 1224-1486, 1224-1487, |
| | 1224-1492, 1227-1848, 1228-1605, 1231-1326, 1235-1690, 1238-1733, 1239-1840, 1239-1850, 1241-1513, |
| | 1243-1821, 1244-1838, 1245-1525, 1248-1863, 1249-1520, 1253-1847, 1255-1860, 1257-1519, 1262-1778, 1265-1863, |
| | 1266-1560, 1269-1849, 1276-1561, 1276-1577, 1276-1876, 1277-1513, 1279-1547, 1286-1503, 1286-1523, |
| | 1286-1586, |
| | 1286-1683, 1288-1432, 1288-1485, 1288-1543, 1288-1564, 1288-1849, 1288-1883, 1293-1499, 1295-1882, 1300-1551, |
| | 1300-1734, 1304-1835, 1310-1850, 1313-1881, 1317-1563, 1318-1882, 1323-1727, 1324-1470, 1334-1583, |
| | 1336-1850, 1338-1581, 1341-1622, 1341-1880, 1343-1809, 1347-1666, 1347-1831, 1348-1565, 1349-1572, 1349-1834, |
| | 1349-1864, 1353-1657, 1354-1608, 1357-1608, 1359-1880, 1363-1820, 1375-1645, 1381-1663, 1382-1821, |
| | 1382-1850, 1385-1847, 1386-1651, 1388-1884, 1392-1851, 1394-1636, 1396-1850, 1399-1883, 1402-1873, 1404-1848, |
| | 1409-1853, 1411-1506, 1411-1815, 1413-1676, 1414-1848, 1421-1816, 1422-1812, 1424-1858, 1426-1853, |
| | 1432-1850, 1433-1807, 1433-1849, 1433-1853, 1436-1849, 1436-1855, 1437-1696, 1438-1722, 1439-1849, 1441-1871, |
| | 1442-1849, 1442-1871, 1443-1780, 1443-1850, 1443-1862, 1443-1863, 1446-1849, 1448-1858, 1450-1856, |
| | 1452-1847, 1452-1855, 1453-1849, 1454-1753, 1455-1822, 1456-1734, 1456-1849, 1458-1827, 1459-1849, 1465-1859, |
| | 1468-1638, 1471-1883, 1472-1756, 1481-1696, 1481-1849, 1485-1850, 1486-1717, 1486-1867, 1488-1850, |
| | 1492-1883, 1495-1849, 1502-1524, 1502-1821, 1506-1849, 1510-1711, 1510-1853, 1515-1735, 1515-1850, 1524-1849, |
| | 1526-1805, 1526-1849, 1528-1812, 1528-1850, 1531-1850, 1532-1849, 1533-1851, 1537-1737, 1537-1796, |
| | 1544-1833, 1545-1760, 1545-1770, 1545-1790, 1545-1853, 1546-1783, 1547-1831, 1547-1844, 1547-1878, 1549-1830, |
| | 1550-1830, 1558-1849, 1560-1855, 1594-1793, 1596-1884, 1602-1855, 1604-1853, 1605-1849, 1648-1865, |
| | 1649-1883, 1654-1883, 1668-1872, 1682-1849, 1689-1883, 1692-1850, 1699-1868, 1711-1877, 1713-1849, 1734-1849, |
| | 1735-1863, 1736-1850, 1738-1883, 1753-1812, 1753-1848, 1753-1849, 1753-1850, 1758-1850, 1760-1864, |
| | 1765-1849 |
| 45/7503401CB1/ | 1-255, 1-328, 1-359, 1-436, 4-332, 10-295, 12-362, 13-317, 20-641, 21-272, 21-281, 21-436, 22-239, 22-616, 22-644, |
| 1581 | 26-341, 27-286, 27-287, 31-265, 34-311, 34-328, 35-299, 35-307, 37-327, 39-154, 46-289, 81-395, 85-380, 112-366, |
| | 143-413, 144-411, 146-399, 149-436, 178-436, 179-436, 185-947, 189-436, 237-429, 427-668, 432-755, 433-1284, |
| | 458-918, 487-650, 487-712, 489-995, 490-1360, 493-743, 494-991, 494-1134, 503-783, 507-910, 516-772, |
| | 517-753, 518-731, 520-763, 522-814, 522-1149, 524-703, 532-1024, 541-699, 543-807, 557-688, 559-818, 568-788, |
| | 569-1290, 586-849, 596-874, 599-882, 623-735, 642-904, 665-1282, 670-904, 676-922, 676-931, 678-1531, 681-1532, |
| | 693-1512, 694-899, 694-1059, 699-867, 701-941, 704-865, 704-997, 716-878, |
| | 716-1207, 717-1264, 725-1155, 734-1061, 737-1514, 741-1355, 743-1241, 748-1563, 759-1194, 770-1224, 775-1547, |
| | 781-1254, 788-1085, 788-1278, 794-1341, 800-1563, 801-1236, 807-1450, 820-1581, 822-1502, 835-1528, |
| | 836-1563, 839-1565, 841-1388, 841-1569, 846-1138, 846-1221, 850-1579, 861-1427, 863-1171, 865-1568, 865-1575, |
| | 874-1568, 875-1518, 878-1569, 882-1101, 883-1569, 884-1508, 885-1123, 888-1568, 897-1569, 898-1234, |
| | 901-1569, 906-1577, 919-1569, 925-1568, 927-1579, 932-1578, 933-1187, 933-1519, 937-1554, 938-1569, 948-1182, |
| | 948-1223, 950-1261, 950-1553, 952-1487, 954-1431, 954-1569, 955-1235, 960-1548, 964-1564, 967-1151, |
| | 970-1210, 973-1489, 975-1232, |
| | 980-1575, 985-1249, 989-1576, 991-1497, 992-1248, 992-1577, 992-1579, 993-1291, 996-1173, 996-1579, 998-1429, |
| | 1004-1224, 1010-1569, 1014-1261, 1019-1242, 1020-1248, 1028-1579, 1036-1579, 1042-1579, 1043-1523, |
| | 1050-1574, 1050-1579, 1055-1526, 1065-1579, 1068-1271, 1069-1579, 1076-1557, 1076-1575, 1078-1499, 1079-1350, |
| | 1081-1354, 1091-1306, 1092-1364, 1092-1565, 1094-1280, 1094-1579, 1096-1579, 1101-1566, 1102-1564, |
| | 1102-1579, 1104-1579, 1106-1569, 1109-1416, 1109-1568, 1109-1569, 1115-1567, 1115-1569, 1116-1566, 1121-1565, |
| | 1121-1567, 1126-1579, 1131-1567, 1132-1407, 1133-1581, 1134-1399, 1134-1420, 1136-1566, 1136-1579, |
| | 1137-1579, 1139-1564, 1139-1569, 1143-1567, 1143-1569, 1143-1579, 1144-1569, 1145-1427, 1146-1564, 1146-1579, |
| | 1148-1570, 1149-1564, 1150-1565, 1151-1562, 1152-1563, 1152-1565, 1154-1564, 1157-1569, 1157-1579, |
| | 1158-1487, 1158-1579, |
| | 1159-1567, 1164-1579, 1167-1564, 1168-1445, 1170-1564, 1173-1569, 1180-1451, 1181-1579, 1182-1569, 1183-1560, |
| | 1184-1564, 1184-1565, 1186-1579, 1186-1581, 1187-1564, 1187-1579, 1188-1563, 1188-1564, 1188-1570, |
| | 1189-1565, 1193-1579, 1195-1570, 1198-1562, 1206-1499, 1206-1569, 1209-1560, 1209-1579, 1214-1564, 1216-1473, |
| | 1220-1422, 1220-1567, 1221-1579, 1223-1493, 1223-1501, 1225-1579, 1226-1507, 1240-1565, 1246-1565, |
| | 1247-1564, 1254-1552, 1260-1567, 1261-1529, 1262-1567, 1268-1571, 1269-1564, 1272-1564, 1272-1565, 1277-1547, |
| | 1277-1562, 1278-1567, 1279-1485, 1288-1530, 1289-1564, 1289-1565, 1305-1581, 1308-1567, 1316-1572, |
| | 1316-1577, 1320-1564, 1322-1579, 1334-1574, 1355-1564, 1355-1565, 1355-1566, 1374-1562, 1374-1564, 1376-1579, |
| | 1387-1569, 1392-1578, 1397-1564, 1414-1567, 1414-1571, 1427-1579, 1434-1564, 1448-1564, 1449-1564, |
| | 1469-1564 |
| 46/7503485CB1/ | 1-282, 9-1724, 29-300, 30-292, 30-293, 34-305, 34-326, 52-266, 52-299, 52-325, 53-330, 53-338, 54-317, 54-328, |
| 1996 | 54-330, 54-335, 55-314, 57-303, 57-321, 57-323, 57-325, 57-328, 59-314, 60-268, 60-285, 60-305, 60-315, 60-316, |
| | 60-318, 60-321, 60-338, 60-342, 61-342, 62-272, 62-319, 63-308, 65-337, 66-298, 70-323, 72-330, 73-325, 106-287, |
| | 142-429, 176-668, 176-826, 238-520, 242-503, 242-532, 242-782, 242-873, 242-912, 325-958, 340-911, 348-649, |
| | 365-597, 371-936, 376-549, 377-641, 378-640, 387-704, 394-671, 397-586, 443-720, 443-1010, 447-705, 452-897, |
| | 455-966, 466-807, 472-1026, 486-610, 486-817, 505-1117, 506-651, 506-803, 528-980, 528-981, 529-1048, 537-1122, |
| | 548-776, 549-810, 550-859, 551-807, 556-1132, 560-778, 562-724, 593-858, 610-669, 611-875, 618-902, 619-921, |
| | 620-1249, 633-868, |
| | 641-876, 643-898, 645-911, 646-842, 646-900, 647-895, 656-864, 668-881, 676-1065, 676-1223, 677-916, 677-1012, |
| | 684-881, 684-965, 687-1135, 688-865, 688-968, 695-938, 702-1312, 708-970, 715-1343, 721-992, 735-949, |
| | 745-1336, 750-1362, 755-992, 757-1263, 777-1057, 777-1352, 788-999, 799-1062, 820-1271, 830-1087, 855-1092, |
| | 855-1298, 856-986, 856-1288, 869-1113, 871-1342, 873-1159, 879-1274, 880-1147, 883-1443, 885-1486, 890-1696, |
| | 902-1529, 908-1135, 910-1197, 918-1068, 918-1084, 918-1105, 918-1145, 918-1168, 918-1169, 920-1454, 921-1719, |
| | 924-1674, 939-1191, 941-1086, 943-1103, 944-1135, 944-1174, 944-1184, 944-1198, 955-1431, 962-1215, |
| | 965-1226, 974-1224, 975-1600, 979-1679, 987-1260, 987-1536, 992-1308, 993-1268, 993-1314, 995-1254, 997-1732, |
| | 999-1275, 1004-1209, 1007-1458, 1007-1651, 1021-1279, 1022-1715, 1034-1256, 1037-1293, 1045-1249, |
| | 1046-1752, 1054-1661, 1055-1228, |
| | 1055-1262, 1055-1269, 1055-1322, 1055-1324, 1056-1333, 1059-1655, 1066-1511, 1068-1718, 1069-1740, 1084-1302, |
| | 1086-1720, 1086-1748, 1093-1336, 1093-1714, 1095-1379, 1097-1380, 1100-1707, 1111-1344, 1118-1252, |
| | 1119-1696, 1123-1747, 1125-1418, 1125-1432, 1126-1386, 1126-1666, 1132-1645, 1132-1723, 1135-1399, 1139-1471, |
| | 1142-1716, 1148-1742, 1158-1388, 1160-1403, 1160-1721, 1164-1748, 1168-1410, 1172-1434, 1177-1746, |
| | 1184-1770, 1187-1724, 1197-1458, 1197-1469, 1211-1439, 1211-1468, 1211-1486, 1212-1742, 1234-1747, 1235-1742, |
| | 1240-1600, 1244-1749, 1247-1747, 1248-1745, 1249-1730, 1250-1724, 1253-1648, 1253-1745, 1254-1728, |
| | 1254-1745, 1258-1720, 1260-1730, 1260-1744, 1261-1729, 1265-1703, 1267-1482, 1268-1758, 1269-1745, 1271-1692, |
| | 1272-1522, 1272-1728, 1275-1728, 1277-1730, 1277-1731, 1278-1728, 1281-1519, 1283-1508, 1288-1727, |
| | 1290-1723, |
| | 1292-1720, 1293-1719, 1294-1720, 1296-1720, 1296-1725, 1296-1728, 1297-1720, 1297-1732, 1298-1673, 1298-1725, |
| | 1298-1752, 1300-1727, 1301-1724, 1301-1728, 1303-1729, 1303-1731, 1304-1720, 1305-1728, 1307-1729, |
| | 1308-1706, 1308-1727, 1308-1741, 1308-1747, 1309-1765, 1316-1720, 1319-1720, 1323-1720, 1325-1741, 1328-1727, |
| | 1330-1735, 1331-1720, 1333-1736, 1336-1732, 1337-1747, 1341-1677, 1346-1722, 1354-1727, 1356-1727, |
| | 1358-1500, 1361-1646, 1361-1732, 1367-1672, 1370-1503, 1370-1720, 1376-1718, 1377-1720, 1378-1720, 1382-1519, |
| | 1391-1734, 1409-1720, 1415-1663, 1421-1650, 1421-1672, 1421-1676, 1421-1759, 1422-1808, 1424-1727, |
| | 1433-1720, 1438-1709, 1439-1721, 1440-1725, 1453-1721, 1456-1714, 1457-1996, 1459-1738, 1459-1741, 1466-1715, |
| | 1468-1727, 1473-1727, 1475-1714, 1476-1728, 1479-1705, 1483-1729, 1512-1729, 1519-1729, 1524-1729, |
| | 1540-1729, 1550-1721, 1550-1783, 1560-1727, 1598-1721, 1610-1750, 1624-1724, 1642-1727 |
| 47/7504076CB1/ | 1-695, 16-646, 17-583, 27-487, 27-561, 46-668, 61-611, 83-448, 91-611, 159-614, 258-632, 258-693, 259-679, 259-845, |
| 1232 | 260-626, 260-632, 285-698, 285-848, 288-745, 298-590, 323-790, 361-791, 368-805, 373-832, 387-815, 393-873, |
| | 403-639, 429-774, 488-655, 497-746, 497-833, 497-1043, 506-813, 533-663, 543-783, 578-773, 583-1232 |
| 48/7500926CB1/ | 1-537, 11-467, 11-475, 22-190, 22-285, 22-295, 22-299, 22-400, 22-458, 22-468, 22-475, 22-478, 22-484, 23-386, |
| 810 | 23-482, 23-484, 26-379, 27-276, 27-434, 38-468, 41-810, 69-355, 164-471, 257-549, 280-469, 299-481, 299-546, |
| | 391-558, 405-809 |
| 49/7503216CB1/ | 1-586, 1-2598, 310-913, 335-913, 391-1023, 391-1043, 393-919, 393-928, 404-660, 580-884, 588-793, 928-1381, |
| 2625 | 930-1254, 930-1349, 930-1392, 931-1349, 937-1589, 953-1492, 953-1568, 955-1604, 959-1482, 972-1519, 975-1620, |
| | 976-1402, 996-1594, 1006-1227, 1009-1465, 1036-1732, 1043-1473, 1044-1640, 1052-1327, 1078-1345, 1100-1597, |
| | 1118-1603, 1136-1418, 1160-1749, 1167-1601, 1173-1548, 1184-1722, 1188-1417, 1193-1665, 1193-1736, |
| | 1196-1640, 1204-1489, 1222-1653, 1238-1506, 1246-2182, 1261-1676, 1267-2183, 1281-1753, 1291-1482, 1313-1789, |
| | 1318-1965, 1325-1792, 1337-1988, 1340-1678, 1341-1912, 1350-1747, 1352-2183, 1356-1633, 1358-1913, |
| | 1401-1969, 1408-1968, 1413-2183, 1418-1891, 1422-1975, 1434-1831, 1434-1904, 1434-2183, 1440-1995, 1451-1947, |
| | 1451-2183, 1452-2183, 1459-2108, 1479-1921, 1507-2183, 1536-1694, 1551-2075, 1579-2155, 1580-1809, |
| | 1580-1878, |
| | 1580-2183, 1584-1890, 1610-1875, 1614-1839, 1614-1891, 1614-1899, 1614-1929, 1614-1935, 1622-2156, 1625-1870, |
| | 1626-1918, 1640-2083, 1655-1962, 1657-1893, 1665-1935, 1675-1919, 1678-2244, 1678-2302, 1680-2112, |
| | 1719-2031, 1719-2341, 1721-2314, 1726-1975, 1735-2131, 1744-1989, 1746-1965, 1748-2130, 1748-2307, 1751-2240, |
| | 1758-1962, 1763-1977, 1763-2349, 1778-1935, 1797-2249, 1799-2327, 1816-2358, 1847-2137, 1866-2113, |
| | 1872-2592, 1879-2136, 1879-2472, 1881-2091, 1888-2475, 1902-2551, 1903-2134, 1911-2149, 1926-2222, 1926-2578, |
| | 1942-2124, 1943-2154, 1946-2559, 1946-2580, 1953-2218, 1953-2369, 1958-2581, 1970-2239, 1972-2207, |
| | 1982-2208, 2190-2612, 2275-2625, 2455-2598 |
| 50/7503233CB1/ | 1-253, 1-272, 1-2432, 15-311, 18-288, 19-257, 21-311, 23-244, 23-275, 24-311, 24-335, 27-335, 28-285, 33-294, 33-299, |
| 2432 | 37-271, 37-279, 38-305, 38-313, 44-313, 44-336, 48-304, 48-315, 51-335, 58-335, 79-319, 144-688, 292-778, |
| | 331-590, 331-787, 331-820, 331-927, 331-958, 331-959, 331-961, 331-965, 333-562, 334-961, 334-963, 335-786, |
| | 340-589, 342-945, 349-625, 349-630, 349-631, 353-852, 362-980, 366-747, 379-830, 381-975, 410-981, 427-1006, |
| | 442-1051, 448-948, 455-715, 456-705, 462-961, 467-813, 469-1146, 475-666, 480-989, 480-1063, 482-924, 488-683, |
| | 489-747, 494-719, 494-762, 522-738, 523-1028, 547-864, 547-1184, 556-1349, 564-1190, 571-745, 577-1154, |
| | 581-848, 593-1148, 609-1033, 615-915, 618-826, 618-1183, 621-1098, 623-1174, 623-1261, 631-1167, 632-881, |
| | 635-831, 635-1107, 636-986, 647-1178, 650-1371, |
| | 653-989, 656-1107, 665-899, 669-923, 669-1347, 682-1275, 692-939, 693-953, 694-1456, 706-1037, 712-984, 714-902, |
| | 719-1121, 723-1367, 733-985, 736-1012, 747-1034, 747-1273, 750-1452, 752-1001, 754-1015, 754-1314, 756-1139, |
| | 756-1140, 758-1299, 771-1333, 776-1065, 777-1360, 777-1440, 778-1260, 780-1413, 782-1076, 786-999, 798-1081, |
| | 805-1423, 806-1254, 835-1249, 838-1336, 851-1470, 852-1455, 854-1356, 854-1407, 855-1499, 856-1510, |
| | 857-1373, 860-1415, 865-1106, 865-1118, 868-1517, 884-1131, 886-1068, 891-1602, 894-1193, 906-1383, 916-1360, |
| | 917-1396, 918-1523, 925-1345, 930-1104, 938-1231, 946-1425, 948-1425, 954-1183, 966-1412, 971-1534, |
| | 973-1407, 976-1257, 977-1219, 987-1728, 992-1548, 993-1245, 993-1250, 994-1346, 995-1297, 997-1598, 1005-1278, |
| | 1006-1236, 1006-1916, 1027-1414, 1028-1414, 1030-1506, 1034-1491, 1035-1597, 1053-1253, 1054-1345, |
| | 1059-1448, |
| | 1063-1261, 1069-1423, 1070-1485, 1073-1231, 1074-1499, 1083-1683, 1099-1750, 1102-1401, 1103-1382, 1109-1284, |
| | 1114-1698, 1129-1414, 1141-1232, 1148-1635, 1154-1610, 1155-1696, 1157-1660, 1157-1785, 1158-1692, |
| | 1166-1839, 1174-1616, 1176-1492, 1186-1372, 1186-1661, 1190-1800, 1191-2008, 1192-1430, 1193-1765, 1202-1781, |
| | 1209-1735, 1209-1809, 1210-1451, 1213-1720, 1217-1431, 1219-1507, 1223-1331, 1224-1454, 1224-1467, |
| | 1230-1680, 1236-1936, 1241-1489, 1245-1808, 1258-1603, 1259-1517, 1264-1757, 1277-1907, 1289-1627, 1289-1864, |
| | 1292-1527, 1293-1702, 1295-1585, 1298-1621, 1299-1746, 1300-1584, 1308-1548, 1309-1833, 1309-2098, |
| | 1310-1851, 1312-1589, 1313-1902, 1314-1882, 1315-1809, 1317-1426, 1319-1765, 1326-1523, 1328-1447, 1351-1864, |
| | 1351-1977, 1354-1921, 1356-1941, 1366-2008, 1369-1844, 1377-1928, 1383-1792, 1389-1999, 1390-1890, |
| | 1394-2056, |
| | 1399-2162, 1402-1934, 1402-2041, 1403-2087, 1404-1907, 1415-1684, 1419-1907, 1421-2365, 1422-1968, 1422-2036, |
| | 1422-2049, 1423-1632, 1423-2066, 1426-1846, 1426-1974, 1432-2046, 1435-1953, 1437-2065, 1456-1902, |
| | 1456-1904, 1458-1587, 1459-1692, 1465-1736, 1467-1724, 1468-1769, 1469-1763, 1472-1701, 1472-1758, 1472-1839, |
| | 1472-2106, 1475-1768, 1478-1988, 1479-1747, 1482-1704, 1482-1758, 1483-2082, 1489-1930, 1493-2172, |
| | 1496-2024, 1507-1804, 1513-1748, 1514-2272, 1521-2025, 1525-1773, 1526-1747, 1528-1744, 1528-2007, 1528-2125, |
| | 1531-2113, 1535-2158, 1544-2377, 1553-1844, 1557-2110, 1559-2187, 1565-2229, 1566-1837, 1573-1824, |
| | 1574-1757, 1579-1816, 1579-1854, 1579-1870, 1579-2156, 1580-1902, 1583-2032, 1589-1987, 1590-2159, 1591-2160, |
| | 1591-2206, 1598-1869, 1599-2077, 1600-1851, 1600-2173, 1604-1991, 1607-1889, 1608-2053, 1615-1872, |
| | 1624-1869, 1624-1874, 1625-1874, 1626-1840, 1626-1864, 1626-1866, 1626-1882, 1628-1869, 1632-1924, |
| | 1638-1879, 1645-2136, 1647-2103, 1647-2247, 1649-1908, 1651-1922, 1651-2191, 1651-2206, 1652-2236, 1653-1968, |
| | 1656-2190, 1657-1902, 1673-1976, 1673-2245, 1674-1930, 1678-1987, 1678-1988, 1682-1883, 1691-1899, |
| | 1694-1841, 1696-1975, 1696-1983, 1696-2331, 1702-1922, 1702-2291, 1704-1996, 1705-1946, 1708-1954, 1708-1977, |
| | 1709-1926, 1709-1961, 1710-2280, 1712-1904, 1712-2118, 1715-2189, 1721-2129, 1727-2251, 1742-2182, |
| | 1748-1990, 1749-2028, 1755-1941, 1756-2115, 1763-1988, 1768-2272, 1768-2373, 1773-2339, 1777-2054, 1778-2020, |
| | 1781-2313, 1788-2288, 1789-2000, 1790-2045, 1798-2052, 1804-2086, 1807-2366, 1812-2097, 1813-2039, |
| | 1813-2404, 1825-2053, 1828-1993, 1829-2122, 1832-2069, 1832-2077, 1835-2353, 1838-2086, 1838-2126, 1840-2024, |
| | 1843-2120, 1844-2398, 1849-2362, 1868-2144, 1872-2160, 1873-2150, 1874-2119, 1877-2127, 1885-2420, |
| | 1886-2145, |
| | 1887-2170, 1894-2149, 1902-2174, 1907-2151, 1907-2157, 1907-2197, 1910-2123, 1912-2231, 1917-2098, 1917-2127, |
| | 1917-2169, 1917-2173, 1917-2198, 1917-2359, 1917-2398, 1919-2146, 1935-2132, 1935-2192, 1940-2122, |
| | 1940-2147, 1942-2190, 1944-2181, 1953-2266, 1959-2266, 1962-2202, 1962-2276, 1962-2279, 1963-2079, 1963-2116, |
| | 1970-2214, 1976-2430, 1977-2088, 1982-2214, 1982-2217, 1984-2225, 1984-2229, 1984-2232, 1984-2240, |
| | 1984-2255, 1984-2260, 1987-2254, 1995-2123, 1998-2266, 2001-2100, 2005-2216, 2021-2296, 2029-2313, 2030-2127, |
| | 2033-2151, 2035-2282, 2036-2243, 2044-2265, 2045-2265, 2080-2267, 2095-2353, 2099-2337, 2099-2349, |
| | 2102-2337, 2104-2337, 2107-2321, 2119-2398, 2121-2278, 2121-2285, 2121-2336, 2126-2349, 2127-2321, 2132-2323, |
| | 2132-2379, 2133-2381, 2133-2400, 2191-2389, 2205-2343, 2260-2398 |
| 51/7726576CB1/ | 1-473, 1-666, 27-604, 48-658, 151-666, 151-688, 151-800, 173-594, 184-666, 216-765, 298-910, 331-945, 378-666, |
| 3969 | 408-668, 408-678, 506-1234, 539-868, 578-1173, 656-1233, 681-1294, 714-1281, 725-1335, 727-1393, 749-1390, |
| | 759-1390, 762-1360, 787-1155, 802-1329, 803-869, 803-885, 822-1245, 879-1266, 883-1138, 883-1221, 883-1334, |
| | 883-1359, 883-1381, 883-1447, 883-1462, 883-1514, 883-1560, 929-1343, 929-1344, 929-1355, 929-1490, 931-1063, |
| | 932-1303, 952-1819, 955-1115, 955-1329, 973-1630, 994-1629, 1032-1710, 1049-1638, 1064-1723, 1068-1677, |
| | 1081-1686, 1114-1263, 1121-2063, 1126-1744, 1182-1724, 1255-1894, 1266-1907, 1274-1720, 1278-1573, |
| | 1298-1891, 1313-1615, 1322-1546, 1322-1601, |
| | 1342-1997, 1351-1581, 1352-1977, 1356-1816, 1366-1653, 1368-1877, 1379-1996, 1387-1989, 1409-1769, 1429-1917, |
| | 1460-1989, 1463-2071, 1468-1904, 1468-1919, 1477-1884, 1478-1919, 1486-2070, 1500-2011, 1552-2282, |
| | 1561-1786, 1561-1987, 1597-1665, 1639-2303, 1646-2253, 1699-1964, 1712-2264, 1714-2013, 1714-2269, 1724-2379, |
| | 1766-2368, 1775-2324, 1829-2379, 1864-2459, 1864-2521, 1946-2275, 1978-2445, 2096-2303, 2096-2643, |
| | 2127-2401, 2183-2525, 2244-2496, 2244-2497, 2344-2601, 2426-2952, 2466-2716, 2468-3032, 2472-3052, 2537-2659, |
| | 2537-3139, 2540-2777, 2540-2782, 2556-3171, 2560-2797, 2623-3268, 2626-3258, 2630-3136, 2638-2875, |
| | 2648-3269, 2731-2866, 2731-3006, 2731-3213, 2738-3283, 2774-3015, 2774-3059, 2774-3398, 2779-3380, 2807-3043, |
| | 2832-2958, |
| | 2835-3100, 2835-3130, 2854-3116, 2868-3256, 2881-3100, 2881-3114, 2881-3241, 2885-3071, 2885-3134, 2887-3465, |
| | 2894-3139, 2896-3165, 2896-3172, 2897-3465, 2904-3165, 2911-3074, 2933-3199, 2933-3465, 2939-3156, |
| | 2939-3366, 2942-3318, 2947-3373, 2957-3465, 2965-3465, 2990-3258, 3008-3465, 3012-3292, 3044-3300, 3047-3350, |
| | 3057-3276, 3087-3340, 3127-3464, 3139-3402, 3153-3411, 3190-3440, 3278-3969, 3283-3465, 3309-3465, |
| | 3413-3465, 3442-3465, 3585-3672, 3586-3656, 3586-3667, 3586-3678, 3586-3685, 3586-3706, 3586-3727, 3586-3771, |
| | 3586-3782, 3586-3804, 3586-3848, 3586-3862, 3586-3924, 3586-3928, 3647-3899, 3647-3900, 3747-3928, |
| | 3829-3928, 3869-3928, 3875-3928 |
| 52/7503507CB1/ | 1-471, 1-2516, 46-537, 113-743, 113-800, 113-850, 113-871, 114-589, 114-856, 117-857, 124-619, 648-1320, 649-984, |
| 2537 | 649-1030, 649-1036, 649-1040, 649-1067, 649-1092, 649-1121, 649-1128, 649-1130, 649-1131, 649-1132, 649-1135, |
| | 649-1147, 649-1149, 649-1167, 649-1174, 651-978, 653-785, 653-1030, 655-1197, 655-1300, 657-1053, 657-1086, |
| | 657-1154, 657-1195, 657-1244, 657-1262, 657-1297, 657-1300, 657-1320, 658-1069, 658-1249, 667-1001, |
| | 667-1128, 667-1135, 672-908, 676-1014, 684-1142, 691-826, 691-880, 691-901, 691-1025, 691-1029, 691-1149, |
| | 691-1156, 691-1173, 693-891, 700-1103, 710-981, 719-967, 759-906, 762-1255, 766-1309, 767-1000, 767-1133, |
| | 793-1322, 839-1255, 840-1345, 864-1328, 954-1265, 956-1265, 1055-1339, 1091-1338, 1178-1348, 1335-1700, |
| | 1335-1950, 1336-1614, 1395-1670, |
| | 1415-1647, 1489-1793, 1493-1722, 1493-1742, 1497-1747, 1502-2107, 1533-1784, 1533-1790, 1533-1791, 1533-2058, |
| | 1533-2125, 1573-2064, 1579-2048, 1585-2098, 1585-2133, 1598-2059, 1599-2008, 1599-2022, 1632-1891, |
| | 1655-1711, 1667-1958, 1668-1955, 1671-1892, 1671-2412, 1726-2201, 1737-2214, 1762-1936, 1780-2083, 1802-2065, |
| | 1802-2364, 1840-2147, 1848-2127, 1854-2080, 1869-2109, 1889-1989, 1889-2169, 1917-2165, 1917-2212, |
| | 1918-2182, 1924-2206, 1953-2219, 1967-2257, 1969-2156, 1998-2421, 2059-2203, 2059-2377, 2070-2286, 2070-2400, |
| | 2104-2315, 2104-2495, 2110-2400, 2110-2534, 2136-2418, 2143-2368, 2160-2427, 2192-2407, 2192-2412, |
| | 2205-2443, 2207-2473, 2207-2474, 2255-2502, 2263-2504, 2282-2448, 2282-2516, 2290-2537, 2326-2462 |
| 53/7503506CB1/ | 1-471, 1-2505, 46-537, 113-743, 113-800, 113-850, 113-871, 114-589, 114-856, 117-857, 124-619, 648-1320, 649-984, |
| 2526 | 649-1030, 649-1036, 649-1040, 649-1067, 649-1092, 649-1121, 649-1128, 649-1130, 649-1131, 649-1132, 649-1135, |
| | 649-1147, 649-1149, 649-1167, 649-1174, 651-978, 653-785, 653-1030, 655-1197, 655-1300, 657-1053, 657-1086, |
| | 657-1154, 657-1195, 657-1244, 657-1262, 657-1297, 657-1300, 657-1320, 658-1069, 658-1249, 667-1001, |
| | 667-1128, 667-1135, 672-908, 676-1014, 684-1142, 691-826, 691-880, 691-901, 691-1025, 691-1029, 691-1149, |
| | 691-1156, 691-1173, 693-891, 700-1103, 710-981, 719-967, 759-906, 762-1255, 766-1309, 767-1000, 767-1133, |
| | 793-1322, 839-1255, 840-1395, 864-1328, 954-1265, 956-1265, 968-1603, 1055-1339, 1091-1338, 1151-1385, 1178-1398, |
| | 1281-1705, 1396-1659, 1396-1689, 1404-1636, 1478-1782, 1482-1711, 1482-1731, 1486-1736, 1491-2096, 1522-1773, |
| | 1522-1779, 1522-1780, 1522-2047, 1522-2114, 1562-2053, 1568-2037, 1574-2087, 1574-2122, 1587-2048, |
| | 1588-1997, 1588-2011, 1621-1880, 1644-1700, 1656-1947, 1657-1944, 1660-1881, 1660-2401, 1715-2190, 1726-2203, |
| | 1751-1925, 1769-2072, 1791-2054, 1791-2353, 1829-2136, 1837-2116, 1843-2069, 1858-2098, 1878-1978, |
| | 1878-2158, 1906-2154, 1906-2201, 1907-2171, 1913-2195, 1942-2208, 1956-2246, 1958-2145, 1987-2410, 2048-2192, |
| | 2048-2366, 2059-2275, 2059-2389, 2093-2304, 2093-2484, 2099-2389, 2099-2523, 2125-2407, 2132-2357, |
| | 2149-2416, 2181-2396, 2181-2401, 2194-2432, 2196-2462, 2196-2463, 2244-2491, 2252-2493, 2271-2437, 2271-2505, |
| | 2279-2526, 2315-2451 |
| 54/7503509CB1/ | 1-471, 1-2443, 46-537, 113-743, 114-589, 124-619, 649-1123, 796-1107, 798-1107, 820-1312, 897-1181, 910-1554, |
| 2464 | 933-1180, 933-1280, 987-1528, 993-1227, 1002-1302, 1017-1266, 1020-1438, 1106-1627, 1109-1627, 1115-1627, |
| | 1126-1627, 1190-1468, 1203-1877, 1211-1635, 1212-1468, 1244-1541, 1322-1597, 1342-1574, 1416-1720, 1420-1649, |
| | 1420-1669, 1424-1674, 1429-2034, 1460-1711, 1460-1717, 1460-1718, 1460-1985, 1460-2052, 1500-1991, |
| | 1506-1975, 1512-2025, 1512-2060, 1525-1986, 1526-1935, 1526-1949, 1559-1818, 1582-1638, 1594-1885, 1595-1882, |
| | 1598-1819, 1598-2339, 1653-2128, 1664-2141, 1689-1863, 1707-2010, 1729-1992, 1729-2291, 1767-2074, |
| | 1775-2054, 1781-2007, 1796-2036, 1816-1916, 1816-2096, 1844-2092, 1844-2139, 1845-2109, 1851-2133, 1880-2146, |
| | 1894-2184, 1896-2083, 1925-2348, 1986-2130, 1986-2304, 1997-2213, 1997-2327, 2031-2242, 2031-2422, |
| | 2037-2327, 2037-2461, 2063-2345, 2070-2295, 2087-2354, 2119-2334, 2119-2339, 2132-2370, 2134-2400, 2134-2401, |
| | 2182-2429, 2190-2431, 2209-2375, 2209-2443, 2217-2464, 2253-2389 |
| 55/7505800CB1/ | 1-211, 1-1447, 3-294, 3-369, 3-401, 3-402, 3-447, 3-517, 3-537, 3-577, 3-603, 3-607, 3-637, 3-639, 3-694, 3-721, 3-846, |
| 1452 | 6-728, 18-133, 39-503, 40-619, 40-767, 50-736, 51-327, 60-778, 80-285, 99-469, 129-866, 210-438, 210-463, |
| | 210-576, 210-735, 227-485, 241-590, 244-516, 248-551, 260-567, 263-396, 265-500, 265-1060, 268-520, 268-835, |
| | 271-567, 278-716, 298-551, 304-1167, 315-638, 341-801, 370-533, 370-595, 372-878, 373-1243, 376-626, 377-874, |
| | 377-1017, 386-666, 390-793, 399-655, 400-636, 401-614, 403-646, 405-697, 405-1032, 407-586, 415-907, 424-582, |
| | 426-690, 440-571, 442-701, 451-671, 452-1173, 469-732, 479-757, 482-765, 506-618, 524-1314, 525-787, 548-1165, |
| | 553-787, 559-805, 559-814, 561-1414, 564-1415, 576-1395, 577-782, 577-942, 582-750, 584-824, 587-748, |
| | 587-880, 592-882, 592-894, 599-807, |
| | 599-916, 599-1090, 600-1147, 601-814, 608-872, 608-1038, 617-944, 620-1397, 624-1238, 626-1124, 627-899, 628-889, |
| | 629-941, 630-806, 631-1446, 640-828, 641-918, 642-1077, 643-856, 653-1107, 655-881, 658-1430, 664-1137, |
| | 671-968, 671-1161, 677-1224, 683-1446, 684-1119, 690-1333, 703-1443, 705-1385, 709-1451, 715-1452, 718-1411, |
| | 719-1446, 722-1448, 722-1451, 724-1271, 724-1452, 725-1002, 729-1021, 729-1104, 733-1452, 734-1449, 743-1452, |
| | 744-1310, 746-1054, 748-1450, 748-1451, 757-1451, 758-1401, 759-1446, 761-1452, 765-984, 766-1452, 767-1391, |
| | 768-1006, 771-1451, 775-905, 780-1452, 781-1117, 784-1452, 789-1452, 791-1450, 802-1452, 808-1451, 810-1451, |
| | 815-1452, 816-1070, 816-1402, |
| | 820-1437, 821-1452, 831-1065, 831-1106, 833-1144, 833-1436, 835-1370, 837-1314, 837-1452, 838-1118, 843-1431, |
| | 847-1447, 850-1034, 853-1093, 856-1372, 858-1115, 863-1450, 868-1132, 872-1452, 874-1380, 875-1131, |
| | 875-1452, 876-1174, 879-1056, 879-1452, 881-1312, 887-1107, 893-1442, 893-1452, 896-1452, 897-1144, 902-1125, |
| | 903-1131, 911-1452, 919-1452, 925-1452, 926-1406, 933-1450, 933-1452, 938-1409, 943-1247, 948-1452, |
| | 951-1154, 952-1452, 959-1440, 959-1450, 961-1382, 962-1233, 964-1237, 974-1189, 975-1247, 975-1448, 977-1163, |
| | 977-1452, 979-1452, 984-1449, 985-1447, 985-1452, 987-1452, 989-1452, 992-1299, 992-1451, 992-1452, |
| | 998-1450, 998-1452, 999-1449, 1004-1448, 1004-1450, 1009-1452, 1014-1450, 1015-1290, 1016-1452, 1017-1282, |
| | 1017-1303, 1019-1449, 1019-1452, 1020-1452, 1022-1447, 1022-1452, 1024-1284, 1026-1450, 1026-1452, 1027-1452, |
| | 1028-1310, 1029-1447, 1029-1452, 1030-1289, 1031-1452, 1032-1447, 1033-1448, 1034-1445, 1035-1446, |
| | 1035-1448, 1037-1447, 1040-1452, |
| | 1041-1370, 1041-1451, 1042-1450, 1045-1452, 1047-1452, 1050-1447, 1051-1328, 1053-1447, 1056-1452, 1063-1334, |
| | 1064-1452, 1065-1452, 1066-1443, 1067-1447, 1067-1448, 1069-1452, 1070-1443, 1070-1447, 1070-1452, |
| | 1071-1446, 1071-1447, 1071-1452, 1072-1448, 1076-1452, 1078-1452, 1081-1445, 1082-1318, 1083-1450, 1089-1382, |
| | 1089-1437, 1089-1452, 1092-1443, 1092-1452, 1097-1447, 1099-1356, 1103-1305, 1103-1450, 1104-1448, |
| | 1106-1376, 1106-1384, 1108-1452, 1109-1390, 1123-1448, 1129-1448, 1130-1447, 1137-1435, 1143-1450, 1144-1412, |
| | 1145-1450, 1150-1452, 1151-1452, 1152-1447, 1155-1447, 1155-1448, 1155-1452, 1160-1430, 1160-1445, |
| | 1160-1447, 1161-1450, 1162-1368, 1171-1413, 1172-1447, 1172-1448, 1188-1452, 1191-1450, 1199-1452, 1200-1448, |
| | 1201-1452, 1203-1447, 1205-1452, 1217-1452, 1238-1447, 1238-1448, 1238-1449, 1242-1452, 1257-1445, |
| | 1257-1447, 1259-1452, 1270-1452, 1275-1452, 1280-1447, 1297-1450, 1297-1452, 1310-1452, 1316-1448, 1317-1447, |
| | 1319-1445, 1331-1447, 1332-1447, 1352-1447 |
| 56/7503141CB1/ | 1-142, 1-281, 1-286, 1-296, 1-304, 1-339, 1-358, 1-365, 1-369, 1-425, 1-454, 1-480, 1-510, 1-520, 1-532, 1-537, 1-565, |
| 1802 | 1-568, 3-194, 3-263, 3-281, 3-293, 4-195, 4-202, 4-232, 4-246, 4-264, 4-269, 4-302, 4-484, 4-543, 6-567, 7-249, |
| | 7-568, 8-223, 8-239, 8-250, 8-279, 8-315, 10-244, 10-446, 10-501, 10-520, 10-528, 10-1789, 11-266, 11-268, 11-283, |
| | 12-243, 12-248, 12-273, 12-292, 12-293, 12-525, 13-278, 13-286, 13-290, 14-129, 14-256, 14-286, 14-307, 19-481, |
| | 21-252, 21-256, 21-309, 21-316, 21-365, 21-369, 22-129, 22-143, 22-176, 22-255, 22-291, 22-292, 22-300, 22-301, |
| | 22-369, 22-413, 22-446, 22-528, 22-559, 22-568, 24-493, 24-568, 27-369, 29-291, 32-250, 33-448, 39-340, 47-301, |
| | 49-369, 51-458, 51-563, 53-252, 64-680, 82-504, 85-568, 101-568, 120-390, 139-301, 160-466, 229-458, 251-472, |
| | 251-537, 254-568, 274-486, 274-491, 296-568, 302-761, 303-379, 303-844, 305-475, 314-564, 318-559, 321-544, |
| | 325-542, 359-568, |
| | 419-568, 566-798, 575-1270, 630-1325, 637-1271, 638-1133, 642-892, 645-1222, 645-1349, 646-887, 652-1026, |
| | 670-1392, 679-971, 701-1264, 705-1394, 709-1340, 713-969, 726-1325, 736-1308, 739-1002, 743-1376, 744-1386, |
| | 757-1037, 757-1042, 757-1065, 758-1394, 769-1023, 780-1332, 781-1027, 781-1281, 782-1063, 783-1440, 788-1638, |
| | 792-1061, 792-1081, 812-1040, 814-1044, 814-1214, 816-919, 817-1093, 822-1348, 825-1052, 828-1399, 831-1111, |
| | 846-1071, 849-1399, 860-1058, 860-1070, 860-1399, 866-1452, 866-1555, 874-1650, 875-1170, 875-1248, |
| | 875-1263, 875-1304, 875-1329, 875-1334, 875-1341, 875-1358, 875-1371, 875-1375, 875-1381, 875-1389, 875-1395, |
| | 875-1396, 875-1403, 875-1405, 875-1412, 875-1418, 875-1431, 875-1469, 875-1472, 875-1474, 875-1491, |
| | 875-1506, 875-1509, 875-1530, 875-1533, 875-1540, 875-1542, 875-1550, 875-1615, 875-1629, 875-1641, 875-1670, |
| | 882-1393, 886-1162, 886-1733, 893-1171, 893-1183, 894-1126, 896-1156, 900-1549, 904-1156, 904-1514, |
| | 907-1395, 921-1768, 923-1696, |
| | 927-1171, 931-1396, 941-1742, 945-1134, 952-1218, 953-1477, 959-1170, 959-1186, 969-1216, 976-1240, 989-1430, |
| | 992-1269, 995-1269, 1003-1343, 1010-1235, 1017-1727, 1023-1279, 1023-1300, 1023-1704, 1026-1304, 1027-1241, |
| | 1027-1325, 1027-1636, 1030-1744, 1040-1755, 1042-1787, 1043-1205, 1043-1308, 1045-1226, 1047-1671, |
| | 1064-1618, 1075-1748, 1076-1761, 1079-1351, 1084-1705, 1086-1719, 1092-1769, 1114-1393, 1121-1630, 1124-1706, |
| | 1124-1755, 1125-1799, 1127-1785, 1133-1395, 1134-1787, 1136-1758, 1138-1622, 1141-1765, 1141-1802, |
| | 1144-1477, 1145-1704, 1148-1786, 1156-1772, 1167-1333, 1168-1441, 1168-1701, 1168-1762, 1168-1786, 1176-1474, |
| | 1180-1423, 1186-1456, 1191-1798, 1200-1787, 1205-1515, 1212-1753, 1224-1761, 1224-1787, 1226-1785, |
| | 1234-1515, 1234-1787, 1236-1433, 1236-1752, 1236-1772, 1236-1785, 1237-1786, 1264-1500, 1264-1790, 1264-1793, |
| | 1275-1796, 1282-1604, 1282-1772, 1285-1795, 1286-1794, 1301-1796, 1302-1588, 1304-1772, 1308-1772, |
| | 1312-1766, 1315-1772, 1321-1772, 1322-1772, 1325-1796, 1327-1772, 1328-1766, 1328-1785, 1337-1772, 1337-1799, |
| | 1340-1780, 1342-1766, 1342-1772, 1343-1772, 1344-1772, 1345-1588, 1345-1772, 1348-1775, 1349-1781, |
| | 1356-1772, 1358-1770, 1359-1773, 1363-1772, 1366-1772, 1368-1802, 1369-1772, 1374-1772, 1375-1776, 1375-1772, |
| | 1379-1765, 1380-1802, 1386-1701, 1386-1714, 1393-1772, 1401-1785, 1401-1802, 1407-1772, 1408-1772, |
| | 1409-1703, 1415-1786, 1415-1787, 1423-1704, 1425-1772, 1433-1787, 1434-1782, 1437-1668, 1441-1692, 1448-1772, |
| | 1464-1772, 1466-1786, 1472-1757, 1477-1611, 1478-1789, 1479-1750, 1484-1759, 1485-1743, 1485-1772, |
| | 1491-1772, 1498-1748, 1498-1772, 1503-1772, 1512-1777, 1515-1786, 1530-1779, 1534-1772, 1553-1772, 1559-1772, |
| | 1568-1786, 1579-1785, 1599-1802, 1611-1802, 1612-1784, 1621-1802, 1629-1802, 1637-1767, 1637-1797, |
| | 1654-1775, 1673-1799, 1682-1767, 1711-1787 |
| 57/7500362CB1/ | 1-133, 1-147, 1-151, 1-1833, 5-120, 13-120, 13-134, 13-147, 89-761, 90-308, 90-309, 90-469, 90-508, 90-545, 90-682, |
| 1833 | 90-767, 154-383, 154-682, 155-769, 170-952, 176-397, 176-462, 179-559, 199-411, 199-416, 210-846, 214-834, |
| | 219-742, 219-809, 221-513, 227-621, 227-640, 227-700, 227-737, 227-762, 227-800, 227-808, 227-809, 227-821, |
| | 227-866, 227-868, 227-882, 227-907, 228-304, 228-710, 228-745, 228-752, 228-761, 228-762, 228-776, 228-779, |
| | 228-786, 228-812, 228-820, 228-853, 228-865, 228-866, 228-873, 228-876, 228-899, 228-902, 228-926, 229-805, |
| | 230-400, 230-898, 239-489, 243-484, 246-469, 249-562, |
| | 250-467, 267-854, 268-966, 271-953, 274-751, 275-936, 284-510, 285-528, 285-560, 297-1056, 334-594, 334-933, |
| | 336-624, 336-901, 337-816, 343-1042, 344-754, 348-745, 348-854, 373-643, 386-1091, 390-607, 420-676, 428-678, |
| | 444-659, 463-724, 468-1108, 474-730, 477-712, 480-723, 484-761, 484-776, 484-812, 491-758, 493-747, 494-1121, |
| | 498-725, 498-738, 498-773, 501-1102, 512-802, 513-810, 519-764, 528-996, 530-1180, 537-1184, 548-921, 551-765, |
| | 552-1073, 565-1212, 567-1203, 574-795, 576-856, 584-807, 590-1146, 594-835, 604-874, 611-870, 614-846, |
| | 614-848, 623-1318, 650-843, 678-1373, 685-1319, 686-1181, |
| | 690-940, 693-1270, 693-1397, 694-935, 698-986, 699-953, 700-976, 700-1074, 718-976, 718-1440, 727-1019, 749-1312, |
| | 753-1442, 757-1388, 761-1017, 774-1373, 784-1356, 787-1050, 791-1424, 792-1434, 805-1085, 805-1090, |
| | 805-1113, 806-1442, 817-1071, 828-1380, 829-1075, 829-1329, 830-1111, 831-1488, 836-1682, 840-1109, 840-1129, |
| | 860-1088, 862-1092, 862-1262, 864-967, 865-1141, 870-1396, 873-1100, 876-1447, 879-1159, 894-1119, 897-1447, |
| | 908-1447, 914-1500, 914-1634, 922-1694, 923-1218, 923-1296, 923-1311, 923-1352, 923-1377, 923-1382, |
| | 923-1389, 923-1406, 923-1419, 923-1423, 923-1429, 923-1437, 923-1443, 923-1444, 923-1451, 923-1453, 923-1460, |
| | 923-1466, 923-1479, 923-1517, 923-1520, 923-1522, 923-1539, 923-1554, 923-1557, 923-1578, 923-1581, |
| | 923-1588, 923-1590, 923-1598, 923-1659, 923-1673, 923-1685, 923-1714, 930-1441, 934-1210, 934-1777, 941-1219, |
| | 941-1231, 942-1174, 944-1204, 948-1609, 952-1204, 952-1562, 955-1443, 969-1812, 971-1740, 975-1219, |
| | 979-1444, 989-1786, 993-1182, |
| | 1001-1525, 1007-1234, 1037-1478, 1040-1317, 1043-1317, 1051-1391, 1058-1283, 1065-1771, 1071-1327, 1071-1348, |
| | 1071-1748, 1074-1352, 1075-1289, 1075-1373, 1075-1680, 1078-1788, 1088-1799, 1091-1253, 1091-1356, |
| | 1095-1715, 1112-1662, 1123-1792, 1124-1805, 1127-1399, 1132-1749, 1134-1763, 1140-1813, 1162-1441, 1169-1674, |
| | 1172-1750, 1172-1799, 1173-1833, 1175-1829, 1181-1443, 1184-1802, 1186-1666, 1189-1809, 1189-1833, |
| | 1192-1525, 1193-1748, 1196-1830, 1204-1816, 1216-1489, 1216-1745, 1216-1806, 1224-1522, 1228-1471, 1234-1504, |
| | 1239-1833, 1253-1563, 1260-1797, 1272-1805, 1274-1814, 1282-1563, 1282-1831, 1284-1481, 1284-1796, |
| | 1284-1829, 1285-1830, 1312-1548, 1323-1833, 1330-1648, 1330-1825, 1334-1833, 1352-1820, 1356-1818, 1360-1810, |
| | 1372-1652, 1373-1833, 1376-1814, 1385-1833, 1406-1814, 1455-1822, 1612-1830, 1643-1833, 1655-1833, |
| | 1656-1828, 1665-1833, 1673-1833, 1681-1833, 1717-1833, 1726-1811, 1737-1819 |
| 58/7503328CB1/ | 1-262, 1-286, 1-308, 1-317, 1-325, 1-496, 1-501, 1-512, 1-621, 2-201, 4-296, 8-233, 8-246, 8-524, 8-535, 8-2463, 15-699, |
| 2465 | 15-774, 15-783, 15-809, 15-854, 15-903, 16-771, 19-265, 22-187, 22-235, 27-457, 27-631, 38-731, 39-627, 44-243, |
| | 44-592, 44-642, 49-645, 50-181, 50-325, 56-175, 56-543, 58-170, 58-329, 60-352, 62-302, 63-206, 64-335, 67-330, |
| | 69-294, 69-360, 69-548, 69-598, 69-613, 71-630, 71-747, 73-560, 73-730, 76-545, 84-685, 88-392, 89-636, 90-312, |
| | 90-681, 97-377, 98-662, 100-375, 100-736, 103-368, 106-369, 109-691, 109-764, 134-386, 139-616, 139-767, |
| | 147-731, 182-675, 182-692, 184-710, 184-719, 190-758, 190-836, 195-451, 195-720, 195-747, 208-611, 232-780, |
| | 282-531, 290-520, 342-798, 345-790, |
| | 351-914, 366-614, 371-675, 407-880, 407-984, 420-914, 439-883, 459-1000, 472-961, 473-810, 480-1017, 488-797, |
| | 491-1048, 510-1060, 524-1084, 549-1100, 574-1139, 578-856, 581-1139, 584-845, 586-789, 587-815, 593-1014, |
| | 593-1136, 593-1139, 593-1141, 600-850, 608-888, 608-1040, 615-1024, 615-1079, 615-1082, 617-1036, 636-884, |
| | 636-1106, 636-1153, 640-1270, 644-1278, 648-1192, 660-1105, 669-956, 669-1192, 669-1305, 677-956, 681-1325, |
| | 688-1010, 688-1075, 688-1279, 689-1265, 698-971, 698-1289, 705-1168, 705-1169, 705-1192, 709-1079, 709-1189, |
| | 719-1188, 726-1291, 730-1279, 734-1293, 747-1075, 748-1023, 764-1192, 764-1352, 765-1079, 765-1192, 767-1278, |
| | 770-1244, 794-1352, 799-1268, 802-1304, 804-1344, 816-1329, 816-1344, 816-1352, 817-1079, 817-1141, |
| | 817-1153, 817-1236, 817-1279, |
| | 817-1280, 817-1350, 818-1236, 829-1111, 846-1062, 849-1222, 863-1289, 872-1093, 893-1114, 896-1352, 900-1157, |
| | 926-1351, 930-1352, 939-1214, 965-1232, 1008-1351, 1023-1305, 1059-1233, 1075-1304, 1125-1352, 1125-1418, |
| | 1125-1466, 1128-1410, 1178-1352, 1355-1904, 1360-2036, 1366-1862, 1376-1725, 1380-1645, 1384-1862, |
| | 1384-1883, 1404-1928, 1419-1639, 1426-1864, 1432-2008, 1433-1662, 1433-2036, 1437-1743, 1467-1752, 1467-1782, |
| | 1467-1788, 1475-2009, 1479-1771, 1490-1904, 1493-1936, 1498-2090, 1508-1815, 1518-1788, 1528-1772, |
| | 1572-2194, 1574-2167, 1579-1828, 1588-1984, 1597-1842, 1599-2178, 1601-1977, 1601-2159, 1601-2175, 1601-2189, |
| | 1610-1884, 1611-1815, 1616-1830, 1616-2202, 1631-1788, 1636-1860, 1650-2102, 1652-2180, 1659-2431, |
| | 1667-2405, 1668-1859, 1669-2211, 1681-2369, 1700-1990, 1719-1966, 1725-2445, 1727-2314, 1732-1989, 1732-2325, |
| | 1735-2434, 1741-2328, 1753-2381, 1755-2404, 1763-2434, 1779-2075, 1779-2431, 1783-2404, 1783-2442, |
| | 1791-2400, 1798-2019, 1798-2398, 1799-2412, 1799-2433, 1806-2222, 1808-2437, 1810-2465, 1811-2434, 1823-2092, |
| | 1825-2060, 1830-2465, 1832-2325, 1835-2061, 1835-2104, 1838-2073, 1839-2435, 1843-2130, 1845-2102, |
| | 1850-2098, 1852-2410, 1859-2325, 1869-2142, 1873-2451, 1878-2382, 1902-2443, 1903-2172, 1903-2438, 1903-2445, |
| | 1903-2459, 1904-2156, 1905-2201, 1906-2378, 1912-2145, 1915-2325, 1919-2266, 1922-2440, 1923-2219, |
| | 1924-2406, 1925-2442, 1929-2169, 1929-2465, 1941-2240, 1943-2465, 1944-2465, 1952-2130, 1952-2199, 1952-2451, |
| | 1956-2189, 1964-2160, 1964-2464, 1964-2465, 1967-2231, 1970-2455, 1971-2167, 1974-2437, 1974-2465, |
| | 1984-2401, 1984-2464, 1984-2465, 1985-2453, 1991-2349, 1992-2449, 1992-2451, 1994-2451, 1995-2242, 1996-2451, |
| | 1996-2465, 2002-2216, 2002-2453, 2004-2250, 2007-2228, 2007-2457, 2011-2453, 2011-2465, 2015-2401, |
| | 2015-2454, 2015-2457, 2016-2401, 2019-2401, 2021-2401, 2021-2450, 2022-2465, 2023-2465, 2024-2465, |
| | 2025-2281, 2025-2465, 2030-2465, 2031-2238, 2033-2450, 2036-2450, 2037-2401, 2038-2401, 2039-2401, 2041-2401, |
| | 2042-2387, 2043-2401, 2043-2451, 2043-2465, 2045-2401, 2047-2455, 2048-2291, 2049-2465, 2052-2401, |
| | 2053-2401, 2055-2401, 2056-2318, 2057-2401, 2058-2318, 2058-2319, 2059-2307, 2061-2465, 2062-2401, 2063-2394, |
| | 2068-2401, 2070-2465, 2079-2291, 2082-2312, 2082-2338, 2086-2451, 2092-2349, 2093-2368, 2097-2352, |
| | 2097-2449, 2097-2451, 2098-2346, 2100-2401, 2101-2450, 2101-2465, 2103-2449, 2103-2451, 2103-2465, 2108-2401, |
| | 2109-2452, 2118-2401, 2121-2401, 2128-2465, 2136-2356, 2136-2427, 2143-2386, 2151-2401, 2158-2465, |
| | 2160-2401, 2166-2434, 2167-2455, 2169-2401, 2178-2401, 2180-2454, 2181-2463, 2188-2401, 2205-2465, 2214-2451, |
| | 2216-2443, 2219-2401, 2219-2451, 2234-2465, 2238-2451, 2239-2465, 2242-2376, 2243-2452, 2253-2464, |
| | 2272-2401, 2279-2401, 2286-2454, 2308-2451, 2323-2401, 2349-2465, 2396-2465 |
| 59/7510464CB1/ | 1-308, 1-325, 1-374, 1-496, 1-501, 1-512, 2-201, 4-296, 6-249, 6-317, 8-233, 8-246, 8-480, 8-535, 8-2555, 15-699, |
| 2560 | 15-760, 15-783, 15-809, 16-771, 19-265, 22-187, 22-235, 27-457, 30-165, 38-731, 43-286, 44-243, 49-645, 50-181, |
| | 50-325, 50-729, 56-175, 56-543, 56-674, 57-639, 57-641, 57-772, 57-774, 58-170, 58-329, 60-352, 62-302, 67-330, |
| | 69-294, 69-360, 69-391, 69-548, 69-613, 70-335, 71-630, 71-747, 73-455, 75-545, 77-917, 80-557, 80-614, 84-685, |
| | 88-165, 88-392, 89-422, 89-730, 90-312, 90-642, 90-681, 96-631, 97-200, 100-375, 100-736, 101-704, 103-368, 106-369, |
| | 109-691, 109-764, 110-304, 126-704, 134-386, 139-616, 139-767, 147-731, 173-781, 182-675, 182-692, 184-710, |
| | 184-719, 187-373, 188-542, 190-758, 190-836, 195-451, 195-677, 195-747, 208-611, 212-921, 232-780, 261-797, |
| | 281-911, 281-934, 282-531, 285-662, 290-520, 303-852, 307-816, 329-583, 342-798, 345-790, 347-816, 347-861, |
| | 366-614, 371-675, 371-921, 372-898, 379-584, 402-932, 404-882, 407-969, 407-984, 443-833, 447-559, 452-838, |
| | 464-1045, |
| | 581-1139, 593-1139, 600-850, 615-1024, 640-1270, 681-1325, 688-1087, 698-1289, 709-1189, 719-1188, 726-1291, |
| | 734-1293, 764-1192, 765-1192, 770-1244, 799-1268, 804-1344, 817-1083, 817-1153, 817-1236, 817-1279, 817-1280, |
| | 829-1111, 840-1379, 840-1455, 846-1062, 846-1369, 857-1404, 859-1406, 862-1507, 863-1289, 872-1093, |
| | 872-1415, 883-1481, 896-1352, 903-1507, 923-1619, 930-1360, 931-1374, 948-1507, 958-1506, 965-1232, 970-1458, |
| | 987-1484, 993-1535, 1005-1490, 1008-1351, 1019-1563, 1023-1305, 1030-1507, 1047-1442, 1054-1488, 1071-1609, |
| | 1076-1507, 1080-1473, 1080-1507, 1080-1552, 1083-1527, 1091-1376, 1091-1445, 1101-1617, 1105-1555, |
| | 1109-1540, 1111-1540, 1119-1650, 1125-1393, 1153-1552, 1154-1507, 1162-1417, 1168-1640, 1187-1467, 1197-1746, |
| | 1200-1676, 1205-1852, 1212-1468, 1212-1517, 1214-1564, 1216-1484, 1222-1507, 1224-1875, 1227-1565, |
| | 1227-1657, 1235-1664, |
| | 1237-1575, 1237-1634, 1239-1446, 1239-2027, 1243-1520, 1245-1800, 1253-1590, 1268-1531, 1274-1609, 1278-1562, |
| | 1280-1731, 1288-1856, 1295-1855, 1305-1778, 1321-1718, 1321-1791, 1327-1882, 1338-1834, 1348-1799, |
| | 1349-1727, 1360-1452, 1365-1720, 1384-2025, 1389-1950, 1396-2128, 1400-1896, 1406-1609, 1406-1692, 1416-1970, |
| | 1418-1896, 1418-1917, 1423-1581, 1436-1854, 1437-2119, 1460-1898, 1471-1777, 1489-2004, 1501-1726, |
| | 1501-1778, 1501-1822, 1512-1757, 1513-1805, 1522-2121, 1524-1959, 1534-2108, 1542-1849, 1544-1780, 1560-2128, |
| | 1562-1806, 1606-1918, 1619-2239, 1631-1876, 1644-1918, 1650-1864, 1665-1822, 1702-1893, 1803-2281, |
| | 1830-2444, 1860-2132, 1902-2504, 1965-2551, 1992-2508, 1994-2264, 1995-2535, 1999-2470, 2004-2237, 2007-2417, |
| | 2015-2311, 2016-2498, 2021-2261, 2021-2560, 2025-2504, 2035-2558, 2036-2560, 2044-2475, 2048-2281, |
| | 2056-2557, 2059-2323, 2062-2547, 2066-2503, 2066-2559, 2076-2493, 2076-2557, 2076-2558, 2077-2545, |
| | 2078-2502, 2081-2500, 2084-2551, 2087-2334, 2088-2560, 2091-2509, 2095-2308, 2096-2342, 2099-2320, 2099-2549, |
| | 2103-2545, 2104-2546, 2106-2549, 2107-2493, 2108-2493, 2111-2493, 2113-2542, 2114-2559, 2115-2557, |
| | 2117-2373, 2117-2560, 2122-2560, 2123-2327, 2125-2542, 2128-2542, 2129-2493, 2130-2493, 2131-2493, 2133-2493, |
| | 2134-2479, 2135-2493, 2135-2504, 2135-2543, 2135-2557, 2137-2493, 2139-2547, 2140-2383, 2141-2557, |
| | 2143-2493, 2144-2493, 2145-2493, 2147-2493, 2149-2493, 2151-2399, 2153-2558, 2155-2486, 2160-2493, 2162-2560, |
| | 2171-2383, 2174-2404, 2174-2430, 2178-2543, 2184-2441, 2185-2460, 2189-2444, 2189-2551, 2190-2438, |
| | 2192-2493, 2193-2560, 2195-2543, 2195-2560, 2201-2544, 2210-2493, 2213-2493, 2228-2448, 2228-2519, 2235-2478, |
| | 2243-2493, 2250-2560, 2252-2493, 2258-2526, 2259-2547, 2261-2493, 2270-2493, 2272-2546, 2273-2555, |
| | 2280-2493, 2306-2543, 2311-2493, 2311-2543, 2326-2557, 2330-2543, 2331-2560, 2334-2468, 2335-2544, 2345-2560, |
| | 2364-2493, 2367-2560, 2371-2493, 2378-2546, 2415-2493, 2488-2557 |
| 60/7510394CB1/ | 1-351, 4-299, 4-2254, 5-266, 5-292, 5-311, 6-639, 9-261, 9-274, 9-276, 10-263, 10-277, 10-305, 12-232, 12-257, 13-245, |
| 2254 | 13-284, 13-294, 15-296, 24-274, 32-263, 50-289, 52-553, 83-218, 108-281, 450-714, 452-914, 452-1035, 454-1108, |
| | 455-984, 455-993, 455-1039, 457-1294, 467-963, 474-1083, 482-1086, 483-896, 487-994, 500-693, 500-796, |
| | 500-1000, 500-1088, 502-737, 502-1062, 512-777, 517-992, 517-1102, 519-896, 519-1068, 520-650, 524-1118, 529-1094, |
| | 543-841, 554-845, 556-1050, 564-797, 565-1423, 574-1164, 575-1051, 578-1086, 584-1190, 588-1161, 590-836, |
| | 597-1118, 607-861, 610-1202, 612-1169, 613-1207, 618-1156, 635-1255, 642-1333, 648-1304, 650-881, 653-895, |
| | 655-915, 656-955, 660-931, 660-1272, 667-1262, 670-1162, 673-1040, 674-1305, 686-1093, 698-968, 705-1248, |
| | 724-970, 728-1091, 728-1122, 729-1224, 731-1000, 731-1278, 736-1091, 742-1341, 748-1321, 754-1018, 754-1029, |
| | 757-1005, 759-1248, 761-1344, 763-1507, 765-1057, 770-1182, 771-1004, 771-1029, 771-1041, 774-1054, |
| | 774-1059, |
| | 776-1272, 776-1484, 779-1364, 781-902, 784-1040, 787-1333, 796-1023, 796-1091, 802-1044, 802-1392, 822-1480, |
| | 828-1085, 830-1454, 840-1099, 841-1302, 841-1350, 841-1374, 841-1582, 841-1586, 841-1607, |
| | 841-1692, 844-1551, 847-1108, 848-1028, 849-1099, 851-1105, 858-1326, 863-1431, 866-1463, 875-1123, 878-1166, |
| | 880-1479, 882-1412, 882-1455, 885-1547, 886-1348, 898-1407, 908-1047, 911-1556, 912-1479, 921-1450, |
| | 931-1468, 940-1227, 950-1183, 952-1375, 953-1558, 954-1203, 954-1616, 960-1264, 962-1660, 963-1645, 970-1582, |
| | 972-1593, 977-1213, 977-1220, 981-1602, 982-1276, 992-1500, 995-1276, 1000-1339, 1000-1634, 1001-1284, |
| | 1003-1248, 1009-1617, 1014-1259, 1018-1144, 1018-1206, 1028-1734, 1031-1311, 1033-1273, 1038-1699, |
| | 1039-1614, 1041-1309, 1042-1580, 1045-1384, 1050-1322, 1054-1323, 1054-1331, 1059-1291, 1060-1553, 1062-1314, |
| | 1064-1277, 1064-1337, 1064-1341, 1064-1352, 1067-1297, 1072-1324, 1072-1651, 1072-1668, 1082-1359, |
| | 1082-1377, 1097-1752, 1099-1531, 1102-1720, 1104-1680, 1108-1367, 1109-1361, 1109-1363, 1109-1407, 1112-1424, |
| | 1115-1315, 1115-1330, 1123-1754, 1124-1371, 1125-1372, 1131-1362, 1133-1367, 1133-1705, 1134-1721, |
| | 1135-1312, |
| | 1135-1421, 1159-1770, 1162-1465, 1172-1479, 1174-1440, 1177-1366, 1179-1402, 1182-1511, 1182-1718, 1187-1440, |
| | 1187-1469, 1187-1482, 1189-1461, 1189-1810, 1191-1840, 1192-1282, 1192-1505, 1203-1733, 1208-1794, |
| | 1211-1912, 1212-1484, 1220-1477, 1220-1717, 1220-1734, 1221-1488, 1222-1653, 1226-1730, 1229-1354, 1231-1515, |
| | 1231-1869, 1232-1819, 1237-1804, 1237-1881, 1241-1777, 1250-1484, 1251-1504, 1265-1443, 1275-1519, |
| | 1278-1918, 1284-1776, 1290-1547, 1295-1544, 1297-1580, 1301-1503, 1302-1993, 1304-1442, 1314-1578, 1314-1776, |
| | 1315-2007, 1321-1567, 1328-1596, 1331-1847, 1332-1901, 1341-1631, 1342-1584, 1342-1961, 1344-2131, |
| | 1348-1979, 1351-1900, 1354-1566, 1355-1602, 1357-1743, 1358-1643, 1360-1647, 1361-1576, 1373-2237, 1384-1666, |
| | 1386-1585, 1390-1622, 1390-2055, 1392-1624, 1392-1791, 1396-1990, 1397-2036, 1404-1796, 1407-1810, |
| | 1409-1670, 1409-1697, 1409-1904, 1410-1643, 1417-1995, 1428-1626, 1429-1705, 1431-1677, 1432-1738, |
| | 1434-1963, 1434-2058, 1437-1709, 1437-1869, 1444-1705, 1444-1707, 1449-1671, 1458-1715, 1462-1708, 1469-2018, |
| | 1473-1729, 1476-2190, 1477-1534, 1481-2201, 1486-1727, 1491-1719, 1491-1823, 1492-2244, 1494-1825, |
| | 1497-1750, 1499-2052, 1501-1758, 1506-1759, 1506-2111, 1507-2093, 1511-1792, 1511-1805, 1511-2174, 1512-1773, |
| | 1513-1807, 1513-2132, 1515-2052, 1527-1765, 1527-1794, 1527-2084, 1531-2125, 1533-1731, 1536-1993, |
| | 1542-2110, 1546-1796, 1548-2192, 1550-1860, 1558-2200, 1567-2105, 1570-2147, 1571-2052, 1571-2166, 1574-1876, |
| | 1576-2058, 1578-1836, 1580-1826, 1587-2059, 1588-2076, 1589-1989, 1589-2254, 1590-2200, 1590-2238, |
| | 1597-1835, 1598-1865, 1604-1854, 1604-1892, 1604-2008, 1604-2052, 1606-1861, 1607-1847, 1607-1859, 1609-2195, |
| | 1610-1833, 1611-2238, 1613-1875, 1613-1876, 1613-1881, 1616-2237, 1617-1994, 1620-1715, 1624-2079, |
| | 1627-2122, 1628-1913, 1628-2239, 1630-1902, 1632-2210, 1633-2227, 1634-1914, 1637-2252, 1638-1909, |
| | 1642-2236, 1644-2249, 1646-1908, 1654-2252, 1655-1949, 1658-2238, 1665-1950, 1665-1966, 1665-2254, 1666-1902, |
| | 1668-1936, 1675-1892, 1675-1912, 1675-2072, 1677-1874, 1677-1932, 1677-1953, 1677-2238, 1677-2251, |
| | 1678-2199, 1682-1888, 1684-2254, 1689-1940, 1689-2047, 1696-2200, 1699-2239, 1702-2254, 1706-1952, 1707-2254, |
| | 1712-2116, 1713-1859, 1723-1972, 1725-2239, 1727-1970, 1736-2055, 1736-2220, 1737-1954, 1738-1961, |
| | 1738-2201, 1738-2224, 1738-2227, 1742-2046, 1743-1997, 1746-1997, 1748-2253, 1752-2209, 1764-2034, 1770-2052, |
| | 1771-2210, 1771-2239, 1774-2246, 1775-2040, 1777-2254, 1781-2240, 1783-2025, 1785-2239, 1788-2254, |
| | 1791-2199, 1791-2254, 1793-2245, 1798-2242, 1800-1895, 1800-2204, 1802-2065, 1803-2237, 1805-2201, 1810-2205, |
| | 1811-2201, 1813-2247, 1815-2242, 1821-2239, 1822-2196, 1822-2238, 1822-2242, 1825-2238, 1825-2244, |
| | 1826-2085, 1827-2111, 1828-2238, 1830-2254, 1831-2238, 1831-2254, 1832-2169, 1832-2239, 1832-2251, |
| | 1832-2252, 1835-2238, 1837-2247, 1839-2245, 1841-2244, 1841-2247, 1842-2238, 1843-2142, 1844-2211, 1845-2123, |
| | 1845-2238, 1847-2216, 1848-2238, 1854-2248, 1857-2027, 1860-2254, 1861-2145, 1870-2085, 1870-2238, |
| | 1874-2239, 1875-2106, 1875-2254, 1877-2239, 1881-2254, 1884-2238, 1891-1913, 1891-2210, 1895-2238, 1899-2100, |
| | 1899-2242, 1904-2124, 1904-2239, 1913-2238, 1915-2194, 1915-2238, 1917-2201, 1917-2239, 1918-2238, |
| | 1920-2239, 1921-2238, 1922-2240, 1926-2126, 1926-2185, 1933-2222, 1934-2149, 1934-2159, 1934-2179, 1934-2242, |
| | 1935-2172, 1936-2213, 1936-2220, 1936-2239, 1938-2219, 1939-2219, 1947-2238, 1949-2244, 1971-2238, |
| | 1983-2182, 1985-2249, 1991-2244, 1993-2242, 1994-2238, 2037-2254, 2038-2254, 2043-2254, 2057-2254, 2071-2238, |
| | 2078-2254, 2081-2239, 2088-2254, 2100-2254, 2102-2238, 2123-2238, 2124-2252, 2125-2239, 2127-2254, |
| | 2142-2201, 2142-2237, 2142-2238, 2145-2254, 2149-2253, 2154-2238 |
| 61/7500745CB1/ | 1-125, 1-249, 1-290, 1-294, 1-334, 2-233, 4-334, 7-250, 7-255, 7-259, 7-271, 7-279, 7-302, 7-2139, 8-269, 8-290, 8-295, |
| 2139 | 8-314, 8-324, 10-334, 12-264, 12-274, 12-277, 12-279, 13-266, 13-275, 13-278, 13-280, 13-281, 13-308, 15-235, |
| | 15-260, 15-270, 16-248, 16-287, 16-297, 16-314, 18-299, 27-277, 35-266, 53-292, 58-329, 71-254, 86-221, 86-314, |
| | 86-334, 111-284, 264-867, 333-597, 335-756, 335-797, 335-820, 335-844, 335-848, 335-918, 337-991, 338-867, |
| | 338-876, 338-922, 350-846, 356-816, 357-900, 357-966, 358-510, 358-616, 362-885, 365-934, 365-969, 366-779, |
| | 370-877, 377-759, 383-576, 383-620, 383-679, 383-940, 383-971, 385-620, 385-945, 395-660, 400-875, 400-985, |
| | 402-779, 402-951, 403-533, 407-1001, 412-977, 426-724, 437-728, 439-933, 447-680, 447-688, 454-996, 455-1066, |
| | 458-934, 461-969, 466-898, 467-1073, |
| | 471-1044, 473-719, 490-744, 493-1085, 495-1052, 496-1090, 501-1039, 507-1106, 508-1036, 511-1102, 514-783, |
| | 518-1138, 525-1216, 531-1187, 533-764, 536-778, 538-683, 538-798, 539-838, 543-814, 550-1145, 553-1045, 556-923, |
| | 569-976, 581-851, 588-1131, 607-853, 611-974, 611-1005, 612-1107, 614-883, 614-1161, 619-974, 625-1224, |
| | 631-1204, 637-901, 637-912, 640-888, 642-1131, 644-1227, 646-1390, 648-940, 653-1065, 654-887, 654-912, 654-924, |
| | 657-942, 657-1056, 658-1482, 659-861, 662-1247, 664-785, 667-923, 670-1216, 679-906, 679-974, 685-927, |
| | 685-1275, 705-1363, 711-873, 711-968, 713-1337, 724-1185, 724-1233, 724-1257, 724-1465, 724-1469, 724-1490, |
| | 724-1546, 724-1575, 727-1434, 730-991, 731-911, 732-982, 734-988, 741-1209, 746-1314, 758-1006, 761-1049, |
| | 763-1362, 765-1295, 765-1338, 768-1430, 769-1231, 781-1290, 791-930, 794-1439, 795-1362, 804-1333, 814-1351, |
| | 816-1044, |
| | 821-1083, 823-1110, 833-1066, 836-1441, 837-1086, 837-1499, 843-1147, 845-1543, 846-1528, 855-1476, 860-1096, |
| | 860-1103, 864-1485, 872-1159, 875-1383, 878-1159, 883-1517, 884-1167, 886-1131, 892-1500, 897-1142, |
| | 901-1027, 901-1089, 911-1617, 916-1156, 922-1497, 924-1192, 926-1171, 928-1267, 933-1205, 937-1206, 937-1214, |
| | 942-1174, 945-1197, 947-1220, 947-1224, 947-1235, 947-1480, 950-1180, 955-1207, 955-1534, 955-1551, |
| | 965-1260, 969-1130, 969-1215, 972-1180, 980-1635, 982-1250, 982-1414, 985-1603, 987-1563, 991-1250, 992-1244, |
| | 992-1246, 992-1290, 995-1307, 998-1198, 998-1213, 998-1260, 1000-1159, 1006-1637, 1008-1255, 1016-1250, |
| | 1016-1588, 1017-1604, 1018-1195, 1018-1304, 1055-1362, 1057-1323, 1060-1249, 1062-1285, 1070-1323, |
| | 1070-1365, 1072-1693, 1074-1723, 1094-1795, 1105-1536, 1109-1613, 1109-1618, 1112-1237, 1114-1752, 1115-1702, |
| | 1120-1687, 1120-1764, 1124-1660, 1133-1367, 1134-1387, 1158-1402, 1167-1659, 1178-1427, 1180-1463, |
| | 1185-1876, 1194-1844, |
| | 1197-1461, 1197-1659, 1198-1890, 1204-1450, 1211-1479, 1214-1730, 1215-1784, 1225-1467, 1225-1910, 1237-1449, |
| | 1238-1485, 1243-1530, 1256-2125, 1265-2070, 1275-1507, 1275-1674, 1279-1873, 1292-1553, 1292-1787, |
| | 1293-1526, 1312-1588, 1314-1560, 1315-1621, 1317-1846, 1317-1941, 1327-1588, 1327-1590, 1332-1554, 1352-1901, |
| | 1359-2073, 1360-1417, 1364-2084, 1364-2098, 1369-1610, 1374-1602, 1375-2127, 1377-1708, 1380-1633, |
| | 1382-1935, 1384-1641, 1389-1642, 1389-1994, 1390-1976, 1394-1675, 1394-1688, 1394-2057, 1395-1656, 1396-1690, |
| | 1396-2015, 1409-1648, 1410-1643, 1410-1648, 1410-1677, 1410-1967, 1418-2092, 1419-1876, 1425-1993, |
| | 1431-2075, 1435-2066, 1436-2063, 1441-2083, 1444-2078, 1450-1988, 1452-2109, 1454-2049, 1454-2081, 1459-1941, |
| | 1461-1719, 1467-2033, 1470-2107, 1472-2137, 1473-2083, 1473-2129, 1480-1718, 1487-1737, 1487-1775, |
| | 1487-1891, 1489-1744, 1490-1742, 1492-2078, 1494-2124, 1496-1764, 1499-2120, 1500-1877, 1503-1598, |
| | 1507-1962, 1511-2112, 1511-2121, 1513-1785, 1515-2093, 1516-2110, 1520-2135, 1525-2119, 1527-2132, 1534-2050, |
| | 1537-2135, 1538-1832, 1541-2121, 1548-1849, 1548-2137, 1549-1785, 1551-1819, 1558-1775, 1558-1795, |
| | 1558-1858, 1558-1955, 1560-1704, 1560-1757, 1560-1815, 1560-2121, 1560-2134, 1567-2137, 1572-1823, 1572-2006, |
| | 1576-2107, 1582-2121, 1585-2137, 1589-1835, 1590-2137, 1595-1999, 1596-1742, 1606-1855, 1608-2121, |
| | 1610-1853, 1613-1894, 1613-2137, 1615-2081, 1619-1938, 1619-2103, 1620-1837, 1621-1844, 1621-2106, 1621-2136, |
| | 1625-1929, 1626-1880, 1629-1880, 1631-2136, 1635-2092, 1647-1917, 1653-1935, 1654-2093, 1654-2121, |
| | 1657-2119, 1658-1923, 1660-2137, 1664-2121, 1666-1908, 1668-2121, 1671-2137, 1674-2137, 1676-2120, 1681-2125, |
| | 1683-1778, 1683-2087, 1685-1948, 1686-2120, 1693-2088, 1694-2084, 1696-2130, 1698-2125, 1704-2121, |
| | 1705-2079, 1705-2123, 1705-2125, 1708-2122, 1708-2127, 1709-1968, 1710-1994, 1711-2121, 1713-2137, |
| | 1714-2121, 1714-2137, 1715-2052, 1715-2121, 1715-2135, 1715-2139, 1718-2123, 1720-2130, 1722-2128, 1724-2119, |
| | 1724-2127, 1725-2121, 1725-2122, 1726-2025, 1727-2094, 1728-2006, 1728-2121, 1730-2099, 1731-2121, |
| | 1737-2121, 1740-1910, 1743-2137, 1744-2028, 1753-1968, 1753-2121, 1757-2121, 1758-1989, 1758-2137, 1760-2121, |
| | 1764-2137, 1767-2121, 1774-1796, 1774-2093, 1778-2121, 1782-1983, 1782-2125, 1787-2007, 1787-2121, |
| | 1796-2121, 1798-2077, 1798-2121, 1800-2084, 1800-2121, 1803-2121, 1804-2121, 1805-2121, 1809-2009, 1809-2068, |
| | 1816-2105, 1817-2032, 1817-2042, 1817-2062, 1817-2125, 1818-2055, 1819-2103, 1819-2116, 1819-2133, |
| | 1821-2102, 1822-2102, 1830-2124, 1832-2127, 1866-2065, 1868-2132, 1874-2127, 1876-2125, 1877-2121, 1920-2137, |
| | 1921-2137, 1926-2137, 1940-2137, 1954-2121, 1961-2137, 1971-2137, 1983-2137, 1985-2121, 2007-2135, |
| | 2008-2121, 2010-2137, 2025-2084, 2025-2120, 2025-2121, 2032-2136, 2037-2124 |
| 62/7500929CB1/ | 1-164, 1-250, 1-259, 1-269, 1-273, 1-354, 1-356, 1-469, 15-597, 43-329, 341-616, 343-598, 343-617, 343-622, 343-648, |
| 648 | 345-594, 347-596, 384-606, 448-613, 451-610, 475-596, 511-601 |

TABLE 5

| Polynucleotide SEQ | | |
|---|---|---|
| ID NO: | Incyte Project ID: | Representative Library |
| 32 | 8268274CB1 | TESTTUT02 |
| 33 | 7500515CB1 | LIVRTMR01 |
| 34 | 2256826CB1 | UTRSNON03 |
| 35 | 7686186CB1 | BRABDIK02 |
| 37 | 7501945CB1 | UTRSTUE01 |
| 38 | 7500264CB1 | LIVRTUT01 |
| 39 | 7499935CB1 | BRABDIK02 |
| 40 | 7982285CB1 | MIXDTME01 |
| 41 | 7758505CB1 | HELAUNT01 |
| 42 | 6885756CB1 | BRAWTDR02 |
| 43 | 7500748CB1 | SCORNON02 |
| 44 | 7500749CB1 | BRSTTUT03 |
| 45 | 7503401CB1 | PLACFEF05 |
| 46 | 7503485CB1 | BRSTNOT01 |
| 47 | 7504076CB1 | SPLNTUE01 |
| 48 | 7500926CB1 | HELATXT01 |
| 49 | 7503216CB1 | FIBRTXS07 |
| 50 | 7503233CB1 | PROSTUT09 |
| 51 | 7726576CB1 | HIPONON02 |
| 52 | 7503507CB1 | KIDEUNE02 |
| 53 | 7503506CB1 | KIDEUNE02 |
| 54 | 7503509CB1 | LIVRTUT01 |
| 55 | 7505800CB1 | MUSCNOT02 |
| 56 | 7503141CB1 | LIVRTMR01 |
| 57 | 7500362CB1 | COLNPOT01 |
| 58 | 7503328CB1 | UTRSNOT11 |
| 59 | 7510464CB1 | BONMTUE02 |
| 60 | 7510394CB1 | BRSTTUT03 |
| 61 | 7500745CB1 | BRSTTUT03 |
| 62 | 7500929CB1 | LUNGNOT09 |

TABLE 6

| Library | Vector | Library Description |
|---|---|---|
| BRSTTUT03 | PSPORT1 | Library was constructed using RNA isolated from breast tumor |
| | | tissue removed from a 58-year-old Caucasian female during |
| | | a unilateral extended simple mastectomy. Pathology indicated multicentric |
| | | invasive grade 4 lobular carcinoma. The mass |
| | | was identified in the upper outer quadrant, and three separate nodules |
| | | were found in the lower outer quadrant of the left |
| | | breast. Patient history included skin cancer, rheumatic heart disease, |
| | | osteoarthritis, and tuberculosis. Family history |
| | | included cerebrovascular disease, coronary artery aneurysm, breast |
| | | cancer, prostate cancer, atherosclerotic coronary artery |
| | | disease, and type I diabetes. |
| COLNPOT01 | pINCY | Library was constructed using RNA isolated from colon polyp tissue |
| | | removed from a 40-year-old Caucasian female during |
| | | a total colectomy. Pathology indicated an inflammatory pseudopolyp; |
| | | this tissue was associated with a focally invasive |
| | | grade 2 adenocarcinoma and multiple tubuvillous adenomas. Patient |
| | | history included a benign neoplasm of the bowel. |
| FIBRTXS07 | pINCY | This subtracted library was constructed using 1.3 million clones from |
| | | a dermal fibroblast library and was subjected to two |
| | | rounds of subtraction hybridization with 2.8 million clones from an |
| | | untreated dermal fibroblast tissue library. The starting |
| | | library for subtraction was constructed using RNA isolated from |
| | | treated dermal fibroblast tissue removed from the breast of |
| | | a 31-year-old Caucasian female. The cells were treated with 9CIS |
| | | retinoic acid. The hybridization probe for subtraction |
| | | was derived from a similarly constructed library from RNA isolated |
| | | from untreated dermal fibroblast tissue from the same |
| | | donor. Subtractive hybridization conditions were based on the |
| | | methodologies of Swaroop et al., NAR (1991) 19: 1954 and |
| | | Bonaldo, et al., Genome Research (1996) 6: 791. |
| HELATXT01 | pINCY | Library was constructed using RNA isolated from HeLa cells |
| | | treated with TNF-a and IL-1b, 10 ng/nl each for 20 hours. The |
| | | HeLa cell line is derived from cervical adenocarcinoma |
| | | removed from a 31-year-old Black female. |
| HELAUNT01 | pINCY | Library was constructed using RNA isolated from HeLa cells. |
| | | The HeLa cell line is derived from cervical adenocarcinoma |
| | | removed from a 31-year-old Black female. |
| HIPONON02 | PSPORT1 | This normalized hippocampus library was constructed from |
| | | 1.13 M independent clones from a hippocampus tissue library. |
| | | RNA was isolated from the hippocampus tissue of a 72-year-old |
| | | Caucasian female who died from an intracranial bleed. |
| | | Patient history included nose cancer, hypertension, and arthritis. |
| | | The normalization and hybridization conditions were |
| | | adapted from Soares et al. (PNAS (1994) 91: 9228). |
| KIDEUNE02 | pINCY | This 5′ biased random primed library was constructed |
| | | using RNA isolated from an untreated transformed embryonal cell |
| | | line (293-EBNA) derived from kidney epithelial tissue (Invitrogen). |
| | | The cells were transformed with adenovirus 5 DNA. |
| PLACFEF05 | PCMV-ICIS | Library was constructed using RNA isolated from placental tissue |
| | | removed from a Caucasian fetus, who died after 16 |
| | | weeks' gestation from fetal demise and hydrocephalus. Patient |
| | | history included umbilical cord wrapped around the head (3 |
| | | times) and the shoulders (1 time). Serology was positive for anti-CMV |
| | | and remaining serologies were negative. Family |
| | | history included multiple pregnancies and live |
| | | births, and an abortion in the mother. |
| PROSTUT09 | pINCY | Library was constructed using RNA isolated from prostate tumor |
| | | tissue removed from a 66-year-old Caucasian male during |
| | | a radical prostatectomy, radical cystectomy, and urinary diversion. |
| | | Pathology indicated grade 3 transitional cell carcinoma. |
| | | The patient presented with prostatic inflammatory disease. |
| | | Patient history included lung neoplasm, and benign |
| | | hypertension. Family history included a malignant breast |
| | | neoplasm, tuberculosis, cerebrovascular disease, atherosclerotic |
| | | coronary artery disease and lung cancer. |
| SCORNON02 | PSPORT1 | This normalized spinal cord library was constructed from |
| | | 3.24 M independent clones from the a spinal cord tissue library. |
| | | RNA was isolated from the spinal cord tissue removed from |
| | | a 71-year-old Caucasian male who died from respiratory |
| | | arrest. Patient history included myocardial infarction, gangrene, |
| | | and end stage renal disease. The normalization and |
| | | hybridization conditions were adapted from Soares |
| | | et al.(PNAS (1994) 91: 9228). |
| SPLNTUE01 | PCDNA2.1 | This 5′ biased random primed library was constructed |
| | | using RNA isolated from spleen tumor tissue removed from a 28- |
| | | year-old male during total splenectomy. Pathology indicated |
| | | malignant lymphoma, diffuse large cell type, B-cell phenotype |
| | | with abundant reactive T-cells and marked granulomatous response |
| | | involving the spleen, where it formed approximately 45 |
| | | nodules, liver, and mulitiple lymph nodes. |
| TESTTUT02 | pINCY | Library was constructed using RNA isolated from testicular tumor |
| | | removed from a 31-year-old Caucasian male during |
| | | unilateral orchiectomy. Pathology indicated embryonal carcinoma. |
| UTRSNON03 | pINCY | This normalized library was constructed from 6.4 M independent |
| | | clones from the UTRSNOT12 library. RNA was isolated |
| | | from uterine myometrial tissue removed from a 41-year-old |
| | | Caucasian female during a vaginal hysterectomy with dilation |
| | | and curettage. The endometrium was secretory and contained |
| | | fragments of endometrial polyps. Benign endo-and |
| | | ectocervical mucosa were identified in the endocervix. Pathology |
| | | for the associated tumor tissue indicated uterine |
| | | leiomyoma. Patient history included ventral hernia and a benign ovarian |
| | | neoplasm. The normalization and hybridization |
| | | conditions were adapted from Soares et al. (PNAS (1994) 91: 9228). |
| UTRSNOT11 | pINCY | Library was constructed using RNA isolated from uterine myometrial |
| | | tissue removed from a 43-year-old female during a |
| | | vaginal hysterectomy and removal of the fallopian tubes and ovaries. |
| | | Pathology for the associated tumor tissue indicated |
| | | that the myometrium contained an intramural and a submucosal |
| | | leiomyoma. Family history included benign hypertension, |
| | | hyperlipidemia, colon cancer, type II diabetes, and |
| | | atherosclerotic coronary artery disease. |
| UTRSTUE01 | PCDNA2.1 | This 5′ biased random primed library was constructed using |
| | | RNA isolated from uterus tumor tissue removed a 37-year-old |
| | | Black female during myomectomy, dilation and curettage, |
| | | right fimbrial region biopsy, and incidental appendectomy. |
| | | Pathology indicated multiple (12) uterine leiomyomata. A fimbrial |
| | | cyst was identified. The patient presented with |
| | | deficiency anemia, an umbilical hernia, and premenopausal |
| | | menorrhagia. Patient history included premenopausal |
| | | menorrhagia and sarcoidosis of the lung. Previous surgeries |
| | | included hysteroscopy, dilation and curettage, and an |
| | | endoscopic lung biopsy. Patient medications included |
| | | Chromagen and Claritin. Family history included acute myocardial |
| | | infarction and atherosclerotic coronary artery disease in the father. |

TABLE 7

| | | | Parameter |
|---|---|---|---|
| Program | Description | Reference | Threshold |
| ProfileScan | An algorithm that searches for structural and sequence | Gribskov, M. et al. (1988) CABIOS 4: 61-66; | Normalized |
| | motifs in protein sequences that match sequence patterns | Gribskov, M. et al. (1989) Methods Enzymol. | quality score ≧ |
| | defined in Prosite. | 183: 146-159; Bairoch, A. et al. (1997) | GCG- |
| | | Nucleic Acids Res. 25: 217-221. | specified |
| | | | “HIGH” |
| | | | value for |
| | | | that particular |
| | | | Prosite motif. |
| | | | Generally, |
| | | | score = |
| | | | 1.4-2.1. |
| Phred | A base-calling algorithm that examines automated | Ewing, B. et al. (1998) Genome Res. | |
| | sequencer traces with high sensitivity and probability. | 8: 175-185; Ewing, B. and P. Green | |
| | | (1998) Genome Res. 8: 186-194. | |
| Phrap | A Phils Revised Assembly Program including SWAT and | Smith, T. F. and M. S. Waterman (1981) Adv. | Score = |
| | CrossMatch, programs based on efficient implementation | Appl. Math. 2: 482-489; Smith, T. F. and M. S. Waterman | 120 or greater; |
| | of the Smith-Waterman algorithm, useful in searching | (1981) J. Mol. Biol. 147: 195-197; | Match length = |
| | sequence homology and assembling DNA sequences. | and Green, P., University of Washington, | 56 or greater |
| | | Seattle, WA. | |
| Consed | A graphical tool for viewing and editing Phrap assemblies. | Gordon, D. et al. (1998) Genome Res. 8: 195-202. | |
| SPScan | A weight matrix analysis program that scans protein | Nielson, H. et al. (1997) Protein Engineering | Score = |
| | sequences for the presence of secretory signal peptides. | 10: 1-6; Claverie, J. M. and S. Audic (1997) | 3.5 or greater |
| | | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos (1996) | |
| | determine orientation. | Protein Sci. 5: 363-371. | |
| TMHMMER | A program that uses a hidden Markov model (HMM) to | Sonnhammer, E. L. et al. (1998) Proc. Sixth Intl. | |
| | delineate transmembrane segments on protein sequences | Conf. on Intelligent Systems for Mol. Biol., | |
| | and determine orientation. | Glasgow et al., eds., The Am. Assoc. for Artificial | |
| | | Intelligence Press, Menlo Park, CA, pp. 175-182. | |
| Motifs | A program that searches amino acid sequences for patterns | Bairoch, A. et al. (1997) Nucleic Acids Res. 25: 217-221; | |
| | that matched those defined in Prosite. | Wisconsin Package Program Manual, version 9, page | |
| | | M51-59, Genetics Computer Group, Madison, WI. | |

TABLE 8

| | | | | | | | | | | | African | | |
|---|---|---|---|---|---|---|---|---|---|---|---|---|---|
| SEQ | | | | | | | | | | Caucasian | Allele 1 | Asian | Hispanic |
| ID | | | | EST | CBI | EST | | | Amino | Allele 1 | fre- | Allele 1 | Allele 1 |
| NO: | PID | EST ID | SNP ID | SNP | SNP | Allele | Allele 1 | Allele 2 | Acid | frequency | quency | frequency | frequency |
| 57 | 7500362 | 1239004H1 | SNP00100193 | 124 | 603 | T | T | C | Y198 | n/a | n/a | n/a | n/a |
| 57 | 7500362 | 1320137H1 | SNP00061186 | 182 | 1013 | G | G | C | G335 | n/d | n/d | n/a | n/d |
| 57 | 7500362 | 1402571H1 | SNP00061186 | 251 | 1012 | G | G | C | A335 | n/d | n/d | n/a | n/d |
| 57 | 7500362 | 1406223H1 | SNP00051751 | 56 | 1340 | C | C | A | T444 | n/d | n/d | n/d | n/d |
| 57 | 7500362 | 1493274H1 | SNP00051750 | 17 | 1074 | C | C | T | H355 | n/a | n/a | n/a | n/a |
| 57 | 7500362 | 1532731H1 | SNP00061186 | 5 | 1011 | G | G | C | Q334 | n/d | n/d | n/a | n/d |
| 57 | 7500362 | 1819683H1 | SNP00051750 | 212 | 1076 | C | C | T | A356 | n/a | n/a | n/a | n/a |
| 57 | 7500362 | 2563605H2 | SNP00006074 | 54 | 1246 | T | T | C | F413 | n/a | n/a | n/a | n/a |
| 57 | 7500362 | 3095949H1 | SNP00051750 | 268 | 1073 | C | C | T | P355 | n/a | n/a | n/a | n/a |
| 57 | 7500362 | 3095949H1 | SNP00061186 | 205 | 1010 | G | G | C | R334 | n/d | n/d | n/a | n/d |
| 57 | 7500362 | 3097881H1 | SNP00051750 | 266 | 1072 | C | C | T | H355 | n/a | n/a | n/a | n/a |
| 57 | 7500362 | 3097881H1 | SNP00061186 | 203 | 1009 | G | G | C | E334 | n/d | n/d | n/a | n/d |
| 57 | 7500362 | 3356734H1 | SNP00100193 | 104 | 601 | T | T | C | Y198 | n/a | n/a | n/a | n/a |
| 57 | 7500362 | 3481734H1 | SNP00100193 | 17 | 597 | T | T | C | N196 | n/a | n/a | n/a | n/a |
| 57 | 7500362 | 3773311H1 | SNP00100193 | 92 | 602 | T | T | C | F198 | n/a | n/a | n/a | n/a |
| 57 | 7500362 | 4071254H1 | SNP00051751 | 39 | 1339 | C | C | A | P444 | n/d | n/d | n/d | n/d |
| 57 | 7500362 | 4436184H1 | SNP00100193 | 110 | 599 | T | T | C | V197 | n/a | n/a | n/a | n/a |
| 57 | 7500362 | 4601138H1 | SNP00006074 | 155 | 1245 | T | T | C | F412 | n/a | n/a | n/a | n/a |
| 57 | 7500362 | 4833716H1 | SNP00051750 | 228 | 1066 | C | C | T | H353 | n/a | n/a | n/a | n/a |
| 57 | 7500362 | 4833716H1 | SNP00061186 | 165 | 1001 | G | G | C | G331 | n/d | n/d | n/a | n/d |
| 57 | 7500362 | 4839758H2 | SNP00006074 | 170 | 1242 | T | T | C | S411 | n/a | n/a | n/a | n/a |
| 57 | 7500362 | 4839758H2 | SNP00051751 | 264 | 1336 | C | C | A | L443 | n/d | n/d | n/d | n/d |
| 57 | 7500362 | 6809701J1 | SNP00051750 | 123 | 1068 | C | C | T | D353 | n/a | n/a | n/a | n/a |
| 57 | 7500362 | 6809701J1 | SNP00061186 | 60 | 1005 | G | G | C | M332 | n/d | n/d | n/a | n/d |
| 57 | 7500362 | 7191933H2 | SNP00051750 | 225 | 1064 | T | C | T | I352 | n/a | n/a | n/a | n/a |
| 57 | 7500362 | 7191933H2 | SNP00061186 | 161 | 992 | G | G | C | S328 | n/d | n/d | n/a | n/d |
| 58 | 7503328 | 055117H1 | SNP00131006 | 104 | 2134 | C | C | T | noncoding | n/a | n/a | n/a | n/a |
| 58 | 7503328 | 1492725H1 | SNP00025167 | 61 | 1794 | G | G | A | noncoding | n/a | n/a | n/a | n/a |
| 59 | 7510464 | 3411052H1 | SNP00131006 | 165 | 2222 | C | C | T | noncoding | n/a | n/a | n/a | n/a |
| 59 | 7510464 | 3512172H1 | SNP00131006 | 181 | 2227 | C | C | T | noncoding | n/a | n/a | n/a | n/a |
| 59 | 7510464 | 3596460H1 | SNP00025166 | 43 | 148 | C | C | G | H28 | n/d | n/d | n/d | n/d |
| 59 | 7510464 | 3775141H1 | SNP00025166 | 76 | 127 | C | C | G | H21 | n/d | n/d | n/d | n/d |
| 59 | 7510464 | 4175359H1 | SNP00131006 | 40 | 2224 | C | C | T | noncoding | n/a | n/a | n/a | n/a |
| 59 | 7510464 | 4465166H1 | SNP00131006 | 41 | 2230 | C | C | T | noncoding | n/a | n/a | n/a | n/a |
| 59 | 7510464 | 4708867H1 | SNP00068560 | 136 | 1378 | T | T | C | Y438 | n/a | n/a | n/a | n/a |
| 59 | 7510464 | 4785219H1 | SNP00141118 | 159 | 687 | C | C | G | N207 | n/a | n/a | n/a | n/a |
| 59 | 7510464 | 4875944H1 | SNP00068560 | 102 | 1380 | T | T | C | H438 | n/a | n/a | n/a | n/a |
| 59 | 7510464 | 572892H1 | SNP00131006 | 100 | 2239 | C | C | T | noncoding | n/a | n/a | n/a | n/a |
| 59 | 7510464 | 6550203H1 | SNP00131006 | 188 | 2243 | C | C | T | noncoding | n/a | n/a | n/a | n/a |
| 60 | 7510394 | 1282428H1 | SNP00075286 | 180 | 1054 | C | C | T | noncoding | n/d | n/d | n/d | n/d |
| 60 | 7510394 | 1282428H1 | SNP00106459 | 90 | 964 | C | C | G | noncoding | n/a | n/a | n/a | n/a |
| 60 | 7510394 | 1299002H1 | SNP00106460 | 204 | 1175 | G | G | C | noncoding | n/d | n/a | n/a | n/a |
| 60 | 7510394 | 1332279H1 | SNP00009699 | 27 | 1457 | G | A | G | noncoding | n/a | n/a | n/a | n/a |
| 60 | 7510394 | 1332279H1 | SNP00097916 | 45 | 1475 | C | C | T | noncoding | n/a | n/a | n/a | n/a |
| 60 | 7510394 | 1373856H1 | SNP00009700 | 181 | 1706 | T | C | T | noncoding | n/a | n/a | n/a | n/a |
| 60 | 7510394 | 1401460H1 | SNP00053363 | 87 | 95 | G | G | A | R30 | n/d | n/a | n/a | n/a |
| 60 | 7510394 | 1483017H1 | SNP00075287 | 63 | 1337 | G | G | A | noncoding | n/a | n/a | n/a | n/a |
| 60 | 7510394 | 1830710H1 | SNP00009700 | 110 | 1707 | T | C | T | noncoding | n/a | n/a | n/a | n/a |
| 60 | 7510394 | 2113488H1 | SNP00053363 | 86 | 94 | G | G | A | V30 | n/d | n/a | n/a | n/a |
| 60 | 7510394 | 2113488H1 | SNP00065694 | 65 | 73 | C | C | T | P23 | n/d | n/d | n/d | n/d |
| 60 | 7510394 | 2481214H1 | SNP00053363 | 81 | 93 | G | G | A | R29 | n/d | n/a | n/a | n/a |
| 60 | 7510394 | 2481214H1 | SNP00065694 | 59 | 71 | C | C | T | P22 | n/d | n/d | n/d | n/d |
| 60 | 7510394 | 2492955H1 | SNP00065694 | 74 | 74 | C | C | T | A23 | n/d | n/d | n/d | n/d |
| 60 | 7510394 | 2530385H1 | SNP00009700 | 5 | 1671 | T | C | T | noncoding | n/a | n/a | n/a | n/a |
| 60 | 7510394 | 2542562H1 | SNP00009700 | 227 | 1712 | C | C | T | noncoding | n/a | n/a | n/a | n/a |
| 60 | 7510394 | 2560813H1 | SNP00075287 | 163 | 1336 | G | G | A | noncoding | n/a | n/a | n/a | n/a |
| 60 | 7510394 | 5074436H1 | SNP00106459 | 136 | 963 | C | C | G | noncoding | n/a | n/a | n/a | n/a |
| 60 | 7510394 | 5594636H1 | SNP00134605 | 86 | 856 | A | A | C | noncoding | n/a | n/a | n/a | n/a |
| 60 | 7510394 | 5879206H1 | SNP00075286 | 37 | 971 | C | C | T | noncoding | n/d | n/d | n/d | n/d |
| 60 | 7510394 | 5879206H1 | SNP00106460 | 158 | 1089 | G | G | C | noncoding | n/d | n/a | n/a | n/a |
| 60 | 7510394 | 5909982H1 | SNP00053363 | 83 | 86 | G | G | A | R27 | n/d | n/a | n/a | n/a |
| 60 | 7510394 | 5909982H1 | SNP00065694 | 61 | 64 | C | C | T | R20 | n/d | n/d | n/d | n/d |
| 60 | 7510394 | 5955251H1 | SNP00075286 | 50 | 1049 | T | C | T | noncoding | n/d | n/d | n/d | n/d |
| 60 | 7510394 | 5955251H1 | SNP00106460 | 171 | 1170 | G | G | C | noncoding | n/d | n/a | n/a | n/a |
| 60 | 7510394 | 5978154H1 | SNP00075287 | 236 | 1334 | G | G | A | noncoding | n/a | n/a | n/a | n/a |
| 60 | 7510394 | 6171175H1 | SNP00075287 | 212 | 1324 | G | G | A | noncoding | n/a | n/a | n/a | n/a |
| 60 | 7510394 | 6171175H1 | SNP00106460 | 50 | 1162 | G | G | C | noncoding | n/d | n/a | n/a | n/a |
| 60 | 7510394 | 6849471H1 | SNP00009699 | 177 | 1436 | A | A | G | noncoding | n/a | n/a | n/a | n/a |
| 60 | 7510394 | 6849471H1 | SNP00097916 | 195 | 1454 | C | C | T | noncoding | n/a | n/a | n/a | n/a |
| 60 | 7510394 | 953943H1 | SNP00106460 | 94 | 1176 | G | G | C | noncoding | n/d | n/a | n/a | n/a |
| 61 | 7500745 | 1282428H1 | SNP00075286 | 180 | 937 | C | C | T | noncoding | n/d | n/d | n/d | n/d |
| 61 | 7500745 | 1282428H1 | SNP00106459 | 90 | 847 | C | C | G | noncoding | n/a | n/a | n/a | n/a |
| 61 | 7500745 | 1299002H1 | SNP00106460 | 204 | 1058 | G | G | C | noncoding | n/d | n/a | n/a | n/a |
| 61 | 7500745 | 1332279H1 | SNP00009699 | 27 | 1340 | G | A | G | noncoding | n/a | n/a | n/a | n/a |
| 61 | 7500745 | 1332279H1 | SNP00097916 | 45 | 1358 | C | C | T | noncoding | n/a | n/a | n/a | n/a |
| 61 | 7500745 | 1373856H1 | SNP00009700 | 181 | 1589 | T | C | T | noncoding | n/a | n/a | n/a | n/a |
| 61 | 7500745 | 1401460H1 | SNP00053363 | 87 | 99 | G | G | A | L30 | n/d | n/a | n/a | n/a |
| 61 | 7500745 | 1483017H1 | SNP00075287 | 63 | 1220 | G | G | A | noncoding | n/a | n/a | n/a | n/a |
| 61 | 7500745 | 2113488H1 | SNP00065694 | 65 | 77 | C | C | T | A23 | n/d | n/d | n/d | n/d |
| 61 | 7500745 | 5594636H1 | SNP00134605 | 86 | 739 | A | A | C | noncoding | n/a | n/a | n/a | n/a |

1. An isolated polypeptide selected from the group consisting of:
a) a polypeptide comprising an amino acid sequence selected from the group consisting of SEQ ID NO:1-31,
b) a polypeptide comprising a naturally occurring amino acid sequence at least 90% identical to an amino acid sequence selected from the group consisting of SEQ ID NO:1, SEQ ID NO:4-6, SEQ ID NO:9-13, SEQ ID NO: 17, SEQ ID NO:20-21, SEQ ID NO:23, SEQ ID NO:27-28, and SEQ ID NO:30-31,
c) a polypeptide comprising a naturally occurring amino acid sequence at least 97% identical to the amino acid sequence of SEQ ID NO:3,
d) a polypeptide consisting essentially of a naturally occurring amino acid sequence at least 95% identical to the amino acid sequence of SEQ ID NO: 18,
e) a polypeptide consisting essentially of a naturally occurring amino acid sequence at least 91% identical to the amino acid sequence of SEQ ID NO: 19,
f) a polypeptide comprising a naturally occurring amino acid sequence at least 91% identical to the amino acid sequence of SEQ ID NO:29,
g) a polypeptide consisting essentially of a naturally occurring amino acid sequence at least 90% identical to an amino acid sequence selected from the group consisting of SEQ ID NO:14-15, SEQ ID NO:22, and SEQ ID NO:24-26,
h) a biologically active fragment of a polypeptide having an amino acid sequence selected from the group consisting of SEQ ID NO: 1-31, and
i) an immunogenic fragment of a polypeptide having an amino acid sequence selected from the group consisting of SEQ ID NO: 1-31.

2. An isolated polypeptide of claim 1 comprising an amino acid sequence selected from the group consisting of SEQ ID NO: 1-31.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO: 32-62.

6. A recombinant polynucleotide comprising a promoter sequence operably linked to a polynucleotide of claim 3.

7. A cell transformed with a recombinant polynucleotide of claim 6.

8. (canceled)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO: 1-31.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:32-62,
b) a polynucleotide comprising a naturally occurring polynucleotide sequence at least 90% identical to a polynucleotide sequence selected from the group consisting of SEQ ID NO:32-41, SEQ ID NO:43-56, and SEQ ID NO:61-62,
c) a polynucleotide comprising a naturally occurring polynucleotide sequence at least 92% identical to the polynucleotide sequence of SEQ ID NO:42,
d) a polynucleotide comprising a naturally occurring polynucleotide sequence at least 97% identical to the polynucleotide sequence of SEQ ID NO:59,
e) a polynucleotide comprising a naturally occurring polynucleotide sequence at least 98% identical to a polynucleotide sequence selected from the group consisting of SEQ ID NO:58 and SEQ ID NO:60,
f) a polynucleotide complementary to a polynucleotide of a),
g) a polynucleotide complementary to a polynucleotide of b),
h) a polynucleotide complementary to a polynucleotide of c),
i) a polynucleotide complementary to a polynucleotide of d),
j) a polynucleotide complementary to a polynucleotide of e), and
k) an RNA equivalent of a)-j).

13. (canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (canceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO: 1-31.

19. (canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (canceled)

22. (canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (canceled)

25. (canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (canceled)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-117. (canceled)


SEQUENCE LISTING (62 sequences)

SEQ ID NO: 1 - protein, 404 aa, Homo sapiens
  misc_feature: Incyte ID No 8268274CD1
METHISCLFPELLAMIFGYLDVRDKGRAAQVCTAWRDAAYHKSVWRGVEAKLHLRRANPSLFPSLQARGIRRVQILSLRRSLSYVIQGMANIESLNLSGCYNLTDNGLGHAFVQEIGSLRALNLSLCKQITDSSLGRIAQYLKGLEVLELGGCSNITNTGLLLIAWGLQRLKSLNLRSCRHLSDVGIGHLAGMTRSAAEGCLGLEQLTLQDCQKLTDLSLKHISRGLTGLRLLNLSFCGGISDAGLLHLSHMGSLRSLNLRSCDNISDTGIMHLAMGSLRLSGLDVSFCDKVGDQSLAYIAQGLDGLKSLSLCSCHISDDGINRMVRQMHGLRTLNIGQCVRITDKGLELIAEHLSQLTGIDLYGCTRITKRGLERITQLPCLKEARGDFSPLFTVRTRGSSRR

SEQ ID NO: 2 - protein, 900 aa, Homo sapiens
  misc_feature: Incyte ID No 7500515CD1
MKPPRPVRTCSKVLVLLSLLAIHQTTTAEKNGIDIYSLTVDSRVSSRFAHTVVTSRVVNRANTVQEATFQMELPKKAFITNFSMIIDGMTYPGIIKEKAEAQAQYSAAVAKGKSAGLVKATGRNMEQFQVSVSVAPNAKITFELVYEELLKRRLGVYELLLKVRPQQLVKHLQMDIHIFEPQGISFLETESTFMTNQLVDALTTWQNKTKAHIRFKPTLSQQQKSPEQQETVLDGNLIIRYDVDRAISGGSIQIENGYFVHYFAPEGLTTMPKNVVFVIDKSGSMSGRKIQQTREALIKILDDLSPRDQFNLIVFSTEATQWRPSLVPASAENVNKARSFAAGIQALGGTNINDAMLMAVQLLDSSNQEERLPEGSVSLIILLTDGDPTVGETNPRSIQNNVREAVSGRYSLFCLGFGFDVSYAFLEKLALDNGGLARRIHEDSDSALQLQDFYQEVANPLLTAVTFEYPSNAVEEVTQNNFRLLFKGSEMVVAGKLQDRGPDVLTATVSGKLPTQNITFQTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSAGAAGSRMNFRPGVLSSRQLGLPGPPDVPDHAAYHPFRRLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 3 - protein, 436 aa, Homo sapiens
  misc_feature: Incyte ID No 2256826CD1
MRRDVNGVTKSRFEMFSNSDEAVINKKLPKELLLRIFSFLDVVTLCRCAQVSRAWNVLALDGSNWQRIDLFDFQRDIEGRVVENISKRCGGFLRKLSLRGCLGVGDNALRTFAQNCRNIEVLNLNGCTKTTDATCTSLSKFCSKLRHLDLASCTSITNMSLKALSEGCPLLEQLNISWCDQVTKDGIQALVRGCGGLKALFLKGCTQLEDEALKYIGAHCPELVTLNLQTCLQITDEGLITICRGCHKLQSLCASGCSNITDAILNALGQNCPRLRILEVARCSQLTDVGFTTLARNCHELEKMDLEECVQITDSTLIQLSIHCPRLQVLSLSHCELITDDGIRHLGNGACAHDQLEVIELDNCPLITDASLEHLKSCHSLERIELYDCQQITRAGIKRLRTHLPNIKVHAYFAPVTPPPSVGGSRQRFCRCCIIL

SEQ ID NO: 4 - protein, 356 aa, Homo sapiens
  misc_feature: Incyte ID No 7686186CD1
MENDVTYPDPYSRPAPDRFIRRWLVITGCIAALMLLWQFLPAIEAWFSPHETQERTVTPRGDLAADEKTTIELFEKSRGSVVYITTAQLVRDVWSRNVFSVPRGTGSGFIWDDAGHVVTNFHVIQGASSATVKLADGRDYQAALVGASPAHDIAVLKIGVGFKRPPAVPVGTSADLKVGQKVFAIGNPFGLDWTLTTGIVSALDRTLSGDASGPAIDHLIQTDAAINPGNSGGPLLDSAGRLIGINTAIYSPSGASAGIGFAVPVDTVMRVVPQLIKTGKYIRPALGIEVDEQLNARLQALTGSKGVFVLRVTPGSAAHRAGLVGVEVTAGGIVPGDRVISIDGIAVDPGIPDRTC

SEQ ID NO: 5 - protein, 432 aa, Homo sapiens [sequence omitted]

SEQ ID NO: 6 - protein, 248 aa, Homo sapiens
  misc_feature: Incyte ID No 7501945CD1
MGPSSLRKTSSGLPLILHYGVILGAPLASSCAGACGTSFPDGLTPEGTQASGDKDIPAINQGLILEETPESSFLIEGDIIRPSPFRLLSATSNKWPMGGSGVVEVPFLLSSKYDEPSRQVILEALAEFERSTCIRFVTYQDQRDFISIIPMYGCFSSVGRSGGMQVVSLAPTCLQKGRGIVLHELMHVLGFWHEHTRADRDRYIHVNWNEILPGFEINFIKSRSSNMLTPYDYSSVMHYGRVPCPQHW

SEQ ID NO: 7 - protein, 388 aa, Homo sapiens
  misc_feature: Incyte ID No 7500264CD1
MASVAQESAGSQRRLPPRHGALRGLLLLCLWLPSGRAALPPAAPLSELHAQLSGVEQLLEEFRRQLQQERPQEELELELRAGGGPQEDCPGPGSGGYSAMPDAIIRTKDSLAAGASFLRAPAAVRGWRQCVAACCSEPRCSVAVVELPRRPAPPAAVLGCYLFNCTARGRNVCKFALHSGYSSYSLSRAPDGAALATARASPRQEKDAPPLSKAGQDVVLHLPTDGVVLDGRESTDDHAIVQYEWALLQGDPSVDMKVPQSGGDSLVEKSQKATAPNKPPALSNTEKRNHSAFWGPESQIIPVMPDSSSSGKNRKEESYIFESKGDGGGGEHPAPETGAVLPLALGLAITALLLLMVACRLRLVKQKLKKARPITSEESDYLINGMYL

SEQ ID NO: 8 - protein, 467 aa, Homo sapiens
  misc_feature: Incyte ID No 7499935CD1
MAAATGPSFWLGNETLKVPLALFALNRQRLCERLRKNPAVQAGSIVVLQGGEETQRYCTDTGVLFRQESFFHWAFGVTEPGCYGVIDVDTGKSTLFVPRLPASHATWMGKIHSKEHFKEKYAVDDVQYVDEIASVLTSQKPSVLLTLRGVNTDSGSVCREASFDGISKFEVNNTILHPEIVECRVFKTDMELEVLRYTNKISSEAHREVMKAVKVGMKEYELESLFEHYCYSRGGMRHSSYTCICGSGENSAVLHYGHAGAPNDRTIQNGDMCLFDMGGEYYCFASDITCSFPANGKFTADQKAVYEAVLRSSRAVMGAMKPGVWWPDMHRLADRIHLEELAHMGILSGSVDAMVQAHLGAVFMPHGLGHFLGIDVHDVGGYPEGVERIYFIDHLLDEALADPARASFLNREVLQRFRGFGGVRIEEDVVVTDSGIELLTCVPRTVEEIEACMAGCDKAFTPFSGPK

SEQ ID NO: 9 - protein, 379 aa, Homo sapiens
  misc_feature: Incyte ID No 7982285CD1
MEGNRDEAEKCVEIAREALNAGNREKAQRFLQKAEKLYPLPSARALLEIIMKNGSTAGNSPHCRKPSGSGDQSKPNCTKDSTSGSGEGGKGYTKDQVDGVLSINKCKNYYEVLGVTKDAGDEDLKKAYRKLALKFHPDKNHAPGATDAFKKIGNAYAVLSNPEKRKQYDLTGNEEQACNHQNNGRFNFHRGCEADITPEDLFNIFFGGGFPSGSVHSFSNGRAGYSQQHQHRHSGHEREEERGDGGFSVFIQLMPIIVLILVSLLSQLMVSNPPYSLYPRSGTGQTIKMQTENLGVVYYVNKDFKNEYKGMLLQKVEKSVEEDYVTNIRNNCWKERQQKTDMQYAAKVYRDDRLRRKADALSMDNCKELERLTSLYKGG

SEQ ID NO: 10 - protein, 737 aa, Homo sapiens
  misc_feature: Incyte ID No 7758505CD1
MGVLKVWLGLALALAEFAVLPHHSEGACVYQDSLLADATIWKPDSCQSCRCHGDIVICKPAVCRNPQCAFEKGEVLQIAANQCCPECVLRTPGSCHHEKKIHEHGTEWASSPCSVCSCNHGEVRCTPQPCPPLSCGHQELAFIPEGSCCPVCVGLGKPCSYEGHVFQDGEDWRLSRCAKCLCRNGVAQCFTAQCQPLFCNQDETVVRVPGKCCPQCSARSCSAAGQVYEHGEQWSENACTTCICDRGEVRCHKQACLPLRCGKGQSRARRHGQCCEECVSPAGSCSYDGVVRYQDEMWKGSACEFCMCDHGQVTCQTGECAKVECARDEELIHLDGKCCPECISRNGYCVYEETGEFMSSNASEVKRIPEGEKWEDGPCKVCECRGAQVTCYEPSCPPCPVGTLALEVKGQCCPDCTSVHCHPDCLTCSQSPDHCDLCQDPTKLLQNGWCVHSCGLGFYQAGSLCIACQPQCSTCTSGLECSSCQPPLLMRHGQCVPTCGDGFYQDRHSCAVCHESCAGCWGPTEKHCLACRDPLHVLRDGGCESSCGKGFYNRQGTCSACDQSCDSCGPSSPRCLTCTEKTVLHDGKCMSECPGGYYADATGRCKVCHNSCASCSGPTPSHCTACSPPKALRQGHCLPRCGEGFYSDHGVCKACHSSCLACMGPAPSHCTGCKKPEEGLQVEQLSGVGIPSGECLAQCRAHFYLESTGLCEGQNLDFCQNLEVISAVCLGISSTEN

SEQ ID NO: 11 - protein, 530 aa, Homo sapiens
  misc_feature: Incyte ID No 6885756CD1
MEDDSLYLGGDWQFNHFSKLTSSRLDAAFAEIQRTSLSEKSPLSSETRFDLCDDLAPVARQLAPREKLPLSSRRPAAVGAGLQKIGNTFYVNVSLQCLTYTLPLSNYMLSREDSQTCHLHKCCMFCTMQAHITWALYRPGHVIQPSQVLAAGFHRGEQEDAHEFLMFTVDAMKKACLPGHKQLDHHSKDTTLIHQIFGAYWRSQIKYLHCHGISDTFDPYLDIALDIQAAQSVKQALEQLVKPKELNGENAYHCGLCLQKAPASKTLTLPTSAKVLILVLKRFSDVTGNKLAKNVQYPKCRDMQPYMSQQNTGPLVYVLYAVLVHAGWSCHNGHYFSYVKAQEGQWYKMDDAEVTASGITSVLSQQAYVLFYIQKSEWERHSESVSRGREPRALGAEDTDRPATQGELKRDHPCLQVPELDEHLVERATQESTLDHWKFPQKQNKTKPEFNVRKVEGTLPPNVLVIHQSKYKCGMKNHHPEQQSSLLNLSSTKPTDQESMNTGTLASLQGSTRRSKGNNKHSKRSLLVCQ

SEQ ID NO: 12 - protein, 511 aa, Homo sapiens
  misc_feature: Incyte ID No 7500748CD1
MAAAMPLALLVLLLLGPGGWCLAEPPRDSLREELVITPLPSGDVAATFQFRTRWDSELQREGVSHYRLFPKALGQLISKYSLRELHLSFTQGFWRTRYWGPPFLQAPSGAELWVWFQDTVTDVDKSWKELSNVLSGIFCASLNFIDSTNTVTPTASFKPLGLANDTDHYFLRYAVLPREVVCTENLTPWKKLLPCSSKAGLSVLLKADRLFHTSYHSQAVHIRPVCRNARCTSISWELRQTLSVVFDAFITGQGKKDWSLFRMFSRTLTEPCPLASESRVYVDITTYNQDNETLEVHPPPTTTYQDVILGTRKTYAIYDLLDTAMINNSRNLNIQLKWKRPPENGYIHYQPAQDRLQPHLLEMLIQLPANSVTKVSIQFERALLKWTEYTPDPNHGFYVSPSVLSALVPSMVAAKPVDWEESPLFNSLFPVSDGSNYFVRLYTEPLLVNLPTPDFSMPYNVICLTCTVVAVCYGSFYNLLTRTFHIEEPRTGGLAKRLANLIRRARGVPPL

SEQ ID NO: 13 - protein, 476 aa, Homo sapiens
  misc_feature: Incyte ID No 7500749CD1
MAAAMPLALLVLLLLGPGGWCLAEPPRDSLREELVITPLPSGDVAATFQFRTRWDSELQREGDTDHYFLRYAVLPREVVCTENLTPWKKLLPCSSKAGLSVLLKADRLFHTSYHSQAVHIRPVCRNARCTSISWELRQTLSVVFDAFITGQGKKDWSLFRMFSRTLTEPCPLASESRVYVDITTYNQDNETLEVHPPPTTTYQDVILGTRKTYAIYDLLDTAMINNSRNLNIQLKWKRPPENEAPPVPFLHAQRYVSGYGLQKGELSTLLYNTHPYRAFPVLLLDTVPWYLRLYVHTLTITSKGKENKPSYIHYQPAQDRLQPHLLEMLIQLPANSVTKVSIQFERALLKWTEYTPDPNHGFYVSPSVLSALVPSMVAAKPVDWEESPLFNSLFPVSDGSNYFVRLYTEPLLVNLPTPDFSMPYNVICLTCTVVAVCYGSFYNLLTRTFHIEEPRTGGLAKRLANLIRRARGVPPL

SEQ ID NO: 14 - protein, 344 aa, Homo sapiens
  misc_feature: Incyte ID No 7503401CD1
MAATEGVGEAAQGGEPGQPAQPPPQPHPPPPQQQHKEEMAAEAGEAVASPMDDGFVSLDSPSYVLYRDRAEWADIDPVPQNDGPNPVVQIIYSDKFRDVYDYFRAVLQRDERSERAFKLTRDAIELNAANYTVWHHRRVLVEWLRDPSQELEFIADILNQDAKNYHAWQHRQWVIQEFKLWDNELQYVDQLLKEDVRNNSVWNQRYFVISNTTGYNDRAVLEREVQYTLEMIKLVPHNESAWNYLKGILQDRGLSKYPNLLNQLLDLQPSHSSPYLIAFLVDIYEDMLENQCDNKEDILNKALELCEILAKEKDTIRKEYWRYIGRSLQSKHSTENDSPTNVQQ

SEQ ID NO: 15 - protein, 122 aa, Homo sapiens
  misc_feature: Incyte ID No 7503485CD1
MSTPARRRLMRDFKRLQEDPPAGVSGAPSENNIMVWNAVIFGPEGTPFEDVYADGSICLDILQNRWSPTYDVSSILTSIQSLLDEPNPNSPANSQAAQLYQENKREYEKRVSAIVEQSWRDC

SEQ ID NO: 16 - protein, 255 aa, Homo sapiens
  misc_feature: Incyte ID No 7504076CD1
MAVGNINELPENILLELFTHVPARQLLLNCRLVCSLWRDLIDLVTLWKRKCLREGFITEDWDQPVADWKIFYFLRSLHRNLLHNPCAEEGFEFWSLDVNGGDEWKVEDLSRDQRKEFPNDQVKKYFVTSYYTCLKSQVVDLKAEGYWEELMDTTRPDIEVKDWFAARPDCGSKYQLCVQLLSSAHAPLGTFQPDPATIQQKSDAKWREVSHTFSNYPPGVRYIWFQHGGVDTHYWAGWYGPRVTNSSITIGPPLP

SEQ ID NO: 17 - protein, 166 aa, Homo sapiens
  misc_feature: Incyte ID No 7500926CD1
MSAWAAASLSRAAARCLLARGPGVRAAPPRDPRPSHPEPRGCGAAPGRTLHFTAAVPAGHNKWSKVRHIKGPKDVERSRIFSKLCLNIRLAVKEGGPNPEHNSNLANILEVCRSKHMPKSTIETALKMEKSKDTYLLYEGRGPGGSSLLIEALSNSSHKCQADLRP

SEQ ID NO: 18 - protein, 591 aa, Homo sapiens
  misc_feature: Incyte ID No 7503216CD1
MPPKVTSELLRQLRQAMRNSEYVTEPIQAYIIPSGDAHQSEYIAPCDCRRAFVSGFDGSAGTAIITEEHAAMWTDGRYFLQAAKQMDSNWTLMKMGLKDTPTQEDWLVSVLPEGSRVGVDPLIIPTDYWKKMAKVLRSAGHHLIPVKENLVDKIWTDRPERPCKPLLTLGLDYTGLFNLRGSDVEHNPVFFSYAIIGLETIMLFIDGDRIDAPSVKEHLLLDLGLEAEYRIQVHPYKSILSELKALCADLSPREKVWVSDKASYAVSETIPKDHRCCMPYTPICIAKAVKNSAESEGMRRAHIKDAVALCELFNWLEKEVPKGGVTEISAADKAEEFRRQQADFVDLSFPTISSTGPNGAIIHYAPVPETNRTLSLDEVYLIDSGAQYKDGTTDVTRTMHFGTPTAYEKECFTYVLKGHIAVSAAVFPTGTKGHLLDSFARSALWDSGLDYLHGTGHGVGSFLNVHEGPCGISYKTFSDEPLEAGMIVTDEPGYYEDGAFGIRIENVVLVVPVKTKYNFNNRGSLTFEPLTLVPIQTKMIDVDSLTDKECDWLNNYHLTCRDVIGKELQKQGRQEALEWLIRETQPISKQH

SEQ ID NO: 19 - protein, 652 aa, Homo sapiens
  misc_feature: Incyte ID No 7503233CD1
MSEEIITPVYCTGVSAQVQKQRARELGLGRHENAIKYLGQDYEQLRVRCLQSGTLFRDEAFPPVPQSLGYKDLGPNSSKTYGYAGIFHFQLWQFGEWVDVVVDDLLPIKDGKLVFVHSAEGNEFWSALLEKAYAKVNGSYEALSGGSTSEGFEDFTGGVTEWYELRKAPSDLYQIILKALERGSLLGCSIDISSVLDMEAITFKKLVKGHAYSVTGAKQVNYRGQVVSLIRMRNPWGEVEWTGAWSDSSSEWNNVDPYERDQLRVKMEDGEFWMSFRDFMREFTRLEICNLTPDALKSRTIRKWNTTLYEGTWRRGSTAGGCRNYPATFWVNPQFKIRLDETDDPDDYGDRESGCSFVLALMQKHRRRERRFGRDMETIGFAVYEVPPELVGQPAVHLKRDFFLANASRARSEQFINLREVSTRFRLPPGEYVVVPSTFEPNKEGDFVLRFFSEKSAGTVELDDQIQANLPDEQVLSEEEIDENFKALFRQLAGEDMEISVKELRTILNRIISKHKDLRTKGFSLESCRSMVNLMDRDGNGKLGLVEFNILWNRIRNYLSIFRKFDLDKSGSMSAYEMRMAIESAGFKLNKKLYELIITRYSEPDLAVDFDNFVCCLVRLETMFRFFKTLDTDLDGVVTFDLFKWLQLTMFA

SEQ ID NO: 20 - protein, 861 aa, Homo sapiens
  misc_feature: Incyte ID No 7726576CD1
MAGPGPGAVLESPRQLLGRVRFLAEAARSLRAGRPLPAALAFVPREVLYKLYKDPAGPSRVLLPVWEAEGLGLRVGAAGPAPGTGSGPLRAARDSIELRRGACVRTTGEELCNGHGLWVKLTKEQLAEHLGDCGLQEGWLLVCRPAEGGARLVPIDTPNHLQRQQQLFGVDYRPVLRWEQVVDLTYSHRLGSRPQPAEAYAEAVQRLLYVPPTWTYECDEDLIHFLYDHLGKEDENLGSVKQYVESIDVSSYTEEFNVSCLTDSNADTYWESDGSQCQHWVRLTMKKGTIVKKLLLTVDTTDDNFMPKRVVVYGGEGDNLKKLSDVSIDETLIGDVCVLEDMTVHLPIIEIRIVECRDDGIDVRLRGVKIKSSRQRELGLNADLFQPTSLVRYPRLEGTDPEVLYRRAVLLQRLIKILDSVLHHLVPAWDHTLGTFSEIKQVKQFLLLSRQRPGLVAQCLRDSESSKPSFMPRLYINRRLAMEHRACPSRDPACKNAVFTQVYEGLKPSDKYEKPLDYRWPMRYDQWWECKFIAEGIIDQGGGFRDSLADMSEELCPSSADTPVPLPFFVRTANQGNGTGEARDMYVPNPSCRDFAKYEWIGQLMGAALRGKEFLVLALPGFVWKQLSGEEVSWSKDFPAVDSVLVKLLEVMEGMDKETFEFKFGKELTFTTVLSDQQVVELIPGGAGIVVGYGDRSRFIQLVQKARLEESKEQVAAMQAGLLKVVPQAVLDLLTWQELEKKVCGDPEVTVDALRKLTRFEDFEPSDSRVQYFWEALNNFTNEDRSRVLRFVTGRSRLPARIYIYPDKLGYETTDALPESSTCSSTLFLPHYASAKVCEEKLRYAAYNCVAIDTDMSPWEE

SEQ ID NO: 21 - protein, 447 aa, Homo sapiens
  misc_feature: Incyte ID No 7503507CD1
MASVAQESAGSQRRLPPRHGALRGLLLLCLWLPSGRAALPPAAPLSELHAQLSGVEQLLEEFRRQLQQERPQEELELELRAGGGPQEDCPGRGSGGYSAMPDAIIRTKDSLAAGASFLRAPAAVRGWRQCVAACCSEPRCSVAVVELPRRPAPPAAVLGCYLFNCTARGRNVCKFALHSGYSSYSLSRAPDGAALATARASPRQEKDAPPLSKAGQDVVLHLPTDGVVLDGRESTDDHAIVQYEWALLQGDPSVDMKVPQSGTLKLSHLQEGTYTFQLTVTDTAGQRSSDNVSVTVLRAAYSTGGCLHTCSRYHFFCDDGCCIDITLACDGVQQCPDGSDEDFCQNLGLDRKMVTHTAASPALPRTTGPSEDAGGDSLVEKSQKATAPNKPPALSNTEKRKVIYLSQRVMEEEGNTQPQKQVQCYPWRWVWLSLLCCFSWLHADYDW

SEQ ID NO: 22 - protein, 468 aa, Homo sapiens
  misc_feature: Incyte ID No 7503506CD1
MASVAQESAGSQRRLPPRHGALRGLLLLCLWLPSGRAALPPAAPLSELHAQLSGVEQLLEEFRRQLQQERPQEELELELRAGGGPQEDCPGRGSGGYSAMPDAIIRTKDSLAAGASFLRAPAAVRGWRQCVAACCSEPRCSVAVVELPRRPAPPAAVLGCYLFNCTARGRNVCKFALHSGYSSYSLSRAPDGAALATARASPRQEKDAPPLSKAGQDVVLHLPTDGVVLDGRESTDDHAIVQYEWALLQGDPSVDMKVPQSGTLKLSHLQEGTYTFQLTVTDTAGQRSSDNVSVTVLRAAYSTGGCLHTCSRYHFFCDDGCCIDITLACDGVQQCPDGSDEDFCQNLGLDRKMVTHTAASPALPRTTGPSEDAGGDSLVEKSQKATAPNKPPALSNTEKRNHSAFWGPESQIIPVMPGAVLPLALGLAITALLLLMVACRLRLVKQKLKKARPITSEESDYLINGMYL

SEQ ID NO: 23 - protein, 236 aa, Homo sapiens
  misc_feature: Incyte ID No 7503509CD1
MASVAQESAGSQRRLPPRHGALRGLLLLCLWLPSGRAALPPAAPLSELHAQLSGVEQLLEEFRRQLQQERPQEELELELRAGGGPQEDCPGPGSGGYSAMPDAIIRTKDSLAAGASFLRAPAAVRGWRQCVAACCSEPRCSVAVVELPRRPAPPAAVLGCYLFNCTARGRNVCKFALHSGYSSYSLSRAPDGAALATARASPRQGASIRNPEAVPPTGGNLHLPADRDGHCRAEKL

SEQ ID NO: 24 - protein, 312 aa, Homo sapiens
  misc_feature: Incyte ID No 7505800CD1
MAATEGVGEAAQGGEPGQPAQPPPQPHPPPPQQQHKEEMAAEAGEAVASPMDDGFVSLDSPSYVLYRHFRRVLLKSLQKDLHEEMNYITAIIEEQPKNYQVWHHRRVLVEWLRDPSQELEFIADILNQDAKNYHAWQHRQWVIQEFKLWDNELQYVDQLLKEDVRNNSVWNQRYFVISNTTGYNDRAVLEREVQYTLEMIKLVPHNESAWNYLKGILQDRGLSKYPNLLNQLLDLQPSHSSPYLIAFLVDIYEDMLENQCDNKEDILNKALELCEILAKEKDTIRKEYWRYIGRSLQSKHSTENDSPTNVQQ

SEQ ID NO: 25 - protein, 452 aa, Homo sapiens
  misc_feature: Incyte ID No 7503141CD1
MAAATGPSFWLGNETLKVPLALFALNRQRLCERLRKNPAVQAGSIVVLQGGEETQRYCTDTGVLFRQESFFHWAFGVTEPGCYGVIDVDTGKSTLFVPRLPASHATWMGKIHSKEHFKEKYAVDDVQYVDEIASVLTSQKPSVLLTLRGVNTDSGSVCREASFDGISKFEVNNTILHPEIVECLFEHYCYSRGGMRHSSYTCICGSGENSAVLHYGHAGAPNDRTIQNGDMCLFDMGGEYYCFASDITCSFPANGKFTADQKAVYEAVLRSSRAVMGAMKPGVWWPDMHRLADRIHLEELAHMGILSGSVDAMVQAHLGAVFMPHGLGHFLGIDVHDVGGYPEGVERIDEPGLRSLRTARHLQPGMVLTVEPGIYFIDHLLDEALADPARASFLNREVLQRFRGFGGVRIEEDVVVTDSGIELLTCVPRTVEEIEACMAGCDKAFTPFSGPK

SEQ ID NO: 26 - protein, 471 aa, Homo sapiens
  misc_feature: Incyte ID No 7500362CD1
MAAATGPSFWLGNETLKVPLALFALNRQRLCERLRKNPAVQAGSIVSFFHWAFGVTEPGCYGVIDVDTGKSTLFVPRLPASHATWMGKIHSKEHFKEKYAVDDVQYVDEIASVLTSQKPSVLLTLRGVNTDSGSVCREASFDGISKFEVNNTILHPEIVECRVFKTDMELEVLRYTNKISSEAHREVMKAVKVGMKEYELESLFEHYCYSRGGMRHSSYTCICGSGENSAVLHYGHAGAPNDRTIQNGDMCLFDMGGEYYCFASDITCSFPANGKFTADQKAVYEAVLRSSRAVMGAMKPGVWWPDMHRLADRIHLEELAHMGILSGSVDAMVQAHLGAVFMPHGLGHFLGIDVHDVGGYPEGVERIDEPGLRSLRTARHLQPGMVLTVEPGIYFIDHLLDEALADPARASFLNREVLQRFRGFGGVRIEEDVVVTDSGIELLTCVPRTVEEIEACMAGCDKAFTPFSGPK

SEQ ID NO: 27 - protein, 458 aa, Homo sapiens
  misc_feature: Incyte ID No 7503328CD1
MAASRKPPRVRVNHQDFQLRNLRIIEPNEVTHSGDTGVETDGRMPPKVTSELLRQLRQAMRNSEYVTEPIQAYIIPSGDAHQSEYIAPCDCRRAFVSGFDGSAGTAIITEEHAAMWTDGRYFLQAAKQMDSNWTLMKMGLKDTPTQEDWLVSVLPEGSRVGVDPLIIPTDYWKKMAKVLRSAGHHLIPVKENLVDKIWTDRPERPCKPLLTLGLDYTGISWKDKVADLRLKMAERNVMWFVVTALDEIAWLFNLRGSDVEHNPVFFSYAIIGLETIMLFIDGDRIDAPSVKEHLLLDLGLEAEYRIQVHPYKSILSELKALCADLSPREKVWVSDKASYAVSETIPKDHRCCMPYTPICIAKAVKNSAESEGMRRAHIKDAVALCELFNWLEKEVPKGGVTEISAADKAEEFRRQQADFVDLSFPTISSQSLRRIGPCPWMRCTLLTRVLNTRMAPQM

SEQ ID NO: 28 - protein, 695 aa, Homo sapiens
  misc_feature: Incyte ID No 7510464CD1
MAASRKPPRVRVNHQDFQLRNLRIIEPNEVTHSGDTGVETDGRMPPKVTSELLRQLRQAMRNSEYVTEPIQAYIIPSGDAHQSEYIAPCDCRRAFVSGFDGSAGTAIITEEHAAMWTDGRYFLQAAKQMDSNWTLMKMGLKDTPTQEDWLVSVLPEGSRVGVDPLIIPTDYWKKMAKVLRSAGHHLIPVKENLVDKIWTDRPERPCKPLLTLGLDYTGISWKDKVADLRLKMAERNVMWFVVTALDEIAWLFNLRGSDVEHNPVFFSYAIIGLETIMLFIDGDRIDAPSVKEHLLLDLGLEAEYRIQVHPYKSILSELKALCADLSPREKVWVSDKASYAVSETIPKDHRCCMPYTPICIAKAVKNSAESEGMRRAHIKDAVALCELFNWLEKEVPKGGVTEISAADKAEEFRRQQADFVDLSFPTISSTGPNGAIIHYAPVPETNRTLSLDEVYLIDSGAQYKDGTTDVTRTMHFGTPTAYEKECFTYVLKGHIAVSAAVFPTGTKGHLLDSFARSALWDSGLDYLHGTGHGVGSFLNVHEGPCGISYKTFSDEPLEAGMIVTDEPGYYEDGAFGIRIENVVLVVPVKTKYNFNNRGSLTFEPLTLVPIQTKMIDVDSLTDKEELWNGILPARSLFCLFQFTVRLAQQLPPDLQGCDWEGIAETGPPGSSRVAHQRDATHLQTALINTSPVLFL

SEQ ID NO: 29 - protein, 140 aa, Homo sapiens
  misc_feature: Incyte ID No 7510394CD1
MAAAMPLALLVLLLLGPGGWCLAEPPRDSLREELVITPLPSGDVAATFQFRTRWDSELQREGVSHYRLFPKALGQLISKYSLRELHLSFTQGFWRTRYWGPPFLQAPSGAELWVWFQDTVTEFSSQLWTLKEGAEVAPGQ

SEQ ID NO: 30 - protein, 191 aa, Homo sapiens
  misc_feature: Incyte ID No 7500745CD1
MAAAMPLALLVLLLLGPGGWCLAEPPRDSLREELVITPLPSGDVAATFQFRTRWDSELQREGVSHYRLFPKALGQLISKYSLRELHLSFTQGFWRTRYWGPPFLQAPSVWINLGRSSVMSSQGSSAPLSTSSTPPTQSLPLPPSNPWVWPMTLTTTFCAMLCCRGRWSAPKTSPPGRSSCPVVPRQASLCC

SEQ ID NO: 31 - protein, 145 aa, Homo sapiens
  misc_feature: Incyte ID No 7500929CD1
MSAWAAASLSRAAARCLLARGPGVRAAPPRDPRPSHPEPRGCGAAPGRTLHFTAAVPAGHNKWSKVRHIKGPKDVERSRIFSKLCLNIRLAVKARRPKDRTCDLEAKGISLVGPPCQLCCCLRAIWMSVPTPSRMQGRTTQLVRL

SEQ ID NO: 32 - DNA, 2129 bp, Homo sapiens
  misc_feature: Incyte ID No 8268274CB1
ggcggcggcggccggagccggaaggcggggaggggccggccgttgggcccgaggcggcggcggcggcggcggcggctggggagaagcgctctcgtcgcctgcccgaggccggagcggcggggcccgcgcctcctccccccagcgccgcggaggggggaggaggaagatggagacccacatctcatgcctgttcccggagctgctggccatgatcttcggctacctggacgtccgggacaaggggcgcgcggcgcaggtgtgcaccgcctggcgggacgccgcctaccacaagtcggtgtggcggggggtggaggccaagctgcacctgcgccgggccaacccgtcgctgttccccagcctgcaggcccggggcatccgccgggtgcagatcctgagcctccgccgcagcctcagctacgtgatccagggcatggccaacatcgagagcctcaacctcagcggctgctacaacctcaccgacaacgggctgggccacgcgtttgtgcaggagatcggctccctgcgcgctctcaacctgagcctctgcaagcagatcactgacagcagcctgggccgcatagcccagtacctcaagggcctggaggtgctggagctgggaggttgcagcaacatcaccaacactggccttctgctcatcgcctggggtctgcagcgcctcaagagccttaacctccgcagctgccgccacctttcggatgtgggcatcgggcacctggccggcatgacgcgcagcgcggcggagggctgcctgggcctggagcagctcacgctacaggactgccagaagctcacagatctttctctaaagcacatctcccgagggctgacgggcctgaggctcctcaacctcagcttctgtgggggaatctcggacgctggcctcctgcacctgtcgcacatgggcagcctgcgcagcctcaacctgcgctcctgtgacaacatcagtgacacgggcatcatgcatctggccatgggcagcctgcgcctctcggggctggatgtttcgttctgtgacaaggtgggagaccagagtctggcttacatagcccaggggctggatggcctcaagtctctctccctctgctcctgccacatcagtgatgatggcatcaaccgcatggtgcggcagatgcacgggctgcgcacgctcaacattggacagtgtgtgcgcatcacggacaagggcctggagctgatcgctgagcacctgagccaactcaccggcatagacctgtacggctgcacccgaatcaccaagcgcggcctggagcgcatcacgcagctgccgtgcctcaaggaggcacgaggggatttttctccattattcactgtgagaactcggggaagctccagaaggtgagggagaggggacaacgacatggttcccgtggatctttaacttccagacttgcccgctctgcgcctctggcactctggtgatgacagctcaggtttccctgcctgtcactgctcgggcagaggctgctgcccagggcttctgctccggtaccttgtgaagctgcattctcctgccggtttctccagttctggggacagtggtttgctctgagacctcgcttcctttatggatccaaggagacttgctttttcagtctgttcagctttttacttgctaggatggaattgcaatttgcaagcttcttcgacaggaaactacaagttccacactttaattttatacatataaatatatacatgtgtacatatatctatgtacaggggtattatatatatacatataagatgatgatatatataatgatgatatgtattactgagaacgtaaaatatcattacatagtgatagctggacacacaaggaattcacaactccccaaagaaaatacatctggatgacctgcctagcagtttccccatgagatagaggaatgtctacgtatttcattccctgttcctgccctgaaacaatttcaatcactgacaaatcattatcattcattaataatgtttactgagtgcccatatgtgaaagaaatccactctacattccacagatgcatttcctctccccacggggtttccattttaatgggaacaatgtagaatatatctgtcttcccttaaaa

SEQ ID NO: 33 - DNA, 3489 bp, Homo sapiens
  misc_feature: Incyte ID No 7500515CB1
ggcagacactggagccacgatgaagcccccaaggcctgtccgtacctgcagcaaagttctcgtcctgctttcactgctggccatccaccagactactactgccgaaaagaatggcatcgacatctacagcctcaccgtggactccagggtctcatcccgatttgcccacacggtcgtcaccagccgagtggtcaatagggccaatactgtgcaggaggccaccttccagatggagctgcccaagaaagccttcatcaccaacttctccatgatcatcgatggcatgacctacccagggatcatcaaggagaaggctgaagcccaggcacagtacagcgcagcagtggccaagggaaagagcgctggcctcgtcaaggccaccgggagaaacatggagcagttccaggtgtcggtcagtgtggctcccaatgccaagatcacctttgagctggtctatgaggagctgctcaagcggcgtttgggggtgtacgagctgctgctgaaagtgcggccccagcagctggtcaagcacctgcagatggacattcacatcttcgagccccagggcatcagctttctggagacagagagcaccttcatgaccaaccagctggtagacgccctcaccacctggcagaataagaccaaggctcacatccggttcaagccaacactttcccagcagcaaaagtccccagagcagcaagaaacagtcctggacggcaacctcattatccgctatgatgtggaccgggccatctccgggggctccattcagatcgagaacggctactttgtacactactttgcccccgagggcctaaccacaatgcccaagaatgtggtctttgtcattgacaagagcggctccatgagtggcaggaaaatccagcagacccgggaagccctaatcaagatcctggatgacctcagccccagagaccagttcaacctcatcgtcttcagtacagaagcaactcagtggaggccatcactggtgccagcctcagccgagaacgtgaacaaggccaggagctttgctgcgggcatccaggccctgggagggaccaacatcaatgatgcaatgctgatggctgtgcagttgctggacagcagcaaccaggaggagcggctgcccgaagggagtgtctcactcatcatcctgctcaccgatggcgaccccactgtgggggagactaaccccaggagcatccagaataacgtgcgggaagctgtaagtggccggtacagcctcttctgcctgggcttcggtttcgacgtcagctatgccttcctggagaagctggcactggacaatggcggcctggcccggcgcatccatgaggactcagactctgccctgcagctccaggacttctaccaggaagtggccaacccactgctgacagcagtgaccttcgagtacccaagcaatgccgtggaggaggtcactcagaacaacttccggctcctcttcaagggctcagagatggtggtggctgggaagctccaggaccgggggcctgatgtgctcacagccacagtcagtgggaagctgcctacacagaacatcactttccaaacggagtccagtgtggcagagcaggaggcggagttccagagccccaagtatatcttccacaacttcatggagaggctctgggcatacctgactatccagcagctgctggagcaaactgtctccgcatccgatgctgatcagcaggccctccggaaccaagcgctgaatttatcacttgcctacagctttgtcacgcctctcacatctatggtagtcaccaaacccgatgaccaagagcagtctcaagttgctgagaagcccatggaaggcgaaagtagaaacaggaatgtccactcagctggagctgctggctcccggatgaatttcagacctggggttctcagctccaggcaacttggactcccaggacctcctgatgttcctgaccatgctgcttaccaccccttccgccgtctggccatcttgcctgcttcagcaccaccagccacctcaaatcctgatccagctgtgtctcgtgtcatgaatatgaaaatcgaagaaacaaccatgacaacccaaaccccagcccccatacaggctccctctgccatcctgccactgcctgggcagagtgtggagcggctctgtgtggaccccagacaccgccaggggccagtgaacctgctctcagaccctgagcaaggggttgaggtgactggccagtatgagagggagaaggctgggttctcatggatcgaagtgaccttcaagaaccccctggtatgggttcacgcatcccctgaacacgtggtggtgactcggaaccgaagaagctctgcgtacaagtggaaggagacgctattctcagtgatgcccggcctgaagatgaccatggacaagacgggtctcctgctgctcagtgacccagacaaagtgaccatcggcctgttgttctgggatggccgtggggaggggctccggctccttctgcgtgacactgaccgcttctccagccacgttggagggacccttggccagttttaccaggaggtgctctggggatctccagcagcatcagatgacggcagacgcacgctgagggttcagggcaatgaccactctgccaccagagagcgcaggctggattaccaggaggggcccccgggagtggagatttcctgctggtctgtggagctgtagttctgatggaaggagctgtgcccaccctgtacacttggcttccccctgcaactgcagggccgcttctggggcctggaccaccatggggaggaagagtcccactcattacaaataaagaaaggtggtgtgagcctgggaaaaaaaaaaaaaaaaaaaaaaaaaaaaaaaaaaaaaaagggggggcccccaaaataaggacccccaaccccgggggatataaatactcggggacaagcgcttaccactggcggaggcgtgtttaatcccacaccccacatggggggggcaacgttatattcccgtattgtcacgaggggcatccccactaaatgaggggcggcgtaattaaactatctcggcaaaaggacccagtggaatgaccccgtgatttatatgtactgacgcagacaacgacacactagctcaacaacacgacagccacatcagtacctcgtcgacatgctgacgaagagtcggaccccacatacacacaactaaaacaaccaaactctacacaacaaactacacacatctaatctccgactcagcaccccaacccacacccataacacacacacacagaacaaccaacaatatcatactatcattaactataaaacgacaaaccctcataacacttatataatgcagtacatcctaatcacaccacaacaacaaaaaaacaaccatcatacatcatccactaacactacattacaaaaccatcaaaaaacgccacacacccaccacactctccactattctct

SEQ ID NO: 34 - DNA, 2996 bp, Homo sapiens
  misc_feature: Incyte ID No 2256826CB1
ctgtttgcgcgcggacggaggagcggtggactcggggcagcggaggggcccccgcgcaccgtgcggtcctgctctcctctccctcgctggtccgcgagcacgcgcgcccttgcatccgccccccagacccccttttcccccccccccggcgcttccgtgtctcgcctcctcccggtgggctcgcctgcctcggaggcgcagggggtcgtggcgccgccgcgcaccggctgctcccgggagcgccgtcagggcgggccccgtgtgggggaggggtggttttggaccttttccgtaggggtcctccctttcggcccctccccctaccgggcgctccgaggccctggcggctctgtccaatgnagcagtggcngttgctggcaggtggggagtgtttttgttttgggttgaagttgaggctgaggagagagccgagctagcgacgagcagtcgttgcggccgccggcgccgcgggaggtggtggaggcctagccggagccgagaggtctcttgttcccgtcccacggtcccggcgtcacccctccggcgcccagtccccgtcccggaactcccgggcctgtcctgggcccccggtctgtgcactccgctcgccgcagcgcccggcccgggccgcacccgccggccccatgaggagggacgtgaacggagtgaccaagagcaggtttgagatgttctcaaatagtgatgaagctgtaatcaataaaaaacttcccaaagaactcctgttacggatattttcttttctagatgttgttaccctgtgccgctgtgctcaggtctccagggcctggaatgttctggctctggatggcagtaactggcagcgaattgacctatttgatttccagagggatattgagggccgagtagtggagaatatttcaaaacgatgtgggggctttttacgaaagttaagtcttcgtggatgtcttggagtgggagacaatgcattaagaacctttgcacaaaactgcaggaacattgaagtactgaatctaaatgggtgtacaaagacaacagacgctacatgtactagccttagcaagttctgttccaaactcaggcaccttgacttggcttcctgtacatcaataacaaacatgtctctaaaagctctgagtgagggatgtccactgttggagcagttgaacatttcctggtgtgaccaagtaaccaaggatggcattcaagcactagtgaggggctgtgggggtctcaaggccttattcttaaaaggctgcacgcagctagaagatgaagctctcaagtacataggtgcacactgccctgaactggtgactttgaacttgcagacttgcttgcaaatcacagatgaaggtctcattactatatgcagagggtgccataagttacaatccctttgtgcctctggctgctccaacatcacagatgccatcctgaatgctctaggtcagaactgcccacggcttagaatattggaagtggcaagatgttctcaattaacagatgtgggctttaccactctagccaggaattgccatgaacttgaaaagatggacctggaagagtgtgttcagataacagatagcacattaatccaactttctatacactgtcctcgacttcaagtattgagtctgtctcactgtgagctgatcacagatgatggaattcgtcacctggggaatggggcctgcgcccatgaccagctggaggtgattgagctggacaactgcccactaatcacagatgcatccctggagcacttgaagagctgtcatagccttgagcggatagaactctatgactgccagcaaatcacacgggctggaatcaagagactcaggacccatttacccaatattaaagtccacgcctacttcgcacctgtcactccacccccatcagtagggggcagcagacagcgcttctgcagatgctgcatcatcctatgacaatggaggtggtcaaccttggcgaactgagtatttaatgacacttctagagctaccgtggagtctctccagtggaagcaaccccagtgttctgagcaagggttacaaagtgagggagggcagtgtccagatccccagagccacacatacatacacatacacacccttacccccatccactctagctttgtgaccatgggactgaagtttgtgatggcttttttatcaagtagattggtaaaatttaaccattcctgttgaggtgcccataagaaaatcataggccaagatagggaggggcattccagcaaaccccgtgttaatgctactgtggtttttaaatttttgtctaggggtttctttggggattttagaacagcatctgctgtcctccggggtcaagaaaagcatggaaagacaatatatgatgtacccagggaccagaaagaaaatttctttgcatcttagaaatggtagacattcattgtgactaaagagcttctatgcttccttgtttccatgccaacatgctgagcatgctcacaaagaaggctcgtccattcctcctgtgttttagtatttggcccagaggtttcctaaatggttgccttgaaatcactgtggtccaaatgtaattcttacacactcaaattatcactgtctgtagcacacttgtgcacctgtcttacattctctgttgctcccccccacactcttgctcagtctgtcacctgttcagtctgcttactcactcaattgttacccttttgctgttgtcgtgtttacagtttgcattttgaatgattagttgggattaccaaacattttttaaaaagatattatcaataaatatttttttaattctaaattttaaaaaaaaaaaaaaaggggggggccgcttaaaaggtcccaagtttgattacgcttgcttccgacgtcatagcgggcggcagaattccgatatcaagcttttggatccggggacttcgggggggcccgttcccaatgcgcctatgtgattgattacgccccacaggcgct

SEQ ID NO: 35 - DNA, 1860 bp, Homo sapiens
  misc_feature: Incyte ID No 7686186CB1
gcctccggactgaccttgcggctgtatggcgcacgccccctgtacccggatgaagcgcccgatctttgggctgtgttcgcgcgaactggcggcgcgggccgggctgcctgccgtgcccggtaccgcactacgtgcccagcggggtcgtcaacgccttcgccaccggttcgaagcatcacgcggccatcgcgctgaccgacggcctgctgcgtagcctcacgccccgcgagttgaccggcgtgctcggccacgaaatcgcgcatattgcgaacgaggatttgcgtgtcatgggcctggccgattccatcagccggctgacccatctgctggccctgctggggcagcttgcgatcgtgctcagcttgccagcgctgctgcttggagtcgcggaagtcaattggcccgcgttgcttctactggcggtcgcgccacagctggccttgctggctcagttgggcttgtccagggtgcgcgaattcgacgccgaccggctcgctgccgaattgaccggcgacccgcacgggctggcctcggcgctcgccaagatcgagcgggtgagccgctcctggcgcgcctggctgctgccccggatgaggcaatccggaaccctcctggttgcgcacgcatccggcgacggctgaacgcattgagcgcttgctggaacttgctccgccgcccgcgatgccgccgtttccatcggcccgtttcgtccccgaggtgaccgtatcaccacgtccgccacgctggcgcaccggcggcctttgacgctgatttcaacatggagaatgacgtgacctaccccgacccctacagccgcccggcgccggaccgcttcatccggcgctggctcgtcatcactggctgcatcgccgcactcatgctgctgtggcagttcctgcccgccatcgaagcctggttcagtccccacgaaacgcaggagcgcacggtgacgccgcgcggcgacctggccgccgacgaaaaaaccaccatcgagctgttcgagaaatcgcgcgggtcggtggtttacatcaccacggcacaactagtgcgtgacgtctggtcgcgcaatgtcttttccgtgccgcgcggcaccggctccggcttcatctgggacgatgccggccacgtggtgaccaacttccacgtgatccagggggcatcgtctgccacggtcaaactggccgacggtcgcgattatcaggctgcgctcgttggcgccagtcctgcgcacgacatcgcggtactcaagattggcgtcggcttcaagcgcccgccggcggtgccggtgggcaccagtgccgatctcaaggtggggcaaaaggtctttgccattggcaatcccttcgggctcgactggacgctcaccaccggcatcgtctcggcgcttgaccgcaccctttccggcgacgccagtggcccggccattgaccacctgatccagaccgacgccgctatcaaccccggcaattccggtggcccgctgctcgattcggctgggcggctgatcggcatcaataccgccatctacagtccgtctggcgcctcggccggcatcggctttgcggtgccggtcgataccgtcatgcgcgtggtgccgcaactcataaagaccggcaagtacatccgtccggcgctgggcatcgaggtggatgagcagctcaacgcgcgtctgcaggcgctgaccggcagtaagggcgtattcgtattgcgcgtgacgccgggctcggcggcgcacagggccgggctcgtcggcgtcgaggtcaccgcaggcggcatcgtgcccggcgatcgcgttatcagcatcgacggtatcgccgtcgacccgggaattccggaccgtacctgctgacctcttcagac

SEQ ID NO: 36 - DNA, 1334 bp, Homo sapiens
  misc_feature: Incyte ID No 72617436CB1
cctgatgatctgtcactgtctcccatcactcccagatgggaccgtctagtctcaggaaaacaagctctgggctcccactgattctacattatggtgtgatcctaggagcgcccctggcctccagctgcgcaggagcctgtggtaccagcttcccagatggcctcacccctgagggaacccaggcctccggggacaaggacattcctgcaattaaccaagggctcatcctggaagaaaccccagagagcagcttcctcatcgagggggacatcatccggccgagtcccttccgactgctgtcagcaaccagcaacaaatggcccatgggtggtagtggtgtcgtggaggtccccttcctgctctccagcaagtacgatgagcccagccgccaggtcatcctggaggctcttgcggagtttgaacgttccacgtgcatcaggtttgtcacctatcaggaccagagagacttcatttccatcatccccatgtatgggtgcttctcgagtgtggggcgcagtggagggatgcaggtggtctccctggcgcccacgtgtctccagaagggccggggcattgtccttcatgagctcatgcatgtgctgggcttctggcacgagcacacgcgggccgaccgggaccgctatatccgtgtcaactggaacgagatcctgccaggctttgaaatcaacttcatcaagtctcggagcagcaacatgctgacgccctatgactactcctctgtgatgcactatgggaggcttgccttcagccggcgtgggctgcccaccatcacaccactttgggcccccagtgtccacatcggccagcgatggaacctgagtgcctcggacatcacccgggtcctccaactctacggctgcagcccaagtggccccaggccccgtgggagagggtcccatgcccacagcactggtaggagccccgctccggcctccctatctctgcagcggcttttggaggcactgtcggcggaatccaggagccccgaccccagtggttccagtgcgggaggccagcccgttcctgcagggcctggggagagcccacatgggtgggagtcccctgccctgaaaaagctcagtgcagaggcctcggcaaggcagcctcagaccctagcttcctccccaagatcaaggcctggagcaggtgcccccggtgttgctcaggagcagtcctggctggccggagtgtccaccaagcccacagtcccatcttcagaagcaggaatccagccagtccctgtccagggaagcccagctctgccagggggctgtgtacctagaaatcatttcaaggggatgtccgaagattaa

SEQ ID NO: 37 - DNA, 2070 bp, Homo sapiens
  misc_feature: Incyte ID No 7501945CB1
gctggaacagtgggtcctcgtcgtgactcagagccaggcccagtgctgccccgatcagaccccgcttccttttcggaatgtggaaggatgcgagtgtgtgcctgagggtgtggtcctgtgggggtgggtggtcttggcccgtgcacacctgcatgttgcgcatggtgttcctgtgggagcgtgtgtgcacgcccacacaagaggcacttcccaggccgtgtgggtctctacttcctcacagcaggcctgcatggcctccctactcttctcccacatcccagggctggggctcacagcgtcccctgttccagagctccttcatgtcccccaaaggcatgtggccctgtgtctggtgcagaggacctgtcaccagggatgggattcattgctctgcacacccacccaactagcacatcttggccgagcttcaattacgggcccagcataggactaaggttcagtggagacctgagaccagtgccccgcccacgccagaagccaaggggtacctgccgacgctgccgggagggggtgctgtgatctgcctgtgagcaggggtgctgaggcctttggaaatggttgtgcgggagccagtcctgcttcaggctcactgggacactcggccatttggagtgtcatccagccaagggttgtctctgcacagtgctacctctaaggatctgtccgtggtctgttaggaactgggctgcacagccggaggtgagcagcttcatcagtattacagccgcaccccattgctagcattactgcctgagctccgcctcctatcagatcagcggtggcattagattctcataggagcttgaaccctgttgtgaactgcattggagggatataggatgtatgctccttatgaaactctaactaatgcctgatgatctgtcactgtctcccatcactcccagatgggaccgtctagtctcaggaaaacaagctctgggctcccactgattctacattatggtgtgatcctaggagcgcccctggcctccagctgcgcaggagcctgtggtaccagcttcccagatggcctcacccctgagggaacccaggcctccggggacaaggacattcctgcaattaaccaagggctcatcctggaagaaaccccagagagcagcttcctcatcgagggggacatcatccggccgagtcccttccgactgctgtcagcaaccagcaacaaatggcccatgggtggtagtggtgtcgtggaggtccccttcctgctctccagcaagtacgatgagcccagccgccaggtcatcctggaggctcttgcggagtttgaacgttccacgtgcatcaggtttgtcacctatcaggaccagagagacttcatttccatcatccccatgtatgggtgcttctcgagtgtggggcgcagtggagggatgcaggtggtctccctggcgcccacgtgtctccagaagggccggggcattgtccttcatgagctcatgcatgtgctgggcttctggcacgagcacacgcgggccgaccgggaccgctatatccatgtcaactggaacgagatcctgccaggctttgaaatcaacttcatcaagtctcggagcagcaacatgctgacgccctatgactactcctctgtgatgcactatgggagggtcccatgcccacagcactggtaggagccccgctccggcctccctatctctgcagcggcttttggaggcactgtcggcggaatccaggagccccgaccccagtggttccagtgcgggaggccagcccgttcctgcagggcctggggagagcccacatgggtgggagtcccctgccctgaaaaagctcagtgcagaggcctcggcaaggcagcctcagaccctggcttcctccccaagatcaaggcctggagcaggtgcccccggtgttgctcaggagcagtcctggctggccggagtgtccaccaagcccacagtcccatcttcagaagcaggaatccagccagtccctgtccagggaagcccagctctgccagggggctgtgtacctagaaatcatttcaaggggatgtccgaagattaa

SEQ ID NO: 38 - DNA, 2265 bp, Homo sapiens
  misc_feature: Incyte ID No 7500264CB1
gggttcgggggcgccgcgctgtgaggccggggcctagagccagccgcggccgcgcaggaggggcccagggcccgcgctcgcccgcgtccccgccttcctcccgcgctcagccccgcctcggctcgctgcccttggctctcgtcgccatggcctccgtcgcccaggagagcgcgggctcgcagcgccggctaccgccgcgtcacggggcgctgcgcgggctgctactgctctgcctgtggctgccaagcggccgtgcggccttgccgcccgcggcgccgctgtccgaactgcacgcgcagctgtcgggcgtggagcagctgctggaggagttccgccggcaactgcagcaggagcggcctcaggaggagctggagctggagctgcgcgcgggcggcggcccccaggaggactgcccgggcccgggcagcggcggctacagcgcaatgcctgacgccatcatccgcaccaaggactccctggcggcgggtgccagcttcctgcgggcgccggcggccgtgcggggctggcggcaatgcgtggcggcctgctgctccgagccgcgctgctccgtggccgtggtggagctgccccggcgccccgcgcccccggcagccgtgctcggctgctacctcttcaactgcacggcgcgcggccgcaacgtctgcaagttcgcgctgcacagcggctacagcagctacagcctcagccgcgcgccggacggcgccgccctggccaccgcgcgcgcctcgccccggcaggaaaaggatgcgcctccacttagcaaggctgggcaggatgtggttctgcatctgcccacagacggggtggttctagacggccgcgagagcacagatgaccacgccatcgtccagtatgagtgggcactgctgcagggggacccgtcagtggacatgaaggtgcctcaatcagggggtgactccttggtggaaaagtctcagaaagccactgccccaaacaagccacctgcattatcaaacacagagaagaggaatcattccgccttttggggaccagagagtcaaatcattcctgtgatgccagatagtagttcctcagggaagaacagaaaagaggaaagttatatatttgagtcaaagggtgatggaggaggaggggaacacccagccccagaaacaggtgcagtgctacccctggcgctgggtttggctatcactgctctgctgcttctcatggttgcatgccgactacgactggtgaaacagaaactgaaaaaagctcgtcccattacatctgaggaatcggactacctcataaatgggatgtatctatagtaatgtaatttcaataccttggggcagggacatgttttgtttataatttatacatctattaagttctggatatttacagcttcttttgtttttaattgggccagaagattctgcaaatcccaaatctttctttattatttattgtaaaaaaagtttccttagaagtcataaaatattttgaaatttagagaggaattcatgattaaagattcctaaaaatataattctgatttatgtaagctgtccctgaaaatagaaatgtgtacttagctgagagaaaattcagcatctcaggaggtggtattaggatgactgtgttaacccattaccttttagaagccaactgttggccccttaccatgctggactgctataggcccagcttccccttgttctgtggcccttttcttcctccttgaagctcccagtattctttttcttttcccctctaaacctgtttctgagagtggatctcaagcaagttcatgccttcaatcagatgttacttagggtgggtatacctaaattataaaccttatgtacaagtcagtaagccttagggaaggtgagtgtgggtccttcctaatccctctgacgtcatgtcatataggtggctgcctccttagactgacctttgggagaaaaaaaccccagactttgaattagtaacagctctaagatggtcatgcagtgagataggaaatcaagatggaagcagagaatctggcatgccaaaaactaacagaaacttagttgaaggcaaagagagcaaggagaacgtttaatacttcattacatcaaatcaacactgctccatggtgagagcacagcaactcatttatatatatatatataggctttgttgatgaaaaacgacaattgaagagaggacgttgagtggattcctgggtacagcttttgtaaaaatgtcaccatggctttcatccaatgg

SEQ ID NO: 39 - DNA, 1834 bp, Homo sapiens
  misc_feature: Incyte ID No 7499935CB1
gtgacgccggtgccgggcgaacatggcggcggccaccggaccctcgttttggctggggaatgaaaccctgaaggtgccgctggcgctctttgccttgaaccggcagcgcctgtgtgagcggctgcggaagaaccctgctgtgcaggccggctccatcgtggtcctgcagggcggggaggagactcagcgctactgcaccgacaccggggtcctcttccgccaggagtccttctttcactgggcgttcggtgtcactgagccaggctgctatggtgtcatcgatgttgacactgggaagtcgaccctgtttgtgcccaggcttcctgccagccatgccacctggatgggaaagatccattccaaggagcacttcaaggagaagtatgccgtggacgacgtccagtacgtagatgagattgccagcgtcctgacgtcacagaagccctctgtcctcctcactttgcgtggcgtcaacacggacagcggcagtgtctgcagggaggcctcctttgacggcatcagcaagttcgaagtcaacaataccattcttcacccagagatcgttgagtgccgagtgtttaagacggatatggagctggaggttctgcgctataccaataaaatctccagcgaggcccaccgtgaggtaatgaaggctgtaaaagtgggaatgaaagaatatgagttggaaagcctcttcgagcactactgctactcccggggcggcatgcgccacagctcctacacctgcatctgcggcagtggtgagaactcagccgtgctacactacggacacgccggagctcccaacgaccgaacgatccagaatggggatatgtgcctgttcgacatgggcggtgagtattactgcttcgcttccgacatcacctgctcctttcccgccaacggcaagttcactgcagaccagaaggccgtctatgaggcagtgctgcggagctcccgtgccgtcatgggtgccatgaagccaggtgtctggtggcctgacatgcaccgcctggctgaccgcatccacctggaggagctggcccacatgggcatcctgagcggcagcgtggacgccatggtccaggctcacctgggggccgtgtttatgcctcacgggcttggccacttcctgggcattgacgtgcacgacgtgggaggctacccagagggcgtggagcgcatctacttcatcgaccacctcctggatgaggccctggcggacccggcccgcgcctccttccttaaccgcgaggtcctgcagcgctttcgcggttttggcggggtccgcatcgaggaggacgtcgtggtgactgacagcggcatagagctgctgacctgcgtgccccgcactgtggaagagattgaagcatgcatggcaggctgtgacaaggcctttacccccttctctggccccaagtagagccagccagaaatcccagcgcacctgggggcctggccttgcaacctcttttcgtgatgggcagcctgctggtcagcactccagtagcgagagacggcacccagaatcagatcccagcttcggcatttgatcagaccaaacagtgctgtttcccggggaggaaacacttttttaattacccttttgcaggctcccacctttaatctgttttataccttgcttattaaatgagcgacttaaaatgattgaaaataatgctgttctttagtagcaactaaaatgtgtcttgctgtcatttatattccttttcccaggaaagaagcatttctgatactttctgtcaaaaatcaatatgcagaatggcatttgcaataaaaggtttcctaaaaaaaaaaaaaaaaaaaaaaa

SEQ ID NO: 40 - DNA, 1524 bp, Homo sapiens
  misc_feature: Incyte ID No 7982285CB1
gggacatataaaaatgccattgtaactactgtagagtaaagtgttagctgcgctgccggaggaaacggaagaaggagcaagctatggaggggaacagggatgaggctgagaaatgtgtcgagatcgcccgggaggccctgaacgccggcaaccgcgagaaggcccagcgcttcctgcagaaggccgagaagctctacccactgccctcggcccgcgcactattggaaataattatgaaaaatggaagcacggctggaaatagccctcattgccgaaaaccatcaggtagtggcgatcaaagcaagcctaattgcacaaaggacagcacatctggtagtggtgaaggtggaaaaggctataccaaagaccaagtagatggagttctcagcataaacaaatgtaaaaattactatgaagtacttggagttacgaaagatgctggtgatgaagatttgaaaaaagcttatagaaagcttgctttgaagtttcatccagacaaaaaccatgcacctggagcaacagatgcttttaaaaagattggaaatgcttatgctgttttaagtaatccagaaaagcgaaaacagtatgacctcacgggcaatgaagaacaagcatgtaaccaccaaaacaatggcagatttaatttccatagaggttgtgaagctgatataactccagaagacttgtttaatatattttttgggggtggatttccttcaggtagtgtacattctttttcaaatggaagagctggttatagccaacaacatcagcatcgacatagtggacatgaaagagaagaggaaagaggagatggaggtttttctgtgtttatccagctgatgcccataattgtattgatcctcgtgtcattattaagccagttgatggtctctaatcctccttattccttatatcccagatctggaactgggcaaactattaaaatgcaaacagaaaacttgggtgttgtttattatgtcaacaaggacttcaaaaatgaatataaaggaatgttattacaaaaggtagaaaagagtgtggaggaagattatgtgactaatattcgaaataactgctggaaagaaagacaacaaaaaacagatatgcagtatgcagcaaaagtataccgtgatgatcgactccgaaggaaggcagatgccttgagcatggacaactgtaaagaattagagcggcttaccagtctttataaaggaggatgaactggaatttttatttataccttttagcgtactctttattttttctgtaagtaagtttggtttcatcatgagggatgaaggaaaagatttgatactgaaaactaaactgaatagttggttcctgaaatcttggactgtttatgacctactggctcctttaaatagtaactgaaaactaaaatggaatattttagttaacgcttctacaagtattttcattttaaaagcttacatgattcctaaactaaagtgtcatgagaaaggattatcacacctgtagcaatttccagttttagtgatt

SEQ ID NO: 41 - DNA, 2973 bp, Homo sapiens
  misc_feature: Incyte ID No 7758505CB1
cgagccgggtggctcgaccaaggagtgggtgtgggtgggtctttcggggctcccggcgacccgcggtgccgacgcaacgccgacgtatggtgccaagcgaactttaaaaagctgcttcggacaaaccagagccaggatttccactgtcggggacccgggatcggaagggtctagcccgagggaaatgctggaagatcccatcggccagtgaccagcaactttccggcgagattttgacgcggagaactgtgctctgcctcctcttattctcccaaagctcacgttggcgtcctgccttgcgggggaactcggcgcgctctctgcctgagcagcgagtgaattgaaccccagcccgctccggcgcctccgggctgatgagtgtcgctctccgcccgtccatctctttttcccggaggtaaaggcccgcggtcccccaccttcagtgcgcccgggttccaagcgccggagccagcgttttggcggagccgcttcttggatgctgaaggctgggctcctccatcgtgggtgccgaggcggcgatgggtgtcctcaaagtgtggctcgggctggccctagcgttggcggaatttgcagtattgcctcatcattccgaaggtgcttgtgtctatcaggattccttgttggcggatgccacaatttggaagcccgattcatgccagagctgccgttgccatggtgatattgttatctgcaaacctgctgtttgcagaaaccctcaatgtgcctttgagaagggagaagtgcttcaaatagctgccaaccaatgctgtcctgagtgtgttttgaggactccaggatcttgccatcatgaaaagaaaatccatgagcatgggacagaatgggcctcttctccatgtagtgtgtgctcttgcaatcatggggaagtccgatgtaccccccaaccatgcccaccgctgtcatgtggacaccaggagctggcattcatccctgaaggaagctgctgcccagtttgtgtgggccttgggaaaccctgttcctatgaaggccatgtgtttcaggatggggaggactggcggctgagccggtgtgccaaatgtctgtgtagaaatggggttgcccagtgcttcacagctcagtgtcagcctctattttgtaaccaggatgagactgtagtccgagtccctggaaaatgttgcccgcagtgctctgcaagatcctgctctgcagctggccaagtatacgagcatggtgagcagtggagcgaaaatgcctgcaccacgtgtatatgtgaccggggtgaggtcaggtgtcacaagcaggcctgcctgcccctgagatgcggaaagggtcagagcagggctcggcgtcatgggcaatgctgtgaggaatgtgtgtctcctgccgggagctgctcctatgatggagttgtgcggtaccaggacgaaatgtggaagggctcggcctgtgagttctgcatgtgtgatcatggccaagtgacctgccagactggagagtgtgccaaagtggagtgtgcccgggatgaagaattaattcacttagatggaaagtgttgtcctgaatgcatttcaaggaatggttattgtgtttatgaagaaactggagaatttatgtcatcaaatgctagtgaagttaaacgtattccagagggagagaagtgggaagatggcccttgcaaggtgtgtgagtgccgaggggctcaggtaacttgctacgagccctcttgcccaccatgtccagtgggcacactggccttagaggtgaagggacagtgctgtccagactgcacatcagttcattgccatccagattgtttgacatgctctcagtctccagaccactgtgacctctgccaagatcctaccaagttactgcagaatggatggtgtgtgcacagctgtggactgggtttttaccaagctggcagtctctgtatagcctgccagccccagtgctccacgtgtaccagtgggctggagtgctcatcctgccagcctcccctgctgatgcggcacgggcagtgtgtgcctacctgtggggacggcttctaccaagatcgccattcctgtgcagtctgccatgagtcctgtgcaggttgctggggcccaacggagaagcactgcttggcctgcagagatcccctccacgtgctgagagatggcggctgtgagagcagctgtggaaaaggcttctacaacaggcagggcacctgtagcgcttgtgaccaatcctgtgacagttgtggccccagtagccccaggtgtcttacctgtactgagaagacagtgctgcatgatgggaaatgcatgtctgaatgccctggcgggtactatgctgatgccactggcaggtgcaaagtttgtcataactcatgtgccagctgctctgggcccacaccctctcactgtacagcctgcagcccccccaaggctctgcgtcaaggccactgtctgccccgctgtggagagggtttctactctgaccacggagtctgcaaagcctgtcactcctcctgcctggcttgtatgggtcccgcaccctctcactgtactgggtgtaagaagccagaggaaggactgcaagtggagcagctgtctggcgtgggcatcccctctggcgagtgtctagcccagtgtagagcccatttttacttggagagcactggcctatgtgaagggcaaaatctggacttctgtcagaatttagaagtgatttctgctgtttgccttggcatatcatctacagagaattgatgacatcctgaataaataatttgactcaatagccaggccatctatgagtggttgaggagatgaaagggaagtattatagtttcctttctgttcccacaagtagccttgctgttgggtgaatagtttgactctaaagctacgtgaaaaaaaaatcattagtttgtatttttcattgtaaacatatgttcattaaaaaaattttataatacacccactacct

SEQ ID NO: 42 - DNA, 2126 bp, Homo sapiens
  misc_feature: Incyte ID No 6885756CB1
gaggagtaatttgattcaggtgttctagaagtcatgatgtgggctgtgtctgttgaattcccagcgatgcaaggggacacaccctgtgactcattccttaattgagtgctgatatttgattggtttatcgcacacctgatgggtgggtggggtgttcgcggttggagggggtgagttatataagggctgatgcggccagagagctggtcatttgaagactctctcggaagagatagcgtcttgctgcaacctgcggtcccagcagaaaaaccttgtgatccttgttgcgggcgacatggaagacgactcactctatttgggaggtgactggcagttcaatcacttttcaaaactcacatcttctcggctagatgcagcttttgctgaaatccagcggacttctctctctgaaaagtcaccactctcatctgagacccgtttcgacctctgtgatgatttggctcctgtggcaagacagcttgctcccagggagaagcttcctctgagtagcaggagacctgctgcggtgggggctgggctccagaagataggaaataccttctatgtgaacgtttccctgcagtgcctgacatacacactgccgctttccaactacatgctgtcccgggaggactctcaaacgtgtcatcttcacaagtgctgcatgttctgtactatgcaagctcacatcacatgggccctctaccgtcctggccatgtcatccagccctcacaggtattggctgctggcttccatagaggtgagcaggaggatgcccatgaatttctcatgtttactgtggatgccatgaaaaaggcatgccttcccgggcacaagcagctagatcatcactccaaggacaccaccctcatccaccaaatatttggagcgtattggagatctcaaatcaagtatctccactgccacggcatttcagacacctttgacccttacctggacatcgccctggatatccaggcagctcagagtgtcaagcaagctttggaacagttggtgaagcccaaagaactcaatggagagaatgcctatcattgtggtctttgtctccagaaggcgcctgcctccaagacgttaactttacccacttctgccaaggtcctcattcttgtattgaagagattctccgatgtcacaggcaacaaacttgccaagaatgtgcaatatcctaagtgccgtgacatgcagccatacatgtctcagcagaacacaggacctcttgtctatgtcctctatgctgtgctggtccacgctgggtggagttgtcacaacggacattacttctcttatgtcaaagctcaagaaggccagtggtataaaatggatgatgccgaggtcactgcctctggcatcacctctgtcctgagtcaacaggcctatgtcctcttttacatccagaagagtgaatgggaaagacacagtgagagtgtgtcaagaggcagggaaccaagagcccttggtgctgaagacacagacaggccagcaacgcaaggagagctcaagagagaccacccttgcctccaggtacccgagttggacgagcacttggtggaaagagccactcaggaaagcaccttagaccactggaaattcccccaaaagcaaaacaaaacgaagcctgagttcaacgtcagaaaagttgaaggtaccctgcctcccaacgtacttgtgattcatcaatcaaaatacaagtgtggtatgaaaaaccatcatcctgaacagcaaagctccctgctaaacctctcttcgacgaaaccgacagatcaggagtccatgaacactggcacactcgcttctctgcaagggagcaccaggagatccaaagggaataacaaacacagcaagagatctctgcttgtgtgccagtgatcacagtggaagtaccgacccacactgaggggtgcacacacacacacacacacaaacacaaatacacccacaagcgcgcacggaaacacacacacacccacacaaacacgaacaccgtcaatcctacataaagtaatgaggagccccagtttctgtctctacaacagggacaattggatagtgatggctgcgtctcaggatgagcccacacatgggaaacatcaagttgggggttcag

SEQ ID NO: 43 - DNA, 1973 bp, Homo sapiens
  misc_feature: Incyte ID No 7500748CB1
ggaagtagccgcaggcatggcggcggctatgccgcttgctctgctcgtcctgttgctcctggggcccggcggctggtgccttgcagaacccccacgcgacagcctgcgggaggaacttgtcatcaccccgctgccttccggggacgtagccgccacattccagttccgcacgcgctgggattcggagcttcagcgggaaggagtgtcccattacaggctctttcccaaagccctggggcagctgatctccaagtattctctacgggagctgcacctgtcattcacacaaggcttttggaggacccgatactgggggccacccttcctgcaggccccatcaggtgcagagctgtgggtctggttccaagacactgtcactgatgtggataaatcttggaaggagctcagtaatgtcctctcagggatcttctgcgcctctctcaacttcatcgactccaccaacacagtcactcccactgcctccttcaaacccctgggtctggccaatgacactgaccactactttctgcgctatgctgtgctgccgcgggaggtggtctgcaccgaaaacctcaccccctggaagaagctcttgccctgtagttccaaggcaggcctctctgtgctgctgaaggcagatcgcttgttccacaccagctaccactcccaggcagtgcatatccgccctgtttgcagaaatgcacgctgtactagcatctcctgggagctgaggcagaccctgtcagttgtatttgatgccttcatcacggggcagggaaagaaagactggtccctcttccggatgttctcccgaaccctcacggagccctgccccctggcttcagagagccgagtctatgtggacatcaccacctacaaccaggacaacgagacattagaggtgcacccacccccgaccactacatatcaggacgtcatcctaggcactcggaagacctatgccatctatgacttgcttgacaccgccatgatcaacaactctcgaaacctcaacatccagctcaagtggaagagacccccagagaatggttacatccactaccagcctgcccaggaccggctgcaaccccacctcctggagatgctgattcagctgccggccaactcagtcaccaaggtttccatccagtttgagcgggcgctgctgaagtggaccgagtacacaccagatcctaaccatggcttctatgtcagcccatctgtcctcagcgcccttgtgcccagcatggtagcagccaagccagtggactgggaagagagtcccctcttcaacagcctgttcccagtctctgatggctctaactactttgtgcggctctacacggagccgctgctggtgaacctgccgacaccggacttcagcatgccctacaacgtgatctgcctcacgtgcactgtggtggccgtgtgctacggctccttctacaatctcctcacccgaaccttccacatcgaggagccccgcacaggtggcctggccaagcggctggccaaccttatccggcgcgcccgaggtgtccccccactctgattcttgccctttccagcagctgcagctgccgtttctctctggggaggggagcccaagggctgtttctgccacttgctctcctcagagttggcttttgaaccaaagtgccctggaccaggtcagggcctacagctgtgttgtccagtacaggagccacgagccaaatgtggcatttgaatttgaattaacttagaaattcatttcctcacctgtagtggccacctctatattgaggtgctcaataagcaaaagtggtcggtggctgctgtattggacagcacagaaaaagatttccatcaccacagaaaggtcggctggcagcactggccaaggtgatggggtgtgctacacagtgtatgtcactgtgtagtggatggagtttactgtttgtggaataaaaacggctgtttccgtggaaaaa

SEQ ID NO: 44 - DNA, 1884 bp, Homo sapiens
  misc_feature: Incyte ID No 7500749CB1
ggcatggcggcggctatgccgcttgctctgctcgtcctgttgctcctggggcccggcggctggtgccttgcagaacccccacgcgacagcctgcgggaggaacttgtcatcaccccgctgccttccggggacgtagccgccacattccagttccgcacgcgctgggattcggagcttcagcgggaaggagacactgaccactactttctgcgctatgctgtgctgccgcgggaggtggtctgcaccgaaaacctcaccccctggaagaagctcttgccctgtagttccaaggcaggcctctctgtgctgctgaaggcagatcgcttgttccacaccagctaccactcccaggcagtgcatatccgccctgtttgcagaaatgcacgctgtactagcatctcctgggagctgaggcagaccctgtcagttgtatttgatgccttcatcacggggcagggaaagaaagactggtccctcttccggatgttctcccgaaccctcacggagccctgccccctggcttcagagagccgagtctatgtggacatcaccacctacaaccaggacaacgagacattagaggtgcacccacccccgaccactacatatcaggacgtcatcctaggcactcggaagacctatgccatctatgacttgcttgacaccgccatgatcaacaactctcgaaacctcaacatccagctcaagtggaagagacccccagagaatgaggcccccccagtgcccttcctgcatgcccagcggtacgtgagtggctatgggctgcagaagggggagctgagcacactgctgtacaacacccacccataccgggccttcccggtgctgctgctggacaccgtaccctggtatctgcggctgtatgtgcacaccctcaccatcacctccaagggcaaggagaacaaaccaagttacatccactaccagcctgcccaggaccggctgcaaccccacctcctggagatgctgattcagctgccggccaactcagtcaccaaggtttccatccagtttgagcgggcgctgctgaagtggaccgagtacacaccagatcctaaccatggcttctatgtcagcccatctgtcctcagcgcccttgtgcccagcatggtagcagccaagccagtggactgggaagagagtcccctcttcaacagcctgttcccagtctctgatggctctaactactttgtgcggctctacacggagccgctgctggtgaacctgccgacaccggacttcagcatgccctacaacgtgatctgcctcacgtgcactgtggtggccgtgtgctacggctccttctacaatctcctcacccgaaccttccacatcgaggagccccgcacaggtggcctggccaagcggctggccaaccttatccggcgcgcccgaggtgtccccccactctgattcttgccctttccagcagctgcagctgccgtttctctctggggaggggagcccaagggctgtttctgccacttgctctcctcagagttggcttttgaaccaaagtgccctggaccaggtcagggcctacagctgtgttgtccagtacaggagccacgagccaaatgtggcatttgaatttgaattaacttagaaattcatttcctcacctgtagtggccacctctatattgaggtgctcaataagcaaaagtggtcggtggctgctgtattggacagcacagaaaaagatttccatcaccacagaaaggtcggctggcagcactggccaaggtgatggggtgtgctacacagtgtatgtcactgtgtagtggatggagtttactgtttgtggaataaaaacggctgtttccgtggaaaaaaaaaaaaaaaaaaaaaaaaaaaaaaaaag

SEQ ID NO: 45 - DNA, 1581 bp, Homo sapiens
  misc_feature: Incyte ID No 7503401CB1
cgccaccacctcagctgcggaccgaggcgagatggcggccaccgagggggtcggggaggctgcgcaagggggcgagcccgggcagccggcgcaacccccgccccagccgcacccaccgccgccccagcagcagcacaaggaagagatggcggccgaggctggggaagccgtggcgtcccccatggacgacgggtttgtgagcctggactcgccctcctatgtcctgtacagggacagagcagaatgggctgatatagatccggtgccgcagaatgatggccccaatcccgtggtccagatcatttatagtgacaaatttagagatgtttatgattacttccgagctgtcctgcagcgtgatgaaagaagtgaacgagcttttaagctaacccgggatgctattgagttaaatgcagccaattatacagtgtggcatcataggcgagtattagtggaatggctaagagatccatctcaggagcttgaatttattgctgatattcttaatcaggatgcaaagaattatcatgcctggcagcatcgacaatgggttattcaggaatttaaactttgggataatgagctgcagtatgtggaccaacttctgaaagaggatgtgagaaataactctgtctggaaccaaagatacttcgttatttctaacaccactggctacaatgatcgtgctgtattggagagagaagtccaatacactctggaaatgattaaactagtaccacataatgaaagtgcatggaactatttgaaagggattttgcaggatcgtggtctttccaaatatcctaatctgttaaatcaattacttgatttacaaccaagtcatagttccccctacctaattgcctttcttgtggatatctatgaagacatgctagaaaatcagtgtgacaataaggaagacattcttaataaagcattagagttatgtgaaatcctagctaaagaaaaggacactataagaaaggaatattggagatacattggaagatcccttcaaagcaaacacagcacagaaaatgactcaccaacaaatgtacagcaataacaccatccagaagaacttgatggaatgcttttattttttattaagggaccctgcaggagtttcacacgagagtggtccttccctttgcctgtggtgtaaaagtgcatcacacaggtattgctttttaacaagaactgatgctccttgggtgctgctgctactcagactagctctaagtaatgtgattcttctaaagcaaagtcattggatgggaggaggaagaaaaagtcccataaaggaacttttgtagtcttatcaacatataatctaatcccttagcatcagctcctccctcagtggtacatgcgtcaagatttgtagcagtaataactgcaggtcacttgtatgtaatggatgtgaggtagccgaagtttggttcagtaagcagggaatacagtcgttccatcagagctggtctgcacactcacattatcttgctatcactgtaaccaactaatgccaaaagaacggttttgtaataaaattatagctgtatctaaaaaaaaaaaaaaagg

SEQ ID NO: 46 - DNA, 1996 bp, Homo sapiens
  misc_feature: Incyte ID No 7503485CB1
gttcagcccccgtctacactggggtggtgcttagccggcgccagaccgaccctcgacttcggagaggcagcgcggttcctctgggtgcttccgcctccccttctcctgcttctccagcctcttcggcctcctcgcccgccgcgggaacccgagaccccagtgtatgccccacccctgaccccgctcgcgacatgtccaccccggctcggcggcgcctcatgcgggacttcaagaggttgcaggaggatcctccagccggagtcagcggggctccgtccgagaacaacataatggtgtggaacgcggtcattttcgggcctgaagggaccccgtttgaggatgtctatgcagatggtagtatatgtctggacatacttcagaaccgttggagtccaacctatgatgtgtcttccattctaacatccatacagtctctgttggatgaacccaatcccaatagtccagcaaacagccaggctgctcagctgtaccaggagaacaaacgggaatatgaaaagcgtgtttctgcaatagtagaacaaagctggcgtgattgttgaccccgggtacagtttaaagaagctggccataagaaaaatatatattgatgtgtttgtcacctccctactcctgtcattacatttactttattaaaagcaaaataactgttgtgctgtttccatcttccttgccaagttttcctaccccttctaccctctccttaaacatcagaaaacaccctctatgaaatcaaatgtactgtacctgggttacttgcaaaaattactaatgcttcagtttttctgttgtatttcatttccagttttcaggcagttatttttattattgtactttaagcttttaagatgaattgttatacaagaggtgcttatgcttagcttgatgaccaggatgttatttttaacaaaatgattgctgaagtgtttcatcctggctggtccttcacttgtgttggatttagaagtgaatgtgtttggaatatggcctacagagaatagaaacaaatccatgtaaacaattttgaaggaggcatgggagctaaaaatcctgtgatactaagatctcagtcatatgaattacaacgtagtatttactggcaagaaggagaaagttgaaggactcagctaaaggagtacagcaattgtagtaactgacacatcctctctttgcaagctgctgactgggcacactcatgccaagtttcagaattattggtcttctgggtttttgctttttaaaagaggtgtgggagcagaggaatggaaacaatcgtgagtttttgagctagggaaagttggagctcctttaatctttttaaaggatcagtgctgccctaagtgaataaactcaattgtccatctttattttagagttttaatgaattcaaggaagggagcatagcatatctgtggcaaactattttccactcaaatcctgagttattgctgcatgctttaatttcttccctttcagcatctgagaaccttaaagccaatgtctgcgatctttttttggatatttatacttttagatatatagtacctttaagtagcagtatgggacaaggcttgtaaatgttttgtctaatgttctattgtcaccttttatgcatttatcacttccaaatctaactttgcacaagtaacccatgtaaaaaaaaaatgtacatttttcaaaagttgtaaataaaaataaccttaaaatttcaaaaaaaaaaaaaaaaaaaaaaaaaaaaaaaaaaaaaaaaaaaaaaaaaaaaaaaaaaaaaaaaaaaaaaaaaaaaaaaattttttcttgtgggacaaaaaaaaaaaagcggcggtgtgtgtgtgttttaatagccaaacaaaattttttttcggtatattttggggggtggcccccccttttttagcaaaatgagaaaaactgtacacgatgtataaaccgcggaggaggaataaaaatatttttatcaaaaagaaaaaaattatca

SEQ ID NO: 47 - DNA, 1232 bp, Homo sapiens
  misc_feature: Incyte ID No 7504076CB1
ggcaagcgcaggtcaggcggtgcagctgggcggccagcggatcgtgccgcggcggccgagcgcagctacaggagggtgtccagaagccacaagccatggctgtggggaacatcaacgagctgcccgagaacatcctgctggagctgttcacgcacgtgcccgcccgccagctgctgctgaactgccgcctggtctgcagcctctggcgggacctcatcgacctcgtgaccctctggaaacgcaagtgcctgcgagagggcttcatcactgaggactgggaccagcccgtggccgactggaagatcttctacttcttacggagcctgcacaggaacctcctgcacaacccgtgcgctgaagaggggttcgagttctggagcctggatgtgaatggaggcgatgagtggaaggtggaggatctctctcgagaccagaggaaggaattccccaatgaccaggtcaagaaatacttcgttacttcatattacacctgcctcaagtcccaggtggtggacctcaaggccgaagggtattgggaggagctgatggataccacacggccggacatcgaggtcaaggactggttcgcagccaggccagattgcgggtccaagtaccagctgtgcgttcagctcctgtcgtccgcgcacgcgcctctggggaccttccagccagacccggcgaccatccagcagaagagcgatgccaagtggagggaggtctcccacacattctccaactacccgcccggcgtccgctacatctggtttcagcacggcggcgtggacactcattactgggccggctggtacggcccgagggtcaccaacagcagcatcaccatcgggcccccgctgccctgacaccccctgagcccccatctgctgaaccctgactgctttacggacattggatgaagccgaagcatttagaatggtgcctggcacacagttggtgcgtgatatggttaagctttgtgtccccacccacatctcatcttgaatgtgacggtttccccggctccctcctgccgccatgtgaagaaggtcgttgcttccccttcaccttccaccaccatgatttagagatggagtttcaccatgattggacacagggtggtctcaatctcctgaacctcgtgatccacccacctcgacctcccatagtgctgagattaacatggcgtggccaccgcgctctaccgcttgtgtcttgacgcgtccccagcctca

SEQ ID NO: 48 - DNA, 810 bp, Homo sapiens
  misc_feature: Incyte ID No 7500926CB1
ggggtccggaactgcttgttccggcagtggaagagacgcgccggcgttggccgctgctgctagcagcttgaaccccagggtcgggaccgatgtcggcttgggctgctgccagcctaagcagggccgctgcccgatgcttgctggcacgaggccccggggtcagggcggctcctccgcgcgacccccggccctcccaccccgagccccggggctgcggtgccgctccgggcaggacgctgcactttaccgcggctgtccccgccgggcacaacaagtggtccaaagtcaggcacatcaagggtccgaaggacgtcgaaaggagtcgcatcttctccaaactctgtttgaacatccgcctggcagtgaaagaaggaggccccaaccctgagcacaacagcaacctggccaatatcttagaggtgtgtcgcagcaaacatatgcccaagtcaacgattgagacagcactgaaaatggagaaatccaaggacacttatttgctgtatgagggtcgaggccctggtggctcttctctgctcatcgaggcattatctaacagtagccacaagtgccaagcagacttgcgaccttgaagccaaaggaatctcacttgtggggcctccttgtcagctctgctgctgtctcagagccatctggatgagtgtcccgacaccctctcggatgcagggcaggaccacccagctggtcagactctgatgttgggtagctggcctctgtggggattgtaagtgccctgaggcgctctgtactagaaactgctcttaatagtaacggtgattattggttgctgca

SEQ ID NO: 49 - DNA, 2625 bp, Homo sapiens
  misc_feature: Incyte ID No 7503216CB1
gcggcggcggcagcagcagcagcagcattagcagcagcagcttctctcaatgctgagtagtatatactcaagataaatgaaaacacatgtccacacaaaagcttgctcatgaattgtcatagaaacattcttcacaatatccaaaagtggaagcaacccaaatgtccatcaactgatgaatggataaacaaagtaatattctgtacgatggaattttaattggcatatttggtaataaaaaggaatcaagtactgatacatcctacaacatggatacatcttgaaaatgctatgctaaatgaaagagggtgaatcaccaggattttcaactgagaaatttaagaataattgaacctaacgaggtgacacactcaggagacacaggtgtggaaacagacggcagaatgcctccaaaggtgacttcagagctgcttcggcagctgagacaagccatgaggaactctgagtatgtgaccgaaccgatccaggcctacatcatcccatcgggagatgctcatcagagtgagtatattgctccatgtgactgtcggcgggcttttgtctctggattcgatggctctgcgggcacagccatcatcacagaagagcatgcagccatgtggactgacgggcgctactttctccaggctgccaagcaaatggacagcaactggacacttatgaagatgggtctgaaggacacaccaactcaggaagactggctggtgagtgtgcttcctgaaggatccagggttggtgtggaccccttgatcattcctacagattattggaagaaaatggccaaagttctgagaagtgccggccatcacctcattcctgtcaaggagaacctcgttgacaaaatctggacagaccgtcctgagcgcccttgcaagcctctcctcacactgggcctggattacacagggctatttaatctccgaggatcagatgtggagcacaatccagtatttttctcctacgcaatcataggactagagacgatcatgctcttcattgatggtgaccgcatagacgcccccagtgtgaaggagcacctgcttcttgacttgggtctggaagccgaatacaggatccaggtgcatccctacaagtccatcctgagcgagctcaaggccctgtgtgctgacctctccccaagggagaaggtgtgggtcagtgacaaggccagctatgctgtgagcgagaccatccccaaggaccaccgctgctgtatgccttacacccccatctgcatcgccaaagctgtgaagaattcagctgagtcagaaggcatgaggcgggctcacattaaagatgctgttgctctctgtgaactctttaactggctggagaaagaggttcccaaaggtggtgtgacagagatctcagctgctgacaaagctgaggagtttcgcaggcaacaggcagactttgtggacctgagcttcccaacaatttccagtacgggacccaacggcgccatcattcactacgcgccagtccctgagacgaataggaccttgtccctggatgaggtgtaccttattgactcgggtgctcaatacaaggatggcaccacagatgtgacgcggacaatgcattttgggacccctacagcctacgagaaggaatgcttcacatatgtcctcaagggccacatagctgtgagtgcagccgttttcccgactggaaccaaaggtcaccttcttgactcctttgcccgttcagctttatgggattcaggcctagattacttgcacgggactggacatggtgttgggtcttttttgaatgtccatgagggtccttgcggcatcagttacaaaacattctctgatgagcccttggaggcaggcatgattgtcactgatgagcccgggtactatgaagatggggcttttggaattcgcattgagaatgttgtccttgtggttcctgtgaagaccaagtataattttaataaccggggaagcctgacctttgaacctctaacattggttccaattcagaccaaaatgatagatgtggattctcttacagacaaagagtgcgactggctcaacaattaccacctgacctgcagggatgtgattgggaaggaattgcagaaacagggccgccaggaagctctcgagtggctcatcagagagacgcaacccatctccaaacagcattaataaatacctccccggttttgtttttgtaaaatgctctggaggaaggaagaaacgtggcagatccctgacatctttcccctttcctttccttcttccctacctcccctttttactttagactttaagaagaacagaaaatcttcttatcctctttgatattttattgcaaacactcagtcttttatgattttttaattgttgagaacaagccaagaataaaattgctgcaccagaaggagggtccctccaaagttgaacacttggtgaaaggaagatgccccgacttctttggccagtgatggggaatcagtgagtgctccatgatggtcatgttccaggtgctagtacatcattcatgatcaccttaatgctcatgagactatatttatgatcagtgaataaaaatgtcagaactgtgaaaaaaaaaaaaaaaaaaaaaaaaaag

SEQ ID NO: 50 - DNA, 2432 bp, Homo sapiens
  misc_feature: Incyte ID No 7503233CB1
gcgctcttcctggttgggccctgccctgagctgccaccgggaagccagcctcagggactgcagcgacccccaaacacccctcccccaggatgtcggaggagatcatcacgccggtgtactgcactggggtgtcagcccaagtgcagaagcagcgggccagggagctgggcctgggccgccatgagaatgccatcaagtacctgggccaggattatgagcagctgcgggtgcgatgcctgcagagtgggaccctcttccgtgatgaggccttccccccggtaccccagagcctgggttacaaggacctgggtcccaattcctccaagacctatggctatgccggcatcttccatttccagctgtggcaatttggggagtgggtggacgtggtcgtggatgacctgctgcccatcaaggacgggaagctagtgttcgtgcactctgccgaaggcaacgagttctggagcgccctgcttgagaaggcctatgccaaggtaaatggcagctacgaggccctgtcagggggcagcacctcagagggctttgaggacttcacaggcggggttaccgagtggtacgagttgcgcaaggctcccagtgacctctaccagatcatcctcaaggcgctggagcggggctccctgctgggctgctccatagacatctccagcgttctagacatggaggccatcactttcaagaagttggtgaagggccatgcctactctgtgaccggggccaagcaggtgaactaccgaggccaggtggtgagcctgatccggatgcggaacccctggggcgaggtggagtggacgggagcctggagcgacagctcctcagagtggaacaacgtggacccatatgaacgggaccagctccgggtcaagatggaggacggggagttctggatgtcattccgagacttcatgcgggagttcacccgcctggagatctgcaacctcacacccgacgccctcaagagccggaccatccgcaaatggaacaccacactctacgaaggcacctggcggcgggggagcaccgcggggggctgccgaaactacccagccaccttctgggtgaaccctcagttcaagatccggctggatgagacggatgacccggacgactacggggaccgcgagtcaggctgcagcttcgtgctcgcccttatgcagaagcaccgtcgccgcgagcgccgcttcggccgcgacatggagactattggcttcgcggtctacgaggtccctccggagctggtgggccagccggccgtacacttgaagcgtgacttcttcctggccaatgcgtctcgggcgcgctcagagcagttcatcaacctgcgagaggtcagcacccgcttccgcctgccacccggggagtatgtggtggtgccctccaccttcgagcccaacaaggagggcgacttcgtgctgcgcttcttctcagagaagagtgctgggactgtggagctggatgaccagatccaggccaatctccccgatgagcaagtgctctcagaagaggagattgacgagaacttcaaggccctcttcaggcagctggcaggggaggacatggagatcagcgtgaaggagttgcggacaatcctcaataggatcatcagcaaacacaaagacctgcggaccaagggcttcagcctagagtcgtgccgcagcatggtgaacctcatggatcgtgatggcaatgggaagctgggcctggtggagttcaacatcctgtggaaccgcatccggaattacctgtccatcttccggaagtttgacctggacaagtcgggcagcatgagtgcctacgagatgcggatggccattgagtcggcaggcttcaagctcaacaagaagctgtacgagctcatcatcacccgctactcggagcccgacctggcggtcgactttgacaatttcgtttgctgcctggtgcggctagagaccatgttccgatttttcaaaactctggacacagatctggatggagttgtgacctttgacttgtttaagtggttgcagctgaccatgtttgcatgaggcagggactcggtcccccttgccgtgctcccctccctcctcgtctgccaagcctcgcctcctaccacaccacaccaggccaccccagctgcaagtgccttccttggagcagagaggcagcctcgtcctcctgtcccctctcctcccagccaccatcgttcatctgctccgggcagaactgtgtggcccctgcctgtgccagccatgggctcgggatggactccctgggccccacccattgccaagccaggaaggcagctttcgcttgttcctgcctcgggacagccccgggtttccccagcatcctgatgtgtcccctctccccacttcagaggccacccactcagcaccaacgggcttggccttgcttgcagactataaact

SEQ ID NO: 51 - DNA, 3969 bp, Homo sapiens
  misc_feature: Incyte ID No 7726576CB1
ccgggcagggtcgcctctaggtgctcacctccgccacttcgccatggcgggtcctggcccgggcgcggtgctggagtccccccggcagctgctgggccgcgtgcgcttcttggcagaggcagcgcggagcctccgcgccgggcggccgctgccagcagcgctggctttcgtgccgcgagaggtgctctacaagctttacaaggacccagcgggaccgtcgcgcgtgcttctgccggtgtgggaggcagagggcctggggctgcgtgtgggcgccgcaggcccagcccccggtaccggctccgggcccctccgcgccgcccgcgacagcattgagctccggcgcggcgcctgcgtgcgcaccacgggcgaggagctgtgcaatggccacgggctctgggtgaagctgacaaaggagcagctggcagagcacctgggcgactgcgggctgcaggaaggctggctgctggtgtgccgcccggcggagggcggagcccgcctggtacccatcgacactcccaaccacctccagcggcagcagcagctctttggcgtggattatcggccggtgctcaggtgggaacaggtggtggacctgacatactcacatcgcctgggatcgagacctcagccggcagaggcatacgcagaagctgtacaaaggctactctatgtacccccgacatggacctacgagtgcgacgaggacctgatccacttcttgtatgaccacctgggcaaggaggatgagaacctgggtagcgtgaagcagtatgtggagagcatagacgtttcctcctacacggaggagttcaacgtgtcctgcctgacagacagcaatgccgatacctactgggagagcgatgggtcccagtgccaacactgggtacggcttactatgaagaagggcaccattgtcaagaagctgctactcacagtggataccacagatgacaactttatgccaaagcgggtggtggtctatgggggtgaaggggacaacctgaagaagctgagtgacgtgagcattgacgagaccctcatcggggatgtctgtgtcctggaggacatgaccgtccacctcccgatcatcgagatccgcatcgtggagtgccgagatgatgggattgatgttcgtctccgaggggtcaagatcaagtcatctagacagcgggaactagggttgaatgcagacctgttccagccaactagtctggtgcgatatccacgcctagaaggcaccgaccctgaagtactgtaccgcagagctgtcctcctgcagagactcatcaagatcctcgatagtgtcctgcaccacctggtacctgcctgggaccacacactgggcaccttcagtgagattaagcaagtgaagcagttcctactgctgtcccgccagcggccaggcctggtggctcagtgcctgcgtgactctgagagcagcaagcccagcttcatgccacgcctatacatcaaccgccgtcttgccatggaacaccgtgcctgcccctctcgagaccctgcctgcaagaatgcagtcttcacccaggtatatgaaggcctcaagccctctgacaaatatgaaaagcccctggactacaggtggcccatgcgctatgaccagtggtgggagtgtaaatttattgcagaaggcatcattgaccaagggggtggtttccgggacagcctggcagatatgtcagaagagctgtgccctagctcagcggatacccccgtgcccctgcccttctttgtacgcacagccaaccagggcaatggcactggtgaggctcgggacatgtatgtacccaacccctcctgccgagactttgccaagtatgaatggatcggacagctgatgggggctgcccttcggggtaaggagttcctggtcctggccctgcctggttttgtgtggaagcagctttctggtgaggaggtgagctggagcaaggacttcccagctgtggactctgtgctggtgaagctcctggaagtgatggaaggaatggacaaggagacgtttgagttcaagtttgggaaggaactaacattcaccactgtactgagtgaccaacaggtggtggagctgatccctgggggtgcaggcatcgtcgtgggatatggggaccgttctcgtttcatccaactggtccagaaggcacggctagaggagagcaaggagcaggtggcagctatgcaggcaggtctgctgaaggtggtaccacaggctgtgctggacttgctgacctggcaagagttggagaagaaagtgtgtggggatccagaggtcactgtggatgctctgcgcaagctcacccggtttgaggacttcgagccatctgactcgcgggtgcagtatttctgggaggcactgaacaacttcaccaacgaggaccggagccgcgtcctgcgctttgtcacgggccgcagtcgcctgccagcacggatctacatctacccagacaagctgggctacgagaccacagacgcgctgcccgagtcttccacttgctccagcaccctcttcctgccacactatgccagtgccaaggtatgcgaggagaagctccgctatgcggcctacaactgcgtggccatcgacactgacatgagcccttgggaggagtgaggcgtgccgccggctgtgggaccagcaagactgcacgtgtccctcttggccttgcccagggcgaagacaccttccctgccctggtttggctgacgtgctcagcaaaaccccatgtgccctgctcctgtgtgcagttggggtaggggcagctggcatggtcaggtaacactagtggcccagccccgcagacccacaagccctacccgtgctggggcttgcttcccgaggtatttcacctcttaagagggaatcttccacaagcccagcacaagctgccaggcctgagctacttgaagggggccatctaggtccccaacccatggactttgcctccattttcagctccgccttttttctcctattttctctctggctttcttcagccatgactcacaactaaaaacataaaacactggaggttagtggaggcccctccccaagcagggagcctgggatgggcagggagtgatagccaaactccttggtcacctgctccaagaaggaagcagtagctgagcacctgccctcacatactgctcttttcccctctccctccacaccagagatgtggtgagctctgttcttctaccaacccagtctcaacacacaaagtgccaccaccttccctgactcagaacccacatccactcaatgtgaactctactaccacgacctccccatattcctcacttctccatcacctccagcctgactccctgtctgccctttcacccccaagattttgcacaggttaaggccagttatggcctttttgaaatctgtaatagctcccctttccccaactctaaagcctagaccttaaacctgttcctagaactctggcccccaccattcctcagtgccacctttctgctgctgaaaggccacagtgatgccccccagtgtgaggcgggaggtgtgccctcttccccagccaagcctttttacccactccccaggtggcagctatgcaggcaggtctgctgaaggtggtaccacaggctgtgctggacttgctgacctggcaagagttggagaagaaagtgtgtggggatccagaggtcactgtggatgctctgcgcaagctcacccggtttgaggacttcgagccatctgactcgcgggtgcagtatttctgggaggcactgaacaacttcaccaacgaggaccggagccgcttcctgcgctttgtcacgggccgcagtcgcctgccagcacggatctacatctacccagacaagctgggctacgagaccacagacgcgctgcccgagtcttccacttgctccagcaccctctcttgccagcacactgcgccgtataagtgagcgagctcgtcc

SEQ ID NO: 52 - DNA, 2537 bp, Homo sapiens
  misc_feature: Incyte ID No 7503507CB1
gggttcgggggcgccgcgctgtgaggccggggcctagagccagccgcggccgcgcaggaggggcccagggcccgcgctcgcccgcgtccccgccttcctcccgcgctcagccccgcctcggctcgctgcccttggctctcgtcgccatggcctccgtcgcccaggagagcgcgggctcgcagcgccggctaccgccgcgtcacggggcgctgcgcgggctgctactgctctgcctgtggctgccaagcggccgtgcggccttgccgcccgcggcgccgctgtccgaactgcacgcgcagctgtcgggcgtggagcagctgctggaggagttccgccggcaactgcagcaggagcggcctcaggaggagctggagctggagctgcgcgcgggcggcggcccccaggaggactgcccgggccggggcagcggcggctacagcgcaatgcctgacgccatcatccgcaccaaggactccctggcggcgggtgccagcttcctgcgggcgccggcggccgtgcggggctggcggcaatgcgtggcggcctgctgctccgagccgcgctgctccgtggccgtggtggagctgccccggcgccccgcgcccccggcagccgtgctcggctgctacctcttcaactgcacggcgcgcggccgcaacgtctgcaagttcgcgctgcacagcggctacagcagctacagcctcagccgcgcgccggacggcgccgccctggccaccgcgcgcgcctcgccccggcaggaaaaggatgcgcctccacttagcaaggctgggcaggatgtggttctgcatctgcccacagacggggtggttctagacggccgcgagagcacagatgaccacgccatcgtccagtatgagtgggcactgctgcagggggacccgtcagtggacatgaaggtgcctcaatcaggaaccctgaagctgtcccacctacaggagggaacctacaccttccagctgaccgtgacggacactgccgggcagagaagctctgacaacgtgtcagtgacagtgcttcgcgcagcctactccacaggaggatgtttgcacacttgctcacgctaccacttcttctgtgacgatggctgctgcattgacatcacgctcgcctgcgatggagtgcagcagtgtcctgatgggtctgatgaagacttctgccagaatctgggcctggaccgcaagatggtaacccacacggcagctagtcctgccctgccaagaaccacagggccgagtgaagatgcagggggtgactccttggtggaaaagtctcagaaagccactgccccaaacaagccacctgcattatcaaacacagagaagaggaaagttatatatttgagtcaaagggtgatggaggaggaggggaacacccagccccagaaacaggtgcagtgctacccctggcgctgggtttggctatcactgctctgctgcttctcatggttgcatgccgactacgactggtgaaacagaaactgaaaaaagctcgtcccattacatctgaggaatcggactacctcataaatgggatgtatctatagtaatgtaatttcaataccttggggcagggacatgttttgtttataatttatacatctattaagttctggatatttacagcttcttttgtttttaattgggccagaagattctgcaaatcccaaatctttctttattatttattgtaaaaaaagtttccttagaagtcataaaatattttgaaatttagagaggaattcatgattaaagattcctaaaaatataattctgatttatgtaagctgtccctgaaaatagaaatgtgtacttagctgagagaaaattcagcatctcaggaggtggtattaggatgactgtgttaacccattaccttttagaagccaactgttggccccttaccatgctggactgctataggcccagcttccccttgttctgtggcccttttcttcctccttgaagctcccagtattctttttcttttcccctctaaacctgtttctgagagtggatctcaagcaagttcatgccttcaatcagatgttacttagggtgggtatacctaaattataaaccttatgtacaagtcagtaagccttagggaaggtgagtgtgggtccttcctaatccctctgacgtcatgtcatataggtggctgcctccttagactgacctttgggagaaaaaaaccccagactttgaattagtaacagctctaagatggtcatgcagtgagataggaaatcaagatggaagcagagaatctggcatgccaaaaactaacagaaacttagttgaaggcaaagagagcaaggagaaagtttaatacttcattacatcaaatcaacactgctccatggtgagagcacagcaactcatttatatatatatatataggctttgttgatgaaaaacaacaattgaagagaggacgttgagtggattcctgggtacagcttttgtaaaaatgtcaccatggctttcatccaatggaatgagtcgatgttttttaat

SEQ ID NO: 53 - DNA, 2526 bp, Homo sapiens
  misc_feature: Incyte ID No 7503506CB1
gggttcgggggcgccgcgctgtgaggccggggcctagagccagccgcggccgcgcaggaggggcccagggcccgcgctcgcccgcgtccccgccttcctcccgcgctcagccccgcctcggctcgctgcccttggctctcgtcgccatggcctccgtcgcccaggagagcgcgggctcgcagcgccggctaccgccgcgtcacggggcgctgcgcgggctgctactgctctgcctgtggctgccaagcggccgtgcggccttgccgcccgcggcgccgctgtccgaactgcacgcgcagctgtcgggcgtggagcagctgctggaggagttccgccggcaactgcagcaggagcggcctcaggaggagctggagctggagctgcgcgcgggcggcggcccccaggaggactgcccgggccggggcagcggcggctacagcgcaatgcctgacgccatcatccgcaccaaggactccctggcggcgggtgccagcttcctgcgggcgccggcggccgtgcggggctggcggcaatgcgtggcggcctgctgctccgagccgcgctgctccgtggccgtggtggagctgccccggcgccccgcgcccccggcagccgtgctcggctgctacctcttcaactgcacggcgcgcggccgcaacgtctgcaagttcgcgctgcacagcggctacagcagctacagcctcagccgcgcgccggacggcgccgccctggccaccgcgcgcgcctcgccccggcaggaaaaggatgcgcctccacttagcaaggctgggcaggatgtggttctgcatctgcccacagacggggtggttctagacggccgcgagagcacagatgaccacgccatcgtccagtatgagtgggcactgctgcagggggacccgtcagtggacatgaaggtgcctcaatcaggaaccctgaagctgtcccacctacaggagggaacctacaccttccagctgaccgtgacggacactgccgggcagagaagctctgacaacgtgtcagtgacagtgcttcgcgcagcctactccacaggaggatgtttgcacacttgctcacgctaccacttcttctgtgacgatggctgctgcattgacatcacgctcgcctgcgatggagtgcagcagtgtcctgatgggtctgatgaagacttctgccagaatctgggcctggaccgcaagatggtaacccacacggcagctagtcctgccctgccaagaaccacagggccgagtgaagatgcagggggtgactccttggtggaaaagtctcagaaagccactgccccaaacaagccacctgcattatcaaacacagagaagaggaatcattccgccttttggggaccagagagtcaaatcattcctgtgatgccaggtgcagtgctacccctggcgctgggtttggctatcactgctctgctgcttctcatggttgcatgccgactacgactggtgaaacagaaactgaaaaaagctcgtcccattacatctgaggaatcggactacctcataaatgggatgtatctatagtaatgtaatttcaataccttggggcagggacatgttttgtttataatttatacatctattaagttctggatatttacagcttcttttgtttttaattgggccagaagattctgcaaatcccaaatctttctttattatttattgtaaaaaaagtttccttagaagtcataaaatattttgaaatttagagaggaattcatgattaaagattcctaaaaatataattctgatttatgtaagctgtccctgaaaatagaaatgtgtacttagctgagagaaaattcagcatctcaggaggtggtattaggatgactgtgttaacccattaccttttagaagccaactgttggccccttaccatgctggactgctataggcccagcttccccttgttctgtggcccttttcttcctccttgaagctcccagtattctttttcttttcccctctaaacctgtttctgagagtggatctcaagcaagttcatgccttcaatcagatgttacttagggtgggtatacctaaattataaaccttatgtacaagtcagtaagccttagggaaggtgagtgtgggtccttcctaatccctctgacgtcatgtcatataggtggctgcctccttagactgacctttgggagaaaaaaaccccagactttgaattagtaacagctctaagatggtcatgcagtgagataggaaatcaagatggaagcagagaatctggcatgccaaaaactaacagaaacttagttgaaggcaaagagagcaaggagaaagtttaatacttcattacatcaaatcaacactgctccatggtgagagcacagcaactcatttatatatatatatataggctttgttgatgaaaaacaacaattgaagagaggacgttgagtggattcctgggtacagcttttgtaaaaatgtcaccatggctttcatccaatggaatgagtcgatgttttttaat

SEQ ID NO: 54 - DNA, 2464 bp, Homo sapiens
  misc_feature: Incyte ID No 7503509CB1
gggttcgggggcgccgcgctgtgaggccggggcctagagccagccgcggccgcgcaggaggggcccagggcccgcgctcgcccgcgtccccgccttcctcccgcgctcagccccgcctcggctcgctgcccttggctctcgtcgccatggcctccgtcgcccaggagagcgcgggctcgcagcgccggctaccgccgcgtcacggggcgctgcgcgggctgctactgctctgcctgtggctgccaagcggccgtgcggccttgccgcccgcggcgccgctgtccgaactgcacgcgcagctgtcgggcgtggagcagctgctggaggagttccgccggcaactgcagcaggagcggcctcaggaggagctggagctggagctgcgcgcgggcggcggcccccaggaggactgcccgggcccgggcagcggcggctacagcgcaatgcctgacgccatcatccgcaccaaggactccctggcggcgggtgccagcttcctgcgggcgccggcggccgtgcggggctggcggcaatgcgtggcggcctgctgctccgagccgcgctgctccgtggccgtggtggagctgccccggcgccccgcgcccccggcagccgtgctcggctgctacctcttcaactgcacggcgcgcggccgcaacgtctgcaagttcgcgctgcacagcggctacagcagctacagcctcagccgcgcgccggacggcgccgccctggccaccgcgcgcgcctcgccccggcagggtgcctcaatcaggaaccctgaagctgtcccacctacaggagggaacctacaccttccagctgaccgtgacggacactgccgggcagagaagctctgacaacgtgtcagtgacagtgcttcgcgcagcctactccacaggaggatgtttgcacacttgctcacgctaccacttcttctgtgacgatggctgctgcattgacatcacgctcgcctgcgatggagtgcagcagtgtcctgatgggtctgatgaagacttctgccagaatctgggcctggaccgcaagatggtaacccacacggcagctagtcctgccctgccaagaaccacagggccgagtgaagatgcagggggtgactccttggtggaaaagtctcagaaagccactgccccaaacaagccacctgcattatcaaacacagagaagaggaatcattccgccttttggggaccagagagtcaaatcattcctgtgatgccagatagtagttcctcagggaagaacagaaaagaggaaagttatatatttgagtcaaagggtgatggaggaggaggggaacacccagccccagaaacaggtgcagtgctacccctggcgctgggtttggctatcactgctctgctgcttctcatggttgcatgccgactacgactggtgaaacagaaactgaaaaaagctcgtcccattacatctgaggaatcggactacctcataaatgggatgtatctatagtaatgtaatttcaataccttggggcagggacatgttttgtttataatttatacatctattaagttctggatatttacagcttcttttgtttttaattgggccagaagattctgcaaatcccaaatctttctttattatttattgtaaaaaaagtttccttagaagtcataaaatattttgaaatttagagaggaattcatgattaaagattcctaaaaatataattctgatttatgtaagctgtccctgaaaatagaaatgtgtacttagctgagagaaaattcagcatctcaggaggtggtattaggatgactgtgttaacccattaccttttagaagccaactgttggccccttaccatgctggactgctataggcccagcttccccttgttctgtggcccttttcttcctccttgaagctcccagtattctttttcttttcccctctaaacctgtttctgagagtggatctcaagcaagttcatgccttcaatcagatgttacttagggtgggtatacctaaattataaaccttatgtacaagtcagtaagccttagggaaggtgagtgtgggtccttcctaatccctctgacgtcatgtcatataggtggctgcctccttagactgacctttgggagaaaaaaaccccagactttgaattagtaacagctctaagatggtcatgcagtgagataggaaatcaagatggaagcagagaatctggcatgccaaaaactaacagaaacttagttgaaggcaaagagagcaaggagaaagtttaatacttcattacatcaaatcaacactgctccatggtgagagcacagcaactcatttatatatatatatataggctttgttgatgaaaaacaacaattgaagagaggacgttgagtggattcctgggtacagcttttgtaaaaatgtcaccatggctttcatccaatggaatgagtcgatgttttttaat

SEQ ID NO: 55 - DNA, 1452 bp, Homo sapiens
  misc_feature: Incyte ID No 7505800CB1
ccgaggcgagatggcggccaccgagggggtcggggaggctgcgcaagggggcgagcccgggcagccggcgcaacccccgccccagccgcacccaccgccgccccagcagcagcacaaggaagagatggcggccgaggctggggaagccgtggcgtcccccatggacgacgggtttgtgagcctggactcgccctcctatgtcctgtacaggcatttccggagagttcttttgaagtcacttcagaaggatctacatgaggaaatgaactacatcactgcaataattgaggagcagcccaaaaactatcaagtttggcatcataggcgagtattagtggaatggctaagagatccatctcaggagcttgaatttattgctgatattcttaatcaggatgcaaagaattatcatgcctggcagcatcgacaatgggttattcaggaatttaaactttgggataatgagctgcagtatgtggaccaacttctgaaagaggatgtgagaaataactctgtctggaaccaaagatacttcgttatttctaacaccactggctacaatgatcgtgctgtattggagagagaagtccaatacactctggaaatgattaaactagtaccacataatgaaagtgcatggaactatttgaaagggattttgcaggatcgtggtctttccaaatatcctaatctgttaaatcaattacttgatttacaaccaagtcatagttccccctacctaattgcctttcttgtggatatctatgaagacatgctagaaaatcagtgtgacaataaggaagacattcttaataaagcattagagttatgtgaaatcctagctaaagaaaaggacactataagaaaggaatattggagatacattggaagatcccttcaaagcaaacacagcacagaaaatgactcaccaacaaatgtacagcaataacaccatccagaagaacttgatggaatgcttttattttttattaagggaccctgcaggagtttcacacgagagtggtccttccctttgcctgtggtgtaaaagtgcatcacacaggtattgctttttaacaagaactgatgctccttgggtgctgctgctactcagactagctctaagtaatgtgattcttctaaagcaaagtcattggatgggaggaggaagaaaaagtcccataaaggaacttttgtagtcttatcaacatataatctaatcccttagcatcagctcctccctcagtggtacatgcgtcaagatttgtagcagtaataactgcaggtcacttgtatgtaatggatgtgaggtagccgaagtttggttcagtaagcagggaatacagtcgttccatcagagctggtctgcacactcacattatcttgctatcactgtaaccaactaatgccaaaagaacggttttgtaataaaattatagctgtatctaaaaa

SEQ ID NO: 56 - DNA, 1802 bp, Homo sapiens
  misc_feature: Incyte ID No 7503141CB1
cgccggtgccgggcgaacatggcggcggccaccggaccctcgttttggctggggaatgaaaccctgaaggtgccgctggcgctctttgccttgaaccggcagcgcctgtgtgagcggctgcggaagaaccctgctgtgcaggccggctccatcgtggtcctgcagggcggggaggagactcagcgctactgcaccgacaccggggtcctcttccgccaggagtccttctttcactgggcgttcggtgtcactgagccaggctgctatggtgtcatcgatgttgacactgggaagtcgaccctgtttgtgcccaggcttcctgccagccatgccacctggatgggaaagatccattccaaggagcacttcaaggagaagtatgccgtggacgacgtccagtacgtagatgagattgccagcgtcctgacgtcacagaagccctctgtcctcctcactttgcgtggcgtcaacacggacagcggcagtgtctgcagggaggcctcctttgacggcatcagcaagttcgaagtcaacaataccattcttcacccagagatcgttgagtgcctcttcgagcactactgctactcccggggcggcatgcgccacagctcctacacctgcatctgcggcagtggtgagaactcagccgtgctacactacggacacgccggagctcccaacgaccgaacgatccagaatggggatatgtgcctgttcgacatgggcggtgagtattactgcttcgcttccgacatcacctgctcctttcccgccaacggcaagttcactgcagaccagaaggccgtctatgaggcagtgctgcggagctcccgtgccgtcatgggtgccatgaagccaggtgtctggtggcctgacatgcaccgcctggctgaccgcatccacctggaggagctggcccacatgggcatcctgagcggcagcgtggacgccatggtccaggctcacctgggggccgtgtttatgcctcacgggcttggccacttcctgggcattgacgtgcacgacgtgggaggctacccagagggcgtggagcgcatcgacgagcccggcctgcggagcctgcgcactgcacggcacctgcagccaggcatggtgctcaccgtggagccgggcatctacttcatcgaccacctcctggatgaggccctggcggacccggcccgcgcctccttccttaaccgcgaggtcctgcagcgctttcgcggttttggcggggtccgcatcgaggaggacgtcgtggtgactgacagcggcatagagctgctgacctgcgtgccccgcactgtggaagagattgaagcatgcatggcaggctgtgacaaggcctttacccccttctctggccccaagtagagccagccagaaatcccagcgcacctgggggcctggccttgcaacctcttttcgtgatgggcagcctgctggtcagcactccagtagcgagagacggcacccagaatcagatcccagcttcggcatttgatcagaccaaacagtgctgtttcccggggaggaaacacttttttaattaattacccttttgcaggctcccacctttaatctgttttataccttgcttattaaatgagcgacttaaaatgattgaaaataatgctgttctttagtagcaactaaaatgtgtcttgctgtcatttatattccttttcccaggaaagaagcatttctgatactttctgtcaaaaatcaatatgcagaatggcatttgcaataaaaggtttcctaaaatgaaaaaaaaaaaaaaaaaaaaaaaaaaaaaa

SEQ ID NO: 57 - DNA, 1833 bp, Homo sapiens
  misc_feature: Incyte ID No 7500362CB1
cgggcgaacatggcggcggccaccggaccctcgttttggctggggaatgaaaccctgaaggtgccgctggcgctctttgccttgaaccggcagcgcctgtgtgagcggctgcggaagaaccctgctgtgcaggccggctccatcgtgtccttctttcactgggcgttcggtgtcactgagccaggctgctatggtgtcatcgatgttgacactgggaagtcgaccctgtttgtgcccaggcttcctgccagccatgccacctggatgggaaagatccattccaaggagcacttcaaggagaagtatgccgtggacgacgtccagtacgtagatgagattgccagcgtcctgacgtcacagaagccctctgtcctcctcactttgcgtggcgtcaacacggacagcggcagtgtctgcagggaggcctcctttgacggcatcagcaagttcgaagtcaacaataccattcttcacccagagatcgttgagtgccgagtgtttaagacggatatggagctggaggttctgcgctataccaataaaatctccagcgaggcccaccgtgaggtaatgaaggctgtaaaagtgggaatgaaagaatatgagttggaaagcctcttcgagcactactgctactcccggggcggcatgcgccacagctcctacacctgcatctgcggcagtggtgagaactcagccgtgctacactacggacacgccggagctcccaacgaccgaacgatccagaatggggatatgtgcctgttcgacatgggcggtgagtattactgcttcgcttccgacatcacctgctcctttcccgccaacggcaagttcactgcagaccagaaggccgtctatgaggcagtgctgcggagctcccgtgccgtcatgggtgccatgaagccaggtgtctggtggcctgacatgcaccgcctggctgaccgcatccacctggaggagctggcccacatgggcatcctgagcggcagcgtggacgccatggtccaggctcacctgggggccgtgtttatgcctcacgggcttggccacttcctgggcattgacgtgcacgacgtgggaggctacccagagggcgtggagcgcatcgacgagcccggcctgcggagcctgcgcactgcacggcacctgcagccaggcatggtgctcaccgtggagccgggcatctacttcatcgaccacctcctggatgaggccctggcggacccggcccgcgcctccttccttaaccgcgaggtcctgcagcgctttcgcggttttggcggggtccgcatcgaggaggacgtcgtggtgactgacagcggcatagagctgctgacctgcgtgccccgcactgtggaagagattgaagcatgcatggcaggctgtgacaaggcctttacccccttctctggccccaagtagagccagccagaaatcccagcgcacctgggggcctggccttgcaacctcttttcgtgatgggcagcctgctggtcagcactccagtagcgagagacggcacccagaatcagatcccagcttcggcatttgatcagaccaaacagtgctgtttcccggggaggaaacacttttttaattacccttttgcaggctcccacctttaatctgttttataccttgcttattaaatgagcgacttaaaatgattgaaaataatgctgttctttagtagcaactaaaatgtgtcttgctgtcatttatattccttttcccaggaaagaagcatttctgatactttctgtcaaaaatcaatatgcagaatggcatttgcaataaaaggtttcctaaaatggtcaaaaaaaaaaaaaa

SEQ ID NO: 58 - DNA, 2465 bp, Homo sapiens
  misc_feature: Incyte ID No 7503328CB1
ggcccttccgcgggtgatcagctggtctgcgctcccctgacgtgggctggggcacgtcaccgccgaatggcagcctccagaaagccaccgcgagtaagggtgaatcaccaggattttcaactgagaaatttaagaataattgaacctaacgaggtgacacactcaggagacacaggtgtggaaacagacggcagaatgcctccaaaggtgacttcagagctgcttcggcagctgagacaagccatgaggaactctgagtatgtgaccgaaccgatccaggcctacatcatcccatcgggagatgctcatcagagtgagtatattgctccatgtgactgtcggcgggcttttgtctctggattcgatggctctgcgggcacagccatcatcacagaagagcatgcagccatgtggactgacgggcgctactttctccaggctgccaagcaaatggacagcaactggacacttatgaagatgggtctgaaggacacaccaactcaggaagactggctggtgagtgtgcttcctgaaggatccagggttggtgtggaccccttgatcattcctacagattattggaagaaaatggccaaagttctgagaagtgccggccatcacctcattcctgtcaaggagaacctcgttgacaaaatctggacagaccgtcctgagcgcccttgcaagcctctcctcacactgggcctggattacacaggcatctcctggaaggacaaggttgcagaccttcggttgaaaatggctgagaggaacgtcatgtggtttgtggtcactgccttggatgagattgcgtggctatttaatctccgaggatcggatgtggagcacaatccagtatttttctcctacgcaatcataggactagagacgatcatgctcttcattgatggtgaccgcatagacgcccccagtgtgaaggagcacctgcttcttgacttgggtctggaagccgagtacaggatccaggtgcatccctacaagtccatcctgagcgagctcaaggccctgtgtgctgacctctccccaagggagaaggtgtgggtcagtgacaaggccagctatgctgtgagcgagaccatccccaaggaccaccgctgctgtatgccttacacccccatctgcatcgccaaagctgtgaagaattcagctgagtcagaaggcatgaggcgggctcacattaaagatgctgttgctctctgtgaactctttaactggctggagaaagaggttcccaaaggtggtgtgacagagatctcagctgctgacaaagctgaggagtttcgcaggcaacaggcagactttgtggacctgagcttcccaacaatttccagccagtccctgagacgaataggaccttgtccctggatgaggtgtaccttattgactcgggtgctcaatacaaggatggcaccacagatgtgacgcggacaatgcattttgggacccctacagcctacgagaaggaatgcttcacatatgtcctcaagggccacatagctgtgagtgcagccgttttcccgactggaaccaaaggtcaccttcttgactcctttgcccgttcagctttatgggattcaggcctagattacttgcacgggactggacatggtgttgggtcttttttgaatgtccatgagggtccttgcggcatcagttacaaaacattctctgatgagcccttggaggcaggcatgattgtcactgatgagcccgggtactatgaagatggggcttttggaattcgcattgagaatgttgtccttgtggttcctgtgaagaccaagtataattttaataaccggggaagcctgacctttgaacctctaacattggttccaattcagaccaaaatgatagatgtggattctcttacagacaaagagtgcgactggctcaacaattaccacctgacctgcagggatgtgattgggaaggaattgcagaaacagggccgccaggaagctctcgagtggctcatcagagagacgcaactcatctccaaacagcattaataaatacctccccggttttgtttttgtaaaatgctctggaggaaggaagaaacgtggcagatccctgacatctttcccctttcctttccttcttccccacctcccctttttactttagactttaagaagaacagaaaatcttcttatcctctttgatattttattgcaaacactcagtcttttatgattttttaattgttgagaacaagccaagaataaaattgctgcaccagaaggagggtccctccaaagttgaacacttggtgaaaggaagatgccccgacttctttggccagtgatggggaatcagtgagtgctccatgatggtcatgttccaggtgctagtacatcattcatgatcaccttaatgctcatgagactatatttatgatcagtgaataaaaatgtcagaactgtgaaaaaaaaaaaaaa

SEQ ID NO: 59 - DNA, 2560 bp, Homo sapiens
  misc_feature: Incyte ID No 7510464CB1
ggcccttccgcgggtgatcagctggtctgcgctcccctgacgtgggctggggcacgtcaccgccgaatggcagcctccagaaagccaccgcgagtaagggtgaatcaccaggattttcaactgagaaatttaagaataattgaacctaacgaggtgacacactcaggagacacaggtgtggaaacagacggcagaatgcctccaaaggtgacttcagagctgcttcggcagctgagacaagccatgaggaactctgagtatgtgaccgaaccgatccaggcctacatcatcccatcgggagatgctcatcagagtgagtatattgctccatgtgactgtcggcgggcttttgtctctggattcgatggctctgcgggcacagccatcatcacagaagagcatgcagccatgtggactgacgggcgctactttctccaggctgccaagcaaatggacagcaactggacacttatgaagatgggtctgaaggacacaccaactcaggaagactggctggtgagtgtgcttcctgaaggatccagggttggtgtggaccccttgatcattcctacagattattggaagaaaatggccaaagttctgagaagtgccggccatcacctcattcctgtcaaggagaacctcgttgacaaaatctggacagaccgtcctgagcgcccttgcaagcctctcctcacactgggcctggattacacaggcatctcctggaaggacaaggttgcagaccttcggttgaaaatggctgagaggaacgtcatgtggtttgtggtcactgccttggatgagattgcgtggctatttaatctccgaggatcagatgtggagcacaatccagtatttttctcctacgcaatcataggactagagacgatcatgctcttcattgatggtgaccgcatagacgcccccagtgtgaaggagcacctgcttcttgacttgggtctggaagccgaatacaggatccaggtgcatccctacaagtccatcctgagcgagctcaaggccctgtgtgctgacctctccccaagggagaaggtgtgggtcagtgacaaggccagctatgctgtgagcgagaccatccccaaggaccaccgctgctgtatgccttacacccccatctgcatcgccaaagctgtgaagaattcagctgagtcagaaggcatgaggcgggctcacattaaagatgctgttgctctctgtgaactctttaactggctggagaaagaggttcccaaaggtggtgtgacagagatctcagctgctgacaaagctgaggagtttcgcaggcaacaggcagactttgtggacctgagcttcccaacaatttccagtacgggacccaacggcgccatcattcactacgcgccagtccctgagacgaataggaccttgtccctggatgaggtgtaccttattgactcgggtgctcaatacaaggatggcaccacagatgtgacgcggacaatgcattttgggacccctacagcctacgagaaggaatgcttcacatatgtcctcaagggccacatagctgtgagtgcagccgttttcccgactggaaccaaaggtcaccttcttgactcctttgcccgttcagctttatgggattcaggcctagattacttgcacgggactggacatggtgttgggtcttttttgaatgtccatgagggtccttgcggcatcagttacaaaacattctctgatgagcccttggaggcaggcatgattgtcactgatgagcccgggtactatgaagatggggcttttggaattcgcattgagaatgttgtccttgtggttcctgtgaagaccaagtataattttaataaccggggaagcctgacctttgaacctctaacattggttccaattcagaccaaaatgatagatgtggattctcttacagacaaagaggagctgtggaatgggattctcccagctagaagcctcttctgcctgttccagttcacagtgcgactggctcaacaattaccacctgacctgcagggatgtgattgggaaggaattgcagaaacagggccgccaggaagctctcgagtggctcatcagagagacgcaacccatctccaaacagcattaataaatacctccccggttttgtttttgtaaaatgctctggaggaaggaagaaacgtggcagatccctgacatctttcccctttcctttccttcttccctacctcccctttttactttagactttaagaagaacagaaaatcttcttatcctctttgatattttattgcaaacactcagtcttttatgattttttaattgttgagaacaagccaagaataaaattgctgcaccagaaggagggtccctccaaagttgaacacttggtgaaaggaagatgccccgacttctttggccagtgatggggaatcagtgagtgctccatgatggtcatgttccaggtgctagtacatcattcatgatcaccttaatgctcatgagactatatttatgatcagtgaataaaaatgtcagaactgtgaaaaaaaaaaaaaaaaa

SEQ ID NO: 60 - DNA, 2254 bp, Homo sapiens
  misc_feature: Incyte ID No 7510394CB1
gcaggcatggcggcggctatgccgcttgctctgctcgtcctgttgctcctggggcccggcggctggtgccttgcagaacccccacgcgacagcctgcgggaggaacttgtcatcaccccgctgccttccggggacgtagccgccacattccagttccgcacgcgctgggattcggagcttcagcgggaaggagtgtcccattacaggctctttcccaaagccctggggcagctgatctccaagtattctctacgggagctgcacctgtcattcacacaaggcttttggaggacccgatactgggggccacccttcctgcaggccccatcaggtgcagagctgtgggtctggttccaagacactgtcactgagtttagcagccagctgtggactttgaaagagggagcagaggtagccccaggacagtgagtggatttgtgtctctatccagtgtggataaatcttggaaggagctcagtaatgtcctctcagggatcttctgcgcctctctcaacttcatcgactccaccaacacagtcactcccactgcctccttcaaacccctgggtctggccaatgacactgaccactactttctgcgctatgctgtgctgccgcgggaggtggtctgcaccgaaaacctcaccccctggaagaagctcttgccctgtagttccaaggcaggcctctctgtgctgctgaaggcagatcgcttgttccacaccagctaccactcccaggcagtgcatatccgccctgtttgcagaaatgcacgctgtactagcatctcctgggagctgaggcagaccctgtcagttgtatttgatgccttcatcacggggcagggaaagaaagactggtccctcttccggatgttctcccgaaccctcacggagccctgccccctggcttcagagagccgagtctatgtggacatcaccacctacaaccaggacaacgagacattagaggtgcacccacccccgaccactacatatcaggacgtcatcctaggcactcggaagacctatgccatctatgacttgcttgacaccgccatgatcaacaactctcgaaacctcaacatccagctcaagtggaagagacccccagagaatgaggcccccccagtgcccttcctgcatgcccagcggtacgtgagtggctatgggctgcagaagggggagctgagcacactgctgtacaacacccacccataccgggccttcccggtgctgctgctggacaccgtaccctggtatctgcggctgtatgtgcacaccctcaccatcacctccaagggcaaggagaacaaaccaagttacatccactaccagcctgcccaggaccggctgcaaccccacctcctggagatgctgattcagctgccggccaactcagtcaccaaggtttccatccagtttgagcgggcgctgctgaagtggaccgagtacacgccagatcctaaccatggcttctatgtcagcccatctgtcctcagcgcccttgtgcccagcatggtagcagccaagccagtggactgggaagagagtcccctcttcaacagcctgttcccagtctctgatggctctaactactttgtgcggctctacacggagccgctgctggtgaacctgccgacaccggacttcagcatgccctacaacgtgatctgcctcacgtgcactgtggtggccgtgtgctacggctccttctacaatctcctcacccgaaccttccacatcgaggagccccgcacaggtggcctggccaagcggctggccaaccttatccggcgcgcccgaggtgtccccccactctgattcttgccctttccagcagctgcagctgccgtttctctctggggaggggagcccaagggctgtttctgccacttgctctcctcagagttggcttttgaaccaaagtgccctggaccaggtcagggcctacagctgtgttgtccagtacaggagccacgagccaaatgtggcatttgaatttgaattaacttagaaattcatttcctcacctgtagtggccacctctatattgaggtgctcaataagcaaaagtggtcggtggctgctgtattggacagcacagaaaaagatttccatcaccacagaaaggtcggctggcagcactggccaaggtgatggggtgtgctacacagtgtatgtcactgtgtagtggatggagtttactgtttgtggaataaaaacggctgtttccgtggaaaaaaaaaaaaaaa

SEQ ID NO: 61 - DNA, 2139 bp, Homo sapiens
  misc_feature: Incyte ID No 7500745CB1
gccgcaggcatggcggcggctatgccgcttgctctgctcgtcctgttgctcctggggcccggcggctggtgccttgcagaacccccacgcgacagcctgcgggaggaacttgtcatcaccccgctgccttccggggacgtagccgccacattccagttccgcacgcgctgggattcggagcttcagcgggaaggagtgtcccattacaggctctttcccaaagccctggggcagctgatctccaagtattctctacgggagctgcacctgtcattcacacaaggcttttggaggacccgatactgggggccacccttcctgcaggccccatcagtgtggataaatcttggaaggagctcagtaatgtcctctcagggatcttctgcgcctctctcaacttcatcgactccaccaacacagtcactcccactgcctccttcaaacccctgggtctggccaatgacactgaccactactttctgcgctatgctgtgctgccgcgggaggtggtctgcaccgaaaacctcaccccctggaagaagctcttgccctgtagttccaaggcaggcctctctgtgctgctgaaggcagatcgcttgttccacaccagctaccactcccaggcagtgcatatccgccctgtttgcagaaatgcacgctgtactagcatctcctgggagctgaggcagaccctgtcagttgtatttgatgccttcatcacggggcagggaaagaaagactggtccctcttccggatgttctcccgaaccctcacggagccctgccccctggcttcagagagccgagtctatgtggacatcaccacctacaaccaggacaacgagacattagaggtgcacccacccctgaccactacatatcaggacgtcatcctaggcactcggaagacctatgccatctatgacttgcttgacaccgccatgatcaacaactctcgaaacctcaacatccagctcaagtggaagagacccccagagaatgaggcccccccagtgcccttcctgcatgcccagcggtacgtgagtggctatgggctgcagaagggggagctgagcacactgctgtacaacacccacccataccgggccttcccggtgctgctgctggacaccgtaccctggtatctgcggctgtatgtgcacaccctcaccatcacctccaagggcaaggagaacaaaccaagttacatccactaccagcctgcccaggaccggctgcaaccccacctcctggagatgctgattcagctgccggccaactcagtcaccaaggtttccatccagtttgagcgggcgctgctgaagtggaccgagtacacgccagatcctaaccatggcttctatgtcagcccatctgtcctcagcgcccttgtgcccagcatggtagcagccaagccagtggactgggaagagagtcccctcttcaacagcctgttcccagtctctgatggctctaactactttgtgcggctctacacggagccgctgctggtgaacctgccgacaccggacttcagcatgccctacaacgtgatctgcctcacgtgcactgtggtggccgtgtgctacggctccttctacaatctcctcacccgaaccttccacatcgaggagccccgcacaggtggcctggccaagcggctggccaaccttatccggcgcgcccgaggtgtccccccactctgattcttgccctttccagcagctgcagctgccgtttctctctggggaggggagcccaagggctgtttctgccacttgctctcctcagagttggcttttgaaccaaagtgccctggaccaggtcagggcctacagctgtgttgtccagtacaggagccacgagccaaatgtggcatttgaatttgaattaacttagaaattcatttcctcacctgtagtggccacctctatattgaggtgctcaataagcaaaagtggtcggtggctgctgtattggacagcacagaaaaagatttccatcaccacagaaaggtcggctggcagcactggccaaggtgatggggtgtgctacacagtgtatgtcactgtgtagtggatggagtttactgtttgtggaataaaaacggctgtttccgtgaaaanaaaaaaaaaaagg

SEQ ID NO: 62 - DNA, 648 bp, Homo sapiens
  misc_feature: Incyte ID No 7500929CB1
gtggaagagacgcgccggcgttggccgctgctgctagcagcttgaaccccagggtcgggaccgatgtcggcttgggctgctgccagcctaagcagggccgctgcccgatgcttgctggcacgaggccccggggtcagggcggctcctccgcgcgacccccggccctcccaccccgagccccggggctgcggtgccgctccgggcaggacgctgcactttaccgcggctgtccccgccgggcacaacaagtggtccaaagtcaggcacatcaagggtccgaaggacgtcgaaaggagtcgcatcttctccaaactctgtttgaacatccgcctggcagtgaaagccaggaggcccaaggacaggacttgcgaccttgaagccaaaggaatctcacttgtggggcctccttgtcagctctgctgctgtctcagagccatctggatgagtgtcccgacaccctctcggatgcagggcaggaccacccagctggtcagactctgatgttgggtagctggcctctgtggggattgtaagtgccctgaggcgctctgtactagaaactgctcttaataataacggtgattattggttgctgcaaaaaaaaaaaaaaaaaaaaaaaaaaaaaaaaaaaaaaaaaaaaaaaaaaaa